(19) United States
(12) Patent Application Publication
(10) Pub. No.: US 2024/0092756 A1
(43) Pub. Date: Mar. 21, 2024

(54) METHODS AND COMPOSITION FOR KRAS MODIFICATIONS

(71) Applicants: 2692372 ONTARIO, INC., Mississauga (CA); DUNAD THERAPEUTICS LTD., Great Cambourne, Cambridge (GB)

(72) Inventors: Patrick T. GUNNING, Mississauga (CA); Jeff OMEARA, Halton Hills (CA); Siawash AHMAR, North York (CA); Graham L. SIMPSON, Great Wymondley (GB); Peter HUNT, Storrington (GB); David Alexander ROSA, Toronto (CA); Ji Sung PARK, Etobicoke (CA)

(21) Appl. No.: 18/253,712

(22) PCT Filed: Nov. 18, 2021
(86) PCT No.: PCT/IB2021/000805

§ 371 Date: May 19, 2023

Related U.S. Application Data
(60) Provisional application No. 63/116,723, filed on Nov. 20, 2020.

(51) Int. Cl. C07D 401/12; C07D 215/12; C07D 215/42; C07D 401/04; C07D 471/14

(52) U.S. Cl. CPC C07D 401/12; C07D 215/12; C07D 215/42; C07D 401/04; C07D 471/14; C07B 2200/09

(57) ABSTRACT

Provided herein are compounds binding to KRAS protein or a mutant thereof, pharmaceutical compositions comprising said compounds, and methods for using said compounds for the treatment of diseases.

### CROSS-REFERENCES

This application claims the benefit of U.S. Provisional Application No. 63/116,723, filed Nov. 20, 2020, which is hereby incorporated by reference in its entirety herein.

### SEQUENCE LISTINGS

The instant application contains a Sequence Listing which has been submitted electronically in ASCII format and is hereby incorporated by reference in its entirety. Said ASCII copy, created on Nov. 17, 2021, is named 59091-704_601_SL.txt and is 5,454 bytes in size.

### BRIEF SUMMARY OF THE DISCLOSURE

The present embodiments including compounds, molecules, chemical groups, compositions, and/or methods disclosed herein are selective for a KRAS protein or a mutant thereof, e.g., selective for KRAS G12C, KRAS C118A, or KRAS G12C/C118A. In some cases, provided herein are small molecule binders (e.g., inhibitors) that bind effectively to a KRAS protein (a.k.a., K-Ras) or a mutant thereof (e.g., KRAS G12C), e.g., by a covalent bond, which are useful for treating cancer, as mutant KRAS proteins are major drivers of human cancers. Also provided herein are pharmaceutical compositions comprising said compounds, and methods for using said compounds for the treatment of diseases such as cancers.

Provided in some embodiments herein is a compound of Formula (I′), or a salt or solvate or tautomer or regioisomer thereof:

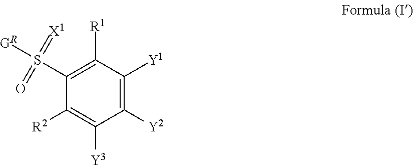

wherein,
G^{R} is substituted or unsubstituted alkyl (e.g., haloalkyl), substituted or unsubstituted heteroalkyl, —N(R⁵)₂, —N(R⁵)G, or G;
   R⁵ is hydrogen, —CN, substituted or unsubstituted alkyl (e.g., alkyl substituted with one or more substituents, each substitutent being independently selected from the group consisting of oxo, hydroxy, alkoxy, heteroalkyl, and amino (e.g., —C(═O)R⁶, —C(═O)OR⁶, or —C(═O)NR³R⁶, wherein each R⁶ is independently hydrogen, substituted or unsubstituted alkyl, or substituted or unsubstituted heteroalkyl)), substituted or unsubstituted heteroalkyl, substituted or unsubstituted aryl, or substituted or unsubstituted heteroaryl;
X¹ is absent, O, or NR;
   R is hydrogen, R⁷, substituted or unsubstituted alkyl, substituted or unsubstituted heteroalkyl, substituted or unsubstituted aryl, substituted or unsubstituted heteroaryl, or G;
each Y¹, Y², and Y³ is independently hydrogen, halo, substituted or unsubstituted alkyl (e.g., haloalkyl) (e.g., with at least two Y being halo or haloalkyl, such as fluoroalkyl, e.g., at least one Y (e.g., Y²) being halo (e.g., Y¹, Y², Y³ all being F)), or G;
R¹ is hydrogen, halogen, or R⁷;
R² is hydrogen, halogen, or R⁷;
   each R⁷ is independently G, —CN, —OR³, —S(═O)ₓR³, —S(═O)(═NR³)R³, —S(═O)₂N(R)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
      R³ is hydrogen, substituted or unsubstituted alkyl, -L¹R⁴, —C(═O)L¹R⁴, —C(═O)OL¹R⁴, or —C(═O)NR⁴L¹R⁴, wherein each L¹ is independently substituted or unsubstituted alkyl, substituted or unsubstituted heteroalkyl, substituted or unsubstituted aryl, or substituted or unsubstituted heteroaryl; and each R⁴ is independently hydrogen, substituted or unsubstituted cycloalkyl, substituted or unsubstituted heterocycloalkyl, substituted or unsubstituted aryl, or substituted or unsubstituted heteroaryl;
      x is 0, 1, or 2; and
G is or comprises a KRAS-binding ligand, is or comprises (e.g., unsaturated) carbocycle, is or comprises (e.g., unsaturated) heterocycle, or is -L²-G¹, wherein L² is a linker (e.g., —O— or —NR⁵—), and G¹ is hydrogen or an organic residue (e.g., is or comprises a KRAS-binding ligand, is or comprises (e.g., unsaturated) carbocycle, or is or comprises (e.g., unsaturated) heterocycle).

In some cases, the present disclosure provides a compound of Formula (I), or a salt or solvate or tautomer or regioisomer thereof:

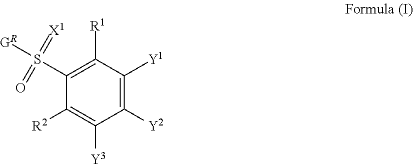

wherein,
G^{R} is alkyl, haloalkyl, heteroalkyl, —N(R⁵)₂, —N(R⁵)G, or G;
G is or comprises a KRAS-binding ligand, is or comprises (e.g., unsaturated) carbocycle, is or comprises (e.g., unsaturated) heterocycle, or is -L²-G¹, wherein L² is a linker (e.g., —O— or —NR⁵—), and G is hydrogen or an organic residue (e.g., is or comprises a KRAS-binding ligand, is or comprises (e.g., unsaturated) carbocycle, or is or comprises (e.g., unsaturated) heterocycle);
X¹ is absent, O or NR;
each Y¹, Y², and Y³ is independently hydrogen, halo, alkyl, haloalkyl (e.g., with at least two Y being halo or haloalkyl, such as fluoroalkyl, e.g., at least one Y (e.g., Y²) being halo) (e.g., Y¹, Y², Y³ all being F), or G;
R is hydrogen, R⁷, alkyl, heteroalkyl, aryl, heteroaryl, or G;
R¹ is R⁷;
R² is hydrogen, halogen, or R⁷;
R³ is hydrogen, -L¹R⁴, —C(═O)L¹R⁴, —C(═O)OL¹R⁴, or —C(═O)NR⁴L¹R⁴, wherein each L is independently alkyl, heteroalkyl, aryl, or heteroaryl; and each R⁴ is independently hydrogen, cycloalkyl, heterocycloalkyl, aryl, or heteroaryl;
R⁵ is hydrogen, —CN, —C(═O)R⁶, —C(═O)OR⁶, —C(═O)NR³R⁶, alkyl, heteroalkyl, aryl, or heteroaryl;
each R⁶ is independently hydrogen, alkyl, or heteroalkyl;
each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂, alkyl, heteroalkyl, cycloalkyl, heterocycloalkyl, aryl, or heteroaryl; and
x is 0, 1, or 2.

In some embodiments, the compound (e.g., of Formula (I′) or Formula (I)) comprises only one G.

In some embodiments, such as when R¹ is halo, then R² and Y¹—Y³ are each independently halogen (e.g., fluoro).

In some embodiments, such as when R¹ is hydrogen or halogen (e.g., fluoro), one of Y¹, Y², or Y³ is G.

In some embodiments, such as when R¹ is hydrogen or halogen (e.g., fluoro), either G^{R} or Y² is G.

In some instances, G is or comprises (e.g., unsaturated) carbocycle, is or comprises (e.g., unsaturated) heterocycle, or is -L2-G1 that it is a KRAS ligand. In some instances, G is -L²-G¹, wherein L² is a linker and G¹ is an organic residue (e.g., is or comprises a KRAS-binding ligand, is or comprises (e.g., unsaturated) carbocycle, or is or comprises (e.g., unsaturated) heterocycle). In some instances, L² is a substituted or unsubstituted unsaturated alkylene (e.g., alkenylene or alkynylene), substituted or unsubstituted arylene, or substituted or unsubstituted heteroarylene, and G¹ is an organic residue (e.g., is or comprises a KRAS-binding ligand). In some instances, L² is a bond, —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and G is an organic residue (e.g., is or comprises a KRAS-binding ligand).

In some instances, G is substituted or unsubstituted unsaturated carbocycle or substituted or unsubstituted unsaturated heterocycle, wherein G and R⁵ on a single N, if present, are optionally taken together to form a substituted or unsubstituted N-containing heterocycloalkyl.

In some instances, G comprises one or more cyclic ring systems selected from substituted or unsubstituted unsaturated carbocycles and substituted or unsubstituted unsaturated heterocycles. In some instances, G¹ comprises one or more cyclic ring systems selected from substituted or unsubstituted carbocycles and substituted or unsubstituted heterocycles. In some instances, G comprises two or more cyclic ring systems selected from substituted or unsubstituted unsaturated carbocycles and substituted or unsubstituted unsaturated heterocycles. In some instances, G¹ comprises two or more cyclic ring systems selected from substituted or unsubstituted carbocycles and substituted or unsubstituted heterocycles. In some instances, two or more cyclic ring systems are connected via a bond. In some instances, the two or more cyclic ring systems are connected via one or more linker and/or bond. In some instances, the linker is —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; and each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₈ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl. In some instances, the cyclic ring system comprises substituted or unsubstituted monocyclic aryl or substituted or unsubstituted monocyclic heteroaryl. In some instances, the cyclic ring system comprises substituted or unsubstituted bicyclic aryl or substituted or unsubstituted bicyclic heteroaryl.

In some instances, G or G¹ is or comprises a KRAS-binding ligand. In some instances, G or G¹ is or comprises a KRAS-binding ligand selected from Table 2. In some instances, G or G¹ is or comprises a KRAS-binding ligand selected from Table 3, Table 4, Table 5, and Table 6.

In some instances, R⁵ is hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some instances, R⁵ is hydrogen, —CN, —CH₃, —CF₃, or cyclopropyl. In some instances, R⁵ is hydrogen.

In some instances, each R⁸ is independently hydrogen, substituted or unsubstituted C₁-C₄ alkyl, or substituted or unsubstituted C₁-C₄ heteroalkyl. In some instances, each R⁸ is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some instances, each R⁸ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some instances, each R⁸ is independently hydrogen, —CH₃, or —OCH₃.

In some instances, X¹ is O, NH, or N(substituted or unsubstituted alkyl). In some instances, X¹ is O, NH, or N(alkyl). In some instances, X¹ is O, NH, or N(CH₃). In some instances, X¹ is O. In some instances, X¹ is NH or N(CH₃).

In some instances, G^{R} is —N(R⁵)₂, X¹ is NR, R is G, and G is a KRAS-binding ligand or -L²-G¹ wherein G¹ is a KRAS-binding ligand.

In some instances, X¹, R¹, R², Y¹, Y², and Y are selected from (e.g., the corresponding X¹, R¹, R², Y¹, Y², and Y³ of a structure provided in) Table 7.

Provided in some embodiments herein is a compound of Formula (I-B), or a salt or solvate or tautomer or regioisomer thereof:

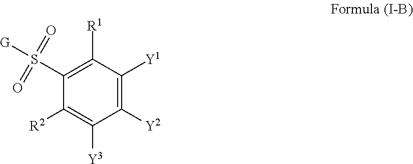

wherein,
G is or comprises a KRAS-binding ligand (e.g., G is -L²-G, wherein L2 is a linker (e.g., substituted or unsubstituted alkyl (e.g., alkyl substituted with pipirizinyl or piperidinyl), substituted or unsubstituted pipirizinyl, substituted or unsubstituted piperidinyl, substituted or unsubstituted azetidinyl (e.g., azetidinyl substituted with amino), or substituted or unsubstituted amino (e.g., —NH—, amino substituted with alkyl (e.g., —CH₂NH— or —CH₂CH₂NH—), or amino substituted with azetidinyl)), and G is a KRAS-binding ligand that binds to with KRAS (e.g., KRAS G12C));
Y¹, Y², and Y³ are each independently hydrogen or halogen (e.g., fluoro) (e.g., wherein Y¹, Y², and Y are fluoro or wherein Y and Y² are fluoro and Y³ is hydrogen);
R¹ is halogen (e.g., fluoro);
R² is halogen (e.g., fluoro), —OR³, or substituted or unsubstituted alkyl (e.g., haloalkyl); and
   R³ is hydrogen or substituted or unsubstituted alkyl (e.g., haloalkyl).

Provided in some embodiments herein is a compound of Formula (I-C), or a salt or solvate or tautomer or regioisomer thereof:

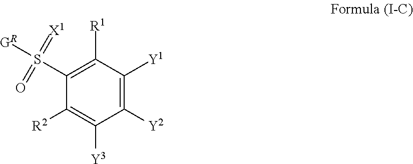

wherein,
G^{R} is substituted or unsubstituted alkyl;
X¹ is absent or O;
Y¹ and Y³ are each independently halogen (e.g., fluoro);
Y² is halogen (e.g., fluoro) or G;
R¹ is halogen (e.g., fluoro);
R² is halogen (e.g., fluoro) or G;
   G is or comprises a KRAS-binding ligand (e.g., G is -L²-G¹, wherein L2 is a linker (e.g., substituted or unsubstituted alkyl (e.g., alkyl substituted with pipirizinyl or piperidinyl), substituted or unsubstituted pipirizinyl, substituted or unsubstituted piperidinyl, substituted or unsubstituted azetidinyl (e.g., azetidinyl substituted with amino), or substituted or unsubstituted amino (e.g., —NH—, amino substituted with alkyl (e.g., —CH₂NH— or —CH₂CH₂NH—), or amino substituted with azetidinyl)), and G is a KRAS-binding ligand that interacts with KRAS (e.g., KRAS G12C)),
   wherein either Y² or R² is G.

In some embodiments, X¹ is absent.

In some embodiments, X¹ is O.

In some embodiments, R¹ is fluoro.

In some embodiments, R² is fluoro.

In some embodiments, Y¹ and Y³ are fluoro.

In some embodiments, R¹, Y¹, and Y³ are fluoro.

In some embodiments, R¹, R², Y¹, and Y³ are fluoro.

In some embodiments, R¹, Y¹, and Y³ are fluoro and G^{R} is G.

In some embodiments, R¹, Y¹, and Y³ are fluoro, R² is R⁷ (e.g., halogen (e.g., fluoro), substituted or unsubstituted alkyl (e.g., haloalkyl), or —OR³ (e.g., R³ being hydrogen or substituted or unsubstituted alkyl (e.g., haloalkyl))), and G^{R} is G.

In some embodiments, R¹, Y¹, and Y³ are fluoro and R² is G.

In some embodiments, R¹, Y¹, and Y³ are fluoro, G^{R} is substituted or unsubstituted alkyl, and R² is G.

In some embodiments, G or G¹ has or comprises a structure of any one of Formula (II), Formula (II-A), Formula (II-B), Formula (III), Formula (III-A), Formula (III-B), Formula (III-C), Formula (III-D), Formula (IV), Formula (IV-A), Formula (IV-B), Formula (V), Formula (V-A), Formula (VI), Formula (VI-A), Formula (VII), Formula (VII-A), or Formula (VII-B), or a structure provided in Table 2, Table 3, Table 4, Table 5, or Table 6.

Provided in some embodiments herein is a compound having a structure represented by Formula (I-A):

D1-L-D2 Formula (I-A)

wherein:
D1 is a radical of a KRAS-binding ligand;
D2 is a warhead radical (e.g., an aromatic (e.g., substituted phenyl) warhead radical); and
L is a linker,

or a pharmaceutically acceptable salt or solvate thereof.

In some embodiments, D2 is a selective (e.g., over other cysteine containing selectivity protein SOS1) warhead (radical). In some embodiments, D2 is selective for KRAS (e.g., KRAS G12C (e.g., over other cysteine containing selectivity protein SOS1)).

In some embodiments, D2 covalently modifies KRAS (e.g., KRAS G12C (SEQ ID NO: 1 or SEQ ID NO: 2) and/or mutant KRAS G12C Lite (SEQ ID NO: 3)).

In some embodiments, D2 does not (substantially) covalently modify KRAS WT protein.

In some embodiments, D2 binds to, disrupts, and/or modifies KRAS G12C (SEQ ID NO: 1 or SEQ ID NO: 2) and/or mutant KRAS G12C Lite (SEQ ID NO: 3) (e.g., in vitro (e.g., using differential scanning fluorimetry (DSF))).

In some embodiments, D2 comprises one or more warhead group, each warhead group being independently selected from the group consisting of (substituted or unsubstituted) sulfonamide, sulfone, sulfoxide, substituted or unsubstituted amino (e.g., a secondary amine (e.g., —NH—) or a tertiary amine (e.g., >N—)), or substituted aryl (e.g., aryl substituted with one or more substituent, each substituent being independently selected from sulfone, sulfoxide, halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (alkyl substituted with halogen (e.g., fluoro) (e.g., —CH₂F, —CHF₂, or —CF₃)))).

In some embodiments, D2 comprises an aryl substituted with one or more substituent, each substituent being independently selected from sulfone, sulfoxide, halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (alkyl substituted with halogen (e.g., fluoro) (e.g., —CH₂F, —CHF₂, or —CF₃))).

In some embodiments, D2 comprises a sulfone, a sulfoxide, or a sulfonamide.

In some embodiments, D2 comprises a sulfone and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 comprises a sulfoxide and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 comprises a sulfonamide and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 is or comprises an aryl substituted with halogen (e.g., fluoro).

In some embodiments, D2 is or comprises an aryl substituted with halogen (e.g., fluoro) and alkyl substituted with halogen (e.g., fluoro) (e.g., —CH₂F, —CHF₂, or —CF₃).

In some embodiments, D2 is or comprises an aryl substituted with halogen (e.g., fluoro) and hydroxy.

In some embodiments, D2 is or comprises an aryl substituted with halogen (e.g., fluoro) and unsubstituted alkoxy (e.g., methoxy).

In some embodiments, D2 is or comprises an aryl substituted with halogen (e.g., fluoro) and alkoxy substituted with halogen (e.g., fluoro) (e.g., —OCH₂F, —OCHF₂, or —OCF₃).

In some embodiments, D2 is or comprises an aryl substituted with halogen (e.g., fluoro) and sulfone.

In some embodiments, D2 is or comprises an aryl substituted with halogen (e.g., fluoro) and sulfoxide.

In some embodiments, D2 comprises a sulfone.

In some embodiments, D2 comprises a sulfonamide.

In some embodiments, D2 comprises a sulfoxide.

In some embodiments, the linker is a non-releasable linker (e.g., the linker does not decompose (e.g., hydrolyze) or release the warhead radical (or a free form thereof), the radical of the KRAS-binding ligand (or a free form thereof), or any other portion of the compound (e.g., a radical of any Formula provided herein) (or a free form thereof)).

In some embodiments, the linker comprises one or more linker group, each linker group being independently selected from the group consisting of —O—, (substituted or unsubstituted) amino, substituted or unsubstituted (e.g., acyclic (e.g., straight or branched) or cyclic) alkyl(ene), substituted or unsubstituted (e.g., acyclic (e.g., straight or branched) or cyclic) heteroalkyl(ene), and substituted or unsubstituted alkoxy.

In some embodiments, the linker comprises one or more linker group, each linker group being independently selected from the group consisting of (substituted or unsubstituted) amino and substituted or unsubstituted (e.g., acyclic (e.g., straight or branched) or cyclic) heteroalkyl(ene).

In some embodiments, the linker is —O—, (substituted or unsubstituted) amino or substituted or unsubstituted (e.g., acyclic (e.g., straight or branched) or cyclic) heteroalkyl(ene).

In some embodiments, L is a bond, substituted or unsubstituted alkyl(ene) (e.g., methylene, alkyl substituted with substituted or unsubstituted pipirizinyl), substituted or unsubstituted heteroalkyl(ene) (e.g., unsubstituted pipirizinyl, substituted pipirizinyl (e.g., pipirizinyl substituted with methyl), unsubstituted azetidinyl, or azetidinyl substituted with amino), or substituted or unsubstituted amino (e.g., —NH—, amino substituted with alkyl (e.g., —CH₂NH— or —CH₂CH₂NH—, or amino substituted with azetidinyl).

In some embodiments, L is a bond, substituted or unsubstituted alkylene (e.g., alkyl substituted with pipirizinyl), substituted or unsubstituted pipirizinyl, substituted or unsubstituted azetidinyl (e.g., azetidinyl substituted with amino), or substituted or unsubstituted amino (e.g., —NH—, amino substituted with alkyl (e.g., —CH₂NH— or —CH₂CH₂NH—), or amino substituted with azetidinyl).

In some embodiments, L is a bond.

In some embodiments, D1 has a structure represented in any of one Tables 2-6 (e.g., and L is a bond).

In some embodiments, D1 has a structure represented by:

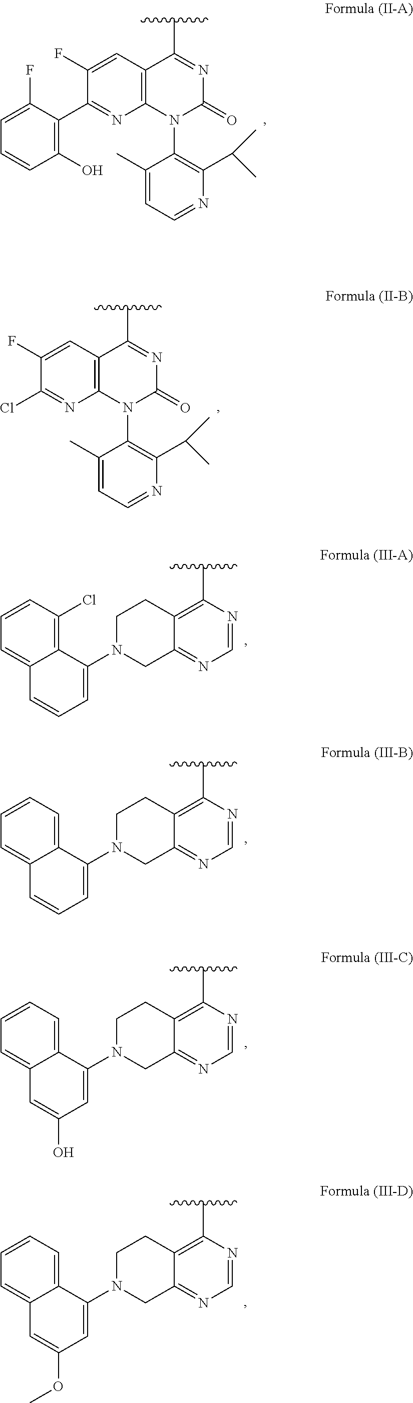 [SMILES: *c1nc(=O)n(-c2c(C)ccnc2C(C)C)c2nc(-c3c(O)cccc3F)c(F)cc12 ; *c1nc(=O)n(-c2c(C)ccnc2C(C)C)c2nc(Cl)c(F)cc12 ; *c1ncnc2c1CCN(c1cccc3cccc(Cl)c13)C2 ; *c1ncnc2c1CCN(c1cccc3ccccc13)C2 ; *c1ncnc2c1CCN(c1cc(O)cc3ccccc13)C2 ; *c1ncnc2c1CCN(c1cc(OC)cc3ccccc13)C2]

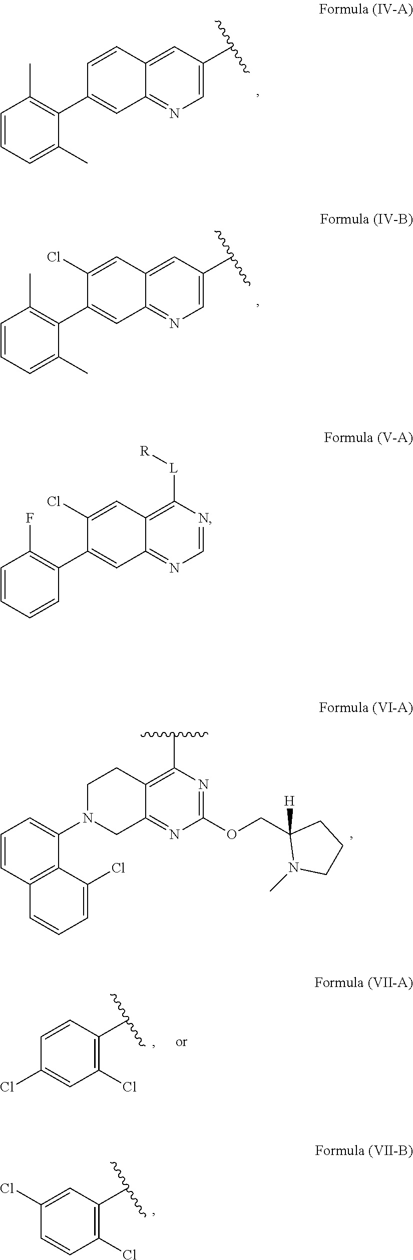

Provided in some embodiments is a compound selected from Table 8.

In some cases, the present disclosure provides a compound or a salt or solvate or tautomer or regioisomer thereof, wherein the compound is a compound from Table 1 or a salt or solvate or tautomer or regioisomer thereof.

In some cases, the present disclosure provides a pharmaceutically acceptable composition comprising a compound of any one of the preceding claims, or a salt or solvate or tautomer or regioisomer thereof, and one or more of pharmaceutically acceptable excipients.

In some cases, the present disclosure provides a KRAS protein or an active fragment thereof modified with a compound of any one of the preceding claims, or a salt or solvate or tautomer or regioisomer thereof, wherein the compound forms a covalent bond with a sulfur atom of a cysteine residue of the KRAS protein or an active fragment thereof (e.g., a polypeptide thereof).

In some cases, the present disclosure provides a method of modifying (e.g., attaching to and/or degrading) KRAS protein or an active fragment thereof with a compound, comprising contacting the polypeptide with a compound of any one of the preceding claims, or a salt or solvate or tautomer or regioisomer thereof, to form a covalent bond with a sulfur atom of a cysteine residue of the KRAS protein or an active fragment thereof (e.g., polypeptide thereof).

In some cases, the present disclosure provides a method of binding a compound to KRAS protein or an active fragment thereof, comprising contacting the KRAS protein or an active fragment thereof (e.g., polypeptide thereof) with a compound of any one of the preceding claims, or a salt or solvate or tautomer or regioisomer thereof.

In some cases, the present disclosure provides a method of disrupting KRAS protein or an active fragment thereof (e.g. a function thereof), comprising contacting the KRAS protein or an active fragment thereof (e.g., polypeptide thereof) with a compound of any one of the preceding claims, or a salt or solvate or tautomer or regioisomer thereof.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the mass spectroscopy trace for the intact mass analysis with His-tagged KRAS G12C Lite protein incubated with Compound 1A (molecular weight: 736.66 g/mol) showing the covalent adduct mass (22,179 Da) for modification by one molecule of Compound 1A.

FIG. 2 shows the mass spectroscopy trace for the intact mass analysis with His-tagged KRAS G12C protein incubated with Compound 1A (molecular weight: 736.66 g/mol) showing the covalent adduct mass (22,070 Da) for modification by one molecule of Compound 1A.

FIG. 3 shows the mass spectroscopy trace for the intact mass analysis with His-tagged KRAS G12C Lite protein incubated with Compound 6A (molecular weight: 786.67 g/mol) showing the covalent adduct mass (22,229 Da) for modification by one molecule of Compound 6A.

FIG. 4 shows the mass spectroscopy trace for the intact mass analysis with His-tagged KRAS G12C protein incubated with Compound 6A (molecular weight: 786.67 g/mol) showing the covalent adduct mass (22,120 Da) for modification by one molecule of Compound 6A.

FIG. 5 shows the mass spectroscopy trace for the intact mass analysis with His-tagged KRAS G12C Lite protein incubated with Compound 7A (molecular weight: 766.68 g/mol) showing the covalent adduct mass (22,209 Da) for modification by one molecule of Compound 7A.

FIG. 6 shows the mass spectroscopy trace for the intact mass analysis with His-tagged KRAS G12C protein incubated with Compound 7A (molecular weight: 766.68 g/mol) showing the covalent adduct mass (22,100 Da) for modification by one molecule of Compound 7A.

FIG. 7 shows the peptide fragment coverage of His-KRAS G12C (SEQ ID NO:1) after trypsin digestion showing coverage of 100% of the sequence and confirming covalent modification of peptide 24LVVVGACGVGK34 at Cys-30 in this construct, which corresponds to Cys-12 in full length protein, by example Compound 1A (by underlining, panel A shows 100% coverage, panel B shows 81% coverage, and panel C shows 9% coverage).

FIG. 8 shows the MSMS spectrum of peptide 24LVVVGACGVGK34 from example Compound 1A treated His-KRAS G12C digest where the Cys is modified by one Compound 1A. The alignment of b and y ions confirms that Cys-30 is the amino acid that is modified by Compound 1A which corresponds to Cys-12 in full-length protein.

FIG. 9 shows the MSMS spectrum of peptide 136CDLPSR141 from example Compound 1A treated His-KRAS G12C digest where the Cys is modified by one Compound 1A. The alignment of b and y ions confirms that Cys-136 is the amino acid that is modified by Compound 1A which corresponds to Cys-118 in full-length protein.

FIG. 10 shows the peptide fragment coverage of His-KRAS G12C (SEQ ID NO:1) after trypsin digestion showing coverage of 100% of the sequence and confirming covalent modification of peptide 24LVVVGACGVGK34 at Cys-30 in this construct, which corresponds to Cys-12 in full length protein, by example Compound 7A (by underlining, panel A shows 100% coverage, panel B shows 79% coverage, and panel C shows 9% coverage).

FIG. 11 shows the MSMS spectrum of peptide 24LVVVGACGVGK34 from example Compound 6A treated His-KRAS G12C digest where the Cys is modified by one Compound 6A. The alignment of b and y ions confirms that Cys-30 is the amino acid that is modified by Compound 6A which corresponds to Cys-12 in full-length protein.

FIG. 12 shows the MSMS spectrum of peptide 136CDLPSR141 from example Compound 7A treated His-KRAS G12C digest where the Cys is modified by one Compound 7A. The alignment of b and y ions confirms that Cys-136 is the amino acid that is modified by Compound 7A which corresponds to Cys-118 in full-length protein.

FIG. 13 shows the peptide fragment coverage of His-KRAS G12C (SEQ ID NO:1) after trypsin digestion showing coverage of 100% of the sequence and confirming covalent modification of peptide 24LVVVGACGVGK34 at Cys-30 in this construct, which corresponds to Cys-12 in full length protein, by example Compound 6A (by underlining, panel A shows 100% coverage, panel B shows 55% coverage, and panel C shows 9% coverage).

FIG. 14 shows the MSMS spectrum of peptide 24LVVVGACGVGK34 from example Compound 6A treated His-KRAS G12C digest where the Cys is modified by one Compound 6A. The alignment of b and y ions confirms that Cys-30 is the amino acid that is modified by Compound 6A which corresponds to Cys-12 in full-length protein.

FIG. 15 shows the MSMS spectrum of peptide 136CDLPSR141 from example Compound 6A treated His-KRAS G12C digest where the Cys is modified by one Compound 6A. The alignment of b and y ions confirms that Cys-136 is the amino acid that is modified by Compound 6A which corresponds to Cys-118 in full-length protein.

FIG. 16 illustrates an example of a warhead portion, a linker portion, and a KRAS-binding ligand portion of a compound provided herein.

### DETAILED DESCRIPTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. In some embodiments, about is within 10% of the stated number or numerical range. In some embodiments, about is within 5% of the stated number or numerical range. In some embodiments, about is within 1% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“KRAS protein” refers to a wild-type KRAS protein or a mutant thereof.

“KRAS-binding ligand” refers to a ligand binding to a KRAS protein or a mutant thereof, for example KRAS GT2C, KRAS C118A, or KRAS GT2C/CTT8A.

“Amino” refers to the —NH₂ moiety.

“Hydroxy” or “hydroxyl” refers to the —OH moiety.

“Alkyl” generally refers to anon-aromatic straight or branched hydrocarbon chain radical consisting solely of carbon and hydrogen atoms, partially or fully saturated, cyclic or acyclic, having from one to fifteen carbon atoms (e.g., C₁-C₁₈ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₂ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an alkyl includes alkenyl, alkynyl, cycloalkyl, carbocycloalkyl, cycloalkylalkyl, haloalkyl, and fluoroalkyl, as defined herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is as defined above. Unless stated otherwise specifically in the specification, an alkoxy group is optionally substituted, as defined above for an alkyl group.

“Alkoxyalkyl” refers to an alkyl moiety comprising at least one alkoxy substituent, where alkyl is as defined above. Unless stated otherwise specifically in the specification, an alkoxyalkyl group is optionally substituted, as defined above for an alkyl group.

“Alkylamino” refers to a moiety of the formula —NHRₐ or —NRₐR_{b} where Rₐ and R_{b} are each independently an alkyl group as defined above. Unless stated otherwise specifically in the specification, an alkylamino group is optionally substituted, as defined above for an alkyl group.

“Alkylaminoalkyl” refers to an alkyl moiety comprising at least one alkylamino substituent. The alkylamino substituent can be on a tertiary, secondary or primary carbon. Unless stated otherwise specifically in the specification, an alkylaminoalkyl group is optionally substituted, as defined above for an alkyl group.

“Aminoalkyl” refers to an alkyl moiety comprising at least one amino substituent. The amino substituent can be on a tertiary, secondary or primary carbon. Unless stated otherwise specifically in the specification, an aminoalkyl group is optionally substituted, as defined above for an alkyl group.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R′—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Arylene” refers to a divalent aryl group which links one part of the molecule to another part of the molecule. Unless stated specifically otherwise, an arylene is optionally substituted, as defined above for an aryl group.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

The term “carbocycle” or “carbocyclic” refers to a ring or ring system where the atoms forming the backbone of the ring are all carbon atoms. The term thus distinguishes carbocyclic group from a “heterocycle” or “heterocyclic” in which the ring backbone contains at least one atom which is different from carbon. In some embodiments, carbocycles are monocyclic, bicyclic, polycyclic, spirocyclic or bridged compounds. Carbocycle includes aromatic and partially or fully saturated ring systems. Heterocycle includes aromatic and partially or fully saturated ring systems. In some embodiments, carbocycle comprises cycloalkyl and aryl. In some embodiments, a carbocycle provided herein is optionally substituted (e.g., carbocycle substituted with one or more carbocycle substitutent, each carbocycle substituent being independently selected from the group consisting of alkyl, oxo, halo, hydroxyl, heteroalkyl, alkoxy, aryl, and heteroaryl). In some embodiments, a heterocycle provided herein is optionally substituted (e.g., heterocycle substituted with one or more heterocycle substitutent, each heterocycle substituent being independently selected from the group consisting of alkyl, oxo, halo, hydroxyl, heteroalkyl, alkoxy, aryl, and heteroaryl).

“Cyclic ring” refers to a carbocycle or heterocycle, including aromatic, non-saturated, and saturated carbocycle and heterocycle. A “cyclic ring” is optionally monocyclic or polycyclic (e.g., bicyclic).

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyl is saturated (i.e., containing single C—C bonds only) or unsaturated (i.e., containing one or more double bonds or triple bonds). Examples of monocyclic cycloalkyls include, e.g., cyclopropyl, cyclobutyl, cyclopentyl, cyclohexyl, cycloheptyl, and cyclooctyl. An unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Cycloalkylalkyl” refers to a radical of the formula —R^{c}-cycloalkyl where R^{c} is an alkylene chain as defined above. The alkylene chain and the cycloalkyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

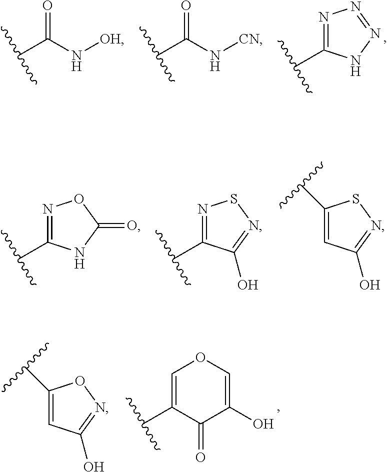

and the like.

“Halo” or “halogen” refers to bromo, chloro, fluoro or iodo substituents. A “haloalkyl” refers to an alkyl radical, as described herein, that is substituted with one or more halo radical, such as described above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), phosphorous (e.g. >P—, >P(═O)—, or —P(═O)₂), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. Representative heteroalkyl groups include, but are not limited to —OCH₂OMe, —OCH₂CH₂OH, —CH₂CH₂OMe, or —OCH₂CH₂OCH₂CH₂NH₂. In some embodiments, heteroalkyl includes alkoxy, alkoxyalkyl, alkylamino, alkylaminoalkyl, aminoalkyl, heterocycloalkyl, heterocycloalkyl, and heterocycloalkylalkyl, as defined herein. Unless stated otherwise specifically in the specification, a heteroalkyl group is optionally substituted, as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic or tetracyclic ring system, which optionally includes fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the ring(s). A fully saturated heterocycloalkyl radical is also referred to as “heterocycloalkyl.” Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocycloalkyl” or “N-attached heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals. Examples of such N-heterocycloalkyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocycloalkyl” or “C-attached heterocycloalkyl” refers to a heterocycloalkyl radical as defined above containing at least one heteroatom and where the point of attachment of the heterocycloalkyl radical to the rest of the molecule is through a carbon atom in the heterocycloalkyl radical. A C-heterocycloalkyl radical is optionally substituted as described above for heterocycloalkyl radicals. Examples of such C-heterocycloalkyl radicals include, but are not limited to, 2-morpholinyl, 2- or 3- or 4-piperidinyl, 2-piperazinyl, 2- or 3-pyrrolidinyl, and the like.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroarylene” refers to a divalent heteroaryl group which links one part of the molecule to another part of the molecule. Unless stated specifically otherwise, a heteroarylene is optionally substituted, as defined above for a heteroaryl group.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

The compounds disclosed herein, in some embodiments, contain one or more asymmetric centers and thus give rise to enantiomers, diastereomers, and other stereoisomeric forms that are defined, in terms of absolute stereochemistry, as (R)— or (S)—. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Recitations of structures described herein also include recitations of tautomers thereof, e.g., a switch of a single bond and adjacent double bond, for example

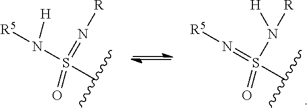

In some instances, the drawing of a compound is provided herein in one tautomeric form; each such drawing herein includes disclosure of such a compound as drawn and of a tautomer thereof (when applicable), including a tautomer as illustrated in the drawing above (when applicable). In some embodiments, the present disclosure provides a tautomer of a compound or fragment herein or an equilibrium of tautomers. A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric form or equilibrium include:

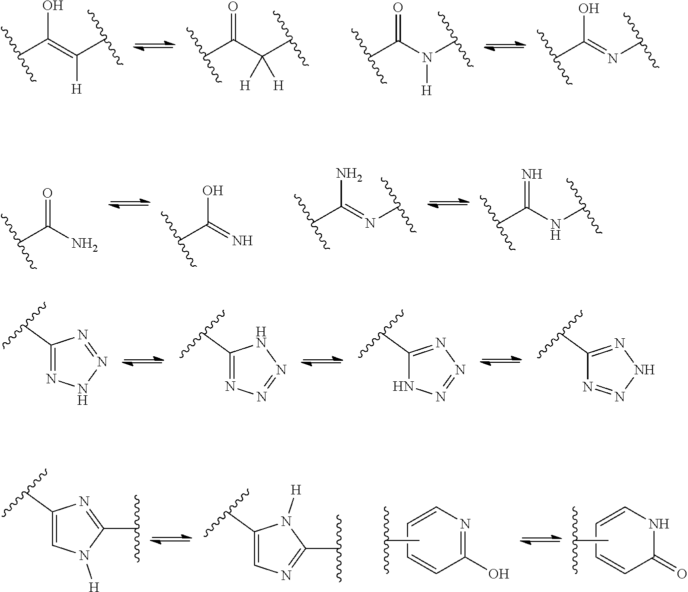

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds, whether radioactive or not, are encompassed within the scope of the present disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

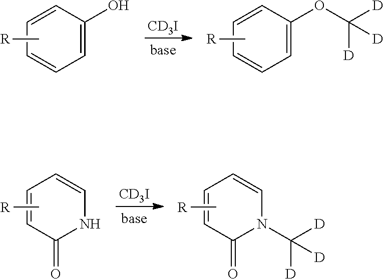

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

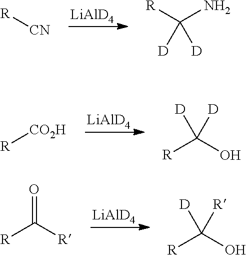

Deuterium gas and palladium catalyst are employed to reduce unsaturated carbon-carbon linkages and to perform a reductive substitution of aryl carbon-halogen bonds as illustrated, by way of example only, in the reaction schemes below.

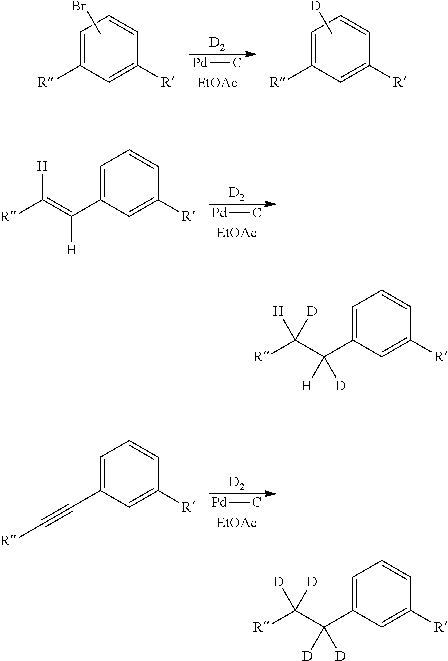

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor compounds described herein is intended to encompass any and all pharmaceutically suitable salt forms. Exemplary pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Chemical modification is an important tool to alter structure and function of proteins. One way to achieve chemical modification of proteins is to use protein binders (e.g., a (e.g., covalent) small molecule inhibitor). As a result, binders (e.g., covalent small molecule binders (e.g., inhibitors) of proteins) are considered to be useful in multiple applications, including therapeutics. Covalent binding (e.g., inhibition) of a target protein may minimize the required systemic drug exposure. In some embodiments, protein (e.g., functional) activity can only be restored by de novo protein synthesis, resulting in a prolonged therapeutic effect long after the compound is cleared from the blood. Strategically placing an electrophilic moiety on the protein binder (e.g., inhibitor) will allow it to undergo attack by a nucleophilic amino acid residue upon binding to the target protein, forming a reversible or irreversible bond that is much stronger than typical noncovalent interactions. However, the ability to form a covalent bond with the target enzyme has raised concerns about indiscriminate reactivity with off-target proteins, even though some of the most prescribed drugs are covalent irreversible binders. This led to the disfavor of covalent modifiers as drug candidates until the recent successful development of irreversible covalent kinase inhibitors ibrutinib and afatinib, which form an irreversible covalent bond between an acrylamide warhead and a nonconserved cysteine residue on the ATP-binding site but also with nontargeted cellular thiols. The ability to form covalent adducts with off-target proteins has been linked to an increased risk of unpredictable idiosyncratic toxicity along with the daily drug dose administered to patients. Accordingly, there is a need to reduce the risk of non-target covalent interactions by incorporating less reactive electrophilic moieties into binders (e.g., to form covalent small molecule binders (e.g, inhibitors)). In some embodiments, described herein is a protein binder, such as a covalent small molecule binder (e.g., inhibitor). In some embodiments, described herein is a covalent small molecule binder which acts functionally as an inhibitor. In some embodiments, described herein is a pharmaceutical composition comprising a protein binder (e.g., a covalent small molecule binder (e.g., inhibitor)) and one or more of pharmaceutically acceptable excipients. In other embodiments, a protein binder (e.g., a covalent small molecule binder (e.g., inhibitor)) is used to treat or prevent a disease or condition in a subject in need thereof.

In some embodiments, a protein binder provided herein, such as a covalent small molecule binder (e.g., inhibitor) is a benzenesulfonamide derivative compound. In some embodiments, a benzenesulfonamide derivative compound as described herein is used to treat or prevent a disease or condition in a subject in need thereof.

In some instances, a protein binder provided herein, such as any compound provided herein, such as a compound of Table 8, binds to, (e.g., covalently) interacts with, modulates (e.g., inhibits), destabilizes, imparts a conformational change, (functionally) disrupts a protein described herein, such as, for example, KRAS. In some instances, a protein binder provided herein binds to KRAS. In some instances, a protein binder provided herein interacts with KRAS. In some instances, a protein binder provided herein covalently interacts with KRAS. In some instances, a protein binder provided herein modulates KRAS. In some instances, a protein binder provided herein inhibits KRAS. In some instances, a protein binder provided herein destabilizes KRAS. In some instances, a protein binder provided herein imparts a conformational change to KRAS (e.g., upon binding). In some instances, a protein binder provided herein disrupts KRAS. In some instances, a protein binder provided herein functionally disrupts KRAS.

In some instances, an inhibitor is a protein binder that degrades and/or disrupts the functionality of a protein described herein, such as KRAS.

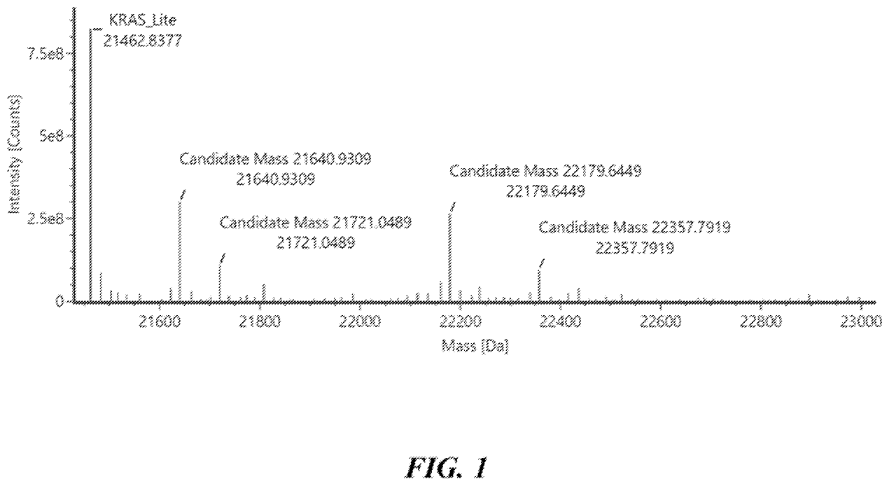

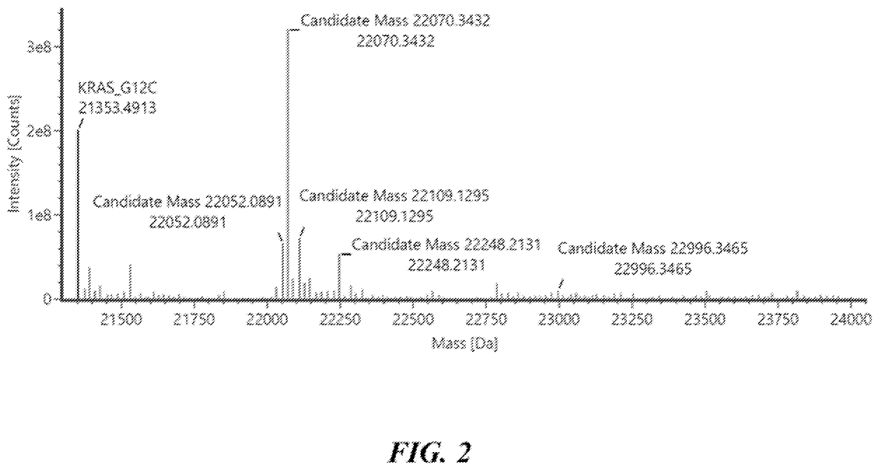

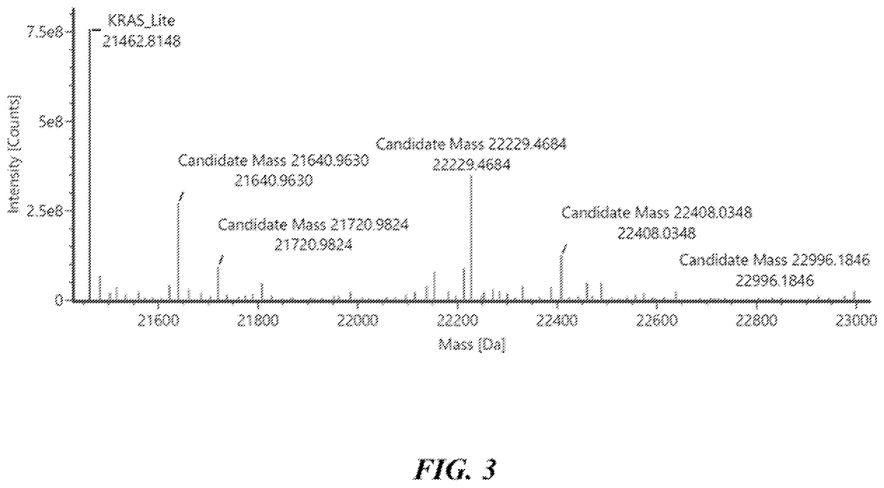

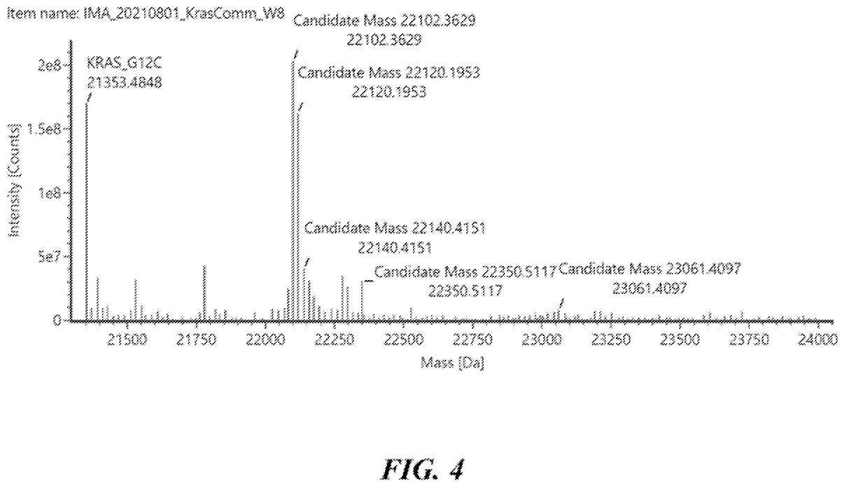

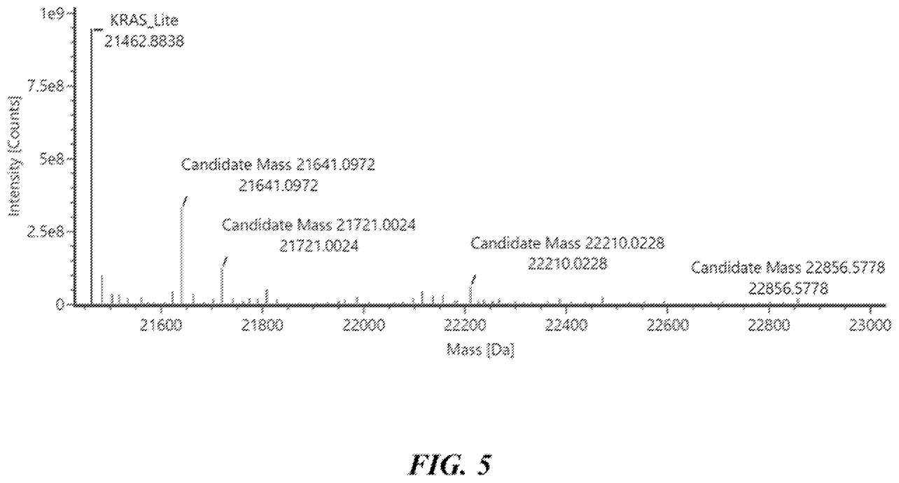

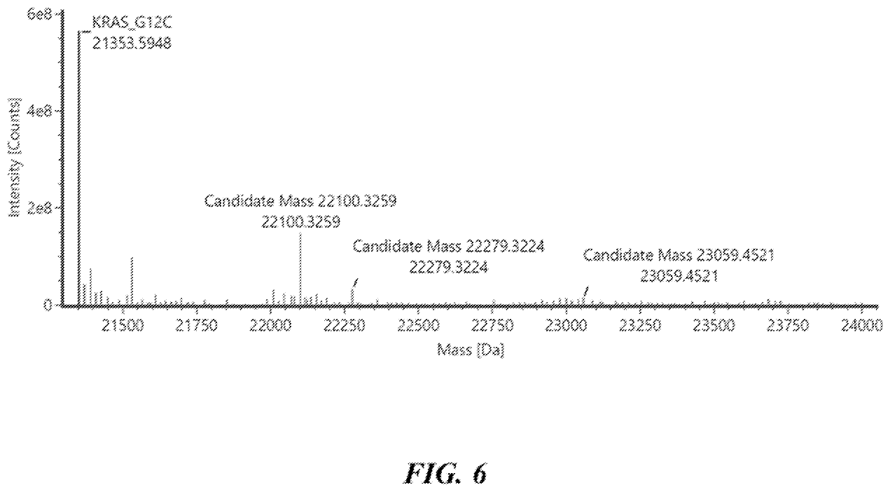

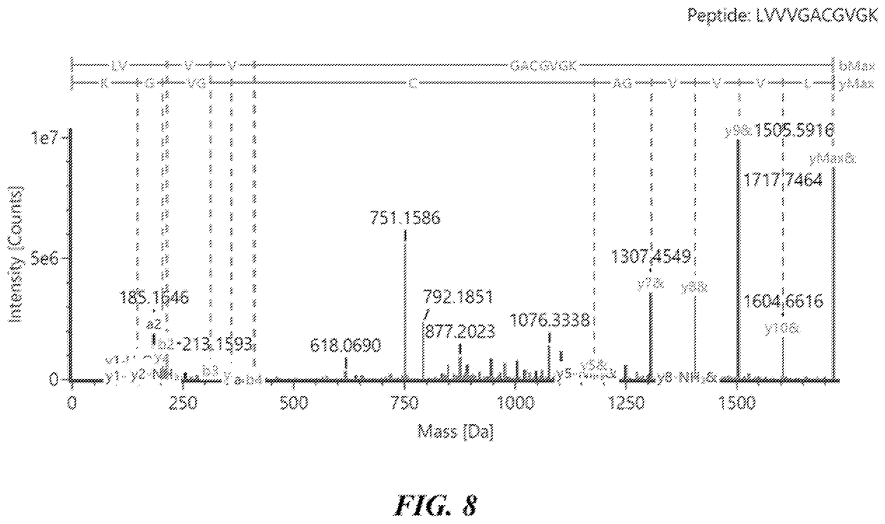

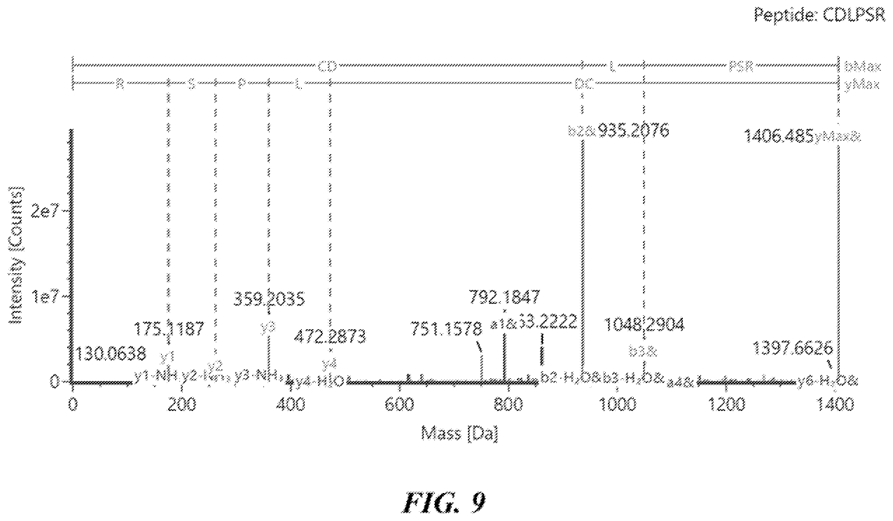

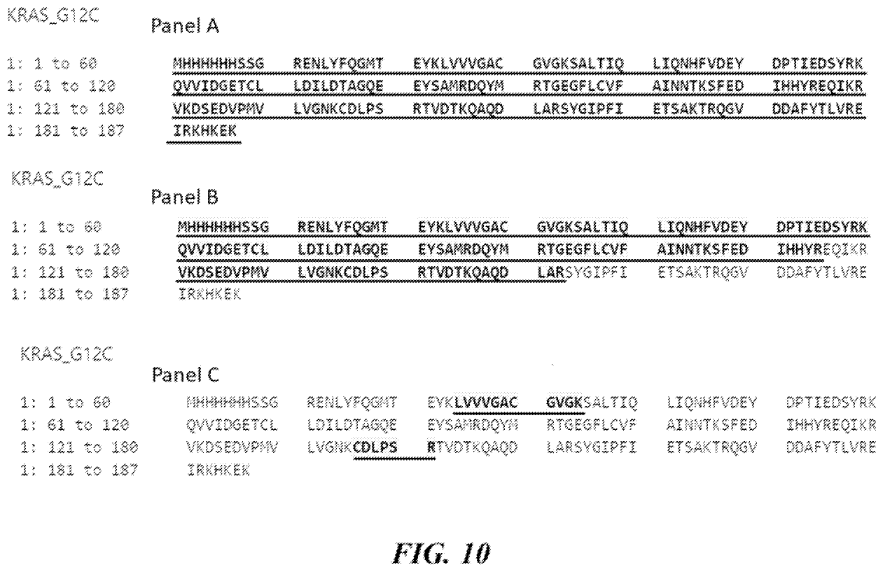

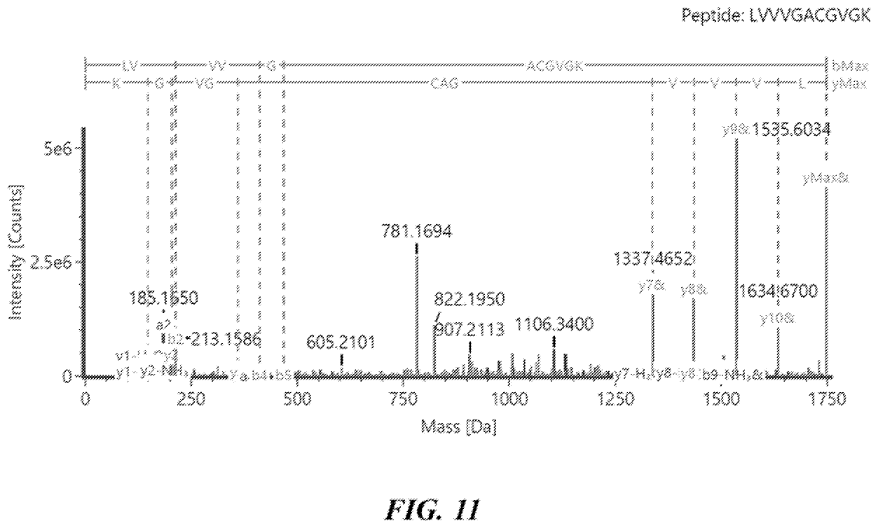

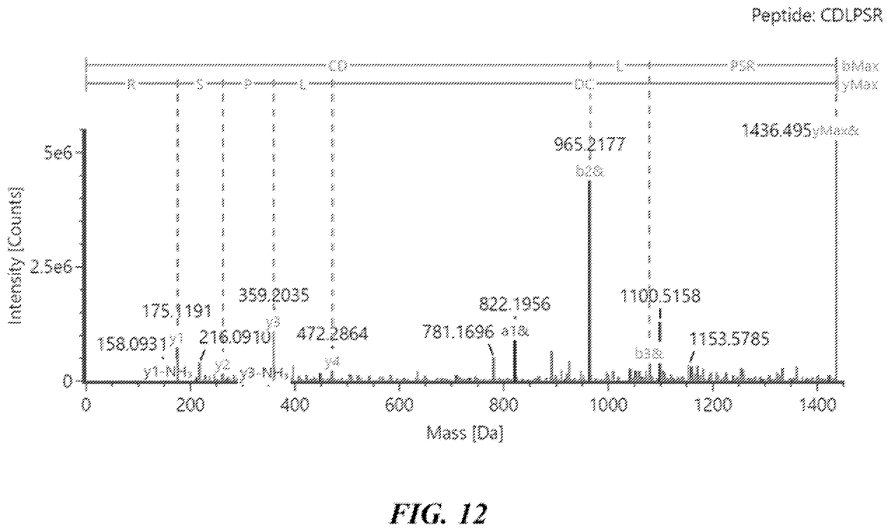

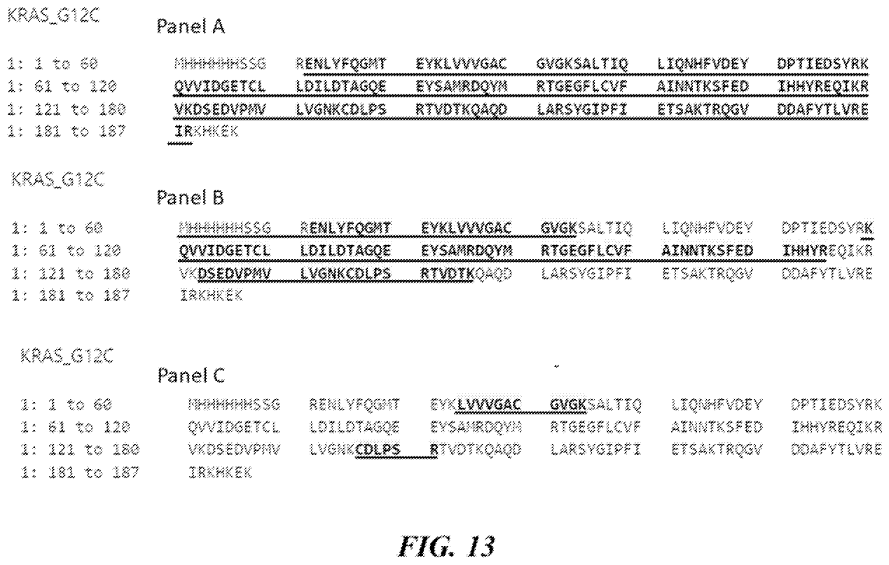

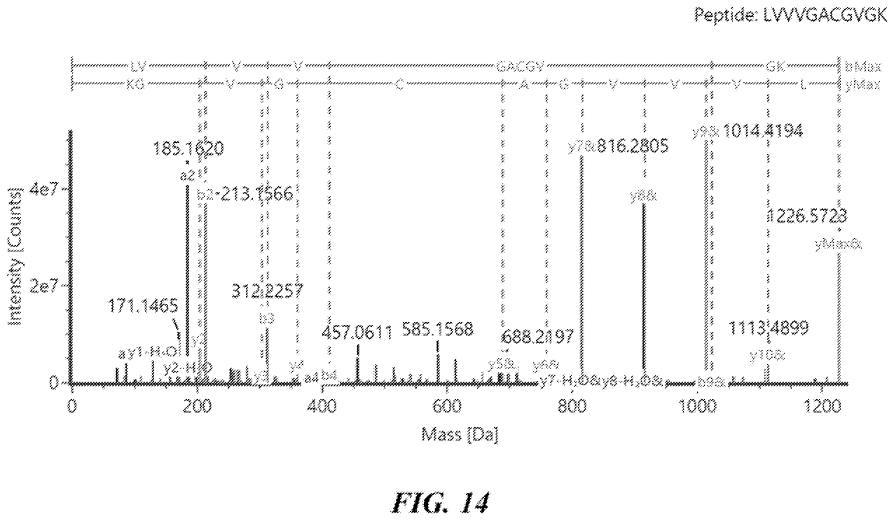

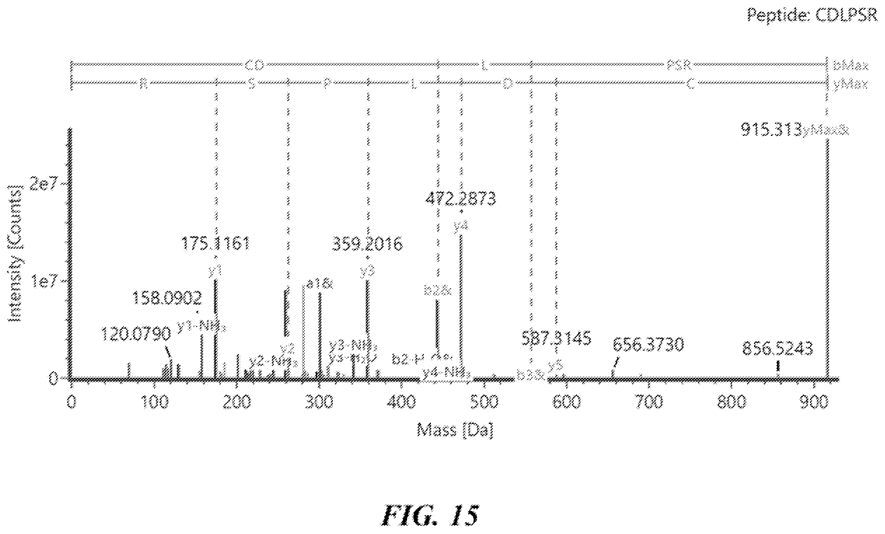

In some instances, a compound provided herein is an irreversible binder (e.g., inhibitor). In some instances, mass spectrometry (e.g., of the protein drug target modified (e.g., KRAS) in the presence of a compound provided herein) is used to determine if a compound is an irreversible binder (e.g., inhibitor), such as shown in FIGS. 1-15. In some instances, a protein (e.g., KRAS) inhibited by a compound provided herein is subjected to mass spectral analysis (e.g., to assess the formation of permanent, irreversible covalent adducts). In some instances, analytical methods to examine peptide fragments (e.g., generated upon tryptic cleavage of a protein (e.g., KRAS)) include, but are not limited to mass spectroscopy. In some instances, such methods identify permanent, irreversible covalent protein adducts (e.g., by observing a mass peak that corresponds to the mass of a control sample plus the mass of an irreversible adduct).

In some instances, such as when a protein described herein interacts (e.g., is bound (e.g., covalently and/or irreversibly bound)) with a compound provided herein, binding of a protein described herein leads to functional inhibition of the protein target (e.g., in a cellular environment).

In some embodiments, a compound provided herein comprises a group (e.g., a warhead) that irreversibly or covalently binds to a protein (e.g., KRAS). In some instances, a warhead provided herein is a functional group that covalently binds to an amino acid residue (such as cysteine, lysine, histidine, or other residues capable of being covalently modified), present in or near the binding pocket of a target protein (e.g., KRAS). In some instances, a warhead provided herein irreversibly inhibits KRAS. In some instances, a warhead provided herein covalently and irreversibly inhibits KRAS either alone or in combination with L (e.g., warhead-L-).

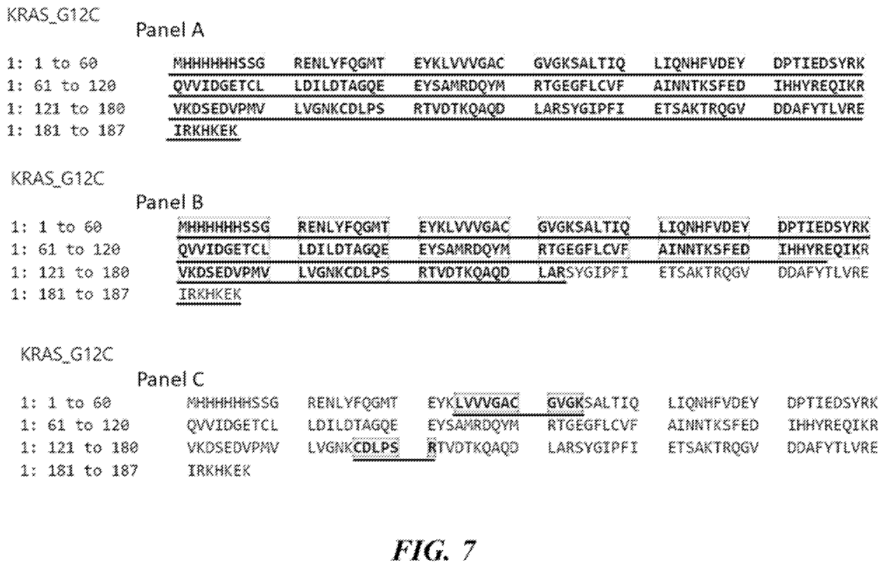

In some embodiments, a compound provided herein irreversibly and covalent modifies KRAS G12C at cysteine-12 and/or cysteine-118 in the full-length protein (for example, see FIGS. 7-15).

In other embodiments, a pharmaceutical composition comprising a benzenesulfonamide derivative compound as described herein and one or more of pharmaceutically acceptable excipients is used to treat or prevent a disease or condition in a subject in need thereof.

In some embodiments, disclosed herein is a method of treating a disease comprising administering to a subject in need thereof a therapeutically effective amount of a benzenesulfonamide derivative compound as described herein.

In other embodiments, disclosed herein is a method of treating a disease comprising administering to a subject in need thereof a therapeutically effective amount of a pharmaceutical composition comprising a benzenesulfonamide derivative compound as described herein and one or more of pharmaceutically acceptable excipients.

In some embodiments, disclosed herein is a KRAS protein or an active fragment thereof (e.g., a polypeptide thereof) modified with a benzenesulfonamide derivative compound as described herein, wherein the compound forms a covalent bond with a sulfur atom of a cysteine residue of the KRAS protein or an active fragment thereof (e.g., a polypeptide thereof). In some embodiments, disclosed herein is a method of modifying (e.g., attaching to and/or degrading) a polypeptide with a benzenesulfonamide derivative compound as described herein, comprising contacting the polypeptide with the compound to form a covalent bond with a sulfur atom of a cysteine residue of the polypeptide. In some embodiments, disclosed herein is a method of binding a compound to a polypeptide, comprising contacting the polypeptide with a benzenesulfonamide derivative compound as described herein.

In one aspect, provided herein is a benzenesulfonamide derivative compound. In some embodiments, a benzenesulfonamide derivative compound is a KRAS binding compound. In some embodiments, a benzenesulfonamide derivative compound is a KRAS inhibitory compound.

Provided in some embodiments herein is a compound having a structure represented by Formula (I-A): D1-L-D2. In some embodiments, D1 is a radical of a KRAS-binding ligand. In some embodiments, D2 is a warhead radical. In some embodiments, L is a linker. In some embodiments, the compound is a pharmaceutically acceptable salt or solvate.

In some embodiments, D2 is a warhead radical (such as having a structure of any one of Formula (II), Formula (II-A), Formula (II-B), Formula (III), Formula (III-A), Formula (III-B), Formula (III-C), Formula (III-D), Formula (IV), Formula (IV-A), Formula (IV-B), Formula (V), Formula (V-A), Formula (VI), Formula (VI-A), Formula (VII), Formula (VII-A), or Formula (VII-B), or a warhead radical provided in Table 2, Table 3, Table 4, Table 5, or Table 6), such as an aromatic warhead radical, such as a substituted phenyl warhead radical, such as a phenyl warhead radical substituted with halogen (e.g., fluorine).

In some embodiments, D2 is a selective warhead. In some embodiments, D2 is a selective over other cysteine containing selectivity protein SOS1. In some embodiments, D2 is selective for KRAS, such as over wild-type KRAS. In some embodiments, D2 is selective for KRAS G12C (SEQ ID NO: 1 or SEQ ID NO:2) and/or mutant KRAS G12C Lite (SEQ ID NO: 3), such as over wild-type (WT) KRAS.

Sequence of KRAS G12C:
(SEQ ID NO: 1)
MHHHHHHSSG RENLYFQGMT EYKLVVVGAC GVGKSALTIQ
LIQNHFVDEY DPTIEDSYRK QVVIDGETCL LDILDTAGQE
EYSAMRDQYM RTGEGFLCVF AINNTKSFED IHHYREQIKR
VKDSEDVPMV LVGNKCDLPS RTVDTKQAQD LARSYGIPFI
ETSAKTRQGV DDAFYTLVRE IRKHKEK
Sequence of KRAS G12C Commercial:
(SEQ ID NO: 2)
HHHHHHSSG RENLYFQGMT EYKLVVVGAC GVGKSALTIQ
LIQNHFVDEY DPTIEDSYRK QVVIDGETCL LDILDTAGQE
EYSAMRDQYM RTGEGFLCVF AINNTKSFED IHHYREQIKR
VKDSEDVPMV LVGNKCDLPS RTVDTKQAQD LARSYGIPFI
ETSAKTRQGV DDAFYTLVRE IRKHKEK
Sequence of KRAS G12C Lite:
(SEQ ID NO: 3)
MHHHHHHSSG RENLYFQGMT EYKLVVVGAC GVGKSALTIQ
LIQNHFVDEY DPTIEDCYRK QVVIDGETSL LDILDTAGQE
EYSAMRDQYM RTGEGFLSVF AINNTKSFED IHHYREQIKR
VKDSEDVPMV LVGNKSDLPS RTVDTKQAQD LARPYGIPFI
ETSAKTRQGV DDAFYTLVRE IRKHKEK

KRAS G12C Lite (SEQ ID NO: 3) is FL KRAS mutated at all the cysteines except G12C (K-Ras(C51S/C80L/C118S) described in reference: Ostrem, J. M. L.; Shokat, K. M. Direct Small-Molecule Inhibitors of KRAS: From Structural Insights to Mechanism-Based Design. Nature Reviews Drug Discovery. Nature Publishing Group Nov. 1, 2016, pp 771-785.

In some embodiments, D2 covalently modifies KRAS. In some embodiments, D2 covalently modifies KRAS G12C (SEQ ID NO:1 or SEQ ID NO:2) and/or mutant KRAS G12C Lite (SEQ ID NO:3).

In some embodiments, D2 does not covalently modify KRAS WT protein. In some embodiments, D2 does not substantially covalently modify KRAS WT protein.

In some embodiments, D2 binds to, disrupts, and/or modifies KRAS G12C (SEQ ID NO: 1 or SEQ ID NO:2) and/or mutant KRAS G12C Lite (SEQ ID NO:3), such as in vitro, such as using differential scanning fluorimetry (DSF), such as described in the Examples.

In some embodiments, D2 comprises one or more warhead group. In some embodiments, D2 comprises one or more warhead group, each warhead group being independently selected from the group consisting of substituted or unsubstituted sulfonamide, substituted or unsubstituted sulfone, substituted or unsubstituted sulfoxide, substituted or unsubstituted amino, or substituted aryl. In some embodiments, D2 comprises one or more warhead group, each warhead group being independently selected from the group consisting of substituted or unsubstituted sulfonamide, substituted or unsubstituted sulfone, substituted or unsubstituted sulfoxide, or substituted aryl.

In some embodiments, D2 comprises a sulfone, a sulfoxide, or a sulfonamide.

In some embodiments, D2 comprises substituted or unsubstituted sulfonamide.

In some embodiments, D2 comprises substituted or unsubstituted sulfone.

In some embodiments, D2 comprises substituted or unsubstituted sulfoxide.

In some embodiments, D2 comprises substituted or unsubstituted amino. In some embodiments, D2 comprises a secondary amine (e.g., —NH—) or a tertiary amine (e.g., >N—)).

In some embodiments, D2 comprises substituted aryl.

In some embodiments, D2 comprises an aryl substituted with one or more substituent, each substituent being independently selected from sulfone, sulfoxide, halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (alkyl substituted with halogen (e.g., fluoro) (e.g., —CH₂F, —CHF₂, or —CF₃))).

In some embodiments, D2 comprises a sulfone and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 comprises a sulfoxide and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 comprises a sulfonamide and an aryl substituted with one or more substituent, each substituent being independently selected from halogen (e.g., fluoro), hydroxy, substituted or unsubstituted alkoxy (e.g., unsubstituted alkoxy (e.g., methoxy) or alkoxy substituted with halogen (e.g., fluoro) (e.g., —OCH₂F, —OCHF₂, or —OCF₃)), substituted or unsubstituted alkyl (e.g., alkyl substituted with halogen (e.g., fluoro) (e.g., —CH₂F, —CHF₂, or —CF₃))).

In some embodiments, D2 comprises an aryl substituted with halogen (e.g., fluoro). In some embodiments, D2 is an aryl substituted with fluoro.

In some embodiments, D2 comprises an aryl substituted with halogen (e.g., fluoro) and alkyl substituted with halogen (e.g., fluoro) (e.g., —CH₂F, —CHF₂, or —CF₃). In some embodiments, D2 comprises an aryl substituted with fluoro and alkyl substituted with halogen fluoro. In some embodiments, D2 comprises an aryl substituted with fluoro and —CH₂F, —CHF₂, or —CF₃.

In some embodiments, D2 comprises an aryl substituted with halogen (e.g., fluoro) and hydroxy. In some embodiments, D2 is an aryl substituted with fluoro and hydroxy.

In some embodiments, D2 comprises an aryl substituted with halogen (e.g., fluoro) and unsubstituted alkoxy (e.g., methoxy). In some embodiments, D2 is an aryl substituted with fluoro and methoxy.

In some embodiments, D2 comprises an aryl substituted with halogen (e.g., fluoro) and alkoxy substituted with halogen (e.g., fluoro) (e.g., —OCH₂F, —OCHF₂, or —OCF₃). In some embodiments, D2 is an aryl substituted with fluoro and alkoxy substituted with fluoro. In some embodiments, D2 is an aryl substituted with fluoro and —OCH₂F, —OCHF₂, or —OCF₃.

In some embodiments, D2 comprises an aryl substituted with halogen (e.g., fluoro) and sulfone. In some embodiments, D2 is an aryl substituted with fluoro and sulfone.

In some embodiments, D2 comprises an aryl substituted with halogen (e.g., fluoro) and sulfoxide. In some embodiments, D2 is an aryl substituted with fluoro and sulfoxide.

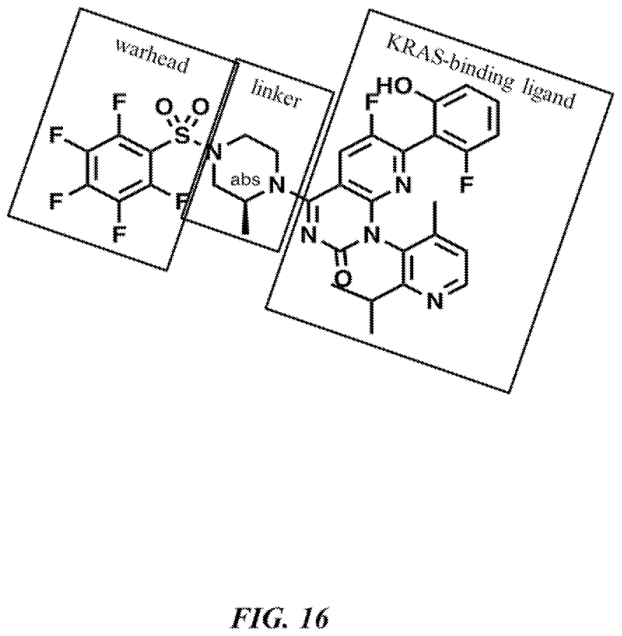

In some embodiments, provided herein is a compound (e.g., of Formula (I-A)), wherein the compound (e.g., of Formula (I-A)) has a warhead (e.g., D2) of any one of the compounds of Table 8, such as wherein the warhead (e.g., D2) is the part of the compound identified with a box around it in FIG. 16.

In some embodiments, D2 comprises one or more activating group, such as an activating group that binds to, disrupts, and/or modifies KRAS either alone or in combination with L (e.g., when D2 is amino (e.g., tertiary amine (e.g., >N—)) and L is substituted or unsubstituted pipirizinyl or substituted or unsubstituted azetidinyl).

In some embodiments, D1 is a radical of a KRAS-binding ligand, such as a KRAS-binding ligand provided elsewhere herein (e.g., G or G¹).

In some embodiments, D1 has a structure represented by Formula (II):

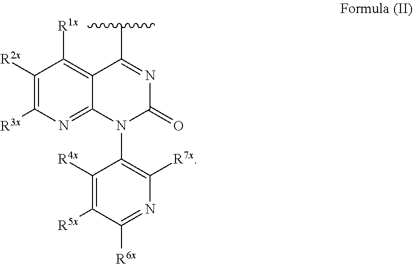

In some embodiments, R^{1x}, R^{2x}, R^{3x}, R^{4x}, R^{5x}, R^{6x}, and R^{7x} are each independently selected from the group consisting of hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl.

In some embodiments, R^{1x}, R^{2x}, R^{3x}, R^{4x}, R^{5x}, R^{6x}, and R^{7x} are each independently selected from the group consisting of hydrogen, halogen, and substituted or unsubstituted alkyl.

In some embodiments, R^{1x} is hydrogen.

In some embodiments, R^{2x} is halogen. In some embodiments, R^{2x} is fluoro.

In some embodiments, R^{3x} is halogen. In some embodiments, R^{3x} is chloro.

In some embodiments, R^{3x} is substituted aryl. In some embodiments, R^{3x} is aryl substituted with halogen. In some embodiments, R^{3x} is aryl substituted with hydroxy. In some embodiments, R^{3x} is aryl substituted with halogen and hydroxy. In some embodiments, R^{3x} is aryl substituted with fluoro and hydroxy.

In some embodiments, R^{4x} is substituted or unsubstituted alkyl. In some embodiments, R^{4x} is methyl.

In some embodiments, R^{5x} is hydrogen.

In some embodiments, R^{6x} is hydrogen.

In some embodiments, R^{7x} is substituted or unsubstituted alkyl. In some embodiments, R^{7x} is isopropyl.

In some embodiments, R^{1x} is hydrogen, R^{2x} is fluoro, R^{3x} is aryl substituted with fluoro and hydroxy, R^{4x} is methyl, R^{5x} is hydrogen, R^{6x} is hydrogen, and R^{7x} is isopropyl.

In some embodiments, D1 has a structure represented by Formula (II-A):

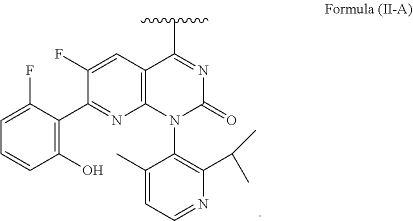 [SMILES: *c1nc(=O)n(-c2c(C)ccnc2C(C)C)c2nc(-c3c(O)cccc3F)c(F)cc12]

In some embodiments, R^{1x} is hydrogen, R^{2x} is fluoro, R^{3x} is chloro, R^{4x} is methyl, R^{5x} is hydrogen, R^{6x} is hydrogen, and R^{7x} is isopropyl.

In some embodiments, D1 has a structure represented by Formula (II-B):

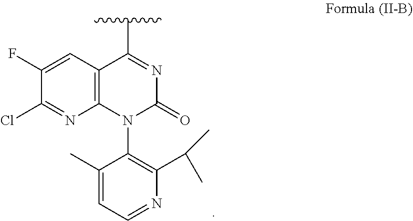 [SMILES: *c1nc(=O)n(-c2c(C)ccnc2C(C)C)c2nc(Cl)c(F)cc12]

In some embodiments, D1 has a structure represented by Formula (III):

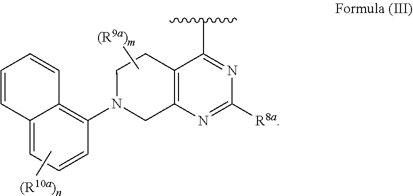

In some embodiments, R^{8a} is hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R^{9a} is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R^{10a} is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, m is 0-6. In some embodiments, n is 0-7.

In some embodiments, R^{8a} is hydrogen.

In some embodiments, n is 0 or 1.

In some embodiments, m is 0.

In some embodiments, R^{10a} is halogen, hydroxy, or unsubstituted alkoxy. In some embodiments, R^{10a} is chloro, hydroxy, or OMe. In some embodiments, R^{10a} is chloro. In some embodiments, R^{10a} is hydroxy. In some embodiments, R^{10a} is OMe.

In some embodiments, R^{8a} is hydrogen, m is 0, n is 1, and R^{10a} is chloro.

In some embodiments, D1 has a structure represented by Formula (III-A):

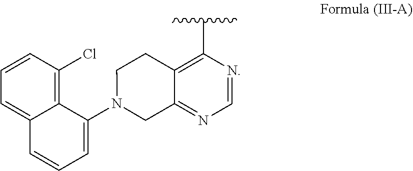 [SMILES: *c1ncnc2c1CCN(c1cccc3cccc(Cl)c13)C2]

In some embodiments, R^{8a} is hydrogen, m is 0, and n is 0.

In some embodiments, D1 has a structure represented by Formula (III-B):

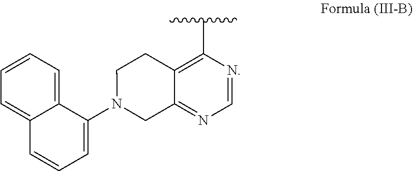 [SMILES: *c1ncnc2c1CCN(c1cccc3ccccc13)C2]

In some embodiments, R^{8a} is hydrogen, m is 0, n is 1, and R^{10a} is hydroxy.

In some embodiments, D1 has a structure represented by Formula (III-C):

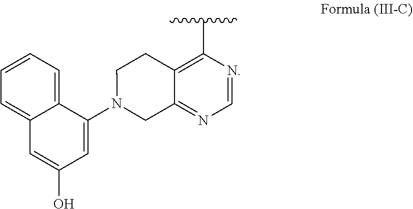 [SMILES: *c1ncnc2c1CCN(c1cc(O)cc3ccccc13)C2]

In some embodiments, R^{8a} is hydrogen, m is 0, n is 1, and R^{10a} is —OMe.

In some embodiments, D1 has a structure represented by Formula (III-D):

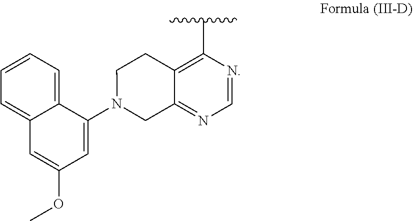 [SMILES: *c1ncnc2c1CCN(c1cc(OC)cc3ccccc13)C2]

In some embodiments, D1 has a structure represented by Formula (IV):

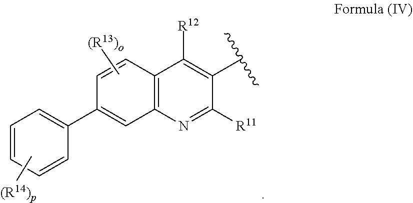

In some embodiments, R¹¹ and R¹² are each independently hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R¹³ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R¹⁴ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, o is 0-3. In some embodiments, p is 0-5.

In some embodiments, R¹¹ is hydrogen.

In some embodiments, R¹² is hydrogen.

In some embodiments, o is 0 or 1.

In some embodiments, R¹³ is halogen (e.g., chloro). In some embodiments, R¹³ is chloro.

In some embodiments, each R¹⁴ is independently alkyl.

In some embodiments, each R¹⁴ is independently unsubstituted alkyl. In some embodiments, p is 2 and each R¹⁴ is methyl.

In some embodiments, R¹¹ is hydrogen, R¹² is hydrogen, o is 0, p is 2, and each R¹⁴ is methyl.

In some embodiments, D1 has a structure represented by Formula (IV-A):

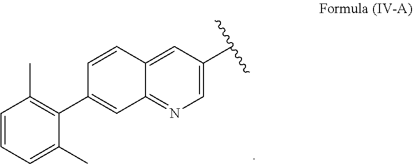 [SMILES: *c1cnc2cc(-c3c(C)cccc3C)ccc2c1]

In some embodiments, R¹¹ is hydrogen, R¹² is hydrogen, o is 1, R¹³ is chloro, p is 2, and each R¹⁴ is independently methyl.

In some embodiments, D1 has a structure represented by Formula (IV-B):

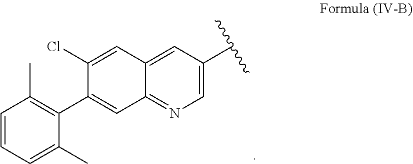 [SMILES: *c1cnc2cc(-c3c(C)cccc3C)c(Cl)cc2c1]

In some embodiments, D1 has a structure represented by Formula (V):

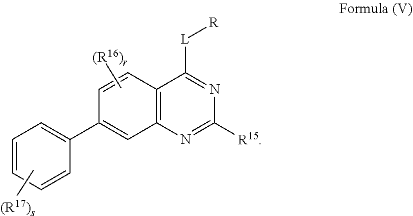

In some embodiments, R¹⁵ is hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R¹⁶ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R¹⁷ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, r is 0-3. In some embodiments, s is 0-5.

In some embodiments, R¹⁵ is hydrogen.

In some embodiments, r is 1 or 2.

In some embodiments, each R¹⁶ is independently halogen. In some embodiments, each R¹⁶ is independently chloro or fluoro.

In some embodiments, r is 1 and R¹⁶ is fluoro.

In some embodiments, R¹⁷ is independently halogen (e.g., fluoro) or hydroxyl. In some embodiments, R¹⁷ is independently fluoro or hydroxyl.

In some embodiments, R¹⁵ is hydrogen, r is 1, R¹⁶ is chloro, s is 1, and R¹⁴ is fluoro.

In some embodiments, D1 has a structure represented by Formula (V-A):

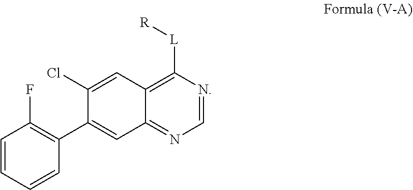

In some embodiments, D1 has a structure represented by Formula (VI):

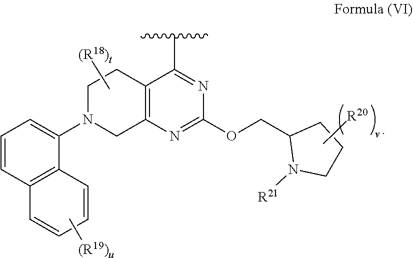

In some embodiments, each R¹⁸ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R¹⁹ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, each R²⁰ is independently selected from the group consisting of halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, R²¹ is hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl. In some embodiments, t is 0-6. In some embodiments, u is 0-7. In some embodiments, v is 0-7.

In some embodiments, t is 0.

In some embodiments, u is 1.

In some embodiments, R¹⁹ is halogen (e.g., chloro). In some embodiments, R¹⁹ is chloro.

In some embodiments, v is 0.

In some embodiments, R²¹ is unsubstituted alkyl (e.g., methyl). In some embodiments, R²¹ is methyl.

In some embodiments, t and v are 0, u is 1, R¹⁹ is chloro, and R²¹ is methyl.

In some embodiments, D1 has a structure represented by Formula (VI-A):

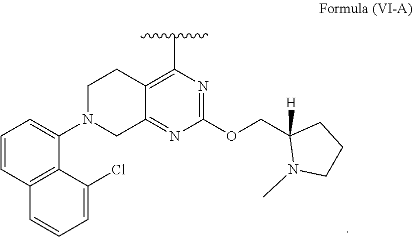 [SMILES: *c1nc(OC[C@@H]2CCCN2C)nc2c1CCN(c1cccc3cccc(Cl)c13)C2]

In some embodiments, D1 has a structure represented by Formula (VII):

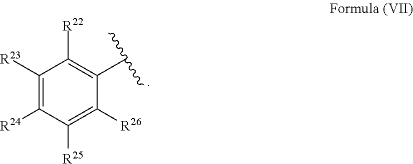

In some embodiments, R²², R²³, R²⁴, R²⁵, and R²⁶ are each independently selected from the group consisting of hydrogen, halogen, hydroxy, substituted or unsubstituted alkyl, substituted or unsubstituted heteroalkyl, substituted or unsubstituted alkoxy, substituted or unsubstituted aryl, and substituted or unsubstituted heteroaryl.

In some embodiments, R²² is hydrogen, hydroxy, or substituted or unsubstituted heteroalkyl. In some embodiments, R²² is hydrogen. In some embodiments, R²² is hydroxy. In some embodiments, R²² is substituted or unsubstituted heteroalkyl.

In some embodiments, R²³ is hydrogen or halogen. In some embodiments, R²³ is hydrogen. In some embodiments, R²³ is halogen. In some embodiments, R²³ is hydrogen or chloro.

In some embodiments, R²⁴ is hydrogen or halogen. In some embodiments, R²⁴ is hydrogen. In some embodiments, R²⁴ is halogen. In some embodiments, R²⁴ is chloro or bromo. In some embodiments, R²⁴ is hydrogen, chloro or bromo.

In some embodiments, R²⁵ is hydrogen, halogen, or substituted alkyl. In some embodiments, R²⁵ is hydrogen. In some embodiments, R²⁵ is halogen. In some embodiments, R²⁵ is chloro. In some embodiments, R²⁵ is substituted alkyl.

In some embodiments, R²⁶ is hydrogen, halogen (e.g., chloro), unsubstituted alkoxy, or substituted alkyl. In some embodiments, R²⁶ is hydrogen. In some embodiments, R²⁶ is halogen. In some embodiments, R²⁶ is chloro. In some embodiments, R²⁶ is unsubstituted alkoxy. In some embodiments, R²⁶ is substituted alkyl.

In some embodiments, R²² is hydrogen, R²³ is hydrogen, R²⁴ is chloro, R²⁵ is hydrogen, and R²⁶ is chloro.

In some embodiments, D1 has a structure represented by Formula (VII-A):

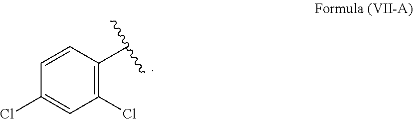 [SMILES: *c1ccc(Cl)cc1Cl]

In some embodiments, R²² is hydrogen, R²³ is chloro, R²⁴ is hydrogen, R²⁵ is hydrogen, and R²⁶ is chloro.

In some embodiments, D1 has a structure represented by Formula (VII-B):

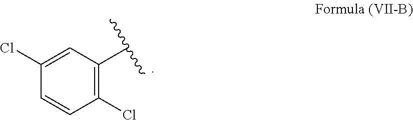 [SMILES: *c1cc(Cl)ccc1Cl]

In some embodiments, D1 has a structure represented in any of Tables 2-6. In some embodiments, D1 has a structure represented in any of Tables 2-6 and L is a bond.

Unless stated specifically otherwise herein, each instance of radical indicates that a hydrogen (i.e., a hydrogen radical (H·)) is removed from a free form of a compound provided herein, such as any KRAS-binding ligand (e.g., D1) or warhead (e.g., D2) described herein. In some instances, the removal of the hydrogen radical from the compound provided herein, such as any KRAS-binding ligand (e.g., D1) or warhead (e.g., D2) described herein, provides a radical of a KRAS-binding ligand or a warhead that is taken together with any point of a linker provided herein (e.g., L, L¹, or L²) to form a bond (e.g., between the linker and the radical of the KRAS-binding ligand or the warhead). In some instances, a carbon atom (e.g., of any KRAS-binding ligand (e.g., a substituted heterocycle or a substituted carbocycle) or warhead described herein) loses an H· to become a point of attachment to L. In some instances, >NH loses an H· to become >N-(point of attachment), such as >N-L-D1, >N-L-D2, >N-D1, or >N-D2. In some instances, —OH loses an H· to become —O-(point of attachment), such as —O-L-D1, —O-L-D2, —O-D1, or —O-D2. In some instances, —S(═O)_{g}H (where g is 1 or 2) loses an H· to become —S(═O)_{g}-(point of attachment), such as —S(═O)_{g}-L-D1, —S(═O)_{g}-L-D2, —S(═O)_{g}-D1, or —S(═O)_{g}-D2. In some instances, the linker is a bond. In some instances, D1-L- is a KRAS-binding ligand.

In some embodiments, provided herein is a compound (e.g., of Formula (I-A)), wherein the compound (e.g., of Formula (I-A)) comprises a KRAS-binding ligand (e.g., D1) of any one of the compounds of Table 8, such as wherein the KRAS-binding ligand (e.g., D1) is the part of the compound identified with a box around it in FIG. 16.

In some embodiments, D1 (a KRAS-binding ligand provided herein) binds to, disrupts, and/or modifies KRAS either alone or in combination with D2 (a warhead radical provided herein) and/or L (a linker provided herein). In some instances, D1 has activity such that a compound provided herein binds to, disrupts, and/or modifies KRAS (e.g., KRAS G12C) at a concentration of about 10 mM or less (e.g., 500 uM or less, 100 uM or less, or 10 uM or less). In some instances, D1 has activity such that a compound provided herein has Ki to KRAS (e.g., KRAS G12C) of about 250 uM or less (e.g., about 50 uM or less or about 1 uM or less).

In some embodiments, L is a linker.

In some embodiments, the linker is a non-releasable linker.

In some instances, the linker does not decompose (e.g., hydrolyze) or release the warhead radical (or a free form thereof), the radical of the KRAS-binding ligand (or a free form thereof), or any other portion of the compound (e.g., a radical of any Formula provided herein) (or a free form thereof)).

In some embodiments, the linker comprises one or more linker group, each linker group being independently selected from the group consisting of a bond, —O—, (substituted or unsubstituted) amino (e.g., —NH—, —NCH₃—, methylamine, or dimethylamine), substituted or unsubstituted (e.g., acyclic (e.g., straight or branched) or cyclic) alkyl(ene) (e.g., straight unsubstituted alkyl (e.g., methylene, ethylene, or the like) or straight alkylene substituted with oxo, amino (e.g., —NH—, —NCH₃—, or methylamine), heterocyclyl (e.g., (methylene) piperidinyl or piperazinyl), and/or aryl (e.g., (methylene) phenyl)), substituted or unsubstituted (e.g., acyclic (e.g., straight or branched) or cyclic) heteroalkyl(ene) (e.g., cyclic heteroalkylene (e.g., piperazinyl or 1,4-diazepanyl) substituted with alkyl (e.g., methyl) and/or oxo, or straight heteroalkylene substituted with oxo, heterocyclyl (e.g., azetidinyl, pyrrolidinyl, piperidinyl, or piperazinyl), aryl (e.g., phenyl), and/or heteroaryl (e.g., substituted or unsubstituted oxazolyl, pyridinyl, imidazolyl, or pyrazolyl)), substituted or unsubstituted alkoxy (e.g., unsubstituted alkoxy (e.g., methoxy, ethoxy, or the like) or alkoxy substituted with oxo, amino (e.g., —NH—, —NCH₃—, substituted (e.g., methylamine) or —NH-azetidinyl-), cycloalkyl (e.g., cyclobutyl substituted with amino (e.g., —NH—, —NCH₃—, or methylamine)), and/or heterocyclyl (e.g., azetidinyl or pyrrolidinyl)), and substituted or unsubstituted aryl (e.g., aryl substituted with alkyl (e.g., methyl)).

In some embodiments, the linker comprises one or more linker group, each linker group being independently selected from the group consisting of —O—, substituted or unsubstituted amino, substituted or unsubstituted alkylene, substituted or unsubstituted heteroalkylene, and substituted or unsubstituted alkoxy.

In some embodiments, the linker comprises one or more linker group, each linker group being independently selected from the group consisting of —O—, substituted or unsubstituted amino and substituted or unsubstituted heteroalkylene. In some embodiments, the linker comprises one or more linker group, each linker group being independently selected from the group consisting of —O—, substituted or unsubstituted amino and substituted or unsubstituted acyclic (e.g., straight or branched) heteroalkylene. In some embodiments, the linker comprises one or more linker group, each linker group being independently selected from the group consisting of —O—, substituted or unsubstituted amino and substituted or unsubstituted cyclic heteroalkylene. In some embodiments, the linker comprises one or more linker group, each linker group being independently selected from the group consisting of —O—, substituted or unsubstituted amino and substituted or unsubstituted heterocyclyl.

In some embodiments, the linker comprises one or more linker group, each linker group being independently selected from the group consisting of substituted or unsubstituted amino and substituted or unsubstituted heteroalkylene. In some embodiments, the linker comprises one or more linker group, each linker group being independently selected from the group consisting of substituted or unsubstituted amino and substituted or unsubstituted acyclic (e.g., straight or branched) heteroalkylene. In some embodiments, the linker comprises one or more linker group, each linker group being independently selected from the group consisting of substituted or unsubstituted amino and substituted or unsubstituted cyclic heteroalkylene. In some embodiments, the linker comprises one or more linker group, each linker group being independently selected from the group consisting of substituted or unsubstituted amino and substituted or unsubstituted heterocyclyl.

In some embodiments, the linker comprises —O—.

In some embodiments, the linker comprises substituted or unsubstituted amino.

In some embodiments, the linker comprises substituted or unsubstituted alkylene. In some embodiments, the linker comprises substituted or unsubstituted acyclic (e.g., straight or branched) alkylene. In some embodiments, the linker comprises substituted or unsubstituted cyclic alkylene.

In some embodiments, the linker comprises substituted or unsubstituted heteroalkylene. In some embodiments, the linker comprises substituted or unsubstituted acyclic (e.g., straight or branched) heteroalkylene. In some embodiments, the linker comprises substituted or unsubstituted cyclic heteroalkylene (e.g., heterocycyl).

In some embodiments, the linker comprises substituted or unsubstituted heterocycyl.

In some embodiments, the linker comprises substituted or unsubstituted alkoxy.

In some embodiments, L is a bond, substituted or unsubstituted alkylene, substituted or unsubstituted heteroalkylene, or substituted or unsubstituted amino.

In some embodiments, L is a bond, substituted or unsubstituted alkylene, substituted or unsubstituted pipirizinyl, substituted or unsubstituted azetidinyl, or substituted or unsubstituted amino.

In some embodiments, L is a bond.

In some embodiments, L is substituted or unsubstituted alkylene. In some embodiments, L is methylene, alkyl substituted with substituted or unsubstituted pipirizinyl. In some embodiments, L is methylene. In some embodiments, L is alkyl substituted with substituted or unsubstituted pipirizinyl. In some embodiments, L is alkyl substituted with substituted pipirizinyl. In some embodiments, L is alkyl substituted with unsubstituted pipirizinyl.

In some embodiments, L is substituted or unsubstituted heteroalkylene. In some embodiments, L is substituted or unsubstituted heterocyclyl. In some embodiments, L is unsubstituted pipirizinyl, substituted pipirizinyl, unsubstituted azetidinyl, or azetidinyl substituted with amino. In some embodiments, L is unsubstituted pipirizinyl. In some embodiments, L is substituted pipirizinyl. In some embodiments, L is pipirizinyl substituted with methyl. In some embodiments, L is unsubstituted azetidinyl. In some embodiments, L is azetidinyl substituted with amino.

In some embodiments, L is substituted or unsubstituted amino. In some embodiments, L is —NH—, amino substituted with alkyl. In some embodiments, L is —NH—. In some embodiments, L is amino substituted with alkyl. In some embodiments, L is —CH₂NH— or —CH₂CH₂NH—. In some embodiments, L is amino substituted with azetidinyl.

In some embodiments, provided herein is a compound (e.g., of Formula (I-A)), wherein the compound (e.g., of Formula (I-A)) comprises a linker (e.g., L) of any one of the compounds of Table 8, such as wherein the linker (e.g., L) is the part of the compound identified with a box around it in FIG. 16.

In some instances, such as when L is substituted or unsubstituted heterocyclyl, L is part of D1 and/or D2.

Provided in some embodiments herein is a compound of Formula (I′), or a salt or solvate or tautomer or regioisomer thereof.

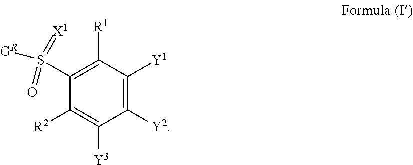

In some embodiments, G^{R} is substituted or unsubstituted alkyl (e.g., haloalkyl), substituted or unsubstituted heteroalkyl, —N(R⁵)₂, —N(R⁵)G, or G. In some embodiments, R⁵ is hydrogen, —CN, substituted or unsubstituted alkyl (e.g., alkyl substituted with one or more substituent, each substitutent being independently selected from the group consisting of oxo, hydroxy, alkoxy, heteroalkyl, and amino (e.g., —C(O)R⁶, —C(═O)R⁶, or —C(O)NR³R⁶. In some embodiments, each R⁶ is independently hydrogen, substituted or unsubstituted alkyl, or substituted or unsubstituted heteroalkyl)), substituted or unsubstituted heteroalkyl, substituted or unsubstituted aryl, or substituted or unsubstituted heteroaryl. In some embodiments, X¹ is absent, O, or NR. In some embodiments, R is hydrogen, R⁷, substituted or unsubstituted alkyl, substituted or unsubstituted heteroalkyl, substituted or unsubstituted aryl, substituted or unsubstituted heteroaryl, or G. In some embodiments, each Y¹, Y², and Y is independently hydrogen, halo, substituted or unsubstituted alkyl (e.g., haloalkyl) (e.g., with at least two of Y¹, Y², and Y³ being halo or haloalkyl, such as fluoroalkyl, e.g., at least one of Y¹, Y², and Y³ (e.g., Y²) being halo (e.g., Y¹, Y², Y³ all being F)), or G. In some embodiments, R¹ is hydrogen, halogen, or R⁷. In some embodiments, R² is hydrogen, halogen, or R⁷. In some embodiments, each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³—OC(═O)N(R³)₂, —C(═O)N(R³)₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R³ is hydrogen, substituted or unsubstituted alkyl, -L¹R⁴, —C(═O)L¹R⁴, —C(═O)OL¹R⁴, or —C(═O)NR⁴L¹R⁴, wherein each L is independently substituted or unsubstituted alkyl, substituted or unsubstituted heteroalkyl, substituted or unsubstituted aryl, or substituted or unsubstituted heteroaryl; and each R⁴ is independently hydrogen, substituted or unsubstituted cycloalkyl, substituted or unsubstituted heterocycloalkyl, substituted or unsubstituted aryl, or substituted or unsubstituted heteroaryl. In some embodiments, x is 0, 1, or 2. In some embodiments, G is or comprises a KRAS-binding ligand, is or comprises (e.g., unsaturated) carbocycle, is or comprises (e.g., unsaturated) heterocycle, or is -L²-G¹. In some embodiments, L² is a linker (e.g., —O— or —NR⁵—). In some embodiments, G¹ is hydrogen or an organic residue (e.g., is or comprises a KRAS-binding ligand, is or comprises (e.g., unsaturated) carbocycle, or is or comprises (e.g., unsaturated) heterocycle).

In some embodiments, such as when R¹ is halo, R² and Y¹—Y³ are each independently halogen. In some embodiments, such as when R¹ is halo, R² and Y¹—Y³ are each fluoro. In some embodiments, such as when R¹ is fluoro, R² and Y¹—Y³ are each fluoro. In some embodiments, R¹, R², and Y¹—Y³ are fluoro.

In some embodiments, provided herein is a compound of Formula (I-B), or a salt or solvate or tautomer or regioisomer thereof.

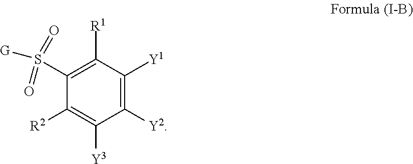

In some embodiments, G is or comprises a KRAS-binding ligand (e.g., such as D1 described herein).

In some embodiments, G is -L²-G¹. In some embodiments, L² is a linker (e.g., any linker described elsewhere herein). In some embodiments, the linker is substituted or unsubstituted alkyl (e.g., alkyl substituted with pipirizinyl or piperidinyl), substituted or unsubstituted pipirizinyl, unsubstituted or substituted piperidinyl, substituted or unsubstituted azetidinyl (e.g., azetidinyl substituted with amino), or substituted or unsubstituted amino (e.g., —NH—, amino substituted with alkyl (e.g., —CH₂NH— or —CH₂CH₂NH—), or amino substituted with azetidinyl). In some embodiments, G¹ is a KRAS-binding ligand that binds to with KRAS (e.g., KRAS G12C)).

In some embodiments, Y¹, Y², and Y³ are each independently hydrogen or halogen. In some embodiments, Y¹, Y², and Y³ are each independently hydrogen or fluoro. In some embodiments, Y¹, Y², and Y³ are fluoro. In some embodiments, Y¹ and Y² are fluoro and Y³ is hydrogen.

In some embodiments, R¹ is halogen. In some embodiments, R¹ is fluoro.

In some embodiments, R² is halogen, —OR³, or substituted or unsubstituted alkyl. In some embodiments, R² is fluoro, —OR³, or haloalkyl. In some embodiments, R is fluoro. In some embodiments, R³ is hydrogen or substituted or unsubstituted alkyl. In some embodiments, R³ is hydrogen or haloalkyl.

In some embodiments, R¹ and R² are halogen. In some embodiments, R¹ and R² are fluoro.

In some embodiments, such as when R¹ is hydrogen or halogen, one of Y¹, Y², or Y³ is G. In some embodiments, such as when R¹ is hydrogen or fluoro, one of Y¹, Y², or Y³ is G. In some embodiments, such as when R¹ is hydrogen, one of Y¹, Y², or Y³ is G. In some embodiments, such as when R¹ is fluoro, one of Y¹, Y², or Y³ is G. In some embodiments, such as when R¹ is hydrogen or fluoro, Y¹ is G. In some embodiments, such as when R¹ is hydrogen or fluoro, Y² is G. In some embodiments, such as when R¹ is hydrogen or fluoro, Y³ is G. In some embodiments, R¹ is hydrogen or fluoro and Y¹ is G. In some embodiments, is hydrogen or fluoro and Y² is G. In some embodiments, R¹ is hydrogen or fluoro and Y³ is G.

In some embodiments, such as when R¹ is hydrogen or halogen, either G^{R} or Y² is G. In some embodiments, such as when R¹ is hydrogen or fluoro, either G^{R} or Y² is G. In some embodiments, such as when R¹ is hydrogen, either G^{R} or Y² is G. In some embodiments, such as when R¹ is fluoro, either G^{R} or Y² is G. In some embodiments, such as when R¹ is hydrogen or fluoro, G^{R} is G. In some embodiments, such as when R¹ is hydrogen or fluoro, Y² is G. In some embodiments, R¹ is hydrogen or fluoro and G^{R} is G. In some embodiments, R¹ is hydrogen or fluoro and Y² is G.

In some embodiments, provided herein is a compound of Formula (I-C), or a salt or solvate or tautomer or regioisomer thereof:

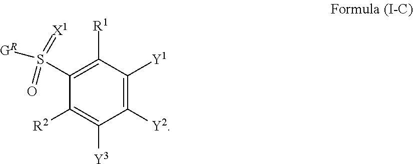

In some embodiments, G^{R} is substituted or unsubstituted alkyl.

In some embodiments, X¹ is absent or O. In some embodiments, X¹ is absent. In some embodiments, X¹ is O. In some embodiments X¹ and the O of O═S<=X¹ are absent.

In some embodiments, Y¹ and Y³ are each independently halogen. In some embodiments, Y¹ and Y³ are each fluoro.

In some embodiments, Y² is halogen or G. In some embodiments, Y² is fluoro. In some embodiments, Y² is G.

In some embodiments, R¹ is halogen. R¹ is fluoro.

In some embodiments, R² is halogen or G. In some embodiments, R is fluoro. In some embodiments, R² is G.

In some embodiments, G is or comprises a KRAS-binding ligand (e.g., such as D1 described herein).

In some embodiments, G is -L²-G¹. In some embodiments, L² is a linker (e.g., any linker described elsewhere herein). In some embodiments, the linker is substituted or unsubstituted alkyl (e.g., alkyl substituted with pipirizinyl or piperidinyl), substituted or unsubstituted pipirizinyl, substituted or unsubstituted piperidinyl, substituted or unsubstituted azetidinyl (e.g., azetidinyl substituted with amino), or substituted or unsubstituted amino (e.g., —NH—, amino substituted with alkyl (e.g., —CH₂NH— or —CH₂CH₂NH—), or amino substituted with azetidinyl). In some embodiments, G¹ is a KRAS-binding ligand that binds to with KRAS (e.g., KRAS G12C)).

In some embodiments, either Y² or R² is G.

In some embodiments, X¹ is absent.

In some embodiments, X¹ is O.

In some embodiments, R¹ is fluoro.

In some embodiments, R² is fluoro.

In some embodiments, Y¹ and Y³ are fluoro.

In some embodiments, R¹, Y¹, and Y³ are fluoro.

In some embodiments, R¹, R², Y¹, and Y³ are fluoro.

In some embodiments, R¹, Y¹, and Y³ are fluoro and G^{R} is G.

In some embodiments, R¹, Y¹, and Y³ are fluoro, R² is R⁷, and G^{R} is G. In some embodiments, R¹, Y¹, and Y³ are fluoro, R² is halogen, substituted or unsubstituted alkyl, or —OR³ (e.g., R³ being hydrogen or substituted or unsubstituted alkyl), and G^{R} is G. In some embodiments, R¹, Y¹, and Y³ are fluoro, R² is fluoro, haloalkyl, or —O-haloaklyl, and G^{R} is G. In some embodiments, R¹, Y¹, and Y³ are fluoro, R² is fluoro, and G^{R} is G. In some embodiments, R¹, Y¹, and Y³ are fluoro, R² is haloalkyl, and G^{R} is G. In some embodiments, R¹, Y¹, and Y³ are fluoro, R² is —O-haloalkyl, and G^{R} is G.

In some embodiments, R¹, Y¹, and Y³ are fluoro and R² is G.

In some embodiments, R¹, Y¹, and Y³ are fluoro, G^{R} is substituted or unsubstituted alkyl, and R² is G.

In some embodiments, G or G¹ has or comprises a structure of any one of any one of Formula (II), Formula (II-A), Formula (II-B), Formula (III), Formula (III-A), Formula (III-B), Formula (III-C), Formula (III-D), Formula (IV), Formula (IV-A), Formula (IV-B), Formula (V), Formula (V-A), Formula (VI), Formula (VI-A), Formula (VII), Formula (VII-A), or Formula (VII-B), or a structure provided in Table 2, Table 3, Table 4, Table 5, or Table 6.

In one aspect, provided herein is a compound, or pharmaceutically acceptable salt or solvate or tautomer or regioisomer thereof, having the structure of Formula (I):

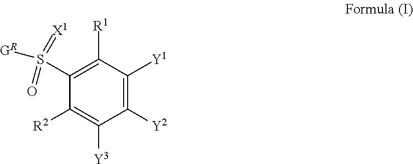

wherein,
G^{R} is alkyl, haloalkyl, heteroalkyl, —N(R⁵)₂, —N(R⁵)G, or G;
G is or comprises a KRAS-binding ligand, is or comprises (e.g., unsaturated) carbocycle, is or comprises (e.g., unsaturated) heterocycle, or is -L²-G¹, wherein L² is a linker (e.g., —O— or —NR⁵—), and G¹ is hydrogen or an organic residue (e.g., is or comprises a KRAS-binding ligand, is or comprises (e.g., unsaturated) carbocycle, or is or comprises (e.g., unsaturated) heterocycle);
X¹ is absent, O or NR;
each Y¹, Y², and Y³ is independently hydrogen, halo, alkyl, haloalkyl (e.g., with at least two Y being halo or haloalkyl, such as fluoroalkyl, e.g., at least one Y (e.g., Y²) being halo) (e.g., Y¹, Y², Y³ all being F), or G;
R is hydrogen, R⁷, alkyl, heteroalkyl, aryl, heteroaryl, or G;
R¹ is R⁷;
R² is hydrogen, halogen, or R⁷;
R³ is hydrogen, -L¹R⁴, —C(═O)L¹R⁴, —C(═O)OL¹R⁴, or —C(═O)NR⁴L¹R⁴, wherein each L¹ is independently alkyl, heteroalkyl, aryl, or heteroaryl; and each R⁴ is independently hydrogen, cycloalkyl, heterocycloalkyl, aryl, or heteroaryl;
R⁵ is hydrogen, —CN, —C(═O)R⁶, —C(═O)OR⁶, —C(═O)NR³R⁶, alkyl, heteroalkyl, aryl, or heteroaryl;
each R⁶ is independently hydrogen, alkyl, or heteroalkyl;
each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂, alkyl, heteroalkyl, cycloalkyl, heterocycloalkyl, aryl, or heteroaryl; and
x is 0, 1, or 2.

In some embodiments, the compound comprises only one G.

In some embodiments, G is or comprises (e.g., unsaturated) carbocycle, is or comprises (e.g., unsaturated) heterocycle, or is -L²-G¹ that it is a KRAS ligand. In some embodiments, Y¹, Y², and Y³ are not all F when X¹ is O, G^{R} is G, and G is L²G¹ (e.g., and L² is amino or —NR⁵). In some embodiments, G is -L²-G¹, wherein L² is a linker, and G¹ is an organic residue (e.g., is or comprises a KRAS-binding ligand, is or comprises (e.g., unsaturated) carbocycle, or is or comprises (e.g., unsaturated) heterocycle). In some embodiments, L² is a substituted or unsubstituted unsaturated alkylene (e.g., alkenylene or alkynylene), substituted or unsubstituted arylene, or substituted or unsubstituted heteroarylene, and G is an organic residue (e.g., is or comprises a KRAS-binding ligand). In some embodiments, L² is a bond, —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₈ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and G is an organic residue (e.g., is or comprises a KRAS-binding ligand).

In some embodiments, G is substituted or unsubstituted unsaturated carbocycle or substituted or unsubstituted unsaturated heterocycle, wherein G and R⁵ on a single N, if present, are optionally taken together to form a substituted or unsubstituted N-containing heterocycloalkyl. In some embodiments, G comprises one or more cyclic ring systems selected from substituted or unsubstituted unsaturated carbocycles and substituted or unsubstituted unsaturated heterocycles. In some embodiments, G comprises two or more cyclic ring systems selected from substituted or unsubstituted unsaturated carbocycles and substituted or unsubstituted unsaturated heterocycles. In some embodiments, G¹ comprises one or more cyclic ring systems selected from substituted or unsubstituted carbocycles and substituted or unsubstituted heterocycles. In some embodiments, G¹ comprises two or more cyclic ring systems selected from substituted or unsubstituted carbocycles and substituted or unsubstituted heterocycles. In some embodiments, the two or more cyclic ring systems are connected via a bond. In some embodiments, the two or more cyclic ring systems are connected via one or more linker and/or bond. In some embodiments, the linker is —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; and each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₅ cycloalkyl, substituted or unsubstituted C₂-C₇ heterocycloalkyl, substituted or unsubstituted aryl, or substituted or unsubstituted heteroaryl. In some embodiments, each R⁸ is independently hydrogen, substituted or unsubstituted C₁-C₄ alkyl, or substituted or unsubstituted C₁-C₄ heteroalkyl. In some embodiments, each R⁸ is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some embodiments, each R⁸ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some embodiments, each R⁸ is independently hydrogen, —CH₃, or —OCH₃. In some embodiments, the cyclic ring system comprises substituted or unsubstituted monocyclic aryl or substituted or unsubstituted monocyclic heteroaryl. In some embodiments, the cyclic ring system comprises substituted or unsubstituted bicyclic aryl or substituted or unsubstituted bicyclic heteroaryl.

In some embodiments, G or G¹ is or comprises a KRAS-binding ligand. In some embodiments, G or G¹ is or comprises a KRAS-binding ligand selected from Table 2. In some embodiments, G or G¹ is or comprises a KRAS-binding ligand selected from Table 3, Table 4, Table 5, and Table 6.

In some embodiments, R⁵ is hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some embodiments, R⁵ is hydrogen, —CN, —CH₃, —CF₃, or cyclopropyl. In some embodiments, R⁵ is hydrogen.

In some embodiments, X¹ is O, NH, or N(substituted or unsubstituted alkyl). In some embodiments, X¹ is O, NH, or N(alkyl). In some embodiments, X¹ is O, NH, or N(CH₃). In some embodiments, X¹ is O. In some embodiments, X¹ is NH or N(CH₃).

In some embodiments X¹ and the O of O═S<=X¹ are absent.

In some embodiments, when G^{R} is —S(═O)(═X¹)G, X¹ is O, then G is not: (R)-3-(4-phenoxyphenyl)-1-(1λ²-piperidin-3-yl)-1H-pyrazolo[3,4-d]pyrimidin-4-amine; 1-(2-(λ²-azaneyl)ethyl)-3-(4-phenoxyphenyl)-1H-pyrazolo[3,4-d]pyrimidin-4-amine; (R)-3-(4-phenoxyphenyl)-1-(λ²-pyrrolidin-3-yl)-1H-pyrazolo[3,4-d]pyrimidin-4-amine; 4-(λ²-azaneyl)-7H-pyrrolo[2,3-d]pyrimidine; N4-(3-(λ²-azaneyl)phenyl)-5-fluoro-N2-(4-(2-methoxyethoxy)phenyl)pyrimidine-2,4-diamine; 4-(λ²-azaneyl)-5-fluoro-N-(4-(2-methoxyethoxy)phenyl)pyrimidin-2-amine; or 3-(4-phenoxyphenyl)-1X²-pyrazolo[5,4-d]pyrimidin-4-amine. In some embodiments, wherein when G^{R} is —S(═O)(═X¹)N(R⁵)G, X¹ is O, then one or more of G and R⁵ is not or does not comprise: substituted or unsubstituted phenyl; substituted or unsubstituted benzyl; 1-naphthyl; pyridin-3-yl; pyridin-4-yl; 2-fluoropyridin-4-yl; or 2,6-difluoropyridin-3-yl.

In some cases, the present disclosure provides a compound or a salt or solvate or tautomer or regioisomer thereof selected from Table 1 or a salt or solvate or tautomer or regioisomer thereof.

In some cases, the present disclosure provides a pharmaceutically acceptable composition comprising a compound disclosed herein, or a salt or solvate or tautomer or regioisomer thereof, and one or more of pharmaceutically acceptable excipients.

In some cases, the present disclosure provides a KRAS protein or an active fragment thereof modified with a compound disclosed herein, or a salt or solvate or tautomer or regioisomer thereof, wherein the compound forms a covalent bond with a sulfur atom of a cysteine residue of the KRAS protein or an active fragment thereof (e.g., a polypeptide thereof).

In some cases, the present disclosure provides a method of modifying (e.g., attaching to and/or degrading) KRAS protein or an active fragment thereof with a compound, comprising contacting the polypeptide with a compound disclosed herein, or a salt or solvate or tautomer or regioisomer thereof, to form a covalent bond with a sulfur atom of a cysteine residue of the KRAS protein or an active fragment thereof (e.g., polypeptide thereof).

In some cases, the present disclosure provides a method of binding a compound to KRAS protein or an active fragment thereof, comprising contacting the KRAS protein or an active fragment thereof (e.g., polypeptide thereof) with a compound disclosed herein, or a salt or solvate or tautomer or regioisomer thereof.

In some cases, the present disclosure provides a method of disrupting KRAS protein or an active fragment thereof (e.g. a function thereof), comprising contacting the KRAS protein or an active fragment thereof (e.g., polypeptide thereof) with a compound of any one of the preceding claims, or a salt or solvate or tautomer or regioisomer thereof.

In some embodiments, G in Formula (I) is -L²-G¹, wherein L² is a >C═X, substituted or unsubstituted unsaturated alkylene (e.g., alkenylene or alkynylene, such as with an unsaturated carbon alpha to the N-atom of Formula (I)), substituted or unsubstituted arylene, or substituted or unsubstituted heteroarylene, wherein X is O, S, or NR³, and G¹ is an organic residue (e.g., a natural ligand of KRAS protein such as GDP or GTP). In some embodiments, L² is a substituted or unsubstituted unsaturated alkylene (e.g., alkenylene or alkynylene, such as with an unsaturated carbon alpha to the N-atom of Formula (I)), substituted or unsubstituted arylene, or substituted or unsubstituted heteroarylene, and G¹ is an organic residue (e.g., a natural ligand of KRAS protein such as GDP or GTP).

In some embodiments, G in Formula (I) is substituted or unsubstituted unsaturated carbocycle or substituted or unsubstituted unsaturated heterocycle, wherein G and R⁵ on a single N are optionally taken together to form a substituted or unsubstituted heterocycloalkyl. In some embodiments, G and R⁵ are optionally taken together to form a substituted or unsubstituted heterocycloalkyl (or substituted or unsubstituted heteroaryl), such as wherein such substituted or unsubstituted heterocycloalkyl (or substituted or unsubstituted heteroaryl) is substituted or unsubstituted heterocycloalkyl-G¹ (or substituted or unsubstituted heteroaryl-G¹).

In some embodiments, G in Formula (I) comprises one or more cyclic ring systems selected from substituted or unsubstituted unsaturated carbocycles and substituted or unsubstituted unsaturated heterocycles. In some embodiments, G in Formula (I) comprises two or more cyclic ring systems selected from substituted or unsubstituted unsaturated carbocycles and substituted or unsubstituted unsaturated heterocycles.

In some embodiments, G comprises two or more cyclic ring systems, such as wherein the ring systems are connected via a bond. In some embodiments, the two or more cyclic ring systems are connected via one or more linker and/or bond (e.g., wherein there are three cyclic ring systems, two of the ring systems are connected via bond, while the other two ring systems are connected by linker).

In some embodiments, G¹ comprises one or more cyclic ring systems selected from substituted or unsubstituted carbocycles and substituted or unsubstituted heterocycles. In some embodiments, G¹ comprises two or more cyclic ring systems selected from substituted or unsubstituted carbocycles and substituted or unsubstituted heterocycles.

In some embodiments, the two or more cyclic ring systems are connected via a bond. In some embodiments, the two or more cyclic ring systems are connected via one or more linker and/or bond.

In some embodiments, the linker is —O—, —NR⁷—, —N(R⁷)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁷—, —NR⁷C(═O)—, —OC(═O)NR⁷—, —NR⁷C(═O)O—, —NR⁷C(═O)NR⁷—, —NR⁷S(═O)₂—, —S(═O)₂NR⁷—, —C(═O)NR⁷S(═O)₂—, —S(═O)₂NR⁷C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁷—, —NR⁷—(C₁-C₄ alkylene)-, —(C₁-C₄ alkylene)-N(R⁷)₂⁺—, or —N(R⁷)₂⁺—(C₁-C₄ alkylene)-; and

each R⁷ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

In some embodiments, the cyclic ring system comprises substituted or unsubstituted monocyclic aryl or substituted or unsubstituted monocyclic heteroaryl. In some embodiments, the cyclic ring system comprises substituted or unsubstituted bicyclic aryl or substituted or unsubstituted bicyclic heteroaryl.

In some embodiments, R⁵ in Formula (I), is hydrogen, —CN, substituted or unsubstituted alkyl, or substituted or unsubstituted heteroalkyl. In some embodiments, R⁵ in Formula (I), is hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some embodiments, R⁵ in Formula (I), is hydrogen, —CN, —CH₃, —CF₃, or cyclopropyl. In some embodiments, R⁵ in Formula (I), is hydrogen. In some embodiments, R⁵ in Formula (I), is independently hydrogen, substituted or unsubstituted alkyl, or substituted or unsubstituted heteroalkyl. In some embodiments, R⁵ in Formula (I), is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some embodiments, R⁵ in Formula (I), is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some embodiments, R⁵ in Formula (I), is independently hydrogen, —CH₃, or —OCH₃. In some embodiments, R⁵ in Formula (I), is independently hydrogen or —CH₃. In some embodiments, R⁵ in Formula (I), is —CH₃.

In some embodiments, X¹ is O, NH, or N(substituted or unsubstituted alkyl). In some embodiments, X¹ is O, NH, or N(unsubstituted alkyl). In some embodiments, X¹ is O, NH, or N(CH₃). In some embodiments, X¹ is O. In some embodiments, X¹ is NH or N(CH₃).

In some embodiments, provided herein is a compound of Formula (I), such as described herein, with the exception that the G of Formula (I) is R⁵ and the X¹ of Formula (I) is NG, wherein G and R⁵ are as described in Formula (I). In other words, in certain embodiments, —S(═O)(═X¹)NR⁵G is substituted with —S(═O)(=NG)NR⁵₂.

In some embodiments, provided herein is a compound of Formula (I), such as described herein, with the exception that the G of Formula (I) is R⁵ and the R¹ of Formula (I) is R^{1a}-G, wherein G and R⁵ are as described in Formula (I) and R^{1a} is a bond or a divalent radical of R¹. In other words, in certain embodiments,

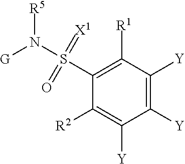

is substituted with

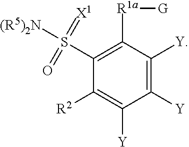

In some embodiments, provided herein is a compound of Formula (I), such as described herein, with the exception that the G of Formula (I) is R⁵ and the R² of Formula (I) is R^{2a}—G, wherein G and R⁵ are as described in Formula (I) and R^{2a} is a bond or a divalent radical of R². In other words, in certain embodiments,

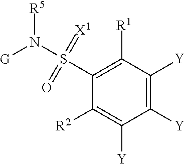

is substituted with

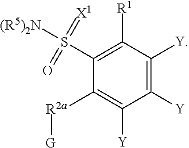

In some embodiments, each Y¹, Y², and Y³ is independently halo or haloalkyl. In some embodiments, each Y is independently halo. In some embodiments, each Y is independently F or Cl. In some embodiments, each Y is F. In some embodiments, each Y is Cl.

In some embodiments, R¹ is —CN, —OR³, —SR³, —N(R³)₂, —C(═O)OR³, —C(═O)N(R³)₂, -substituted or unsubstituted C₁-C₆ alkyl, substituted or unsubstituted C₁-C₆ haloalkyl, substituted or unsubstituted C₁-C₆ heteroalkyl, substituted or unsubstituted C₂-C₇ heterocycloalkyl, substituted or unsubstituted aryl, or substituted or unsubstituted heteroaryl. In some embodiments, R¹ is —CN, —OR³, —SR³, —N(R³)₂, —C(═O)OR³, -substituted or unsubstituted C₁-C₆ alkyl, substituted or unsubstituted C₁-C₆ haloalkyl, substituted or unsubstituted C₁-C₆ heteroalkyl, or substituted or unsubstituted aryl. In some embodiments, R¹ is —CN, —OR³, —SR³, —N(R³)₂, —C(═O)OR³, -substituted or unsubstituted C₁-C₆ alkyl, substituted or unsubstituted C₁-C₆ haloalkyl, substituted or unsubstituted C₁-C₆ heteroalkyl, or substituted or unsubstituted phenyl. In some embodiments, R¹ is —CN, —OR³, —SR³, —N(R³)₂, —C(═O)OR³, or —C(═O)N(R³)₂. In some embodiments, R¹ is —CN, —OR³, or —SR³. In some embodiments, R¹ is —CN. In some embodiments, R¹ is —OR³. In some embodiments, R¹ is —SR³. In some embodiments, R¹ is —N(R³)₂. In some embodiments, R¹ is —C(═O)OR³ or —C(═O)N(R³)₂.

In some embodiments, each R³ in R¹ is independently H, substituted or unsubstituted C₁-C₆ alkyl, substituted or unsubstituted C₁-C₆ haloalkyl, substituted or unsubstituted C₁-C₆ heteroalkyl, substituted or unsubstituted C₃-C₇ cycloalkyl, or substituted or unsubstituted C₂-C₇ heterocycloalkyl. In some embodiments, each R³ in R¹ is independently H, substituted or unsubstituted C₁-C₄ alkyl, substituted or unsubstituted C₁-C₄ haloalkyl, substituted or unsubstituted C₁-C₄ heteroalkyl, substituted or unsubstituted C₃-C₆ cycloalkyl, or substituted or unsubstituted C₂-C₅ heterocycloalkyl. In some embodiments, each R³ in R¹ is independently H, CH₃, CH₂CH₃, CH₂CH₂CH₃, CH(CH₃)₂, CH₂F, CHF₂, CF₃, CF₂CH₃, CH₂CF₃, cyclopropyl, cyclobutyl, benzyl, phenyl. In some embodiments, each R³ in R¹ is independently H. In some embodiments, R³ in R¹ is CH₃. In some embodiments, R³ in R¹ is CH₂CH₃. In some embodiments, R³ in R¹ is CH₂CH₂CH₃. In some embodiments, R³ in R¹ is CH(CH₃)₂. In some embodiments, R³ in R¹ is CH₂F. In some embodiments, R³ in R¹ is CHF₂. In some embodiments, R³ in R¹ is CF₃. In some embodiments, R³ in R¹ is CF₂CH₃. In some embodiments, R³ in R¹ is CH₂CF₃. In some embodiments, R³ in R¹ is cyclopropyl. In some embodiments, R³ in R¹ is cyclobutyl. In some embodiments, R³ in R¹ is benzyl. In some embodiments, R³ in R¹ is phenyl.

In some embodiments, the compound described herein has a structure provided in Table 1.

TABLE 1

| # | Compound structure and name |
|---|---|
| 1-5 | 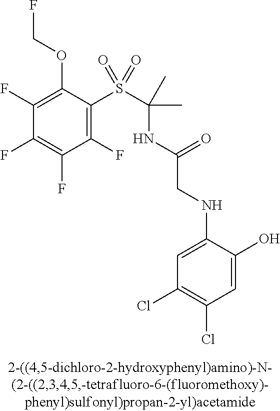 [SMILES: CC(C)(NC(=O)CNc1cc(Cl)c(Cl)cc1O)S(=O)(=O)c1c(F)c(F)c(F)c(F)c1OCF] |
| | 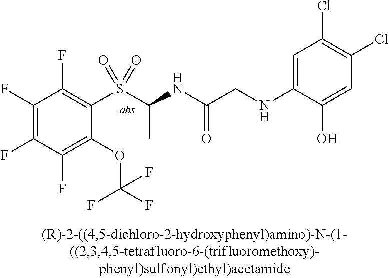 [SMILES: C[C@H](NC(=O)CNc1cc(Cl)c(Cl)cc1O)S(=O)(=O)c1c(F)c(F)c(F)c(F)c1OC(F)(F)F] |
| | 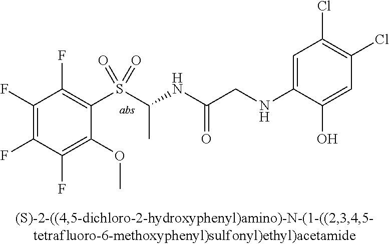 [SMILES: COc1c(F)c(F)c(F)c(F)c1S(=O)(=O)[C@@H](C)NC(=O)CNc1cc(Cl)c(Cl)cc1O] |
| | 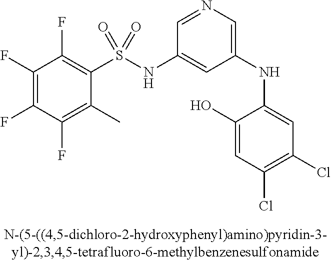 [SMILES: Cc1c(F)c(F)c(F)c(F)c1S(=O)(=O)Nc1cncc(Nc2cc(Cl)c(Cl)cc2O)c1] |
| | 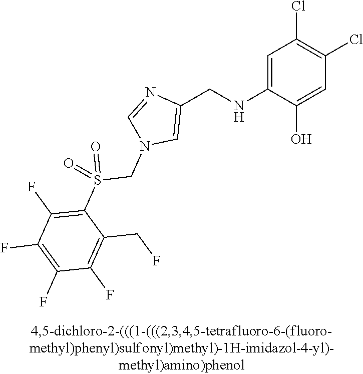 [SMILES: O=S(=O)(Cn1cnc(CNc2cc(Cl)c(Cl)cc2O)c1)c1c(F)c(F)c(F)c(F)c1CF] |
| 6-10 | 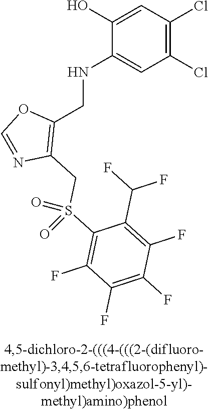 [SMILES: O=S(=O)(Cc1ncoc1CNc1cc(Cl)c(Cl)cc1O)c1c(F)c(F)c(F)c(F)c1C(F)F] |
| | 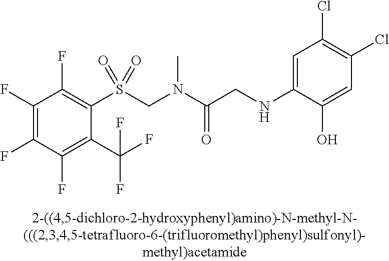 [SMILES: CN(CS(=O)(=O)c1c(F)c(F)c(F)c(F)c1C(F)(F)F)C(=O)CNc1cc(Cl)c(Cl)cc1O] |
| | 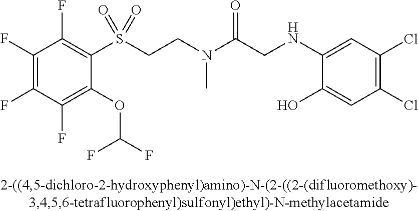 [SMILES: CN(CCS(=O)(=O)c1c(F)c(F)c(F)c(F)c1OC(F)F)C(=O)CNc1cc(Cl)c(Cl)cc1O] |
| | 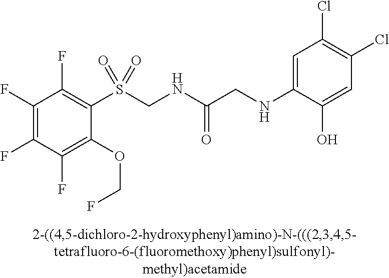 [SMILES: O=C(CNc1cc(Cl)c(Cl)cc1O)NCS(=O)(=O)c1c(F)c(F)c(F)c(F)c1OCF] |
| | 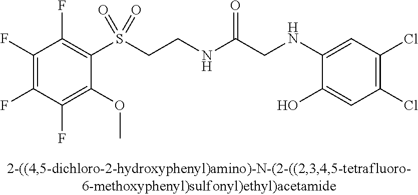 [SMILES: COc1c(F)c(F)c(F)c(F)c1S(=O)(=O)CCNC(=O)CNc1cc(Cl)c(Cl)cc1O] |
| 11-15 | 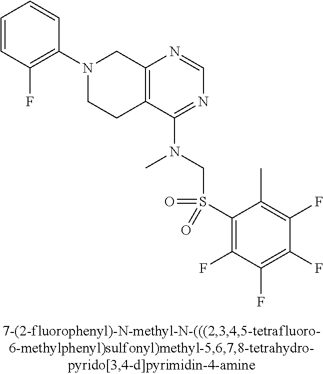 [SMILES: Cc1c(F)c(F)c(F)c(F)c1S(=O)(=O)CN(C)c1ncnc2c1CCN(c1ccccc1F)C2] |
| | 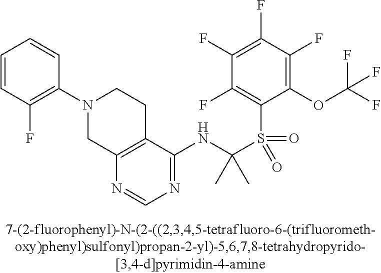 [SMILES: CC(C)(Nc1ncnc2c1CCN(c1ccccc1F)C2)S(=O)(=O)c1c(F)c(F)c(F)c(F)c1OC(F)(F)F] |
| | 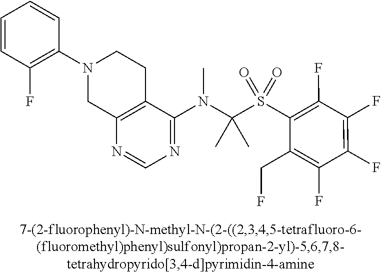 [SMILES: CN(c1ncnc2c1CCN(c1ccccc1F)C2)C(C)(C)S(=O)(=O)c1c(F)c(F)c(F)c(F)c1CF] |
| | 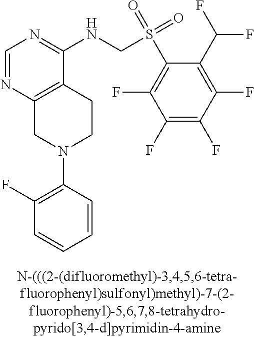 [SMILES: O=S(=O)(CNc1ncnc2c1CCN(c1ccccc1F)C2)c1c(F)c(F)c(F)c(F)c1C(F)F] |
| | 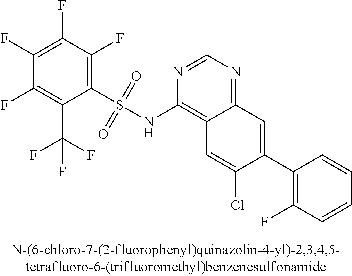 [SMILES: O=S(=O)(Nc1ncnc2cc(-c3ccccc3F)c(Cl)cc12)c1c(F)c(F)c(F)c(F)c1C(F)(F)F] |
| 16-20 | 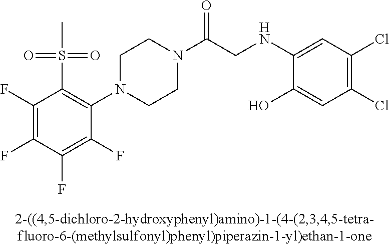 [SMILES: CS(=O)(=O)c1c(F)c(F)c(F)c(F)c1N1CCN(C(=O)CNc2cc(Cl)c(Cl)cc2O)CC1] |
| | 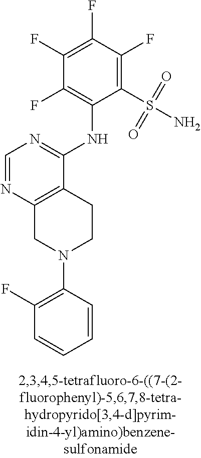 [SMILES: NS(=O)(=O)c1c(F)c(F)c(F)c(F)c1Nc1ncnc2c1CCN(c1ccccc1F)C2] |
| | 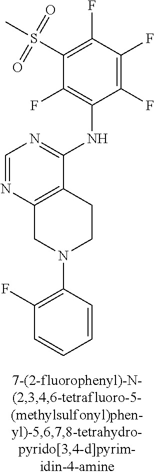 [SMILES: CS(=O)(=O)c1c(F)c(F)c(F)c(Nc2ncnc3c2CCN(c2ccccc2F)C3)c1F] |
| | 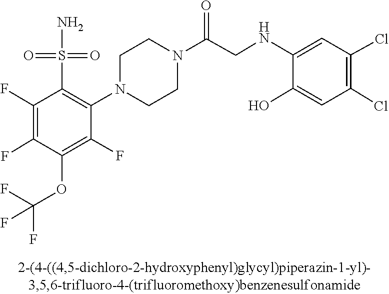 [SMILES: NS(=O)(=O)c1c(F)c(F)c(OC(F)(F)F)c(F)c1N1CCN(C(=O)CNc2cc(Cl)c(Cl)cc2O)CC1] |
| | 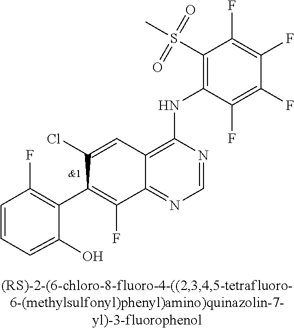 |
| 21-25 | 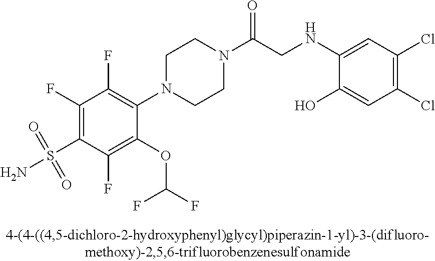 [SMILES: NS(=O)(=O)c1c(F)c(F)c(N2CCN(C(=O)CNc3cc(Cl)c(Cl)cc3O)CC2)c(OC(F)F)c1F] |
| | 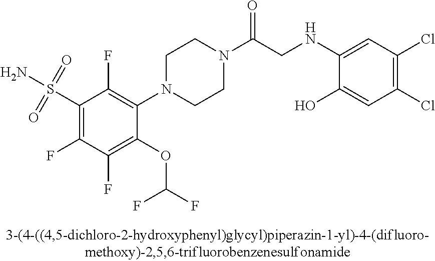 [SMILES: NS(=O)(=O)c1c(F)c(F)c(OC(F)F)c(N2CCN(C(=O)CNc3cc(Cl)c(Cl)cc3O)CC2)c1F] |
| | 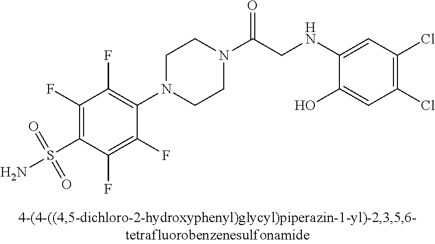 [SMILES: NS(=O)(=O)c1c(F)c(F)c(N2CCN(C(=O)CNc3cc(Cl)c(Cl)cc3O)CC2)c(F)c1F] |
| | 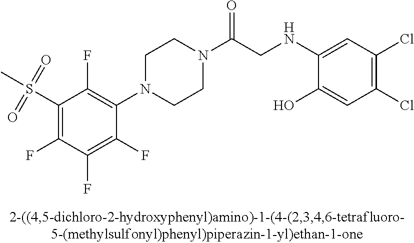 [SMILES: CS(=O)(=O)c1c(F)c(F)c(F)c(N2CCN(C(=O)CNc3cc(Cl)c(Cl)cc3O)CC2)c1F] |
| | 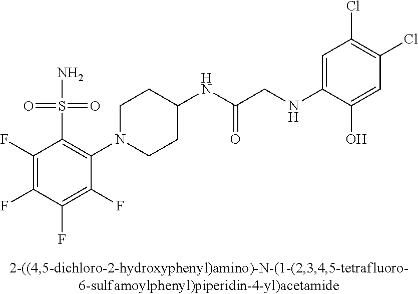 [SMILES: NS(=O)(=O)c1c(F)c(F)c(F)c(F)c1N1CCC(NC(=O)CNc2cc(Cl)c(Cl)cc2O)CC1] |
| 26-30 | 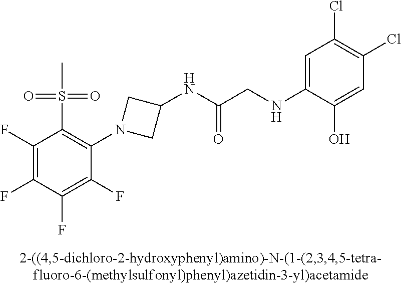 [SMILES: CS(=O)(=O)c1c(F)c(F)c(F)c(F)c1N1CC(NC(=O)CNc2cc(Cl)c(Cl)cc2O)C1] |
| | 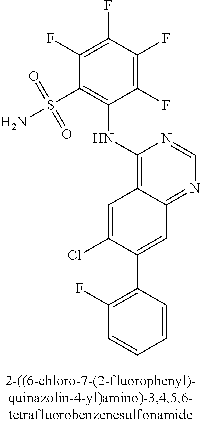 [SMILES: NS(=O)(=O)c1c(F)c(F)c(F)c(F)c1Nc1ncnc2cc(-c3ccccc3F)c(Cl)cc12] |
| | 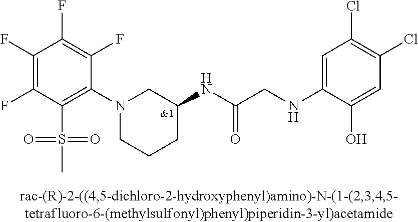 [SMILES: CS(=O)(=O)c1c(F)c(F)c(F)c(F)c1N1CCC[C@H](NC(=O)CNc2cc(Cl)c(Cl)cc2O)C1] |
| | 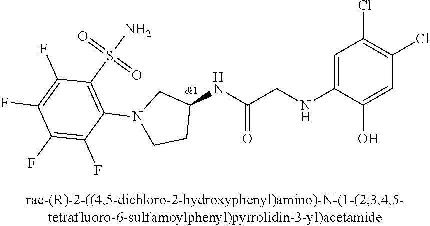 [SMILES: NS(=O)(=O)c1c(F)c(F)c(F)c(F)c1N1CC[C@H](NC(=O)CNc2cc(Cl)c(Cl)cc2O)C1] |
| | 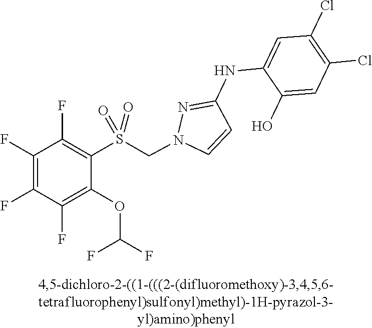 [SMILES: O=S(=O)(Cn1ccc(Nc2cc(Cl)c(Cl)cc2O)n1)c1c(F)c(F)c(F)c(F)c1OC(F)F] |
| 31-35 | 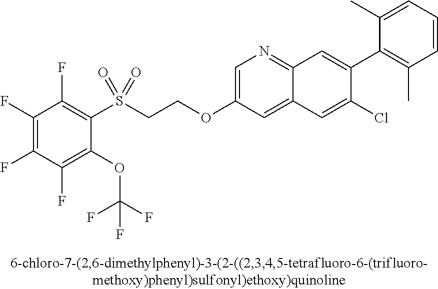 [SMILES: Cc1cccc(C)c1-c1cc2ncc(OCCS(=O)(=O)c3c(F)c(F)c(F)c(F)c3OC(F)(F)F)cc2cc1Cl] |
| | 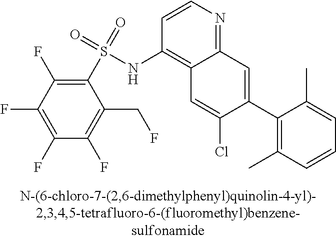 [SMILES: Cc1cccc(C)c1-c1cc2nccc(NS(=O)(=O)c3c(F)c(F)c(F)c(F)c3CF)c2cc1Cl] |
| | 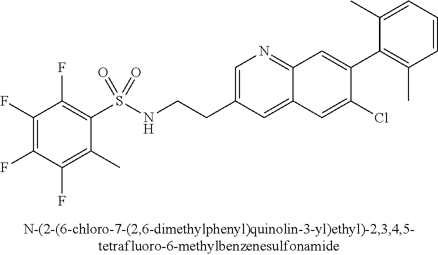 [SMILES: Cc1cccc(C)c1-c1cc2ncc(CCNS(=O)(=O)c3c(C)c(F)c(F)c(F)c3F)cc2cc1Cl] |
| | 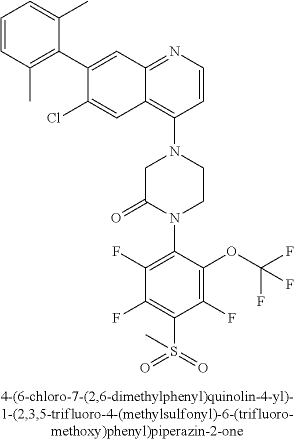 [SMILES: Cc1cccc(C)c1-c1cc2nccc(N3CCN(c4c(F)c(F)c(S(C)(=O)=O)c(F)c4OC(F)(F)F)C(=O)C3)c2cc1Cl] |
| | 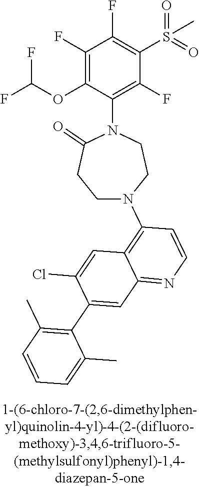 [SMILES: Cc1cccc(C)c1-c1cc2nccc(N3CCC(=O)N(c4c(F)c(S(C)(=O)=O)c(F)c(F)c4OC(F)F)CC3)c2cc1Cl] |
| 36-40 | 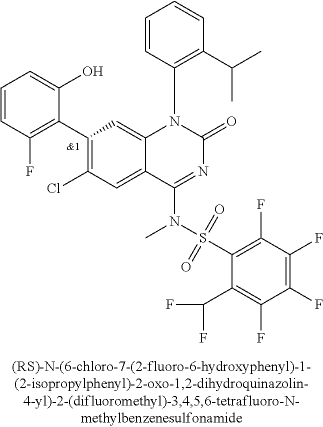 |
| | 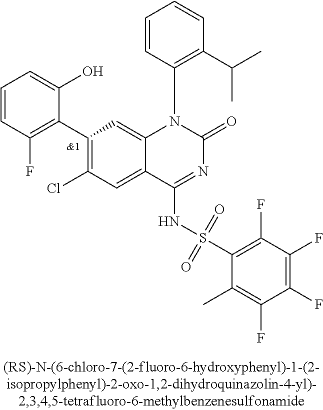 |
| | 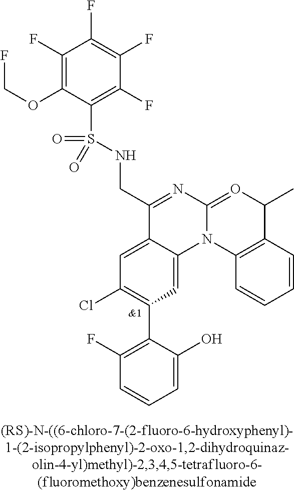 |
| | 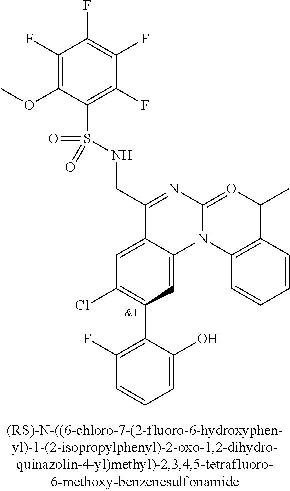 |
| | 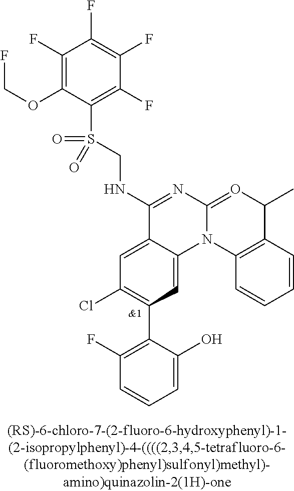 |
| 41-45 | 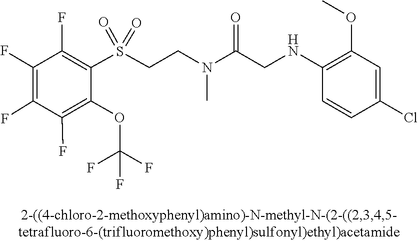 [SMILES: COc1cc(Cl)ccc1NCC(=O)N(C)CCS(=O)(=O)c1c(F)c(F)c(F)c(F)c1OC(F)(F)F] |
| | 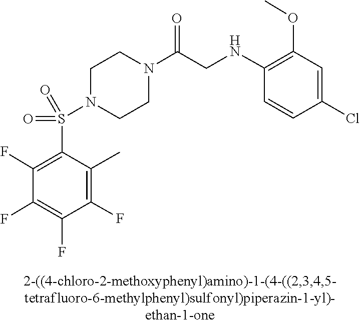 [SMILES: COc1cc(Cl)ccc1NCC(=O)N1CCN(S(=O)(=O)c2c(C)c(F)c(F)c(F)c2F)CC1] |
| | 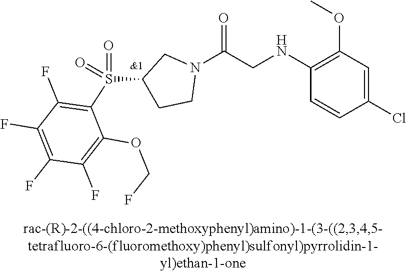 [SMILES: COc1cc(Cl)ccc1NCC(=O)N1CC[C@H](S(=O)(=O)c2c(F)c(F)c(F)c(F)c2OCF)C1] |
| | 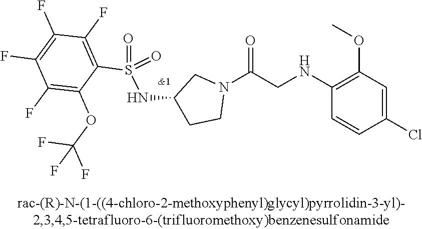 [SMILES: COc1cc(Cl)ccc1NCC(=O)N1CC[C@H](NS(=O)(=O)c2c(F)c(F)c(F)c(F)c2OC(F)(F)F)C1] |
| | 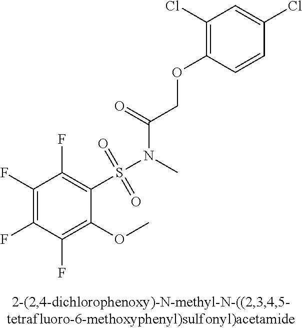 [SMILES: COc1c(F)c(F)c(F)c(F)c1S(=O)(=O)N(C)C(=O)COc1ccc(Cl)cc1Cl] |
| 46-50 | 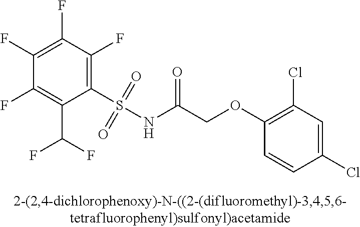 [SMILES: O=C(COc1ccc(Cl)cc1Cl)NS(=O)(=O)c1c(F)c(F)c(F)c(F)c1C(F)F] |
| | 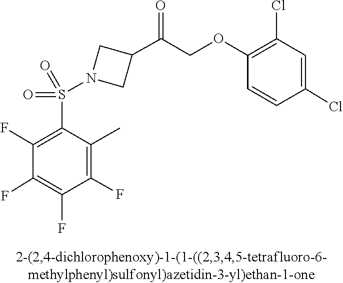 [SMILES: Cc1c(F)c(F)c(F)c(F)c1S(=O)(=O)N1CC(C(=O)COc2ccc(Cl)cc2Cl)C1] |
| | 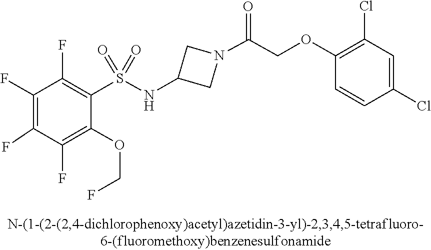 [SMILES: O=C(COc1ccc(Cl)cc1Cl)N1CC(NS(=O)(=O)c2c(F)c(F)c(F)c(F)c2OCF)C1] |
| | 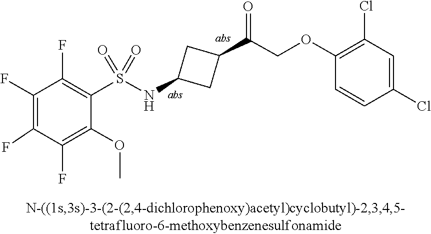 [SMILES: COc1c(F)c(F)c(F)c(F)c1S(=O)(=O)N[C@H]1C[C@@H](C(=O)COc2ccc(Cl)cc2Cl)C1] |
| | 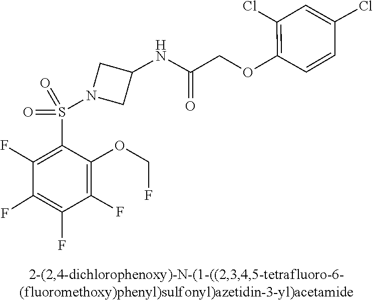 [SMILES: O=C(COc1ccc(Cl)cc1Cl)NC1CN(S(=O)(=O)c2c(F)c(F)c(F)c(F)c2OCF)C1] |
| 51-55 | 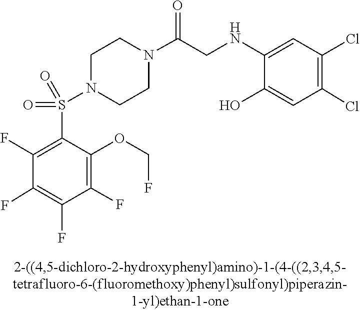 [SMILES: O=C(CNc1cc(Cl)c(Cl)cc1O)N1CCN(S(=O)(=O)c2c(F)c(F)c(F)c(F)c2OCF)CC1] |
| | 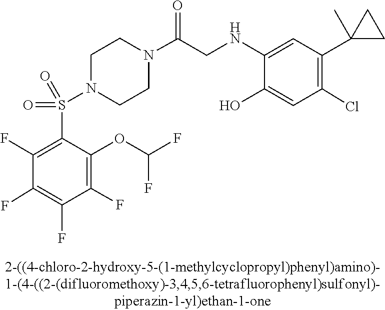 [SMILES: CC1(c2cc(NCC(=O)N3CCN(S(=O)(=O)c4c(F)c(F)c(F)c(F)c4OC(F)F)CC3)c(O)cc2Cl)CC1] |
| | 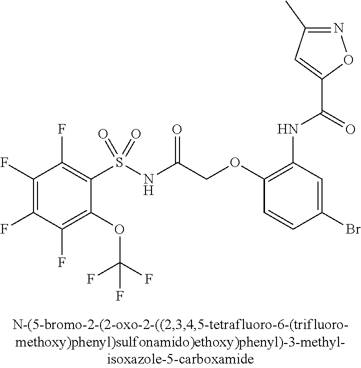 [SMILES: Cc1cc(C(=O)Nc2cc(Br)ccc2OCC(=O)NS(=O)(=O)c2c(F)c(F)c(F)c(F)c2OC(F)(F)F)on1] |
| | 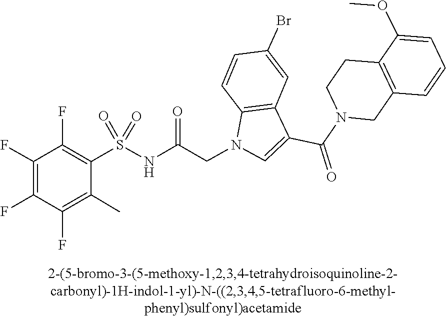 [SMILES: COc1cccc2c1CCN(C(=O)c1cn(CC(=O)NS(=O)(=O)c3c(C)c(F)c(F)c(F)c3F)c3ccc(Br)cc13)C2] |
| | 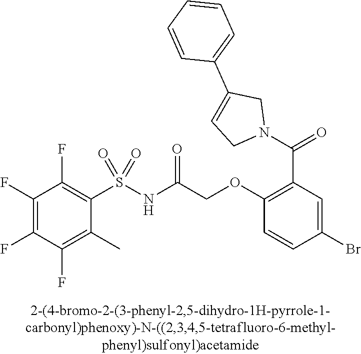 [SMILES: Cc1c(F)c(F)c(F)c(F)c1S(=O)(=O)NC(=O)COc1ccc(Br)cc1C(=O)N1CC=C(c2ccccc2)C1] |
| 56-60 | 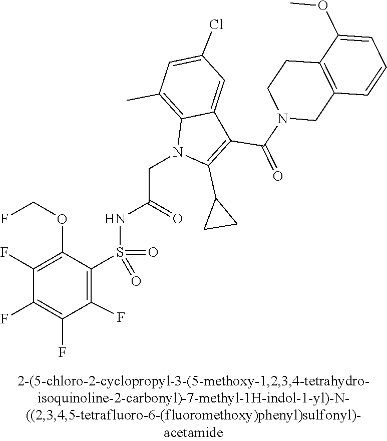 [SMILES: COc1cccc2c1CCN(C(=O)c1c(C3CC3)n(CC(=O)NS(=O)(=O)c3c(F)c(F)c(F)c(F)c3OCF)c3c(C)cc(Cl)cc13)C2] |
| | 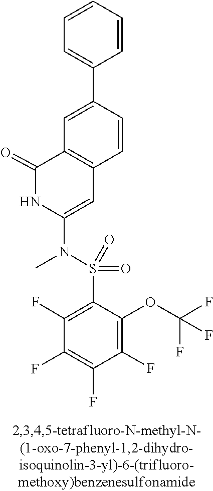 [SMILES: CN(c1cc2ccc(-c3ccccc3)cc2c(=O)[nH]1)S(=O)(=O)c1c(F)c(F)c(F)c(F)c1OC(F)(F)F] |
| | 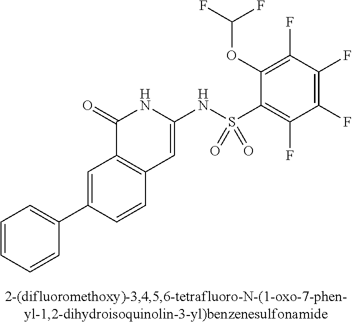 |
| | 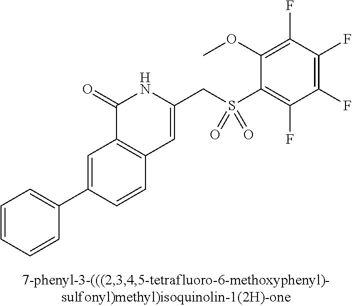 |
| | 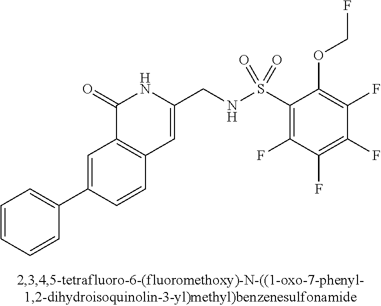 [SMILES: O=c1[nH]c(CNS(=O)(=O)c2c(F)c(F)c(F)c(F)c2OCF)cc2ccc(-c3ccccc3)cc12] |
| 61-65 | 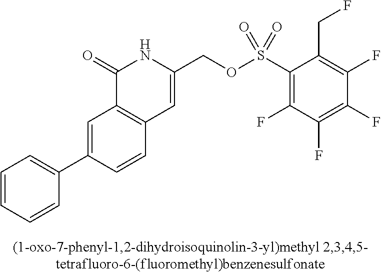 [SMILES: O=c1[nH]c(COS(=O)(=O)c2c(F)c(F)c(F)c(F)c2CF)cc2ccc(-c3ccccc3)cc12] |
| | 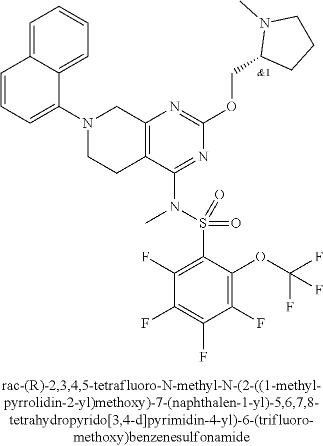 [SMILES: CN1CCC[C@@H]1COc1nc2c(c(N(C)S(=O)(=O)c3c(F)c(F)c(F)c(F)c3OC(F)(F)F)n1)CCN(c1cccc3ccccc13)C2] |
| | 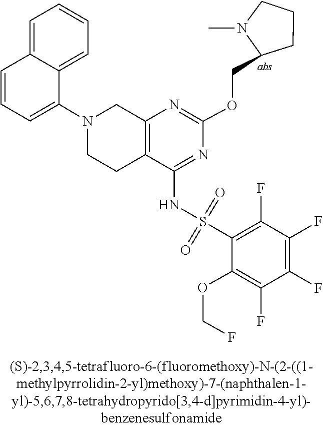 [SMILES: CN1CCC[C@H]1COc1nc2c(c(NS(=O)(=O)c3c(F)c(F)c(F)c(F)c3OCF)n1)CCN(c1cccc3ccccc13)C2] |
| | 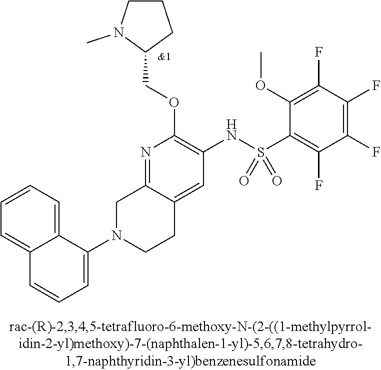 [SMILES: COc1c(F)c(F)c(F)c(F)c1S(=O)(=O)Nc1cc2c(nc1OC[C@H]1CCCN1C)CN(c1cccc3ccccc13)CC2] |
| | 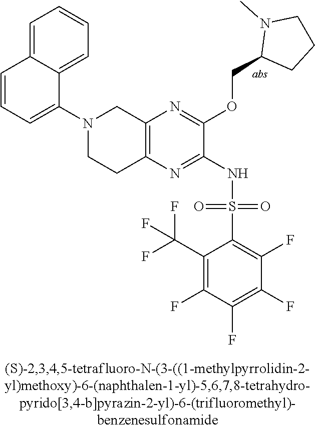 [SMILES: CN1CCC[C@H]1COc1nc2c(nc1NS(=O)(=O)c1c(F)c(F)c(F)c(F)c1C(F)(F)F)CCN(c1cccc3ccccc13)C2] |
| 66-70 | 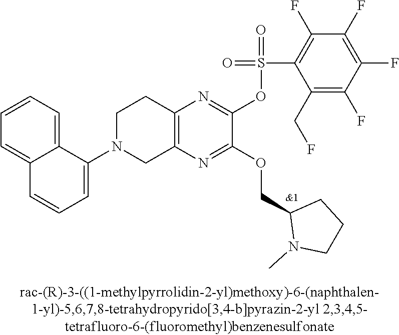 [SMILES: CN1CCC[C@@H]1COc1nc2c(nc1OS(=O)(=O)c1c(F)c(F)c(F)c(F)c1CF)CCN(c1cccc3ccccc13)C2] |
| | 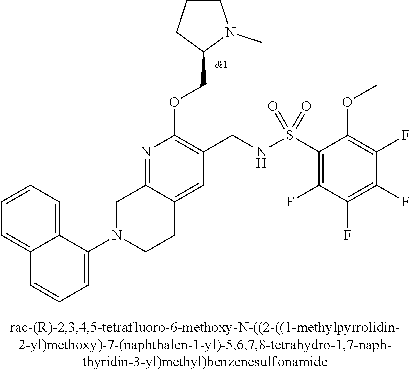 [SMILES: COc1c(F)c(F)c(F)c(F)c1S(=O)(=O)NCc1cc2c(nc1OC[C@H]1CCCN1C)CN(c1cccc3ccccc13)CC2] |
| | 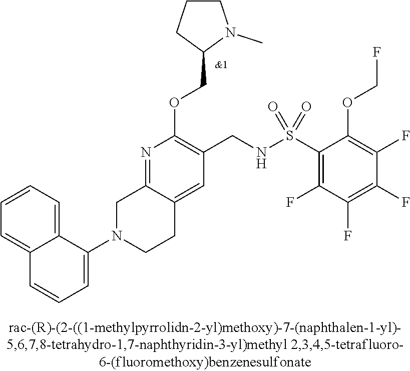 [SMILES: CN1CCC[C@@H]1COc1nc2c(cc1CNS(=O)(=O)c1c(F)c(F)c(F)c(F)c1OCF)CCN(c1cccc3ccccc13)C2] |
| | 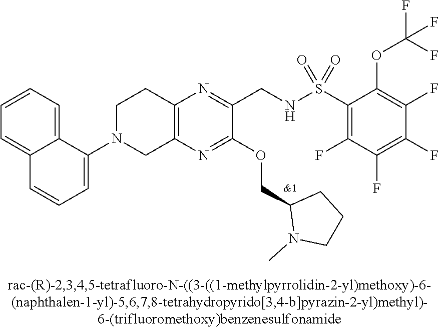 [SMILES: CN1CCC[C@@H]1COc1nc2c(nc1CNS(=O)(=O)c1c(F)c(F)c(F)c(F)c1OC(F)(F)F)CCN(c1cccc3ccccc13)C2] |
| | 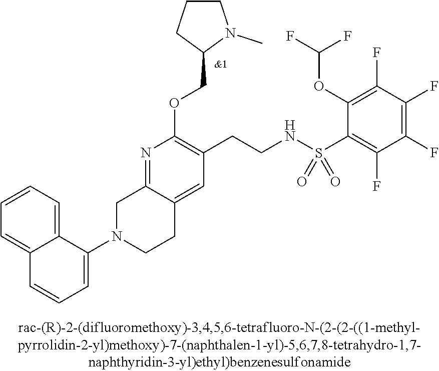 [SMILES: CN1CCC[C@@H]1COc1nc2c(cc1CCNS(=O)(=O)c1c(F)c(F)c(F)c(F)c1OC(F)F)CCN(c1cccc3ccccc13)C2] |
| 71-75 | 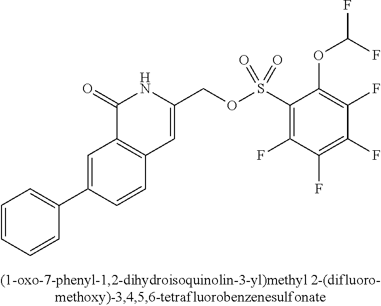 [SMILES: O=c1[nH]c(COS(=O)(=O)c2c(F)c(F)c(F)c(F)c2OC(F)F)cc2ccc(-c3ccccc3)cc12] |
| | 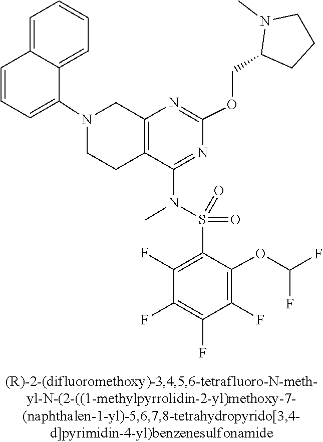 [SMILES: CN1CCC[C@@H]1COc1nc2c(c(N(C)S(=O)(=O)c3c(F)c(F)c(F)c(F)c3OC(F)F)n1)CCN(c1cccc3ccccc13)C2] |
| | 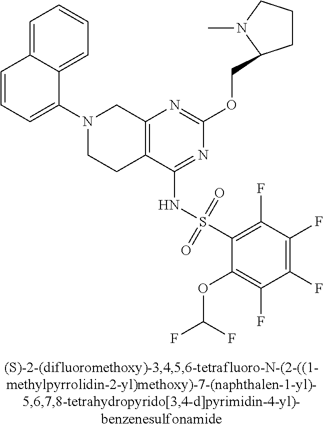 [SMILES: CN1CCC[C@H]1COc1nc2c(c(NS(=O)(=O)c3c(F)c(F)c(F)c(F)c3OC(F)F)n1)CCN(c1cccc3ccccc13)C2] |
| | 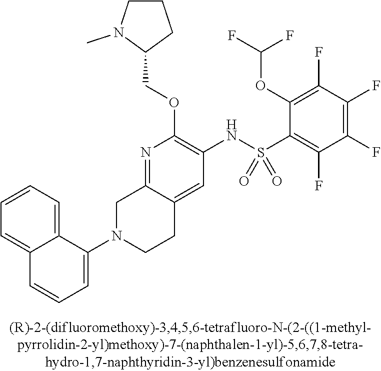 |
| | 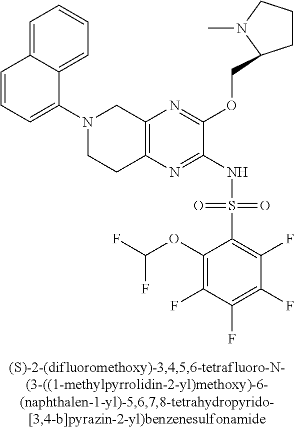 [SMILES: CN1CCC[C@H]1COc1nc2c(nc1NS(=O)(=O)c1c(F)c(F)c(F)c(F)c1OC(F)F)CCN(c1cccc3ccccc13)C2] |
| 76-80 | 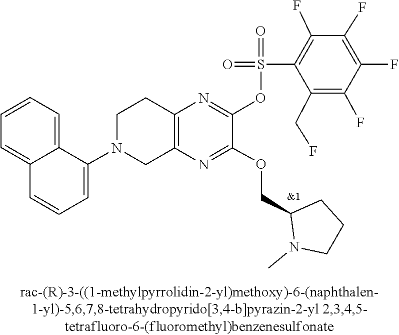 [SMILES: CN1CCC[C@@H]1COc1nc2c(nc1OS(=O)(=O)c1c(F)c(F)c(F)c(F)c1CF)CCN(c1cccc3ccccc13)C2] |
| | 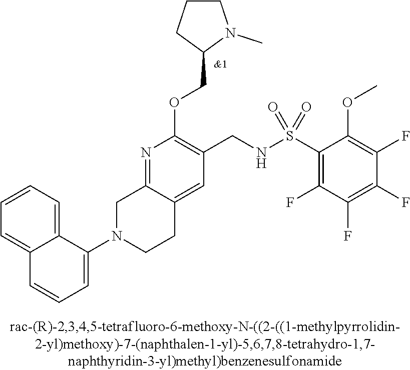 |
| | 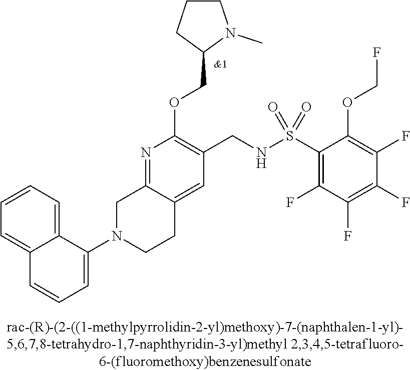 |
| | 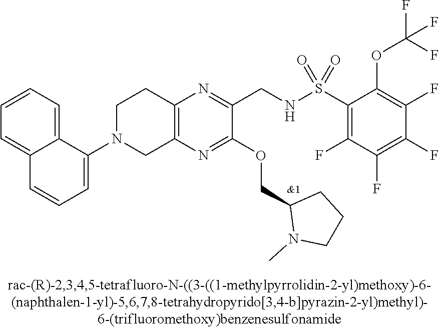 [SMILES: CN1CCC[C@@H]1COc1nc2c(nc1CNS(=O)(=O)c1c(F)c(F)c(F)c(F)c1OC(F)(F)F)CCN(c1cccc3ccccc13)C2] |
| | 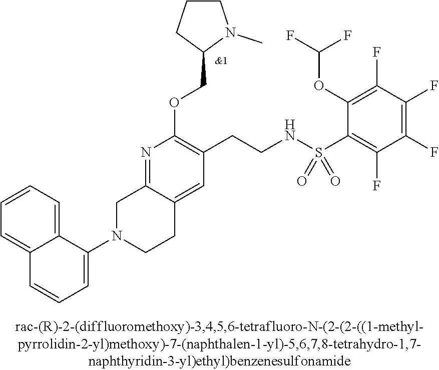 [SMILES: CN1CCC[C@@H]1COc1nc2c(cc1CCNS(=O)(=O)c1c(F)c(F)c(F)c(F)c1OC(F)F)CCN(c1cccc3ccccc13)C2] |
| 81-86 | 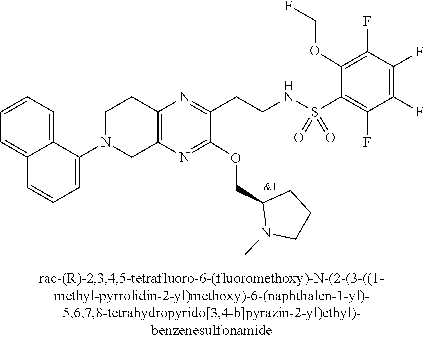 [SMILES: CN1CCC[C@@H]1COc1nc2c(nc1CCNS(=O)(=O)c1c(F)c(F)c(F)c(F)c1OCF)CCN(c1cccc3ccccc13)C2] |
| | 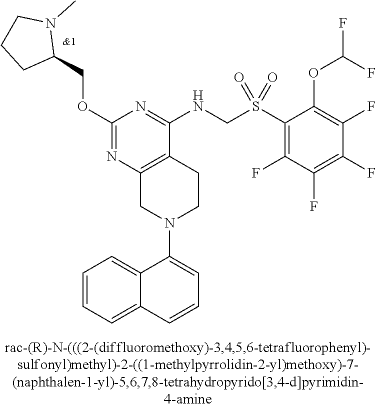 [SMILES: CN1CCC[C@@H]1COc1nc2c(c(NCS(=O)(=O)c3c(F)c(F)c(F)c(F)c3OC(F)F)n1)CCN(c1cccc3ccccc13)C2] |
| | 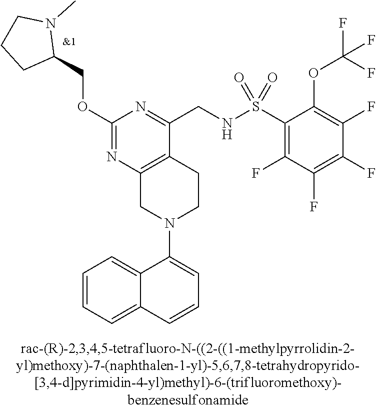 [SMILES: CN1CCC[C@@H]1COc1nc(CNS(=O)(=O)c2c(F)c(F)c(F)c(F)c2OC(F)(F)F)c2c(n1)CN(c1cccc3ccccc13)CC2] |
| | 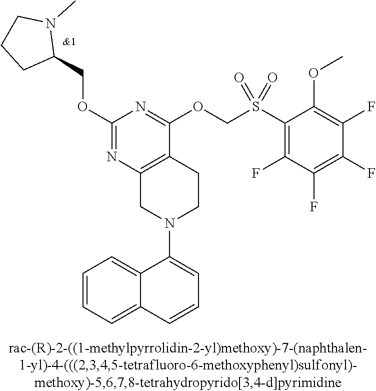 [SMILES: COc1c(F)c(F)c(F)c(F)c1S(=O)(=O)COc1nc(OC[C@H]2CCCN2C)nc2c1CCN(c1cccc3ccccc13)C2] |
| | 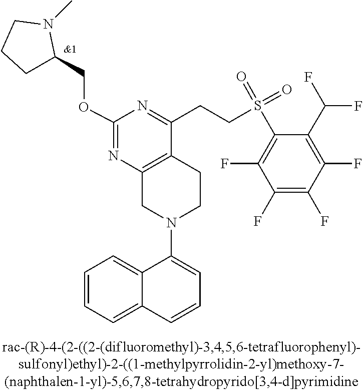 [SMILES: CN1CCC[C@@H]1COc1nc(CCS(=O)(=O)c2c(F)c(F)c(F)c(F)c2C(F)F)c2c(n1)CN(c1cccc3ccccc13)CC2] |
| | 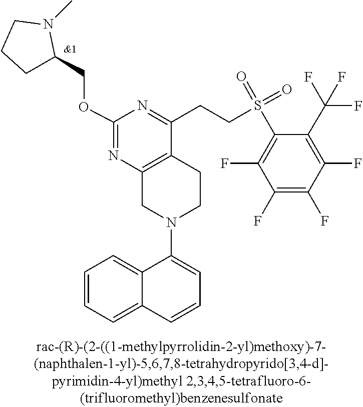 [SMILES: CN1CCC[C@@H]1COc1nc(CCS(=O)(=O)c2c(F)c(F)c(F)c(F)c2C(F)(F)F)c2c(n1)CN(c1cccc3ccccc13)CC2] |

TABLE 2

| # | G or G¹ |
|---|---|
| 1 | 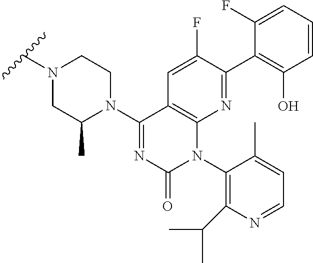 [SMILES: *N1CCN(c2nc(=O)n(-c3c(C)ccnc3C(C)C)c3nc(-c4c(O)cccc4F)c(F)cc23)[C@@H](C)C1] |
| 2 | 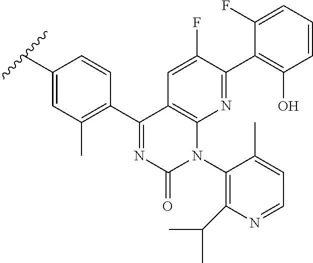 [SMILES: *c1ccc(-c2nc(=O)n(-c3c(C)ccnc3C(C)C)c3nc(-c4c(O)cccc4F)c(F)cc23)c(C)c1] |
| 3 | 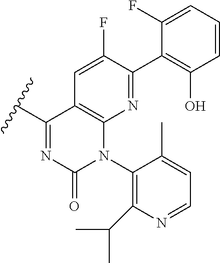 [SMILES: *c1nc(=O)n(-c2c(C)ccnc2C(C)C)c2nc(-c3c(O)cccc3F)c(F)cc12] |
| 4 | 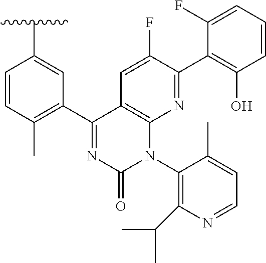 [SMILES: *c1ccc(C)c(-c2nc(=O)n(-c3c(C)ccnc3C(C)C)c3nc(-c4c(O)cccc4F)c(F)cc23)c1] |
| 5 | 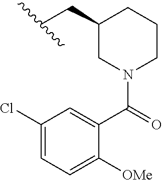 [SMILES: *C[C@H]1CCCN(C(=O)c2cc(Cl)ccc2OC)C1] |
| 6 | 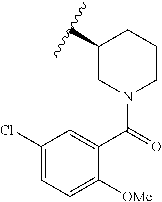 [SMILES: *[C@H]1CCCN(C(=O)c2cc(Cl)ccc2OC)C1] |
| 7 | 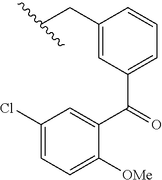 [SMILES: *Cc1cccc(C(=O)c2cc(Cl)ccc2OC)c1] |
| 8 | 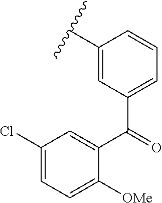 [SMILES: *c1cccc(C(=O)c2cc(Cl)ccc2OC)c1] |

TABLE 3

| # | G or G¹ |
|---|---|
| 1 | 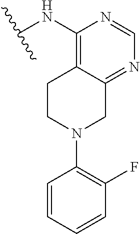 [SMILES: *Nc1ncnc2c1CCN(c1ccccc1F)C2] |
| 2 | 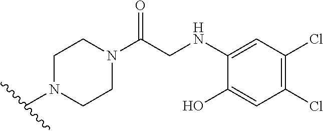 [SMILES: *N1CCN(C(=O)CNc2cc(Cl)c(Cl)cc2O)CC1] |
| 3 | 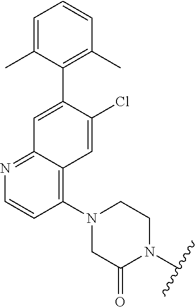 [SMILES: *N1CCN(c2ccnc3cc(-c4c(C)cccc4C)c(Cl)cc23)CC1=O] |
| 4 | 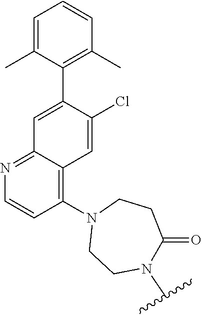 [SMILES: *N1CCN(c2ccnc3cc(-c4c(C)cccc4C)c(Cl)cc23)CCC1=O] |
| 5 | 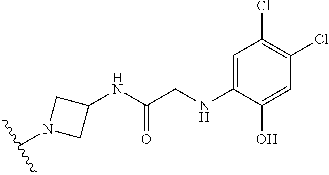 [SMILES: *N1CC(NC(=O)CNc2cc(Cl)c(Cl)cc2O)C1] |
| 6 | 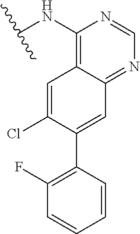 [SMILES: *Nc1ncnc2cc(-c3ccccc3F)c(Cl)cc12] |
| 7 | 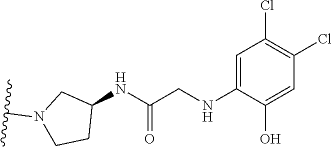 [SMILES: *N1CC[C@H](NC(=O)CNc2cc(Cl)c(Cl)cc2O)C1] |
| 8 | 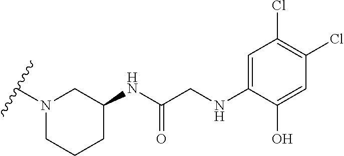 [SMILES: *N1CCC[C@H](NC(=O)CNc2cc(Cl)c(Cl)cc2O)C1] |
| 9 | 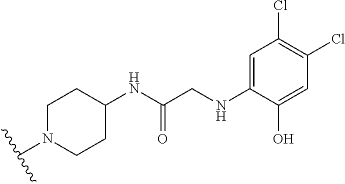 [SMILES: *N1CCC(NC(=O)CNc2cc(Cl)c(Cl)cc2O)CC1] |
| 10 | 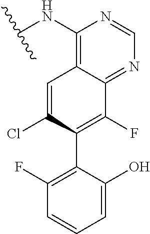 |

TABLE 4

| Group # | G or G¹ |
|---|---|
| 1 | 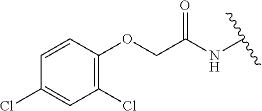 [SMILES: *NC(=O)COc1ccc(Cl)cc1Cl] |
| 2 | 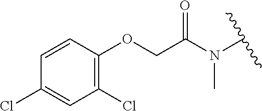 [SMILES: *N(C)C(=O)COc1ccc(Cl)cc1Cl] |
| 3 | 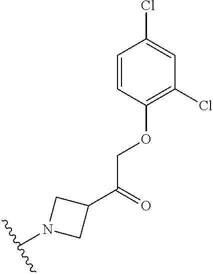 [SMILES: *N1CC(C(=O)COc2ccc(Cl)cc2Cl)C1] |
| 4 | 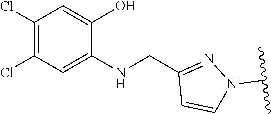 [SMILES: *n1ccc(CNc2cc(Cl)c(Cl)cc2O)n1] |
| 5 | 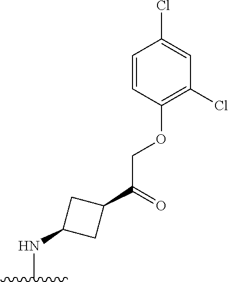 [SMILES: *N[C@H]1C[C@@H](C(=O)COc2ccc(Cl)cc2Cl)C1] |
| 6 | 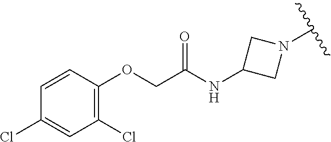 [SMILES: *N1CC(NC(=O)COc2ccc(Cl)cc2Cl)C1] |
| 7 | 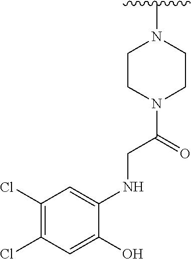 [SMILES: *N1CCN(C(=O)CNc2cc(Cl)c(Cl)cc2O)CC1] |
| 8 | 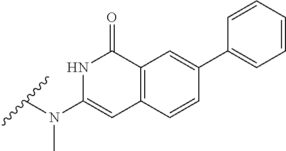 [SMILES: *N(C)c1cc2ccc(-c3ccccc3)cc2c(=O)[nH]1] |
| 9 | 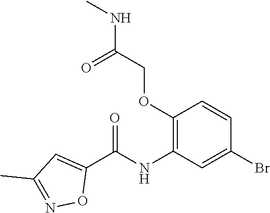 [SMILES: CNC(=O)COc1ccc(Br)cc1NC(=O)c1cc(C)no1] |
| 10 | 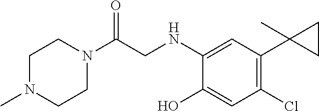 [SMILES: CN1CCN(C(=O)CNc2cc(C3(C)CC3)c(Cl)cc2O)CC1] |
| 11 | 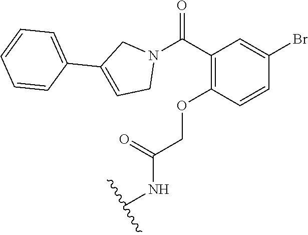 [SMILES: *NC(=O)COc1ccc(Br)cc1C(=O)N1CC=C(c2ccccc2)C1] |
| 12 | 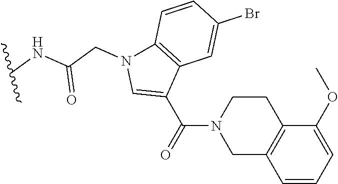 [SMILES: *NC(=O)Cn1cc(C(=O)N2CCc3c(cccc3OC)C2)c2cc(Br)ccc21] |
| 13 | 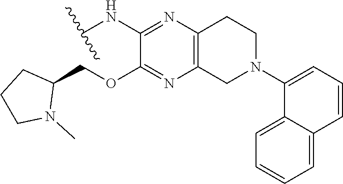 [SMILES: *Nc1nc2c(nc1OC[C@@H]1CCCN1C)CN(c1cccc3ccccc13)CC2] |
| 14 | 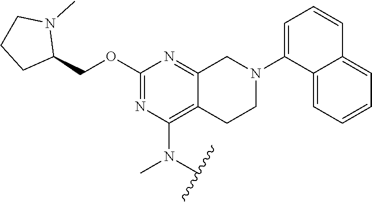 [SMILES: *N(C)c1nc(OC[C@H]2CCCN2C)nc2c1CCN(c1cccc3ccccc13)C2] |
| 15 | 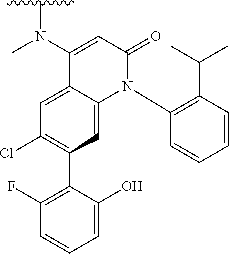 |
| 16 | 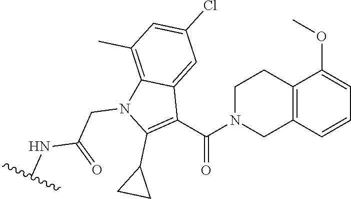 [SMILES: *NC(=O)Cn1c(C2CC2)c(C(=O)N2CCc3c(cccc3OC)C2)c2cc(Cl)cc(C)c21] |
| 17 | 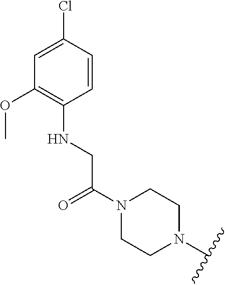 [SMILES: *N1CCN(C(=O)CNc2ccc(Cl)cc2OC)CC1] |

TABLE 5

| Group # | G or G¹ |
|---|---|
| 1 | 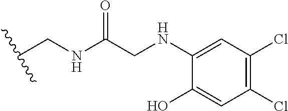 [SMILES: *CNC(=O)CNc1cc(Cl)c(Cl)cc1O] |
| 2 | 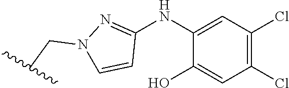 [SMILES: *Cn1ccc(Nc2cc(Cl)c(Cl)cc2O)n1] |
| 3 | 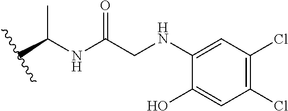 [SMILES: *[C@@H](C)NC(=O)CNc1cc(Cl)c(Cl)cc1O] |
| 4 | 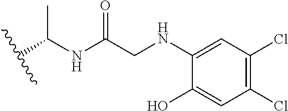 [SMILES: *[C@H](C)NC(=O)CNc1cc(Cl)c(Cl)cc1O] |
| 5 | 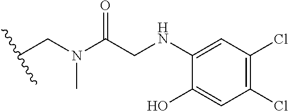 [SMILES: *CN(C)C(=O)CNc1cc(Cl)c(Cl)cc1O] |
| 6 | 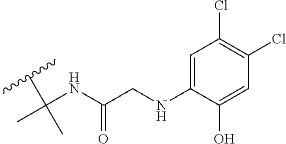 [SMILES: *C(C)(C)NC(=O)CNc1cc(Cl)c(Cl)cc1O] |
| 7 | 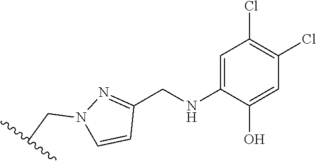 [SMILES: *Cn1ccc(CNc2cc(Cl)c(Cl)cc2O)n1] |
| 8 | 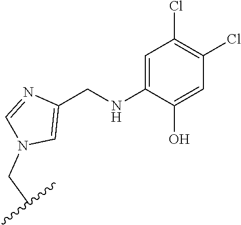 [SMILES: *Cn1cnc(CNc2cc(Cl)c(Cl)cc2O)c1] |
| 9 | 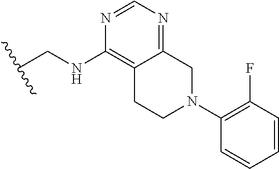 [SMILES: *CNc1ncnc2c1CCN(c1ccccc1F)C2] |
| 10 | 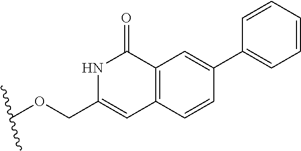 [SMILES: *OCc1cc2ccc(-c3ccccc3)cc2c(=O)[nH]1] |
| 11 | 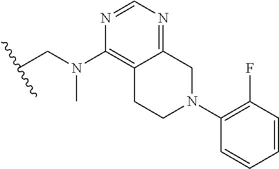 [SMILES: *CN(C)c1ncnc2c1CCN(c1ccccc1F)C2] |
| 12 | 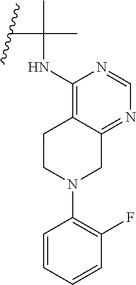 [SMILES: *C(C)(C)Nc1ncnc2c1CCN(c1ccccc1F)C2] |
| 13 | 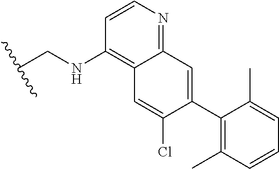 [SMILES: *CNc1ccnc2cc(-c3c(C)cccc3C)c(Cl)cc12] |
| 14 | 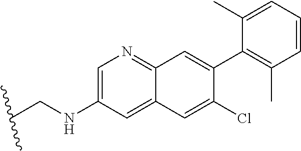 [SMILES: *CNc1cnc2cc(-c3c(C)cccc3C)c(Cl)cc2c1] |
| 15 | 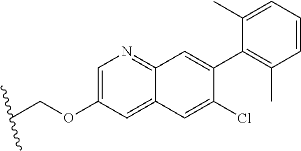 [SMILES: *COc1cnc2cc(-c3c(C)cccc3C)c(Cl)cc2c1] |
| 16 | 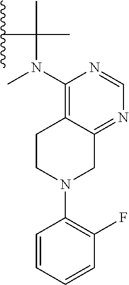 [SMILES: *C(C)(C)N(C)c1ncnc2c1CCN(c1ccccc1F)C2] |
| 17 | 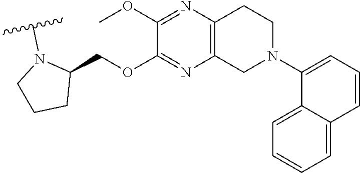 [SMILES: *N1CCC[C@@H]1COc1nc2c(nc1OC)CCN(c1cccc3ccccc13)C2] |
| 18 | 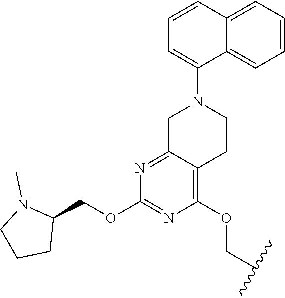 [SMILES: *COc1nc(OC[C@H]2CCCN2C)nc2c1CCN(c1cccc3ccccc13)C2] |
| 19 | 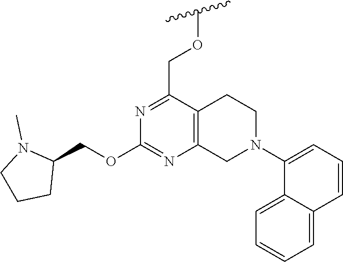 [SMILES: *OCc1nc(OC[C@H]2CCCN2C)nc2c1CCN(c1cccc3ccccc13)C2] |
| 20 | 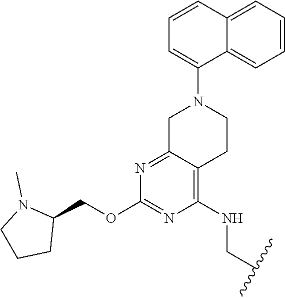 [SMILES: *CNc1nc(OC[C@H]2CCCN2C)nc2c1CCN(c1cccc3ccccc13)C2] |
| 21 | 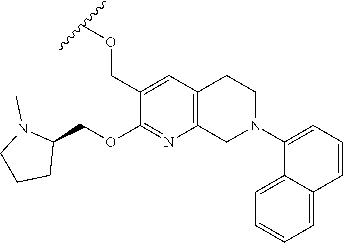 [SMILES: *OCc1cc2c(nc1OC[C@H]1CCCN1C)CN(c1cccc3ccccc13)CC2] |
| 22 | 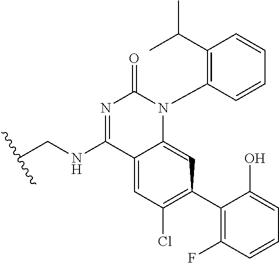 |

TABLE 6

| Group # | G or G¹ |
|---|---|
| 1 | 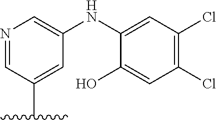 [SMILES: *c1cncc(Nc2cc(Cl)c(Cl)cc2O)c1] |
| 2 | 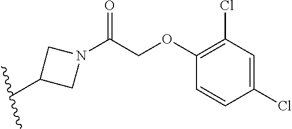 [SMILES: *C1CN(C(=O)COc2ccc(Cl)cc2Cl)C1] |
| 3 | 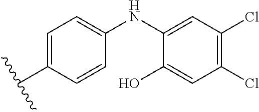 [SMILES: *c1ccc(Nc2cc(Cl)c(Cl)cc2O)cc1] |
| 4 | 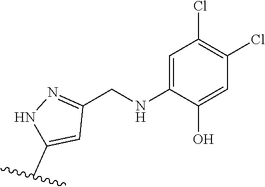 [SMILES: *c1cc(CNc2cc(Cl)c(Cl)cc2O)n[nH]1] |
| 5 | 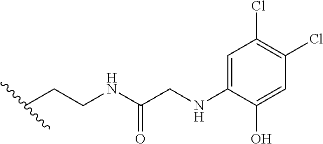 [SMILES: *CCNC(=O)CNc1cc(Cl)c(Cl)cc1O] |
| 6 | 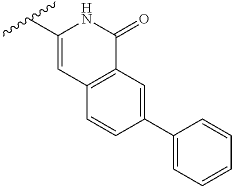 [SMILES: *c1cc2ccc(-c3ccccc3)cc2c(=O)[nH]1] |
| 7 | 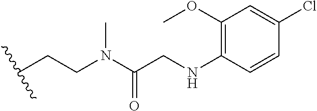 [SMILES: *CCN(C)C(=O)CNc1ccc(Cl)cc1OC] |
| 8 | 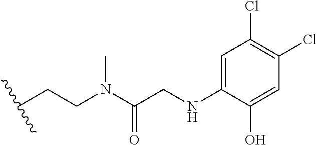 [SMILES: *CCN(C)C(=O)CNc1cc(Cl)c(Cl)cc1O] |
| 9 | 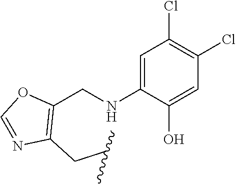 [SMILES: *Cc1ncoc1CNc1cc(Cl)c(Cl)cc1O] |
| 10 | 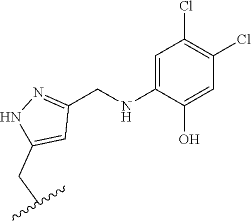 [SMILES: *Cc1cc(CNc2cc(Cl)c(Cl)cc2O)n[nH]1] |
| 11 | 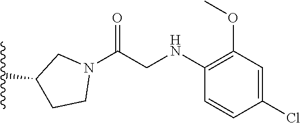 [SMILES: *[C@H]1CCN(C(=O)CNc2ccc(Cl)cc2OC)C1] |
| 12 | 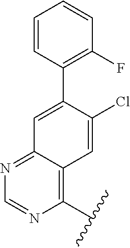 [SMILES: *c1ncnc2cc(-c3ccccc3F)c(Cl)cc12] |
| 13 | 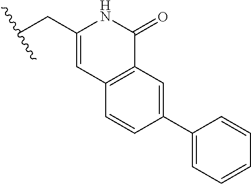 [SMILES: *Cc1cc2ccc(-c3ccccc3)cc2c(=O)[nH]1] |
| 14 | 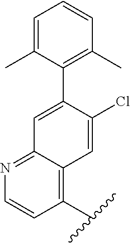 [SMILES: *c1ccnc2cc(-c3c(C)cccc3C)c(Cl)cc12] |
| 15 | 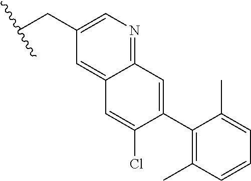 [SMILES: *Cc1cnc2cc(-c3c(C)cccc3C)c(Cl)cc2c1] |
| 16 | 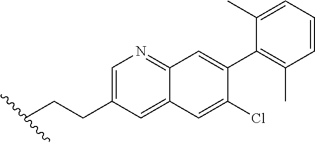 [SMILES: *CCc1cnc2cc(-c3c(C)cccc3C)c(Cl)cc2c1] |
| 17 | 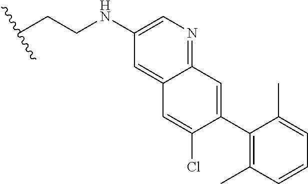 [SMILES: *CCNc1cnc2cc(-c3c(C)cccc3C)c(Cl)cc2c1] |
| 18 | 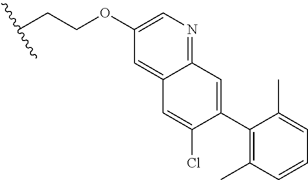 [SMILES: *CCOc1cnc2cc(-c3c(C)cccc3C)c(Cl)cc2c1] |
| 19 | 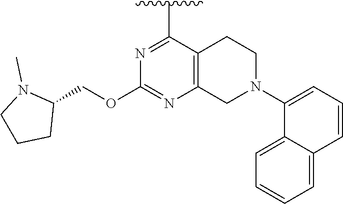 [SMILES: *c1nc(OC[C@@H]2CCCN2C)nc2c1CCN(c1cccc3ccccc13)C2] |
| 20 | 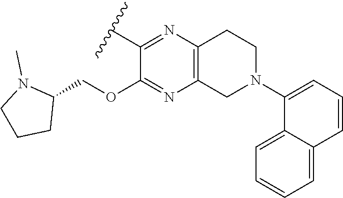 [SMILES: *c1nc2c(nc1OC[C@@H]1CCCN1C)CN(c1cccc3ccccc13)CC2] |
| 21 | 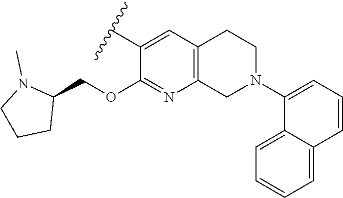 [SMILES: *c1cc2c(nc1OC[C@H]1CCCN1C)CN(c1cccc3ccccc13)CC2] |
| 22 | 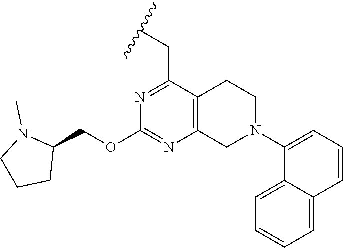 [SMILES: *Cc1nc(OC[C@H]2CCCN2C)nc2c1CCN(c1cccc3ccccc13)C2] |
| 23 | 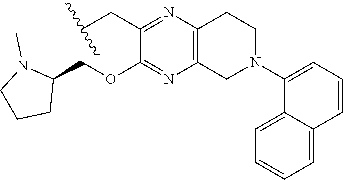 [SMILES: *Cc1nc2c(nc1OC[C@H]1CCCN1C)CN(c1cccc3ccccc13)CC2] |
| 24 | 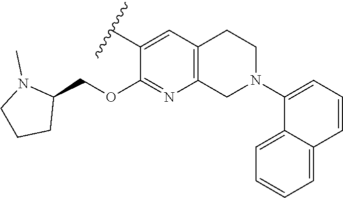 [SMILES: *c1cc2c(nc1OC[C@H]1CCCN1C)CN(c1cccc3ccccc13)CC2] |
| 25 | 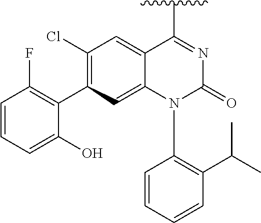 |
| 26 | 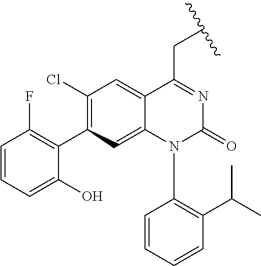 |
| 27 | 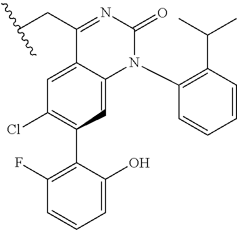 |
| 28 | 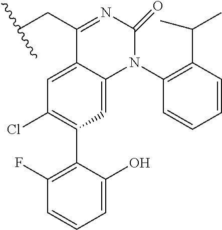 |
| 29 | 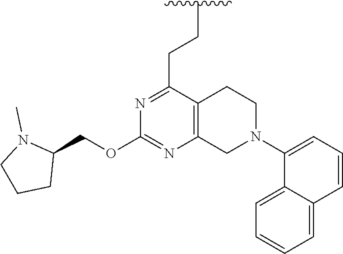 [SMILES: *CCc1nc(OC[C@H]2CCCN2C)nc2c1CCN(c1cccc3ccccc13)C2] |
| 30 | 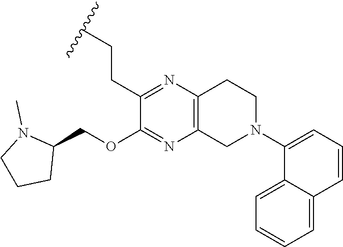 [SMILES: *CCc1nc2c(nc1OC[C@H]1CCCN1C)CN(c1cccc3ccccc13)CC2] |
| 31 | 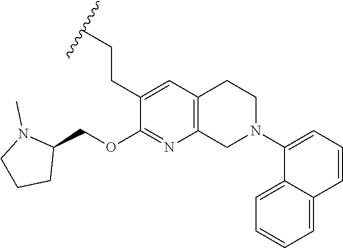 [SMILES: *CCc1cc2c(nc1OC[C@H]1CCCN1C)CN(c1cccc3ccccc13)CC2] |

TABLE 7
Exemplary Warhead groups of Formula (I), e.g., when G or G¹ are as
claimed or shown in Tables 2-6

| # | 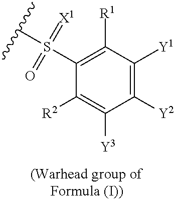 |
|---|---|
| 1-4 | 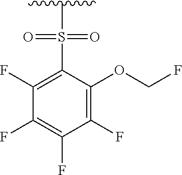 [SMILES: *S(=O)(=O)c1c(F)c(F)c(F)c(F)c1OCF] |
| | 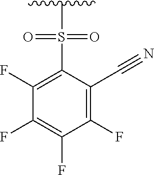 [SMILES: *S(=O)(=O)c1c(F)c(F)c(F)c(F)c1C#N] |
| | 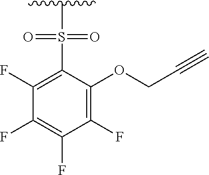 [SMILES: *S(=O)(=O)c1c(F)c(F)c(F)c(F)c1OCC#C] |
| | 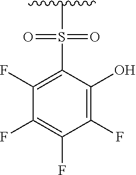 [SMILES: *S(=O)(=O)c1c(O)c(F)c(F)c(F)c1F] |
| | 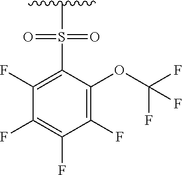 [SMILES: *S(=O)(=O)c1c(F)c(F)c(F)c(F)c1OC(F)(F)F] |
| 5-8 | 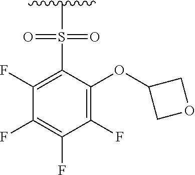 [SMILES: *S(=O)(=O)c1c(F)c(F)c(F)c(F)c1OC1COC1] |
| | 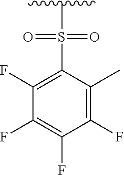 [SMILES: *S(=O)(=O)c1c(C)c(F)c(F)c(F)c1F] |
| | 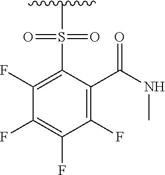 [SMILES: *S(=O)(=O)c1c(F)c(F)c(F)c(F)c1C(=O)NC] |
| | 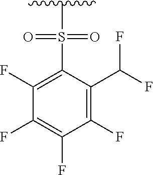 [SMILES: *S(=O)(=O)c1c(F)c(F)c(F)c(F)c1C(F)F] |
| | 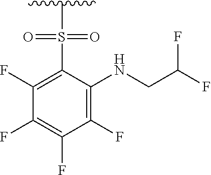 [SMILES: *S(=O)(=O)c1c(F)c(F)c(F)c(F)c1NCC(F)F] |
| 9-12 | 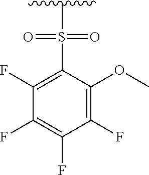 [SMILES: *S(=O)(=O)c1c(F)c(F)c(F)c(F)c1OC] |
| | 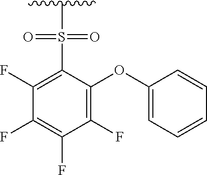 [SMILES: *S(=O)(=O)c1c(F)c(F)c(F)c(F)c1Oc1ccccc1] |
| | 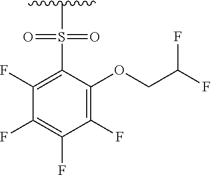 [SMILES: *S(=O)(=O)c1c(F)c(F)c(F)c(F)c1OCC(F)F] |
| | 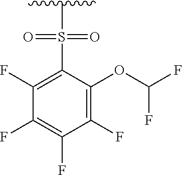 [SMILES: *S(=O)(=O)c1c(F)c(F)c(F)c(F)c1OC(F)F] |
| | 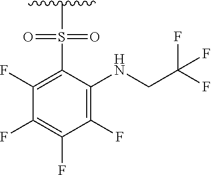 [SMILES: *S(=O)(=O)c1c(F)c(F)c(F)c(F)c1NCC(F)(F)F] |
| 13-16 | 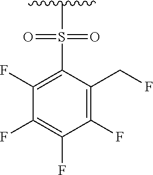 [SMILES: *S(=O)(=O)c1c(F)c(F)c(F)c(F)c1CF] |
| | 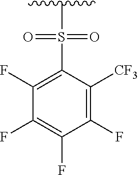 [SMILES: *S(=O)(=O)c1c(F)c(F)c(F)c(F)c1C(F)(F)F] |
| | 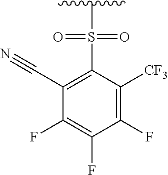 [SMILES: *S(=O)(=O)c1c(C#N)c(F)c(F)c(F)c1C(F)(F)F] |
| | 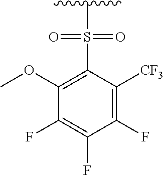 [SMILES: *S(=O)(=O)c1c(OC)c(F)c(F)c(F)c1C(F)(F)F] |
| | 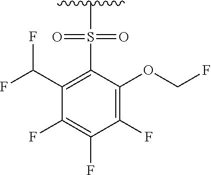 [SMILES: *S(=O)(=O)c1c(OCF)c(F)c(F)c(F)c1C(F)F] |

In some embodiments, the compound described herein has a structure provided in Table 8.

TABLE 8

| Compound | |
|---|---|
| Number | Structure |
| 1A | 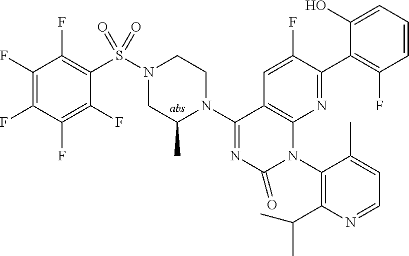 [SMILES: Cc1ccnc(C(C)C)c1-n1c(=O)nc(N2CCN(S(=O)(=O)c3c(F)c(F)c(F)c(F)c3F)C[C@@H]2C)c2cc(F)c(-c3c(O)cccc3F)nc21] |
| 2A | 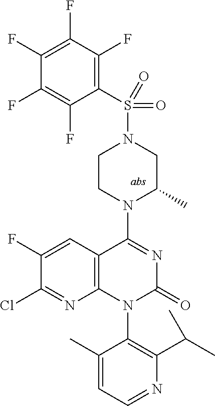 [SMILES: Cc1ccnc(C(C)C)c1-n1c(=O)nc(N2CCN(S(=O)(=O)c3c(F)c(F)c(F)c(F)c3F)C[C@@H]2C)c2cc(F)c(Cl)nc21] |
| 3A | 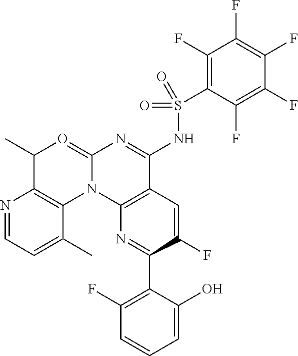 |
| 4A | 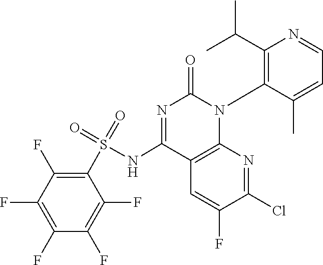 [SMILES: Cc1ccnc(C(C)C)c1-n1c(=O)nc(NS(=O)(=O)c2c(F)c(F)c(F)c(F)c2F)c2cc(F)c(Cl)nc21] |
| 5A | 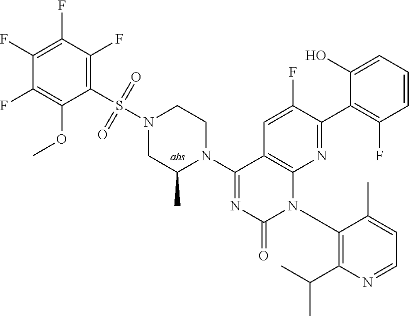 [SMILES: COc1c(F)c(F)c(F)c(F)c1S(=O)(=O)N1CCN(c2nc(=O)n(-c3c(C)ccnc3C(C)C)c3nc(-c4c(O)cccc4F)c(F)cc23)[C@@H](C)C1] |
| 6A | 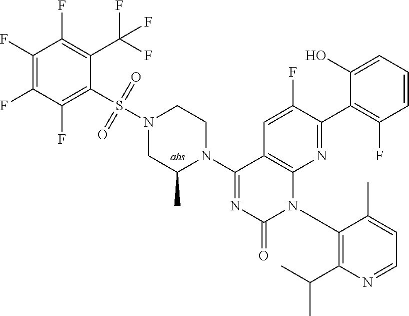 [SMILES: Cc1ccnc(C(C)C)c1-n1c(=O)nc(N2CCN(S(=O)(=O)c3c(F)c(F)c(F)c(F)c3C(F)(F)F)C[C@@H]2C)c2cc(F)c(-c3c(O)cccc3F)nc21] |
| 7A | 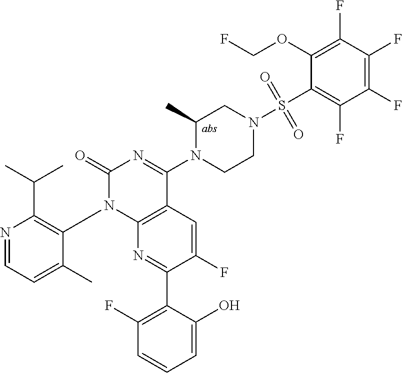 [SMILES: Cc1ccnc(C(C)C)c1-n1c(=O)nc(N2CCN(S(=O)(=O)c3c(F)c(F)c(F)c(F)c3OCF)C[C@@H]2C)c2cc(F)c(-c3c(O)cccc3F)nc21] |
| 8A | 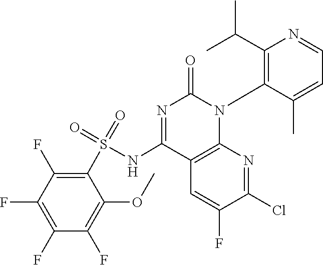 [SMILES: COc1c(F)c(F)c(F)c(F)c1S(=O)(=O)Nc1nc(=O)n(-c2c(C)ccnc2C(C)C)c2nc(Cl)c(F)cc12] |
| 9A | 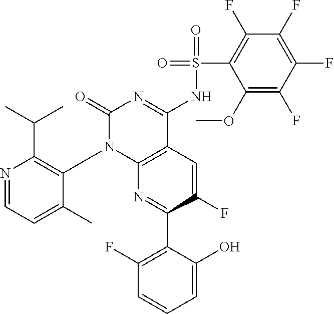 |
| 10A | 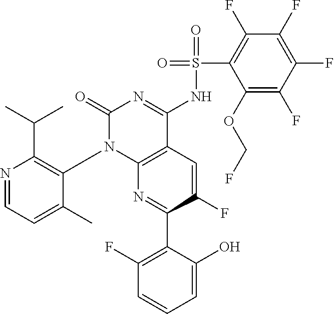 |
| 11A | 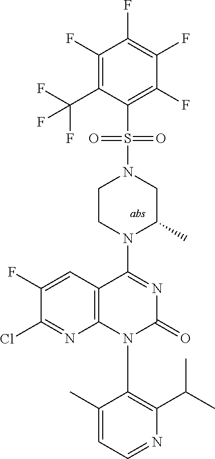 [SMILES: Cc1ccnc(C(C)C)c1-n1c(=O)nc(N2CCN(S(=O)(=O)c3c(F)c(F)c(F)c(F)c3C(F)(F)F)C[C@@H]2C)c2cc(F)c(Cl)nc21] |
| 12A | 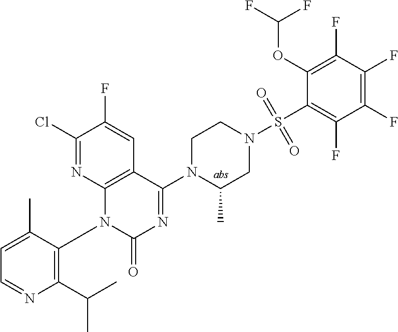 [SMILES: Cc1ccnc(C(C)C)c1-n1c(=O)nc(N2CCN(S(=O)(=O)c3c(F)c(F)c(F)c(F)c3OC(F)F)C[C@@H]2C)c2cc(F)c(Cl)nc21] |
| 13A | 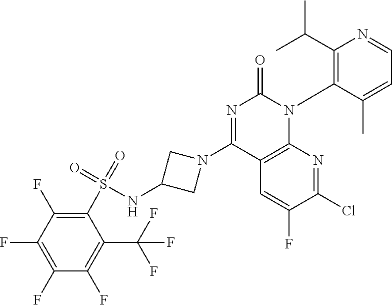 [SMILES: Cc1ccnc(C(C)C)c1-n1c(=O)nc(N2CC(NS(=O)(=O)c3c(F)c(F)c(F)c(F)c3C(F)(F)F)C2)c2cc(F)c(Cl)nc21] |
| 14A | 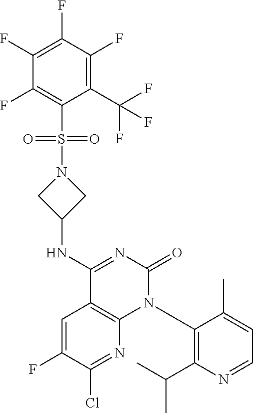 [SMILES: Cc1ccnc(C(C)C)c1-n1c(=O)nc(NC2CN(S(=O)(=O)c3c(F)c(F)c(F)c(F)c3C(F)(F)F)C2)c2cc(F)c(Cl)nc21] |
| 15A | 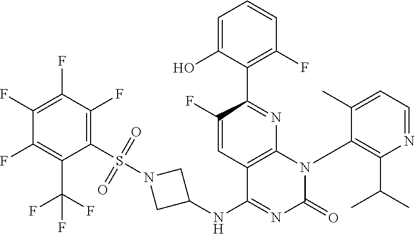 |
| 16A | 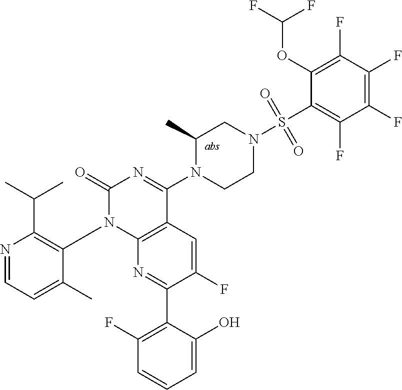 [SMILES: Cc1ccnc(C(C)C)c1-n1c(=O)nc(N2CCN(S(=O)(=O)c3c(F)c(F)c(F)c(F)c3OC(F)F)C[C@@H]2C)c2cc(F)c(-c3c(O)cccc3F)nc21] |
| 17A | 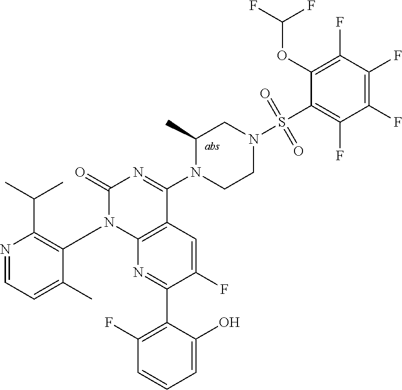 [SMILES: Cc1ccnc(C(C)C)c1-n1c(=O)nc(N2CCN(S(=O)(=O)c3c(F)c(F)c(F)c(F)c3OC(F)F)C[C@@H]2C)c2cc(F)c(-c3c(O)cccc3F)nc21] |
| 18A | 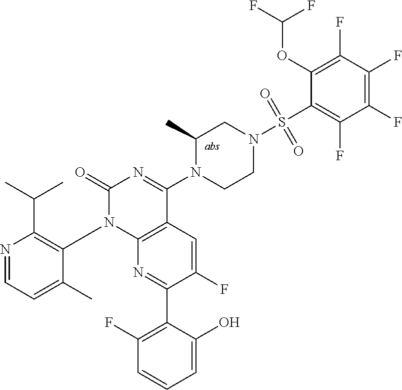 [SMILES: Cc1ccnc(C(C)C)c1-n1c(=O)nc(N2CCN(S(=O)(=O)c3c(F)c(F)c(F)c(F)c3OC(F)F)C[C@@H]2C)c2cc(F)c(-c3c(O)cccc3F)nc21] |
| 19A | 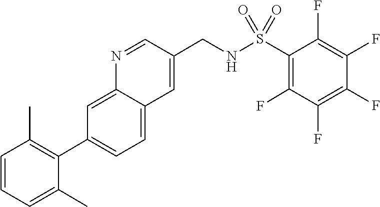 [SMILES: Cc1cccc(C)c1-c1ccc2cc(CNS(=O)(=O)c3c(F)c(F)c(F)c(F)c3F)cnc2c1] |
| 20A | 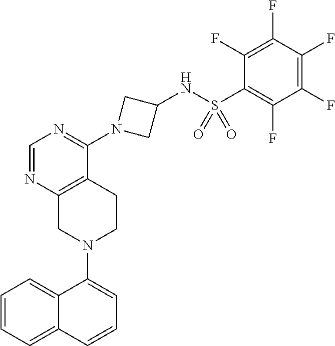 [SMILES: O=S(=O)(NC1CN(c2ncnc3c2CCN(c2cccc4ccccc24)C3)C1)c1c(F)c(F)c(F)c(F)c1F] |
| 21A | 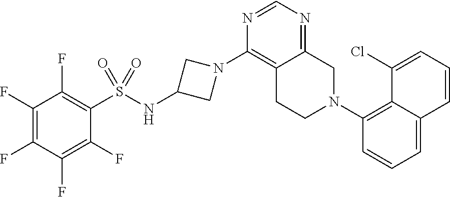 [SMILES: O=S(=O)(NC1CN(c2ncnc3c2CCN(c2cccc4cccc(Cl)c24)C3)C1)c1c(F)c(F)c(F)c(F)c1F] |
| 22A | 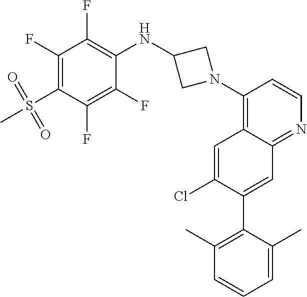 |
| 23A | 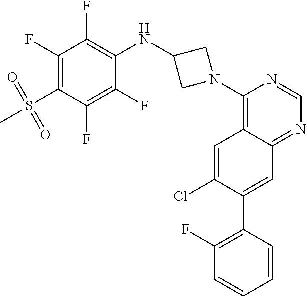 [SMILES: CS(=O)(=O)c1c(F)c(F)c(NC2CN(c3ncnc4cc(-c5ccccc5F)c(Cl)cc34)C2)c(F)c1F] |
| 24A “Racemate” | 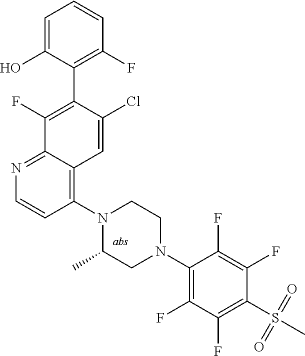 [SMILES: C[C@H]1CN(c2c(F)c(F)c(S(C)(=O)=O)c(F)c2F)CCN1c1ccnc2c(F)c(-c3c(O)cccc3F)c(Cl)cc12] |
| 25A “Isomer 1” | 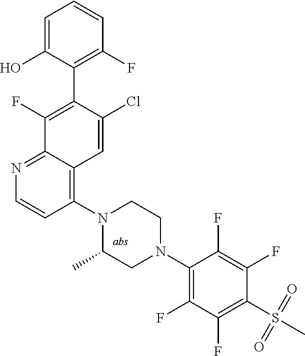 [SMILES: C[C@H]1CN(c2c(F)c(F)c(S(C)(=O)=O)c(F)c2F)CCN1c1ccnc2c(F)c(-c3c(O)cccc3F)c(Cl)cc12] |
| 26A “Isomer 2” | 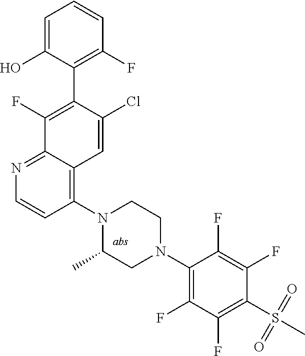 [SMILES: C[C@H]1CN(c2c(F)c(F)c(S(C)(=O)=O)c(F)c2F)CCN1c1ccnc2c(F)c(-c3c(O)cccc3F)c(Cl)cc12] |
| 27A | 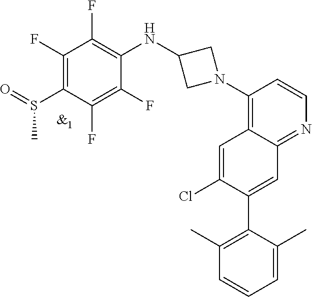 [SMILES: Cc1cccc(C)c1-c1cc2nccc(N3CC(Nc4c(F)c(F)c([S@](C)=O)c(F)c4F)C3)c2cc1Cl] |
| 28A | 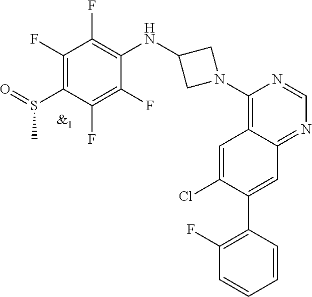 [SMILES: C[S@](=O)c1c(F)c(F)c(NC2CN(c3ncnc4cc(-c5ccccc5F)c(Cl)cc34)C2)c(F)c1F] |
| 29A | 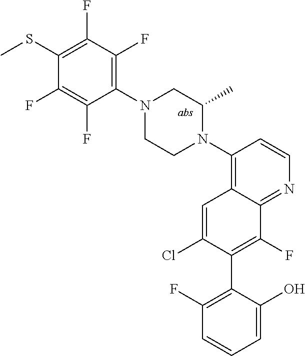 [SMILES: CSc1c(F)c(F)c(N2CCN(c3ccnc4c(F)c(-c5c(O)cccc5F)c(Cl)cc34)[C@@H](C)C2)c(F)c1F] |
| 30A | 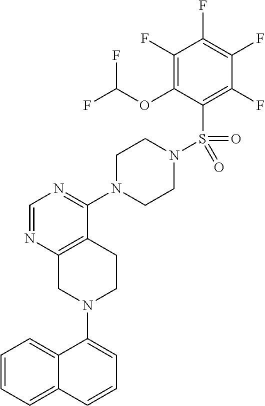 [SMILES: O=S(=O)(c1c(F)c(F)c(F)c(F)c1OC(F)F)N1CCN(c2ncnc3c2CCN(c2cccc4ccccc24)C3)CC1] |
| 31A | 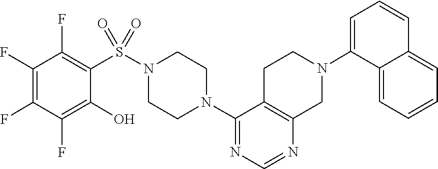 [SMILES: O=S(=O)(c1c(O)c(F)c(F)c(F)c1F)N1CCN(c2ncnc3c2CCN(c2cccc4ccccc24)C3)CC1] |
| 32A | 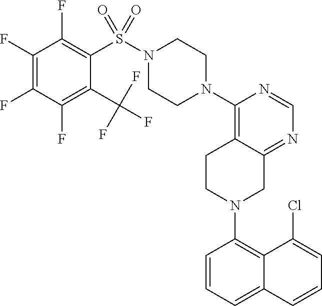 [SMILES: O=S(=O)(c1c(F)c(F)c(F)c(F)c1C(F)(F)F)N1CCN(c2ncnc3c2CCN(c2cccc4cccc(Cl)c24)C3)CC1] |
| 33A | 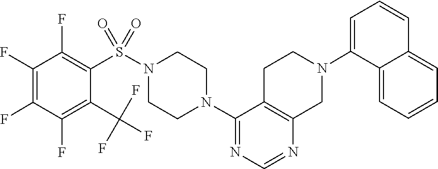 [SMILES: O=S(=O)(c1c(F)c(F)c(F)c(F)c1C(F)(F)F)N1CCN(c2ncnc3c2CCN(c2cccc4ccccc24)C3)CC1] |
| 34A | 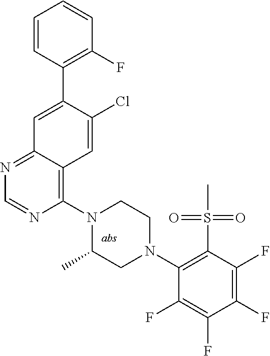 [SMILES: C[C@H]1CN(c2c(F)c(F)c(F)c(F)c2S(C)(=O)=O)CCN1c1ncnc2cc(-c3ccccc3F)c(Cl)cc12] |
| 35A | 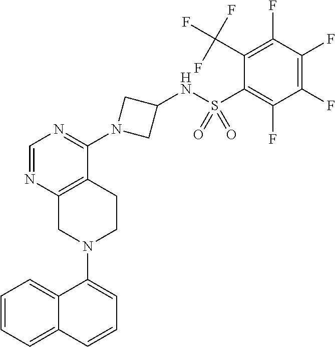 [SMILES: O=S(=O)(NC1CN(c2ncnc3c2CCN(c2cccc4ccccc24)C3)C1)c1c(F)c(F)c(F)c(F)c1C(F)(F)F] |
| 36A | 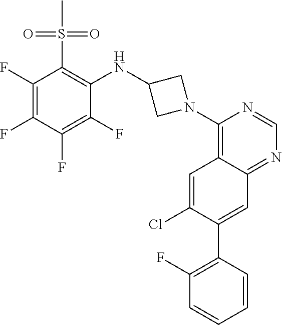 [SMILES: CS(=O)(=O)c1c(F)c(F)c(F)c(F)c1NC1CN(c2ncnc3cc(-c4ccccc4F)c(Cl)cc23)C1] |
| 37A | 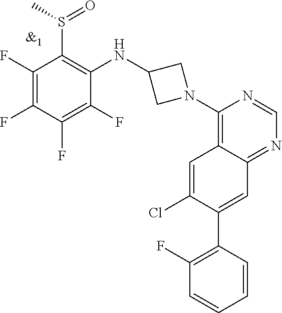 [SMILES: C[S@](=O)c1c(F)c(F)c(F)c(F)c1NC1CN(c2ncnc3cc(-c4ccccc4F)c(Cl)cc23)C1] |
| 38A “Isomer 1” | 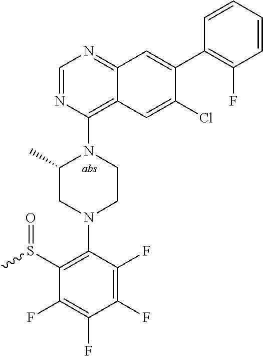 |
| 39A “Isomer 2” | 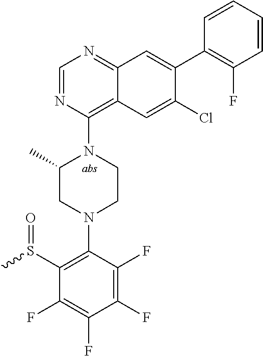 |
| 40A | 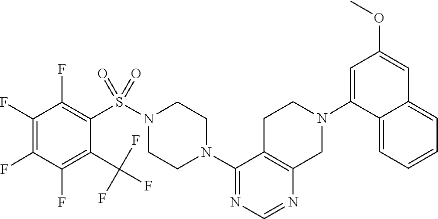 [SMILES: COc1cc(N2CCc3c(ncnc3N3CCN(S(=O)(=O)c4c(F)c(F)c(F)c(F)c4C(F)(F)F)CC3)C2)c2ccccc2c1] |
| 41A | 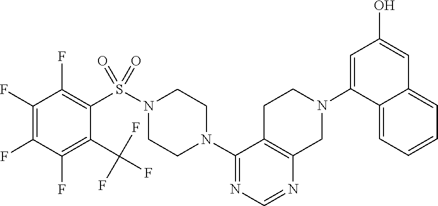 [SMILES: O=S(=O)(c1c(F)c(F)c(F)c(F)c1C(F)(F)F)N1CCN(c2ncnc3c2CCN(c2cc(O)cc4ccccc24)C3)CC1] |
| 42A | 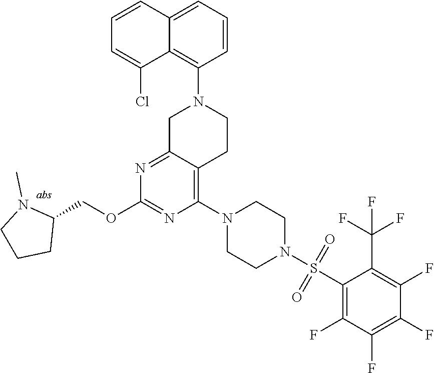 [SMILES: CN1CCC[C@H]1COc1nc2c(c(N3CCN(S(=O)(=O)c4c(F)c(F)c(F)c(F)c4C(F)(F)F)CC3)n1)CCN(c1cccc3cccc(Cl)c13)C2] |
| 43A | 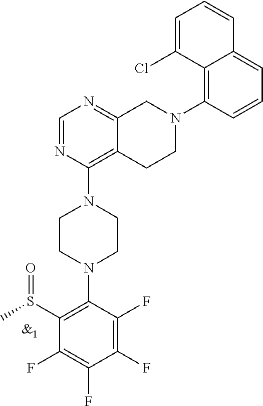 |
| 44A | 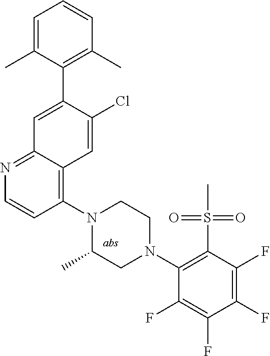 [SMILES: Cc1cccc(C)c1-c1cc2nccc(N3CCN(c4c(F)c(F)c(F)c(F)c4S(C)(=O)=O)C[C@@H]3C)c2cc1Cl] |
| 45A | 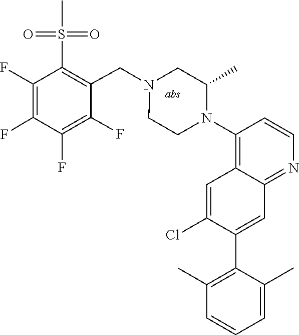 [SMILES: Cc1cccc(C)c1-c1cc2nccc(N3CCN(Cc4c(F)c(F)c(F)c(F)c4S(C)(=O)=O)C[C@@H]3C)c2cc1Cl] |
| 46A | 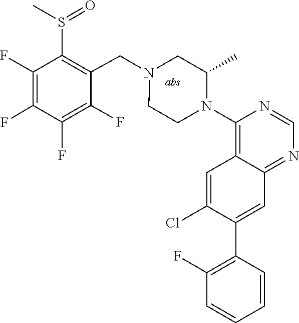 [SMILES: C[C@H]1CN(Cc2c(F)c(F)c(F)c(F)c2S(C)=O)CCN1c1ncnc2cc(-c3ccccc3F)c(Cl)cc12] |
| 47A | 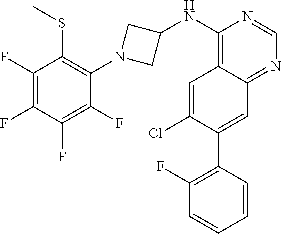 [SMILES: CSc1c(F)c(F)c(F)c(F)c1N1CC(Nc2ncnc3cc(-c4ccccc4F)c(Cl)cc23)C1] |
| 48A | 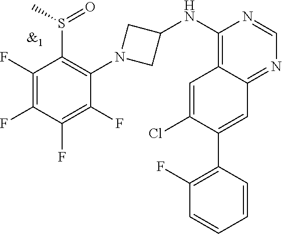 [SMILES: C[S@](=O)c1c(F)c(F)c(F)c(F)c1N1CC(Nc2ncnc3cc(-c4ccccc4F)c(Cl)cc23)C1] |
| 49A | 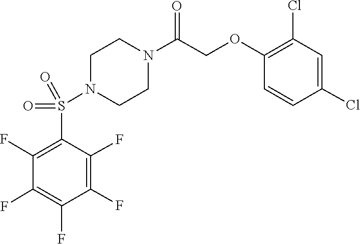 [SMILES: O=C(COc1ccc(Cl)cc1Cl)N1CCN(S(=O)(=O)c2c(F)c(F)c(F)c(F)c2F)CC1] |
| 50A | 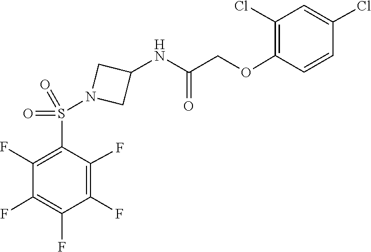 [SMILES: O=C(COc1ccc(Cl)cc1Cl)NC1CN(S(=O)(=O)c2c(F)c(F)c(F)c(F)c2F)C1] |
| 51A | 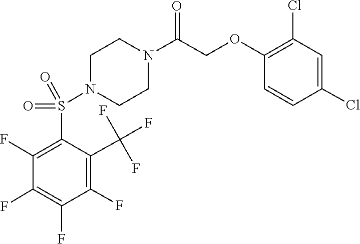 [SMILES: O=C(COc1ccc(Cl)cc1Cl)N1CCN(S(=O)(=O)c2c(F)c(F)c(F)c(F)c2C(F)(F)F)CC1] |
| 52A | 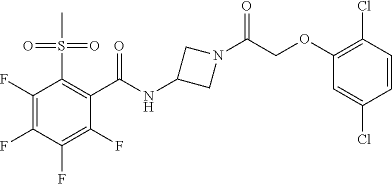 [SMILES: CS(=O)(=O)c1c(F)c(F)c(F)c(F)c1C(=O)NC1CN(C(=O)COc2cc(Cl)ccc2Cl)C1] |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Comwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Comwall, U.K.), Lancaster Synthesis (Windham, NH), Maybridge Chemical Co. Ltd. (Comwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the benzenesulfonamide derivative compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, a compound herein e.g., benzenesulfonamide derivative compound, is administered as a pure chemical. In other embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one compound described herein (e.g., benzenesulfonamide derivative compound), or a stereoisomer, pharmaceutically acceptable salt, hydrate, or solvate or tautomer or regioisomer thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a compound disclosed in Table 1, or a pharmaceutically acceptable salt or solvate or tautomer or regioisomer thereof.

One embodiment provides a method of preparing a pharmaceutical composition comprising mixing a compound of Formula (I), or a compound disclosed in Table 1, or a pharmaceutically acceptable salt or solvate or tautomer or regioisomer thereof, and a pharmaceutically acceptable carrier.

In certain embodiments, the benzenesulfonamide derivative compound as described by Formula (I), or a compound disclosed in Table 1, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the compound as described by Formula (I), or a compound disclosed in Table 1, or pharmaceutically acceptable salt or solvate or tautomer or regioisomer thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a compound disclosed in Table 1, or a pharmaceutically acceptable salt or solvate or tautomer or regioisomer thereof, for use in a method of treatment of the human or animal body.

One embodiment provides a compound of Formula (I), or a compound disclosed in Table 1, or a pharmaceutically acceptable salt or solvate or tautomer or regioisomer thereof, for use in a method of treatment of cancer or neoplastic disease.

One embodiment provides a use of a compound of Formula (I), or a compound disclosed in Table 1, or a pharmaceutically acceptable salt or solvate or tautomer or regioisomer thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt or solvate or tautomer or regioisomer thereof.

In some embodiments, described herein is a method of treating cancer in a patient in need thereof comprising administering to the patient a compound disclosed in Table 1, or a pharmaceutically acceptable salt or solvate or tautomer or regioisomer thereof.

In some embodiments, also described herein is a method of treating cancer in a patient in need thereof comprising administering to the patient a pharmaceutical composition comprising a compound of Formula (I), or a pharmaceutically acceptable salt or solvate or tautomer or regioisomer thereof, and a pharmaceutically acceptable excipient.

In some embodiments, also described herein is a method of treating cancer in a patient in need thereof comprising administering to the patient a pharmaceutical composition comprising a compound disclosed in Table 1, or a pharmaceutically acceptable salt or solvate or tautomer or regioisomer thereof, and a pharmaceutically acceptable excipient. In some embodiments, the cancer is selected from chronic and acute myeloid leukemia. In some embodiments, the cancer is selected from chronic lymphocytic leukemia and small lymphocytic lymphoma.

Provided herein is the method wherein the pharmaceutical composition is administered orally. Provided herein is the method wherein the pharmaceutical composition is administered by injection.

One embodiment provides a KRAS protein or an active fragment thereof (e.g., a polypeptide) modified with a benzenesulfonamide derivative compound as described herein, wherein the compound forms a covalent bond with a sulfur atom of a cysteine residue of KRAS protein.

One embodiment provides a method of modifying (e.g., attaching to and/or degrading) a polypeptide with a benzenesulfonamide derivative compound as described herein, comprising contacting the polypeptide with the compound to form a covalent bond with a sulfur atom of a cysteine residue of the polypeptide.

One embodiment provides a method of binding a compound to KRAS or an active fragment thereof (e.g., a polypeptide), comprising contacting the polypeptide with a benzenesulfonamide derivative compound as described herein.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

In some embodiments, the compounds disclosed herein are synthesized according to the following examples. As used below, and throughout the present description, the following abbreviations, unless otherwise indicated, shall be understood to have the following meanings:
° C. degrees Celsius
δ chemical shift in parts per million downfield from tetramethylsilane
ACN acetonitrile
bs or brs broad singlet
DCM dichloromethane (CH₂Cl₂)
dd doublet of doublets
DMF dimethylformamide
DMSO dimethylsulfoxide
EA or EtOAC ethyl acetate
ESI electrospray ionization
Et ethyl
FA formic acid
g gram(s)
h/hr/hrs hour(s)
HPLC high performance liquid chromatography
Hz hertz
J coupling constant (in NMR spectrometry)
LCMS liquid chromatography mass spectrometry
p micro
m multiplet (spectral); meter(s); milli
M molar
M+/− parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
Rbf round-bottom flask
RT room temperature
s singlet (spectral)
t triplet (spectral)
T temperature
TFA trifluoroacetic acid
THF tetrahydrofuran

Exemplary compounds of the application are synthesized using the methods described herein, or other methods, which are known in the art. Unless otherwise noted, reagents and solvents are obtained from commercial suppliers

Anhydrous solvents, methanol, acetonitrile, dichloromethane, tetrahydrofuran and dimethylformamide, are purchased from Sigma Aldrich and used directly from Sure-Seal bottles. Reactions are performed under an atmosphere of dry nitrogen in oven-dried glassware and are monitored for completeness by thin-layer chromatography (TLC) using silica gel (visualized by UV light, or developed by treatment with KMnO₄ stain and ninhydrin stain) or by LC/MS. NMR spectra are recorded in Bruker Avance III spectrometer at 23° C., unless stated otherwise, operating at 400 MHz for ¹H NMR, 376 MHz ¹⁹F and 100 MHz ¹³C NMR spectroscopy either in CDCl₃, CD₃OD, CD₃CN, or DMSO-d₆. Chemical shifts (d) are reported in parts per million (ppm) after calibration to residual isotopic solvent. Coupling constants (J) are reported in Hz. Mass spectrometry is performed with an Agilent G6110A single quad mass spectrometer with an ESI source associated with an Agilent 1100 capillary HPLC system. In some instances, the HPLC is equipped with a Phenomenex Luna 5 μm C18 150 mm×4.6 mm column. Before biological testing, inhibitor purity is evaluated by reversed-phase HPLC (rpHPLC).

The following conditions were employed for analysis by rpHPLC:

Method I: Mobile phase is a linear gradient consisting of a changing solvent composition of 10% to 90% ACN in H₂O with 0.1% TFA (v/v) over 7 minutes, followed by 5 minutes of 100% ACN. Method was run on a Welch Xtimate 5 μm C18, 150×4.6 mm column; column was maintained at a column temperature of 30° C.; flow rate was 1.0 mL/min. All retention times (RT) are explicitly denoted in minutes unless specifically stated otherwise.

Method II: Mobile phase is a linear gradient consisting of a changing solvent composition of 3% to 98% ACN solution (comprised of 9 parts ACN and 1 part MilliQ water containing 0.1% FA by volume) in H₂O with 0.1% FA (v/v) over 2.7 minutes, followed by 0.7 minutes of 100% ACN. Method was run on a Waters X-Bridge 2.5 μm C18, 50×2.1 mm; column was maintained at a column temperature of 30° C.; flow rate was of 0.8 mL/min. All retention times (RT) are explicitly denoted in minutes unless specifically stated otherwise.

Method III: Mobile phase is a linear gradient consisting of a changing solvent composition of 3% to 98% ACN in 5 mM ammonium bicarbonate in H₂O w (v/v) over 2.7 minutes, followed by 0.8 minutes of 100% ACN. Method was run at a on a Waters X-Bridge 3.5 μm C18, 50×2.1 mm; column was maintained at a column temperature of 35° C.; flow rate was 1.0 mL/min. All retention times (RT) are explicitly denoted in minutes unless specifically stated otherwise.

Resolved atropisomers were purified by CHIRALPAK IG (250×50 mm, 5 μm) with the uniform gradient of mobile phase, which was composed of 50% MeOH in liquid CO₂ with 150 mL/min over 20 minutes on WATERS SFC 350. The enantiomeric and diastereomeric excesses were determined by chiral SFC using CHIRALPAK IG (250×4.6 mm, 5 μm). Method run at a column temperature of 40° C. and a flow rate of 3.0 mL/min.

For reporting HPLC data, percentage purity is given after the retention time for each condition. All biologically evaluated compounds are >95% chemical purity as measured by HPLC.

In some embodiments, compounds of the present disclosure are synthesized using similar protocols based on the general procedures A-M, and Examples 1-10 below.

### Warhead Preparation: General Procedure AA

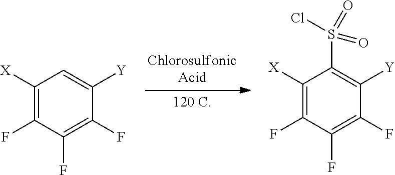

A substituted fluoro-arene (1 eq) was added to a cold solution of chlorosulfonic acid cooled to 0° C. The reaction vessel was outfitted with a water jacketed reflux condenser and subsequently heated to 120° C. using a sand bath for 1-16 hrs. Once starting material was consumed, the reaction was cooled to room temperature then poured slowly over crushed ice. The resulting mixture was partitioned between DCM and 1M HCl and the organic phase separated. The remaining aqueous phase was extracted twice more with DCM. The combined organic phases were washed with brine, dried over sodium sulfate, and concentrated in vacuo to afford the desired arylsulfonylchloride.

### Warhead Preparation: General Procedure BA

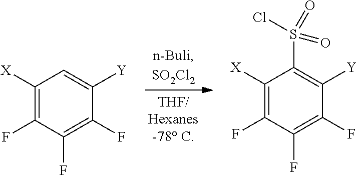

A substituted fluoro-arene (1 eq) was dissolved in anhydrous THF under a positive pressure of argon. The resulting solution was cooled to −78° C. Once at temperature, n-butyllithium (2.5 M in hexane, 1.2 eq) was added dropwise to limit excess evolution of heat. After 30 minutes, a solution of sulfuryl chloride (1.1 eq) in hexanes (0.1 M) was added quickly via syringe. After 1 hour, water was added to quench the reaction and the resulting mixture partitioned between ethyl acetate and cold water. The organic phase was separated, washed with cold water twice, dried over sodium sulfate and concentrated in vacuo to afford the anticipated sulfonylchloride.

### Warhead Preparation: General Procedure CA

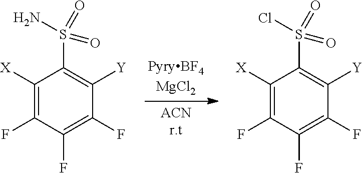

Under an inert atmosphere of argon, an appropriate aryl sulfonamide was added to a suspension of pyrylium tetrafluoroborate (2 eq) and magnesium chloride (2.5 eq) in acetonitrile (0.1 M) stirring at room temperature. The reaction was heated to 75° C. for 6 hours then cooled to room temperature. Once cooled, the mixture was filtered through a short plug of silica and the filtrate concentrated under reduced pressure. The concentrate was separated using flash column chromatography techniques to afford the desired sulfonylchloride.

### Warhead Preparation: General Procedure DA

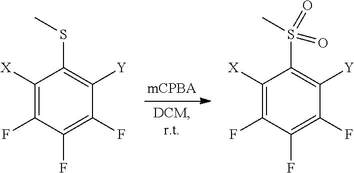

To a solution of thioether (1 eq) in DCM (0.1M-0.3M) at room temperature was added 3-Chloroperoxybenzoic acid (4 eq, 77% purity). Reaction progress was monitored by TLC. Once the starting material was consumed, the reaction was quenched with a 1M aqueous solution of sodium hydroxide. The organic phase was sepateated and the remaining aqueous extracted twice with dichloromethane. The combined organic phases were washed with brine, dried over anhydrous sodium sulfate and concentrated under vacuum. The crude material was separated using flash column chromatography techniques to afford the desired methylsulfone.

### Synthesis of Sulfonamides: General Procedure EA

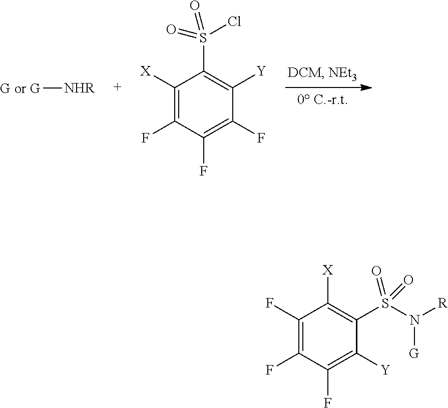

An appropriate sulfonylchloride (0.9-1.2 eq) was incubated with its corresponding pyrazololopyrimidine (1 eq) in anhydrous DCM (0.1 M-0.25 M) under an atmosphere of argon. The resulting mixture was cooled to 0° C. and stirred for 15 minutes. Neat triethylamine (3-5 eq) was slowly added to the mixture and it was stirred at 0° C. for a further 3-16 hrs. The reaction quenched with 0.1M HCl (aq) and vigorously stirred for 10-15 min, after which the organic layer was separated. The aqueous layer was extracted with DCM one further time. The combined organic layers were dried over sodium sulfate, filtered, and evaporated. The crude material was purified by either normal-phase flash column chromatography on silica gel or reverse-phase chromatography.

### Synthesis of Direct-Linked Sulfones: General Procedure FA

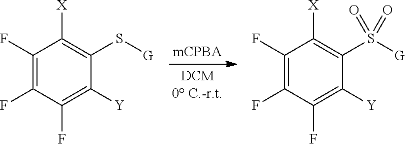

Methods to oxidize analogous thioethers to the corresponding sulfone are known in the art (WO2019/141694). The G linked sulfone can be prepared from the corresponding thioether in the presence of 3-Chloroperoxybenzoic acid (mCPBA, 4 eq.) in DCM under inert conditions (argon or nitrogen). The reaction can be worked up with water, brine and DCM, and the desired sulfone isolated using normal-phase flash column chromatography on silica gel or reverse-phase chromatography.

### Synthesis of Direct-Linked Sulfoximines: General Procedure GA

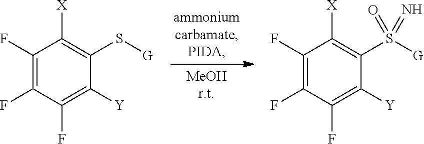

Methods to oxidize analogous thioethers to the corresponding sulfoximine are known in the art (Chem. Comm., 2017, 12, p. 2064-2067). The G linked sulfoximine can be prepared from the corresponding thioether in the presence of ammonium carbamate (1.5 eq.), iodobenzenediacetate (PIDA, 2.1 eq.) in methanol at room temperature. The reaction can be worked up with water, brine and DCM, and the desired sulfoximine isolated using normal-phase flash column chromatography on silica gel or reverse-phase chromatography.

### Synthesis of Sulfonimidamides: General Procedure HA

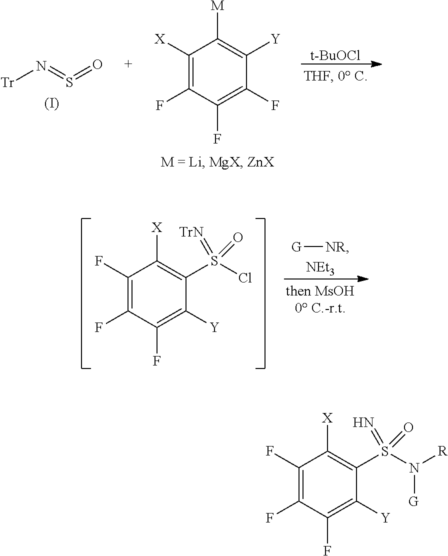

The starting material, compound (I), can be prepared according to previously reported procedures (Angew. Chem. Int. Ed. 2017, 56, 14937). An oven-dried flask charged with (I) (1.0 equiv.) and THF (0.1 M) is cooled to 0° C. Then the corresponding organometallic reagent (1.0 equiv.) can be added dropwise and stirred at 0° C. for 5 min. Next, in a dark fume hood, tert-butyl hypochlorite (1.05 equiv.) is added and the reaction mixture is allowed to stir for 15 min, followed by the addition of triethylamine (1.0 equiv.) and the corresponding ligand (G or GNR) (1.0-1.2 equiv.). The reaction mixture is left stirring at room temperature for 16 h. Finally, methanesulfonic acid (5.0 equiv.) is added, and the reaction stirred vigorously for 15 min at room temperature. The reaction is quenched by diluting it with DCM and the addition of a saturated aqueous solution of sodium bicarbonate. The two layers are partitioned and the aqueous layer is extracted with DCM (×3). Combined organic layers are dried over magnesium sulfate (MgSO₄), filtered and concentrated in vacuo. Crude samples can be purified by either normal-phase flash column chromatography on silica gel or reverse-phase chromatography.

### Synthesis of Para-Direct-Linked Sulfones: General Procedure IA

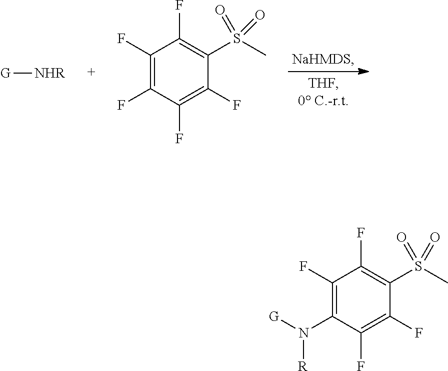

Procedures detailing the addition of a nucleophile to the 4-position of the pentafluorobenzene methyl sulfone are known in the field. In one embodiment, G-NHR can be deprotonated in THF using sodium hexamethyldisilazane to prepare the corresponding sodium amide. The sodium amide can then be added to a cold solution (0° C.) of 1,2,3,4,5-pentafluoro-6-(methylsulfonyl)benzene in THF to prepare the anticipated para-substituted tetrafluorobenzene sulfone. The reaction can be worked up with water, brine and EtOAc, and the product isolated using normal-phase flash column chromatography on silica gel or reverse-phase chromatography.

### Synthesis of Ortho-Direct-Linked Sulfones: General Procedure JA

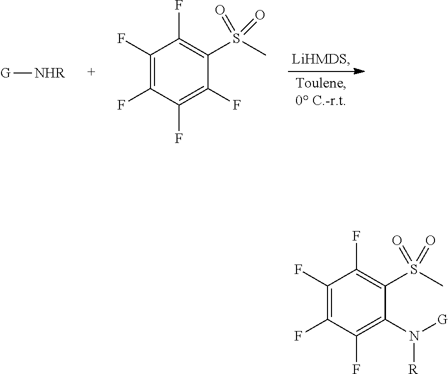

Procedures detailing the addition of a nucleophile to the 2-position of the pentafluorobenzene methyl sulfone are known (e.g., using NaH as in WO2006/81332, 2006). In one embodiment, G-NHR can be deprotonated in THF using lithium hexamethyldisilazane to prepare the corresponding lithium amide. The lithium amide can then be added to a cold solution (0° C.) of 1,2,3,4,5-pentafluoro-6-(methylsulfonyl)benzene in toluene to prepare the anticipated ortho-substituted tetrafluorobenzene sulfone. The reaction can be worked up with water, brine and EtOAc, and the product isolated using normal-phase flash column chromatography on silica gel or reverse-phase chromatography.

### Synthesis of Alpha-Bromo-Methylsulfone: General Procedure KA

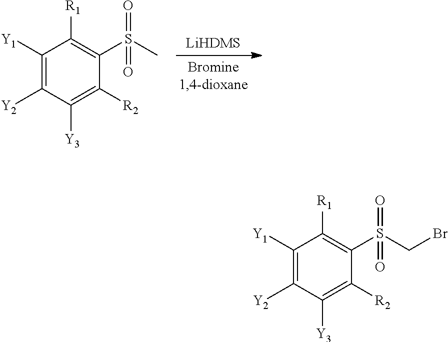

Procedures detailing the bromination of methyl sulfone are known (Chem. Commun., 2019, 55, 2912). In one embodiment, methylsulfone is dissolved in anhydrous 1,4-dioxane and to the resulting solution is added LiHMDs and stirred for 1.5 hours under N₂. The deprotonated sulfone solution is then added to a solution of Br² in anhydrous 1,4-dioxane. The reaction can be worked up with slow addition of aqueous saturated Na₂SO₃ and product isolated using normal-phase flash column chromatography on silica gel.

### Synthesis of C-Linked Methylsulfone: General Procedure LA

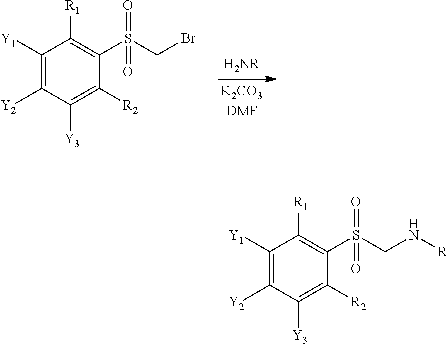

Into a solution of bromomethyl sulfone in anhydrous DMF is added appropriate amine at 0° C. The reaction mixture is then added with K₂CO₃ in a dropwise manner at 0° C. and the resulting solution is stirred while gradually warming to room temperature. Upon completion, the reaction is worked up with water and extracted three times with EtOAc. The collected organic layer is washed with brine, dried with sodium sulfate, filtered and evaporated under reduced pressure. The product is isolated using normal-phase flash column chromatography on silica gel.

### Synthesis of Ethylene-Linked Sulfone: General Procedure MA

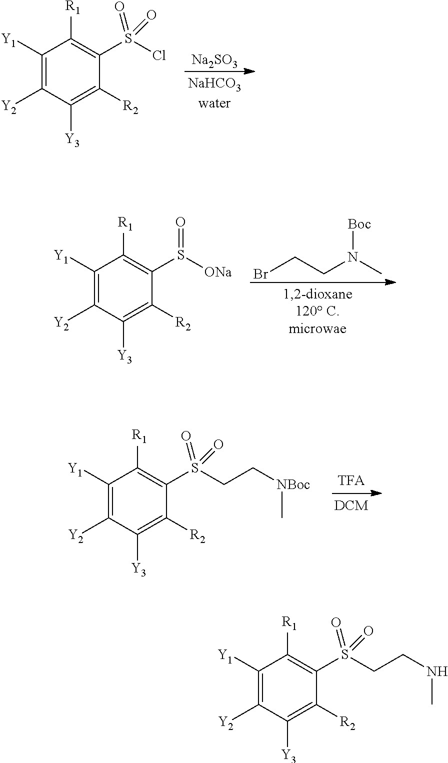

Procedures detailing transformation of sulfonyl chloride to sulfinate salt are known (Organic Letters 2019 21 (17), 7174-7178). In one embodiment, to a solution of Na₂CO₃ and NaHCO₃ in water was added sulfonyl chloride at room temperature. The reaction is then stirred at 70° C. Upon completion, the reaction mixture is evaporated and the resulting mixture is suspended in EtOH and stirred for 20 minutes at room temperature. Any suspension as a result is filtered and the solid is washed with EtOH, and the collected filtrate is evaporated under reduced pressure to yield the desired sulfinate sodium salt.

Procedures detailing alkylation of sodium sulfinate are known (Organic Letters 2019 21 (17), 7174-7178). In one embodiment, the sodium sulfinate is dissolved in anhydrous DMF under Ar and then treated with commercially available tert-butyl (2-bromoethyl)(methyl)carbamate. The resulting mixture is stirred while heating at 80° C. The reaction is worked up with water at room temperature and extracted with EtOAc. The combined organic layer is washed with brine, dried with sodium sulfate, filtered and evaporated under reduced pressure. The product is isolated using normal-phase flash column chromatography on silica gel.

Procedures for the removal of tert-butoxycarbonyl group involves dissolution of the sulfone in anhydrous DCM and the resulting solution is added with TFA at room temperature. Upon completion of the reaction, the mixture is evaporated under reduced pressure. The product is diluted with EtOAc and washed with saturated sodium carbonate solution, dried with sodium sulfate, filtered and evaporate under reduced pressure.

### Example 1A: 2-(difluoromethyl)-3,4,5,6-tetrafluoro-N-(6-fluoro-7-(2-fluoro-6-hydroxyphenyl)-1-(2-isopropylphenyl)-2-oxo-1,2-dihydropyrido[2,3-d]pyrimidin-4-yl)-N-methylbenzenesulfonamide (Compound 36, Table 1)

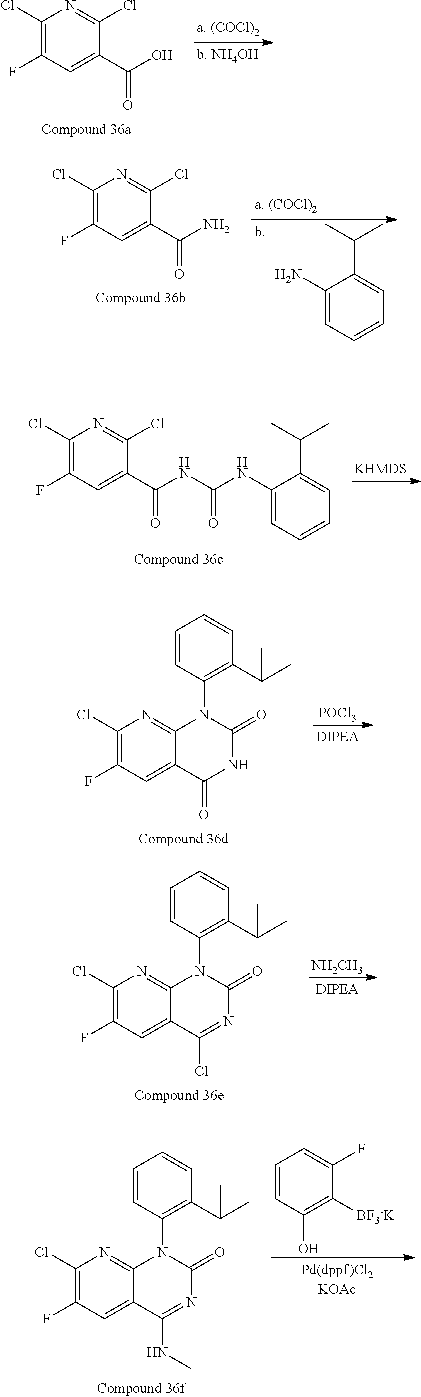

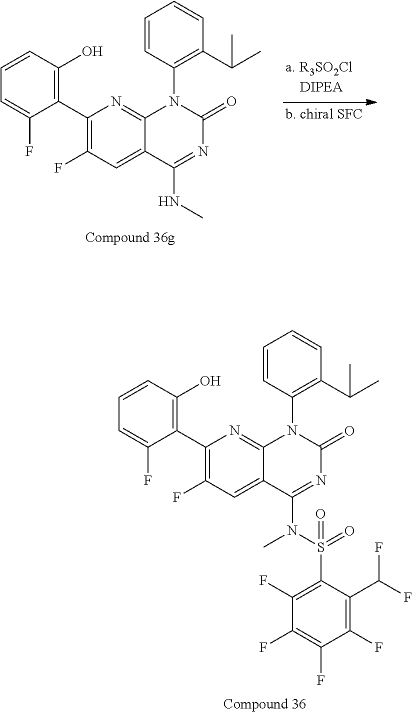 [SMILES: CNc1nc(=O)n(-c2ccccc2C(C)C)c2nc(-c3c(O)cccc3F)c(F)cc12 ; CC(C)c1ccccc1-n1c(=O)nc(N(C)S(=O)(=O)c2c(F)c(F)c(F)c(F)c2C(F)F)c2cc(F)c(-c3c(O)cccc3F)nc21]

Compound 36b can be prepared as described in Journal of Medicinal Chemistry 2020 63 (1), 52-65 by converting the commercially available 2,6-dichloro-5-fluoronicotinic acid (Compound 36a) into acid chloride using oxalyl chloride followed by addition of NH₄C. Compound 36b can be sequentially transformed into Compound 36c using oxalyl chloride and 2-isopropylaniline using a procedure analogous to the one known in the art. Compound 36c can be converted to Compound 36d by base-mediated cyclization using KHMDS, which can then be chlorinated using POCl₃ to afford Compound 36e. The Compound 36f can be formed by nucleophilic aromatic substitution of Compound 36e with methylamine. The Compound 36f can be coupled with (2-fluoro-6-hydroxyphenyl)potassium trifluoroborate using Pd(dppf)Cl₂ based on the procedure described in the one known in the art to afford the Compound 36g. Compound 36 can be prepared from sulfonylation of Compound 36g with 2-(difluoromethyl)-3,4,5,6-tetrafluorobenzenesulfonyl chloride (prepared as described in General Procedure BA) by using General Procedure EA. The title compound is then purified using chiral supercritical fluid chromatography to afford the product.

### Example 2A: 2,3,4,5-tetrafluoro-N-(6-fluoro-7-(2-fluoro-6-hydroxyphenyl)-1-(2-isopropylphenyl)-2-oxo-1,2-dihydropyrido[2,3-d]pyrimidin-4-yl)-6-methylbenzenesulfonamide (Compound 37, Table 1)

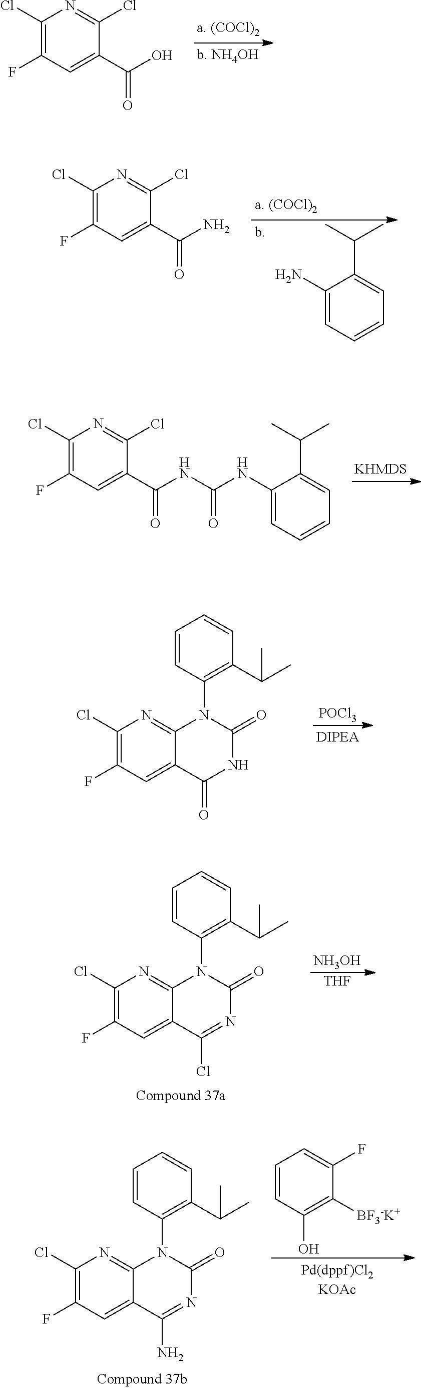

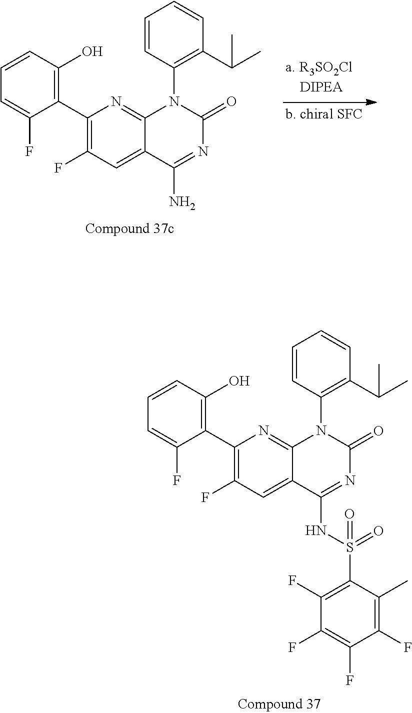 [SMILES: CC(C)c1ccccc1-n1c(=O)nc(N)c2cc(F)c(-c3c(O)cccc3F)nc21 ; Cc1c(F)c(F)c(F)c(F)c1S(=O)(=O)Nc1nc(=O)n(-c2ccccc2C(C)C)c2nc(-c3c(O)cccc3F)c(F)cc12]

Compound 37a can be prepared according to the procedure as described in Journal of Medicinal Chemistry 2020 63 (1), 52-65. Compound 37b can be prepared from Compound 37a via nucleophilic aromatic substitution using ammonium hydroxide in THF. The isolated Compound 37b can be converted to Compound 37c with the procedure adapted from Journal of Medicinal Chemistry 2020 63 (1), 52-65. Lastly, Compound 37 can be prepared from sulfonylation of Compound 37c with 2,3,4,5-tetrafluoro-6-methylbenzenesulfonyl chloride (prepared as described in General Procedure BA) by using General Procedure EA. The title compound can be purified using chiral supercritical fluid chromatography to afford the product

### Example 3A: 2,3,4,5-tetrafluoro-N-((6-fluoro-7-(2-fluoro-6-hydroxyphenyl)-1-(2-isopropylphenyl)-2-oxo-1,2-dihydropyrido[2,3-d]pyrimidin-4-yl)methyl)-6-(fluoromethoxy)benzenesulfonamide (Compound 38, Table 1)

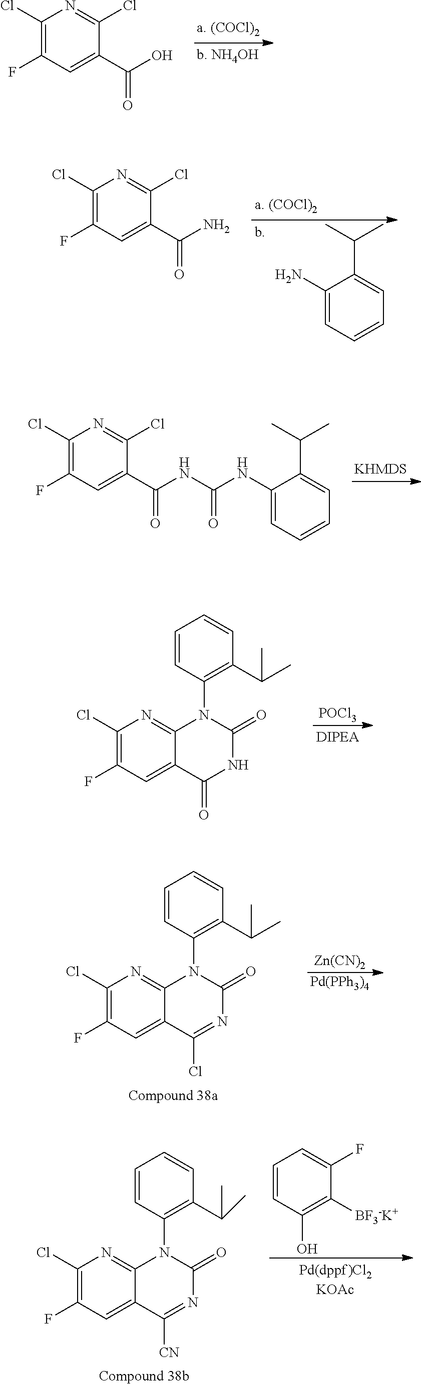

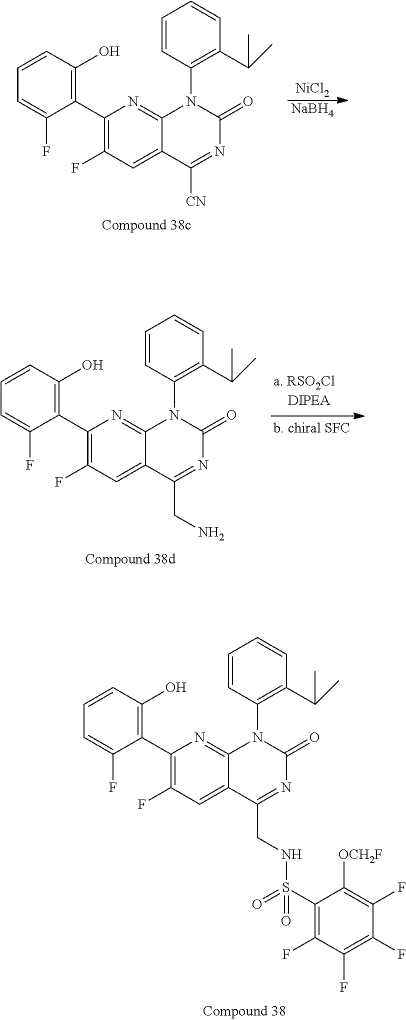

Compound 38a can be prepared based on the procedure adapted from Journal of Medicinal Chemistry 2020 63 (1), 52-65. Compound 38b can be obtained by treating Compound 38a with Zn(CN)₂ using Pd(PPh₃)₄ in DMF (procedure adapted from US2015/148358, 2015). Compound 38c can be obtained from Compound 38b using a procedure analogous to the one known in the art. The isolated Compound 38c can be reduced to Compound 38d using NiCl₂ and NaBH₄ based on a procedure from Tetrahedron 2003 59, 5417-5423. Lastly, the Compound 38 can be prepared from sulfonylation of Compound 38d with 2,3,4,5-tetrafluoro-6-(fluoromethoxy)benzenesulfonyl chloride (prepared as described in General Procedure BA) by using General Procedure EA. The title compound can be purified using chiral supercritical fluid chromatography to afford the product

### Example 4A: 6-fluoro-7-(2-fluoro-6-hydroxyphenyl)-1-(2-isopropylphenyl)-4-((((2,3,4,5-tetrafluoro-6-(fluoromethoxy)phenyl)sulfonyl)methyl)amino)pyrido[2,3-d]pyrimidin-2(1H)-one (Compound 40, Table 1)

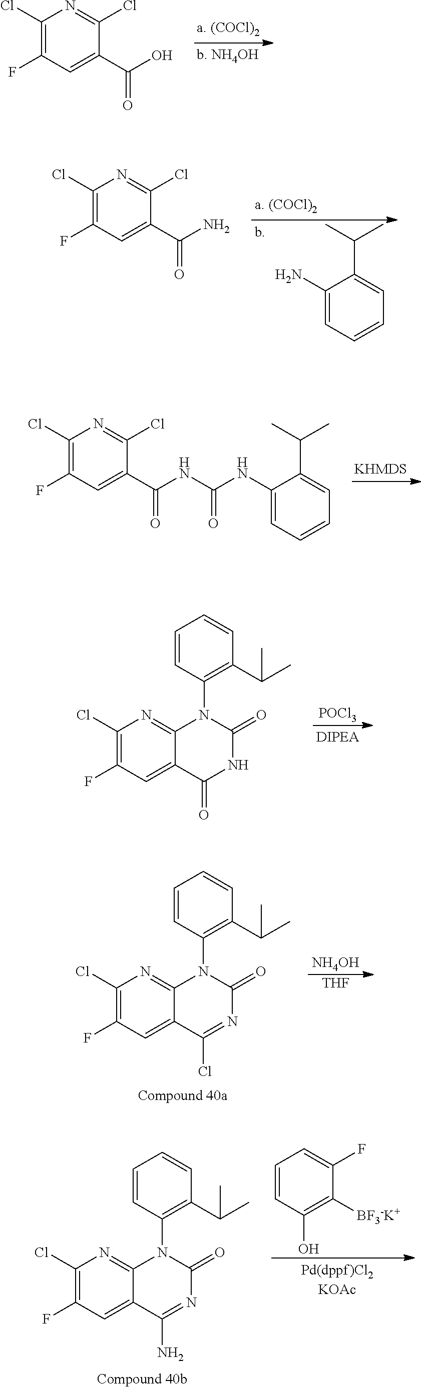

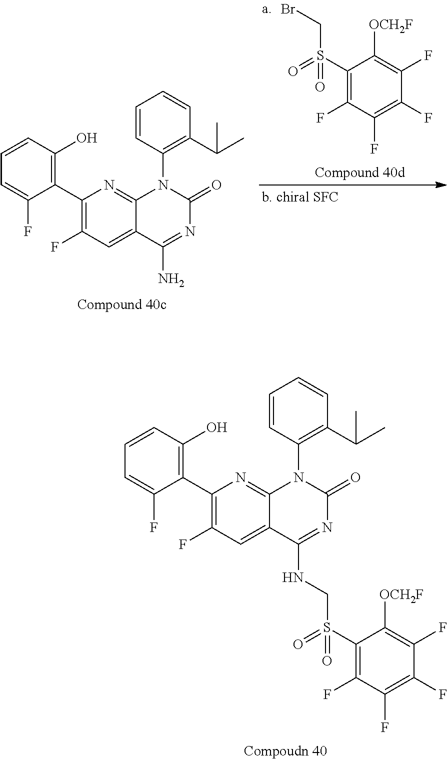

Compound 40a can be prepared based on the procedure adapted from Journal of Medicinal Chemistry 2020 63 (1), 52-65. After nucleophilic aromatic substitution of Compound 40a with NH₄OH to afford Compound 40b, the Compound 40b can be converted to Compound 40c by the procedure analogous to the one known in the art. The Compound 40d can be prepared by General Procedure KA. Compound 40 can be obtained from Compound 40c and Compound 40d using General Procedure LA. The title compound can be purified using chiral supercritical fluid chromatography to afford the product

### Example 5A: 2-((4-chloro-2-methoxyphenyl)amino)-N-methyl-N-(2-((2,3,4,5-tetrafluoro-6-(trifluoromethoxy)phenyl)sulfonyl)ethyl)acetamide (Compound 41, Table 1)

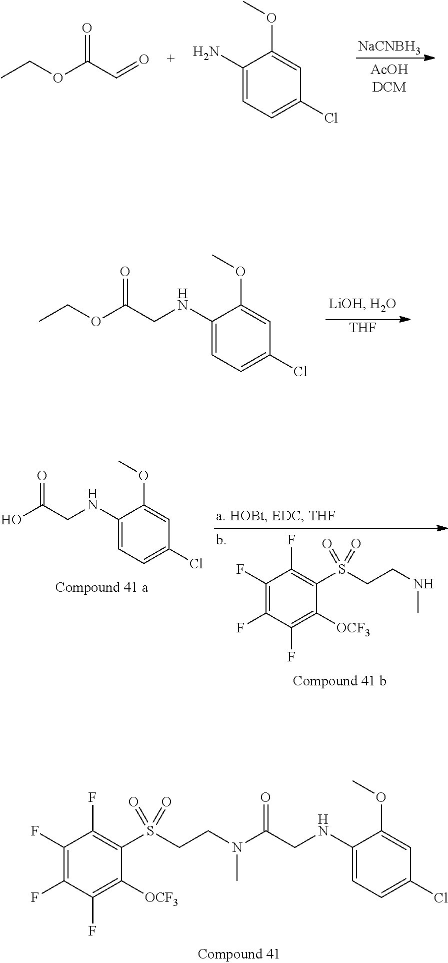

Compound 41a can be prepared by reductive amination of commercially available ethyl 2-oxoacetate with 4-chloro-2-methoxyaniline in the presence of NaCNBH₃ and AcOH in DCM, followed by base hydrolysis using LiOH (the procedure adapted from Nature 2013 503, 548-551). Compound 41 b can be obtained by using the General Procedure MA. Compound 41 can be obtained by activating Compound 41a with HOBt and EDC followed by coupling with Compound 41b according to the procedure adapted from Nature 2013 503, 548-551.

### Example 6A: 2-(2,4-dichlorophenoxy)-N-methyl-N-((2,3,4,5-tetrafluoro-6-methoxyphenyl)sulfonyl)acetamide (Compound 45, Table 1)

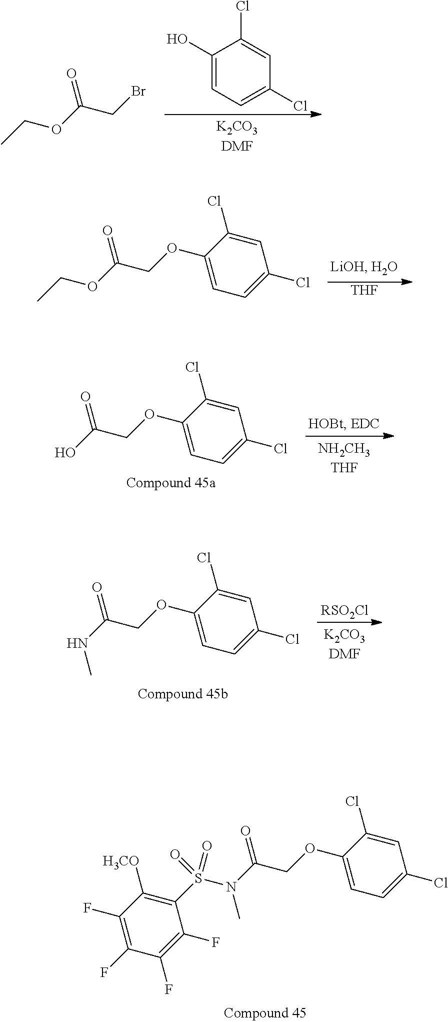

Compound 45a can be prepared by the procedure adapted from Nature 2013 503, 548-551, in which commercially available ethyl 2-bromoacetate is treated with 2,4-dichlorophenol and K₂CO₃ in DMF followed by hydrolysis of the ethyl ester in the presence of aqueous LiOH in THF. Compound 45b can be obtained by the procedure analogous to the one known in the field, whereby Compound 45a was coupled with methylamine using HOBt and EDC. Lastly, Compound 45 can be obtained from Compound 45b and 2,3,4,5-tetrafluoro-6-methoxybenzenesulfonyl chloride (prepared as described in General Procedure BA) by using General Procedure EA.

### Example 7A: (S)-4-((1-methylpyrrolidin-2-yl)methoxy)-7-(naphthalen-1-yl)-5,6,7,8-tetrahydropyrido[3,4-d]pyrimidin-2-yl 2,3,4,5-tetrafluoro-6-(fluoromethyl)benzenesulfonate (Compound 66, Table 1)

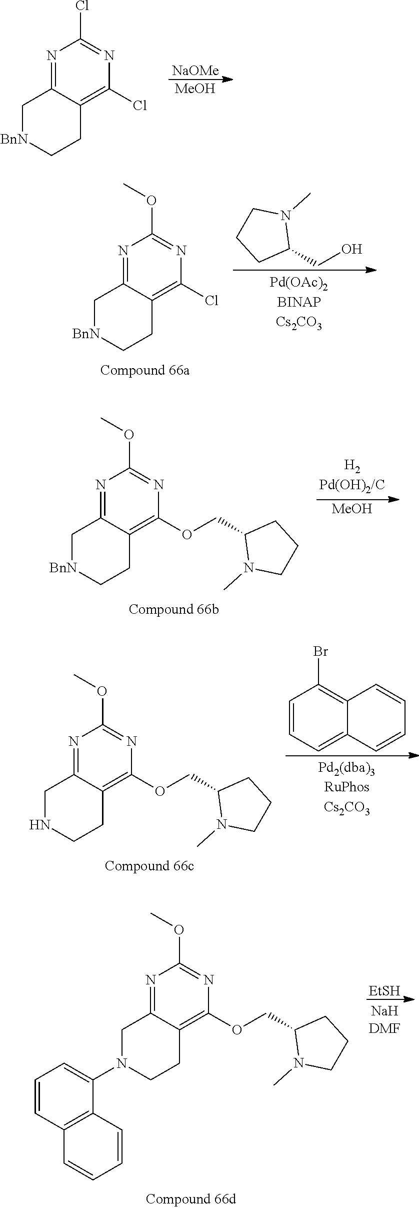

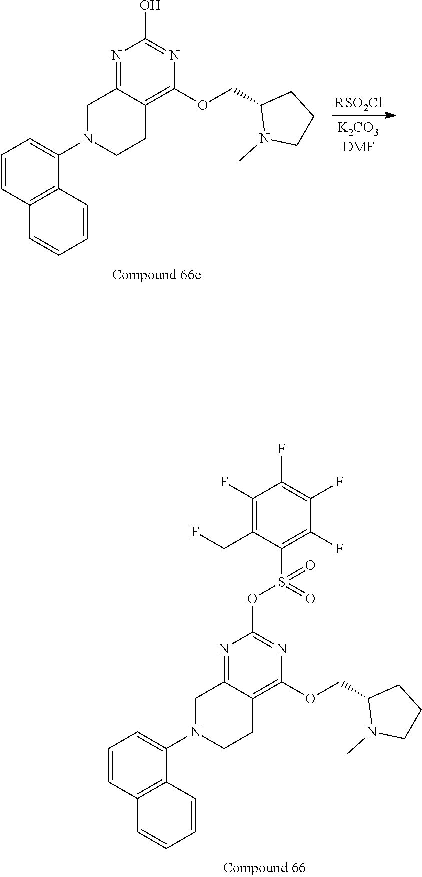

Compound 66a can be prepared by nucleophilic aromatic substitution of commercially available 7-benzyl-2,4-dichloro-5,6,7,8-tetrahydropyrido[3,4-d]pyrimidine with NaOMe as described in Journal of Medicinal Chemistry 2020 63 (13), 6679-6693. Compound 66b can be prepared by coupling Compound 66a with (S)-(1-methylpyrrolidin-2-yl)methanol using Pd(OAc)₂, BINAP and Cs₂CO₃. Compound 66b can be reduced to Compound 66c using Pd(OH)₂/C and H₂. The Compound 66d is then demethylated to Compound 66e using EtSH and NaH in DMF. Compound 66 can be prepared from Compound 66e by treating it with 2,3,4,5-tetrafluoro-6-(fluoromethyl)benzenesulfonyl chloride (prepared as described in General Procedure BA) and K₂CO₃ in DMF as outlined in General Procedure EA.

### Example 8A: (S)-2-(difluoromethoxy)-3,4,5,6-tetrafluoro-N-(2-((1-methylpyrrolidin-2-yl)methoxy)-7-(naphthalen-1-yl)-5,6,7,8-tetrahydropyrido[3,4-d]pyrimidin-4-yl)benzenesulfonamide (Compound 72, Table 1)

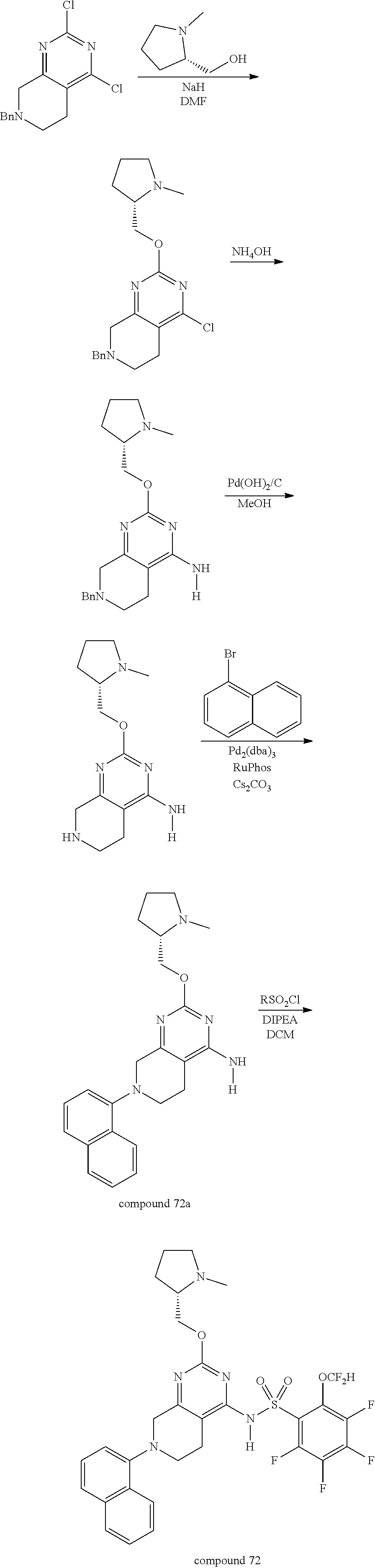

Compound 72a can be prepared as described in Journal of Medicinal Chemistry 2020 63 (13), 6679-6693. Compound 72 can be prepared from Compound 72a by treating it with 2-(difluoromethoxy)-3,4,5,6-tetrafluorobenzenesulfonyl chloride (prepared as described in General Procedure BA) and DIPEA in DCM as outlined in General Procedure EA.

### Example 9: 4-((S)-4-((2-(difluoromethyl)-3,4,5,6-tetrafluorophenyl)sulfonyl)-2-methylpiperazin-1-yl)-6-fluoro-7-(2-fluoro-6-hydroxyphenyl)-1-(2-isopropylphenyl)pyrido[2,3-d]pyrimidin-2(1H)-one (Compound 1, Table 2)

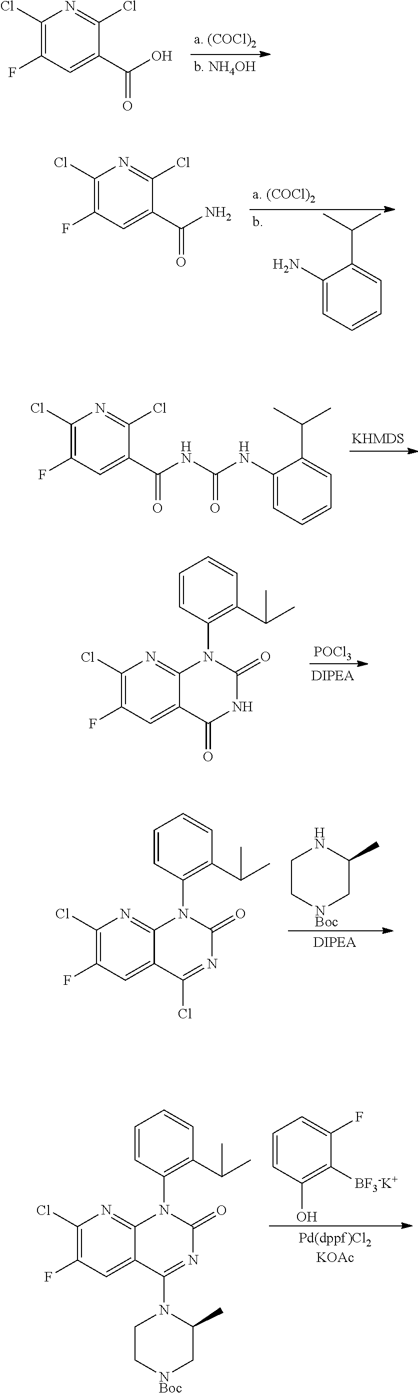

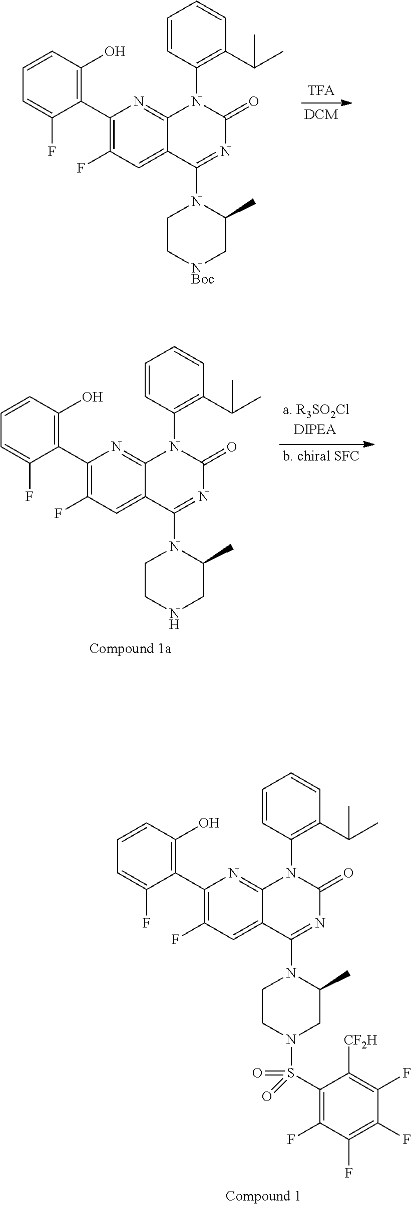 [SMILES: CC(C)c1ccccc1-n1c(=O)nc(N2CCN(C(=O)OC(C)(C)C)C[C@@H]2C)c2cc(F)c(-c3c(O)cccc3F)nc21 ; CC(C)c1ccccc1-n1c(=O)nc(N2CCNC[C@@H]2C)c2cc(F)c(-c3c(O)cccc3F)nc21 ; CC(C)c1ccccc1-n1c(=O)nc(N2CCN(S(=O)(=O)c3c(F)c(F)c(F)c(F)c3C(F)F)C[C@@H]2C)c2cc(F)c(-c3c(O)cccc3F)nc21]

Compound 1a can be prepared as described in Journal of Medicinal Chemistry 2020 63 (13), 6679-6693. Compound 1 can be prepared from Compound 1a by treating it with 2-(difluoromethyl)-3,4,5,6-tetrafluorobenzenesulfonyl chloride (prepared as described in General Procedure BA) and DIPEA in DCM as outlined in General Procedure EA.

### Example 10A: N-(3-(5-chloro-2-methoxybenzoyl)benzyl)-2,3,4,5-tetrafluoro-6-methoxybenzenesulfonamide (Compound 7, Table 2)

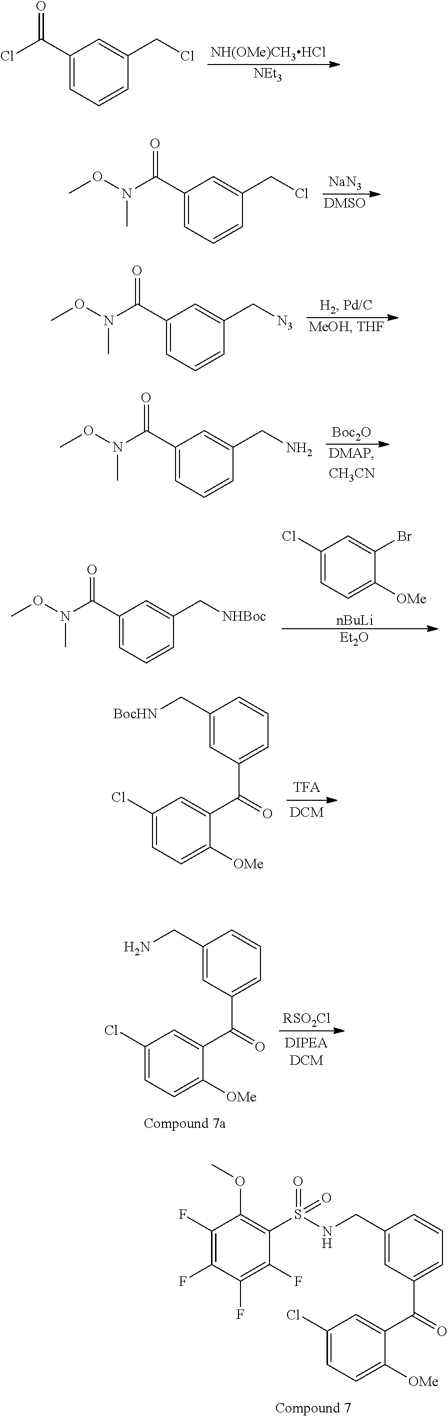

Compound 7a can be prepared as described in Journal of Medicinal Chemistry 2006 49 (2), 727-739. Compound 7 can be prepared from Compound 7a by treating it with 2,3,4,5-tetrafluoro-6-methoxybenzenesulfonyl chloride (prepared as described in General Procedure BA) and DIPEA in DCM as outlined in General Procedure EA.

### General Procedure A:

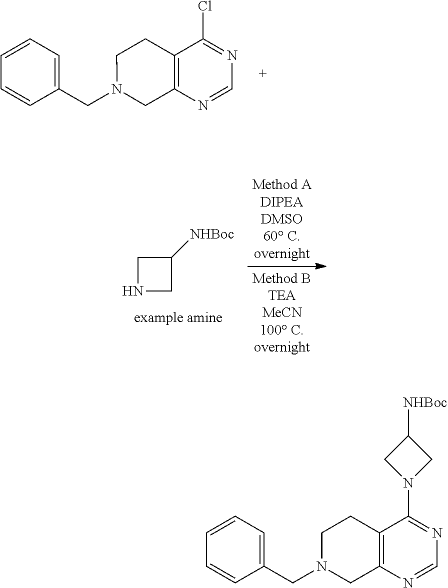 [SMILES: Clc1ncnc2c1CCN(Cc1ccccc1)C2 ; CC(C)(C)OC(=O)NC1CNC1 ; CC(C)(C)OC(=O)NC1CN(c2ncnc3c2CCN(Cc2ccccc2)C3)C1]

Method A: Commercially available reactant (1 eq.) and appropriate amine (1.5 eq.) were dissolved in anhydrous DMSO at ambient temperature. Then, N,N-Diisopropylethylamine (3 eq.) was added to the solution in a dropwise manner. The resulting solution was stirred at 60° C. for 12 h under N₂ atmosphere. After the completion of the reaction, water was added to the resulting solution and the resulting reaction mixture was extracted with EtOAc three times. Then, the collected organic layer was washed with saturated solution of sodium chloride and then dried over sodium sulfate. The separated organic layer was filtered, and volatiles were then removed from the combined filtrate under reduced pressure. The resulting crude product was purified by normal phase column chromatography eluting in gradient from 60%-100% EtOAc in Hexanes to afford the product.

Method B: Commercially available reactant (1 eq.) and appropriate amine (1.5 eq.) were dissolved in anhydrous acetonitrile at ambient temperature. Then, triethylamine (6 eq.) was added to the solution in a dropwise manner. The resulting solution was stirred at 100° C. for 12 h under an N₂ atmosphere. After the completion of the reaction, water was added to the resulting solution and extracted with EtOAc three times. Then the collected organic layer was washed with saturated solution of sodium chloride and then dried over sodium sulfate. The separated organic layer was filtered, and volatiles were then removed from the combined filtrate under reduced pressure to afford the product.

### General Procedure B:

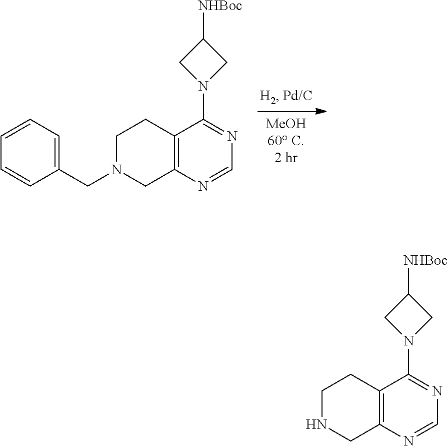 [SMILES: CC(C)(C)OC(=O)NC1CN(c2ncnc3c2CCN(Cc2ccccc2)C3)C1 ; CC(C)(C)OC(=O)NC1CN(c2ncnc3c2CCNC3)C1]

Reactant (1 eq.) was dissolved in anhydrous MeOH. Then, palladium on carbon (10% w/w, 2.21 eq.) was added to the solution at room temperature. The resulting solution was stirred at 50° C. for 2 hours. After the completion the resulting solution has been filtered through celite. The filtered, and volatiles were then removed from by evaporation under reduced pressure to afford the product.

### General Procedure C:

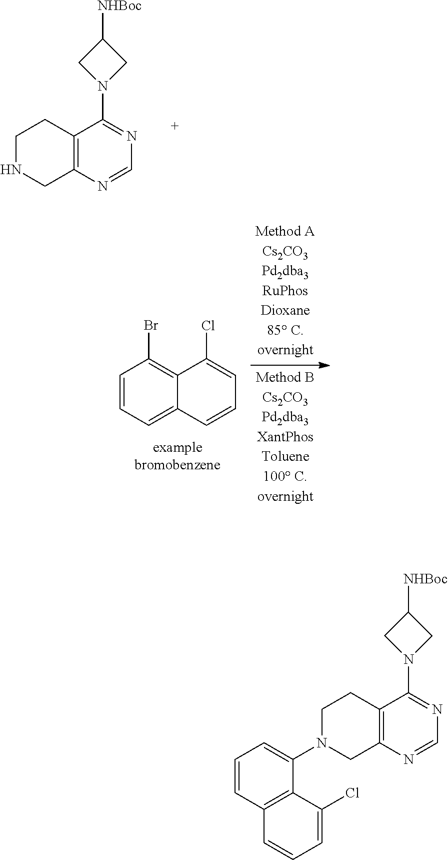 [SMILES: CC(C)(C)OC(=O)NC1CN(c2ncnc3c2CCNC3)C1 ; Clc1cccc2cccc(Br)c12 ; CC(C)(C)OC(=O)NC1CN(c2ncnc3c2CCN(c2cccc4cccc(Cl)c24)C3)C1]

Method A: Reactant (1 eq.), appropriate bromobenzene R₃ (1.3 eq.), cesium carbonate (2.5 eq.), (1E,4E)-1,5-di(phenyl)penta-1,4-dien-3-one; palladium (0.15 eq.), and [2-[2,6-bis(1-methylethoxy)phenyl]phenyl]-di(cyclohexyl)phosphane (0.3 eq.) were combined and dissolved in anhydrous 1,4-dioxane. The mixture was purged with nitrogen three times. The resulting solution was stirred at 85° C. for 12 hours under N₂. After the completion, the resulting solution was quenched with water and extracted with EtOAc three times. Then the collected organic layer was washed with saturated solution of sodium chloride and then dried over sodium sulfate. The separated organic layer was filtered, and volatiles were then removed from the combined filtrate under reduced pressure to afford the product.

Method B: Reactant (1 eq.), appropriate bromobenzene R₃ (1.3 eq.), cesium carbonate (2.5 eq.), (1E,4E)-1,5-di(phenyl)penta-1,4-dien-3-one; palladium (0.2 eq.), and Xantphos (0.4 eq.) were combined and dissolved in anhydrous toluene. The mixture was purged with nitrogen three times. The resulting solution was stirred at 100° C. for 12 hours under N₂. After the completion, the resulting solution was quenched with water and extracted with EtOAc three times. Then, the collected organic layer was washed with saturated solution of sodium chloride and then dried over sodium sulfate. The separated organic layer was filtered, and volatiles were then removed from the combined filtrate under reduced pressure to afford the product.

### General Procedure D:

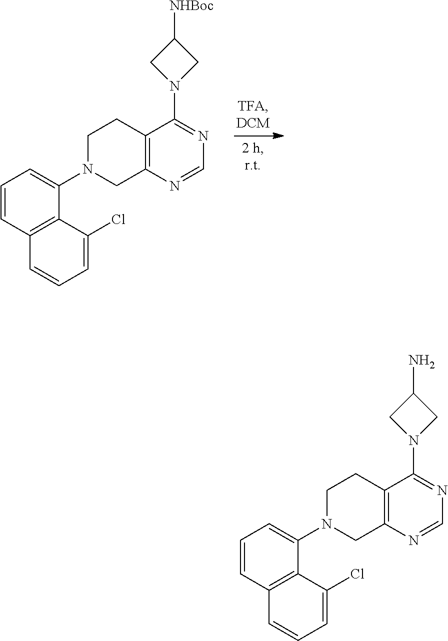 [SMILES: CC(C)(C)OC(=O)NC1CN(c2ncnc3c2CCN(c2cccc4cccc(Cl)c24)C3)C1 ; NC1CN(c2ncnc3c2CCN(c2cccc4cccc(Cl)c24)C3)C1]

Reactant (1 eq.) was dissolved in anhydrous DCM. Then, trifluoroacetic acid (30 eq.) was added dropwise at ambient temperature. The combined solution was stirred at room temperature for 2.5 hours. The resulting solution was then quenched and neutralize with saturated sodium bicarbonate and extracted with DCM three times. The collected organic layer was washed with water and saturated sodium sulfate solution. The separated organic layer was dried over sodium sulfate, filtered, and volatiles were then removed from the combined filtrate under reduced pressure.

### General Procedure E:

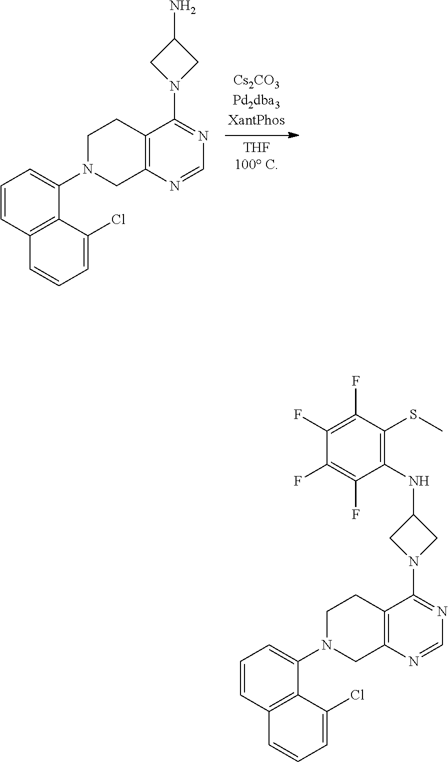 [SMILES: NC1CN(c2ncnc3c2CCN(c2cccc4cccc(Cl)c24)C3)C1 ; CSc1c(F)c(F)c(F)c(F)c1NC1CN(c2ncnc3c2CCN(c2cccc4cccc(Cl)c24)C3)C1]

Reactant (1 eq.), (2-bromo-3,4,5,6-tetrafluorophenyl)(methyl)sulfane (1.1 eq.), cesium carbonate (2 eq.), (1E,4E)-1,5-di(phenyl)penta-1,4-dien-3-one; palladium (0.1 eq.), Xantphos (0.1 eq.) were combined and dissolved in anhydrous THF at room temperature. The mixture was purged with nitrogen three times and then the resulting solution was stirred at 100° C. for 12 h under N₂. The resulting solution was quenched with water and extracted with EtOAc three times. The collected organic layer was dried over sodium sulfate, filtered, and volatiles were then removed from the combined filtrate under reduced pressure to afford the product.

### General Procedure F:

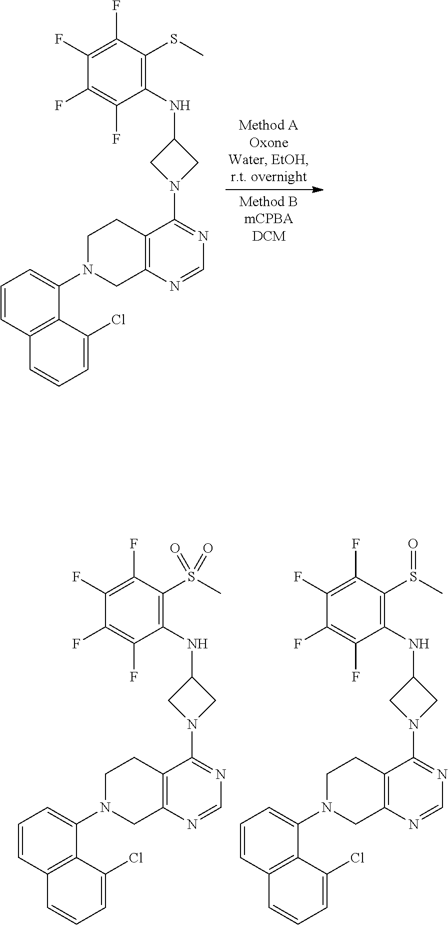

Method A: Oxone, monopersulfate compound (4.5 eq) was dissolved in water. Then, this solution was added with a solution of reactant (1 eq.) in anhydrous ethanol in a dropwise manner. The combined solution was stirred at room temperature for 12 hours. The resulting solution was extracted with EtOAc three times, and the collected organic layer was dried over sodium sulfate, filtered, and volatiles were then removed from the combined filtrate under reduced pressure to afford the mixture containing two products. The crude product was purified by reverse-phase column chromatography eluting in gradient from 10%-60% MeCN in Mili-Q water (+0.1% FA) to separate the two products.

Method B: Reactant (1 eq.) was dissolved in anhydrous DCM and the 3-chloranylbenzenecarboperoxoic acid (3 eq.) was added in portions to this solution. The resulting mixture was stirred at rt for 24 h. The mixture was quenched with sodium bicarbonate solution and extracted with DCM three times. Then, the collected organic layer was washed with saturated solution of sodium chloride and then dried over sodium sulfate. The separated organic layer was filtered, and volatiles were then removed from the combined filtrate under reduced pressure.

### General Procedure G:

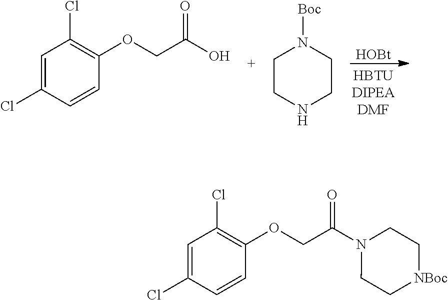 [SMILES: O=C(O)COc1ccc(Cl)cc1Cl ; CC(C)(C)OC(=O)N1CCNCC1 ; CC(C)(C)OC(=O)N1CCN(C(=O)COc2ccc(Cl)cc2Cl)CC1 ; "HOBt"]

Acid (1 eq.) was dissolved in anhydrous DMF (0.18 M). After 5 minutes of stirring at r.t., neat [benzotriazol-1-yloxy(dimethylamino)methylene]-dimethyl-ammonium; hexafluorophosphate (1.2 eq.), HOBt (1.2 eq.), and N-ethyl-N-isopropyl-propan-2-amine (2 eq.) were added. To the suspension was added appropriate Boc-protected amine (1.1 eq.). After stirring for 16 hrs, the reaction was partitioned between EtOAc and a saturated aqueous solution of ammonium chloride. The organic phase was recovered and the aqueous phase was extracted with EtOAc twice. The organic extracts were combined and washed with brin, dried over anhydrous sodium sulfate, filtered, and concentrated under reduced pressure. The crude residue was purified on a pad of silica gel eluting in gradient from 35%-75% EtOAc in hexanes to afford the product.

### General Procedure H:

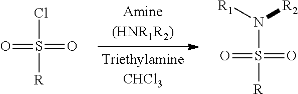

Sulfonyl chloride (1 eq.) was added with anhydrous chloroform (0.3 M). The resulting solution was stirred at 0° C., followed by dropwise addition of appropriate starting amine (1.1 eq.) and triethylamine (3 eq.). The reaction was quenched with 0.1 M HCl and the aqueous phase was extracted thrice with dichloromethane. The combined organic layer was washed once with saturated sodium chloride solution, dried with sodium sulfate, and concentrated in vacuo. The crude sample was absorbed onto silica gel and purified using flash chromatography using a Hexane:Ethyl acetate gradient.

### Example 2: 2-(2,4-dichlorophenoxy)-1-(4-((perfluorophenyl)sulfonyl)piperazin-1-yl)ethan-1-one (Compound 49A)

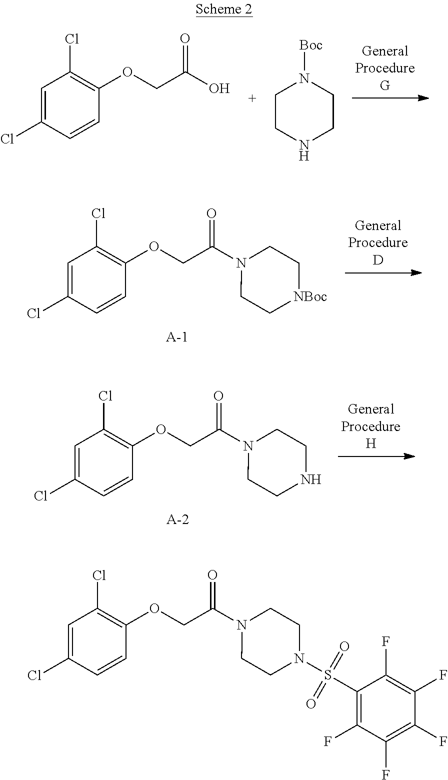

Compound 49A and related examples can be prepared according to the route in Scheme 2 starting from commercially available 2-(2,4-dichlorophenoxy)acetic acid. The acid was coupled with tert-butyl piperazine-1-carboxylate using HOBt and HBTU (General Procedure G). The obtained A-1 was then deprotected using a mixture of TFA and DCM as outlined in General Procedure D. Lastly, Compound 49A can be prepared from A-2 and pentafluorobenzenesulfonyl chloride by using General Procedure H.

### Example 3: 7-chloro-6-fluoro-1-(2-isopropyl-4-methylpyridin-3-yl)-4-((S)-2-methyl-4-((perfluorophenyl)sulfonyl)piperazin-1-yl)pyrido[2,3-d]pyrimidin-2(1H)-one (Compound 2A) and 6-fluoro-7-(2-fluoro-6-hydroxyphenyl)-1-(2-isopropyl-4-methylpyridin-3-yl)-4-((S)-2-methyl-4-((perfluorophenyl)sulfonyl)piperazin-1-yl)pyrido[2,3-d]pyrimidin-2(1H)-one (Compound 1A)

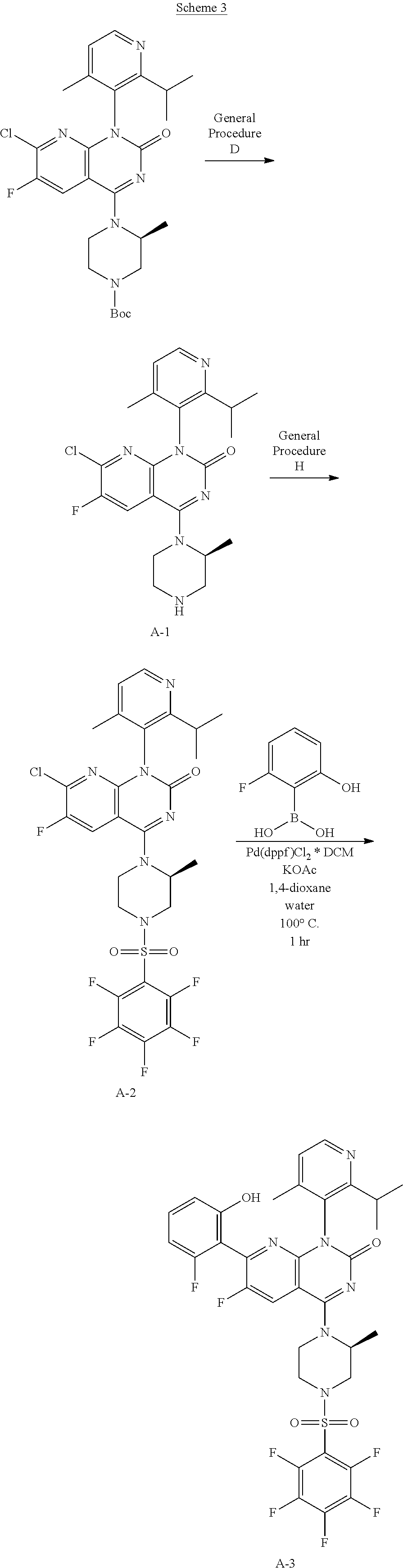

Compound 1A and Compound 2A and related examples can be prepared according to the route in Scheme 3. The starting material can be prepared as described in Journal of Medicinal Chemistry 2020 63 (13), 6679-6693. The obtained starting material can be deprotected using General Procedure D to generate A-1 which can be substituted to pentafluorobenzenesulfonyl chloride based on General Procedure H. The compound A-3 can be prepared by adding Pd(dppf)Cl₂·DCM (0.1 eq.), (2-fluoro-6-hydroxy-phenyl)boronic acid (2 eq.), KOAc (5 eq.) and 1,4-dioxane to a microwave vial under N₂. To this vial was then added with a solution of A-2 in 1,4-dioxane, followed by water. The reaction mixture was stirred under microwave irradiation at 100° C. for 1 hr and then filtered through a pad of Celite. The collected organic mixture was concentrated under reduced pressure. The crude product was purified by reverse-phase column chromatography eluting in gradient from 10%-60% MeCN in Mili-Q Water (+0.1% FA) to afford the product.

### Example 4: 2,3,4,5-tetrafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-6-methoxybenzenesulfonamide (Compound 9A)

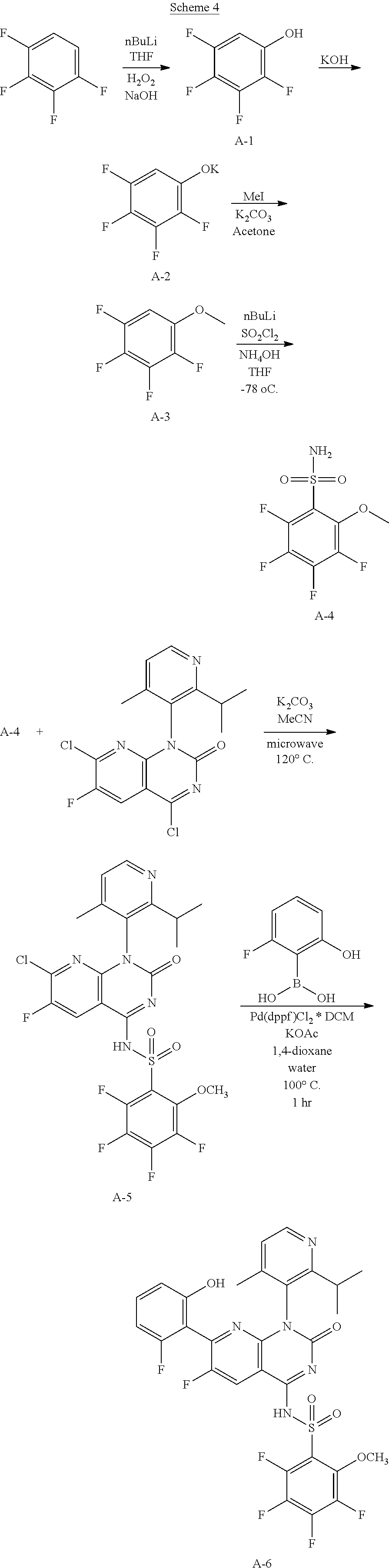

Compound 9A and related examples can be prepared according to the route in Scheme 3, which is further described below.

Preparation of 2,3,4,5-tetrafluorophenol (A-1). A solution of n-butyllithium (2.5 M, 1.1 equiv) in hexane was added to a stirred solution of 1,2,3,4-tetrafluorobenzene (1.0 equiv) in dry THF (1.3 M) at −78° C. The resulting light-yellow solution was maintained at −78° C. for 1 hr, after which a solution of trimethyl borate (1.0 equiv) in THF (1.3 M) was added dropwise over a period of 10 min, and the mixture stirred for 1 hour at −78° C. After 1 hr, hydrogen peroxide (50% wt/wt) (17.5 M, 6.0 equiv) was added and the mixture was warmed up to rt. After 20 min, an aqueous solution of sodium hydroxide (2 M, 10.0 equiv) was added, and a three-layer system was obtained. The top layer was discarded, and the two lower layers were acidified with concentrated HCl until a pH of <2 was obtained. The acidified lower fraction was extracted twice with DCM, and the organic layer was washed once with brine, dried over anhydrous sodium sulfate, and evaporated under reduced pressure to obtain the product as a yellow oil (60%), which was used directly in next step without further purification.

Preparation of 2,3,4,5-tetrafluorophenolate (A-2). To a round-bottom flask equipped with a stir bar was added a 6 M aqueous solution of KOH. The flask was cooled to 0° C. and then 2,3,4,5-tetrafluorophenol (1.0 equiv) was added dropwise via pipette. The resulting mixture was stirred at 0° C. for 30 min. While at 0° C., an off-white solid began to precipitate out of the solution. The solid was isolated via vacuum filtration and the filter cake dried in a 140° C. oven for 2 hrs to afford the anticipated product. The obtained crude was purified by normal phase column chromatography, eluting 1:1 hexane:EtOAc to give 2,3,4,5-tetrafluorophenoxy)potassium (1.5 g, 61.01%).

Preparation of 1,2,3,4-tetrafluoro-5-methoxybenzene (A-3). To a dry microwave vial equipped with a stir bar and purged once with N₂ was added 2,3,4,5-(tetrafluorophenoxy)potassium (1.0 equiv), and K₂CO₃ (1.0 equiv) were suspended in acetone (0.5 M) at 0° C. The mixture was then stirred while slowly adding iodomethane (1.2 equiv) dropwise at rt. After overnight stirring, the reaction mixture was filtered through short pad of silica and washed with ether. The collected filtrate was concentrated under reduced pressure with intermittent heating and the product was used in the next step without further purification (91%).

Preparation of 2,3,4,5-tetrafluoro-6-methoxybenzenesulfonamide (A-4). 1,2,3,4-tetrafluoro-5-methoxy-benzene (1.0 equiv) was dissolved in THF (0.5 M) and then n-Butyllithium 2.5 M solution in hexanes (1.1 equiv) was added slowly at −78° C. and the reaction mixture was stirred for 20 min under argon atmosphere. Then, to the obtained light-yellow solution was slowly added cold pre-dried over MgSO₄ anhydrous hexanes (0.5 M) solution of sulfuryl chloride (1.1 equiv) at −78° C. with vigorous stirring. The mixture was stirred at −78° C. for 20 min and water (1.67 M) was slowly added. The mixture was allowed to warm to about 0° C. and the aqueous layer was immediately separated. Organic layer was dried over MgSO₄ and evaporated resulting in the sulfonyl chloride as a clear light-yellow oil. THF (0.5 M) was slowly introduced to the oil under argon at −78° C. followed by ammonium hydroxide (28-30%) (1.0 equiv) until pH 7 and the bath was removed. After stirring for an additional 0.5 h the mixture was evaporated resulting in a white solid. It was triturated with hexane twice and dried under reduced pressure to afford the product as a pale-yellow solid (208.4 mg, 48%).

Preparation of N-(7-chloro-6-fluoro-1-(2-isopropyl-4-methylpyridin-3-yl)-2-oxo-1,2-dihydropyrido[2,3-d]pyrimidin-4-yl)-2,3,4,5-tetrafluoro-6-methoxybenzenesulfonamide (A-5) A solution of 4,7-dichloro-6-fluoro-1-(2-isopropyl-4-methyl-3-pyridyl)pyrido[2,3-d]pyrimidin-2-one (1.0 equiv), 2,3,4,5-tetrafluoro-6-methoxy-benzenesulfonamide (1.0 equiv), and Potassium Carbonate (3.0 equiv) in anhydrous ACN (0.09 M) was stirred in a capped microwave vial at 120° C. for 4 hrs. The vial was cooled down to rt and the reaction was quenched with water, then the aqueous layer was extracted three times with ethyl acetate. The combined organic layers were washed with saturated brine, dried over anhydrous sodium sulfate, filtered and all the volatiles was removed under reduced pressure. The obtained crude was purified by normal phase column chromatography eluting 1:1 hexane:EtOAc to afford the pure product as a yellow solid. The product was further purified by prep HPLC eluting with 50-80% of ACN (0.1% FA) in water (0.1% FA) within 30 min resulting in the product as a white free-flowing solid (7%).

### Example 5: N-(1-(2-(2,4-dichlorophenoxy)acetyl)azetidin-3-yl)-2,3,4,5-tetrafluoro-6-(methylsulfonyl)benzamide (Compound 52A)

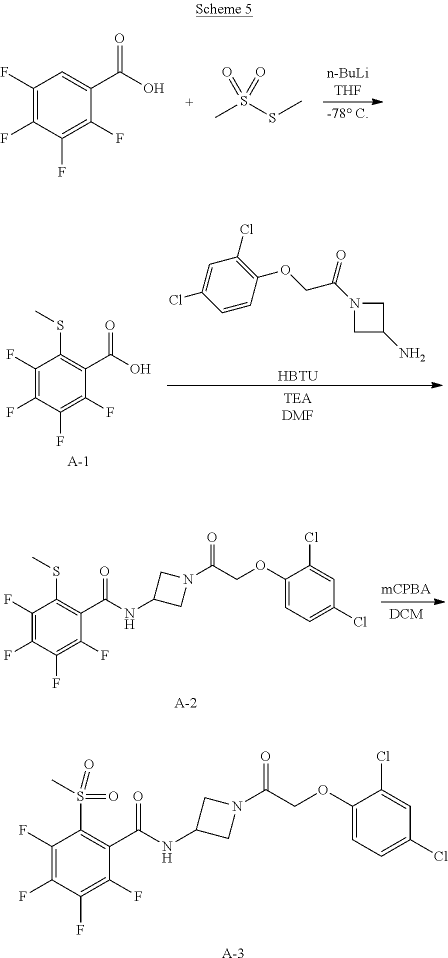

Compound 52A and related examples can be prepared according to the route in Scheme 5, which is further described below.

Preparation of 2,3,4,5-tetrafluoro-6-methylsulfanyl-benzoic acid (A-1). To an oven-dried rbf charged with 2,3,4,5-tetrafluorobenzoic acid (500 mg, 2.58 mmol) and THF (12 mL) was added dropwise n-butyl lithium (2.5 M, 7.73 mmol, 3.09 mL) at −78° C. After 20 min, the aryl lithium species was added to a cold (0° C.) solution of methylsulfonylsulfanylmethane (975.34 mg, 7.73 mmol, 729.50 μL) in THF (5 mL) via cannula. After the addition was complete, the reaction mixture was warmed up to ambient temperature and stirred at this temperature for another 2 hr. The mixture was quenched with 1M HCl, and the aqueous phase was extracted (2×) with EtOAc. The combined organic layers were washed with brine, dried over anhydrous Na₂SO₄, and concentrated down under vacuum. The crude sample was purified by normal phase column chromatography (Hex/EtOAc, gradient from 10-50% EtOAc) to yield 2,3,4,5-tetrafluoro-6-methylsulfanyl-benzoic acid (417 mg, 1.74 mmol, 67.39% yield).

Preparation of N-[1-[2-[2,5-bis(chloranyl)phenoxy]acetyl]azetidin-3-yl]-2,3,4,5-tetrakis(fluoranyl)-6-methylsulfanyl-benzamide (A-2). To a vial charged with 2,3,4,5-tetrafluoro-6-methylsulfanyl-benzoic acid (45 mg, 187.36 μmol) and DMF (1.5 mL) was added 1-(3-azanylazetidin-1-yl)-2-[2,4-bis(chloranyl)phenoxy]ethanone (51.54 mg, 187.34 μmol), followed by HBTU (71.06 mg, 187.36 μmol) and Triethylamine (20.86 mg, 206.10 μmol, 28.73 μL). The reaction mixture was stirred at r.t. for 12 hr. The reaction was quenched with water and EtOAc was added to dilute the reaction mixture. The mixture was washed with water twice, washed with brine, dried over Na₂SO₄, filtered and all the volatile was removed under reduced pressure. The crude was purified by normal phase column chromatography (Hex/EtOAc, gradient from 20-100% EtOAc) to give N-[1-[2-[2,5-bis(chloranyl)phenoxy]acetyl]azetidin-3-yl]-2,3,4,5-tetrakis(fluoranyl)-6-methylsulfanyl-benzamide (44.5 mg, 89.48 μmol, 47.76% yield).

Preparation of N-[1-[2-[2,5-bis(chloranyl)phenoxy]acetyl]azetidin-3-yl]-2,3,4,5-tetrakis(fluoranyl)-6-methylsulfonyl-benzamide (A-3). To a solution of N-[1-[2-[2,5-bis(chloranyl)phenoxy]acetyl]azetidin-3-yl]-2,3,4,5-tetrakis(fluoranyl)-6-methylsulfanyl-benzamide (40 mg, 80.44 μmol) in Dichloromethane, anhydrous (2 mL) was added portion wise 3-Chloroperbenzoic acid (41.64 mg, 241.31 μmol) at r.t. The reaction kept on stirring at room temperature overnight. NaHCO₃ was added to quench, and the resulting mixture was extracted with DCM (3×), washed with brine, dried over Na₂SO₄, filtered and solvent was removed under reduced pressure. The crude was purified by C18 reverse phase column chromatography (ACN/H₂O, 0.1% Formic acid, gradient from 30-100% ACN) to yield N-[1-[2-[2,5-bis(chloranyl)phenoxy]acetyl]azetidin-3-yl]-2,3,4,5-tetrakis(fluoranyl)-6-methylsulfonyl-benzamide (15.7 mg, 29.37 μmol, 36.51% yield, 99% purity).

### Example 6: (S)-6-chloro-7-(2,6-dimethylphenyl)-4-(2-methyl-4-(2,3,4,5-tetrafluoro-6-(methylsulfonyl)benzyl)piperazin-1-yl)quinoline (Compound 45A)

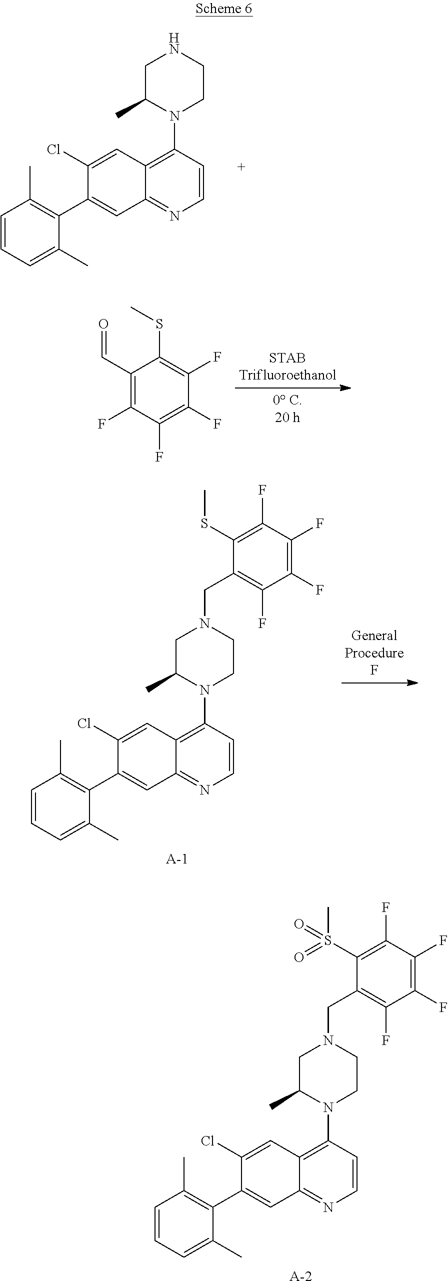 [SMILES: Cc1cccc(C)c1-c1cc2nccc(N3CCNC[C@@H]3C)c2cc1Cl ; CSc1c(F)c(F)c(F)c(F)c1C=O ; CSc1c(F)c(F)c(F)c(F)c1CN1CCN(c2ccnc3cc(-c4c(C)cccc4C)c(Cl)cc23)[C@@H](C)C1 ; "A-1" ; Cc1cccc(C)c1-c1cc2nccc(N3CCN(Cc4c(F)c(F)c(F)c(F)c4S(C)(=O)=O)C[C@@H]3C)c2cc1Cl ; "A-2"]

Compound 45A and related examples can be prepared according to the route in Scheme 6, which is further described below.

Preparation of (S)-6-chloro-7-(2,6-dimethylphenyl)-4-(2-methyl-4-(2,3,4,5-tetrafluoro-6-(methylthio) benzyl)piperazin-1-yl)quinoline (A-1). To a stirred solution of (S)-6-chloro-7-(2,6-dimethylphenyl)-4-(2-methylpiperazin-1-yl)quinoline (0.45 g, 1.23 mmol) in 2,2,2 Trifluroethanol (4.0 mL) was added 2,3,4,5-tetrafluoro-6-(methylthio)benzaldehyde (0.96 g, 4.31 mmol) at room temperature. The resulting reaction mixture was stirred at room temperature for 16 h. After consumption of both starting material (imine formation), sodium tri-acetoxyborohydride (0.78 g, 3.69 mmol) was added at 0° C. The resulting reaction mixture was allowed to stir for another 16 h. After completion of reaction, the mixture was concentrated under reduced pressure. The resulting crude was purified by flash column chromatography, eluted with 15-20% EtOAc in hexane to afford title compound as a brown sticky liquid (0.4 g, 0.69 mmol, 57% Yield).

Preparation of (S)-6-chloro-7-(2,6-dimethylphenyl)-4-(2-methyl-4-(2,3,4,5-tetrafluoro-6-(methylsulfonyl) benzyl)piperazin-1-yl)quinoline (A-2). To a stirred solution of (S)-6-chloro-7-(2,6-dimethylphenyl)-4-(2-methyl-4-(2,3,4,5-tetrafluoro-6-(methylthio)benzyl)piperazin-1-yl)quinoline (0.05 g, 0.08 mmol) in THF:MeOH:Water (8:1:1, 2.7 mL) was added oxone (0.13 g, 0.43 mmol) at 0° C. The resulting reaction mixture was stirred at room temperature for 3 h. After the completion of reaction, the reaction mixture was diluted with aq. NaHCO₃ (30 mL) and extracted with DCM (3×30 mL). The combined organic phases were dried over anhydrous Na₂SO₄, filtered and concentrated under reduced pressure. The obtained crude was purified by flash column chromatography, eluted with 35-40% EtOAc in hexane to afford title compound as a white solid (0.008 g, 0.013 mmol, 4% yield).

### Example 7: (2-(6-chloro-8-fluoro-4-((S)-2-methyl-4-(2,3,5,6-tetrafluoro-4-(methylsulfonyl)phenyl)piperazin-1-yl)quinolin-7-yl)-3-fluorophenol (Compound 24A) and its atropisomers (Compound 25A and Compound 26A)

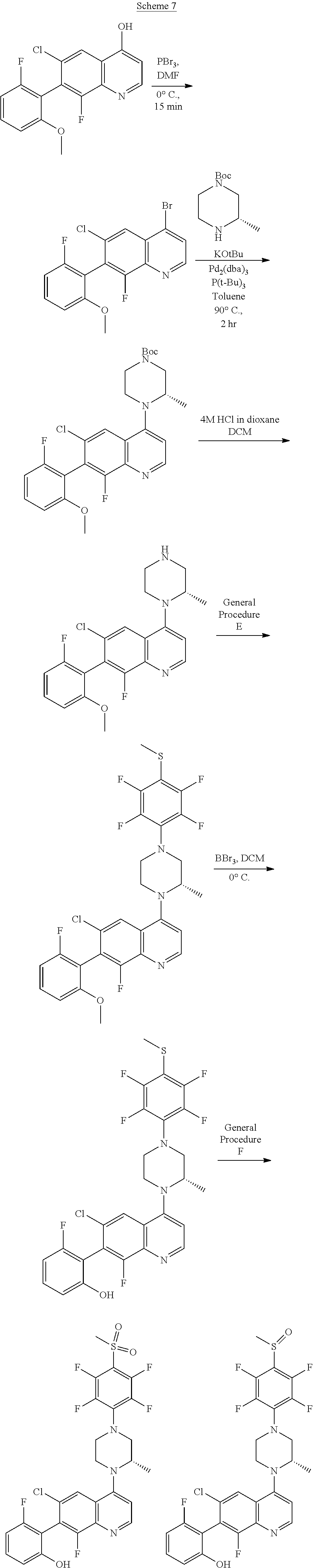

Synthesis of 4-bromo-6-chloro-8-fluoro-7-(2-fluoro-6-methoxyphenyl)quinoline. To a stirred solution of 6-chloro-8-fluoro-7-(2-fluoro-6-methoxyphenyl)quinolin-4-ol (5.0 g, 15.57 mmol) in DMF (60 mL) was added Phosphorus tribromide (−log, 38.94 mmol) at 0° C. The resulting reaction mixture was stirred at room temperature for 15 min. After completion of reaction, the reaction mixture was poured into an ice-cold water. The obtained precipitate was filtered off. Isolated solid was dissolved in EtOAc (250 mL×2). The combined organic phases were dried over anhydrous Na₂SO₄, filtered and concentrated under reduced pressure. The resulting crude was purified by silica gel column chromatography, eluted with 28% EtOAc in hexane to afford title compound as a yellow solid (2.12 g, 5.53 mmol, 36% yield).

Synthesis of tert-butyl (3S)-4-(6-chloro-8-fluoro-7-(2-fluoro-6-methoxyphenyl)quinolin-4-yl)-3-methylpiperazine-1-carboxylate. To a stirred solution of tert-butyl (S)-3-methylpiperazine-1-carboxylate (0.79 g, 3.95 mmol) in toluene (5 mL) were added Potassium tert-butoxide (0.55 g, 4.94 mmol) and 4-bromo-6-chloro-8-fluoro-7-(2-fluoro-6-methoxyphenyl)quinoline (1.26 g, 6.59 mmol) at room temperature. The resulting reaction mixture was purged with N₂ for 15 minutes followed by addition of Pd₂(dba)₃ (0.30 g, 0.32 mmol) and Tri-tert-butylphosphine (0.066 g, 0.32 mmol) at room temperature. The resulting reaction mixture was stirred at 90° C. for 2 h. After completion of reaction, the reaction mixture was cooled to ambient temperature and diluted with water (100 mL). The resulting suspension was extracted with EtOAc (3×100 mL). The combined organic phases were dried over anhydrous Na₂SO₄, filtered and concentrated under reduced pressure. The resulting crude was purified by silica gel column chromatography, eluted with 28% EtOAc in hexane to afford title compound as a yellow solid (1.32 g, 2.62 mmol, 40% yield).

Synthesis of 6-chloro-8-fluoro-7-(2-fluoro-6-methoxyphenyl)-4-((S)-2-methylpiperazin-1-yl)quinolone. To a stirred solution of tert-butyl (3S)-4-(6-chloro-8-fluoro-7-(2-fluoro-6-methoxy phenyl)quinolin-4-yl)-3-methylpiperazine-1-carboxylate (1.32 g, 2.62 mmol) in DCM (7 mL) was added 4M HCl in 1,4-dioxane (5.6 mL) at room temperature. The resulting reaction mixture was stirred at room temperature for 2 h. After the completion of reaction, the reaction mixture was concentrated under reduce vacuum. The obtained residue was diluted with aq NaHCO₃ (50 mL) and extracted with EtOAc (3×50 mL). The combined organic phases were dried over anhydrous Na₂SO₄, filtered and concentrated under reduced pressure, to afford title compound as a brown solid (0.99 g, 2.47 mmol, Quantitative).

Synthesis of 6-chloro-8-fluoro-7-(2-fluoro-6-methoxyphenyl)-4-((S)-2-methyl-4-(2,3,5,6-tetrafluoro-4-(methylthio)phenyl)piperazin-1-yl)quinoline. The product was prepared using General Procedure E.

Synthesis of 2-(6-chloro-8-fluoro-4-((S)-2-methyl-4-(2,3,5,6-tetrafluoro-4-(methylthio)phenyl) piperazin-1-yl)quinolin-7-yl)-3-fluorophenol (Compound 29A). To the stirred solution of 6-chloro-8-fluoro-7-(2-fluoro-6-methoxyphenyl)-4-((S)-2-methyl-4-(2,3,5,6-tetrafluoro-4-(methylthio)phenyl)piperazin-1-yl)quinoline (0.30 g, 0.50 mmol) in DCM (3 mL) was added BBr₃ (0.18 g, 0.75 mmol) at 0° C. The resulting reaction mixture was stirred at room temperature for 3 h. After the completion of reaction, the reaction mixture was diluted with saturated solution of NaHCO₃ (50 mL) and extracted with DCM (3×50 mL). The combined organic phases were dried over anhydrous Na₂SO₄, filtered and concentrated under reduced pressure, to afford title compound as an off-white solid (0.25 g, 0.43 mmol, 86% yield).

Synthesis of 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 24A). The product was prepared using General Procedure F. Atropisomers (Compound 25A and Compound 26A) were separated by Waters SFC eluting with 50% of liquid CO₂ in MeOH over 20 minutes on CHIRALPAK IG 250×50 mm 5 um to yield 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 25A) & (Compound 26A).

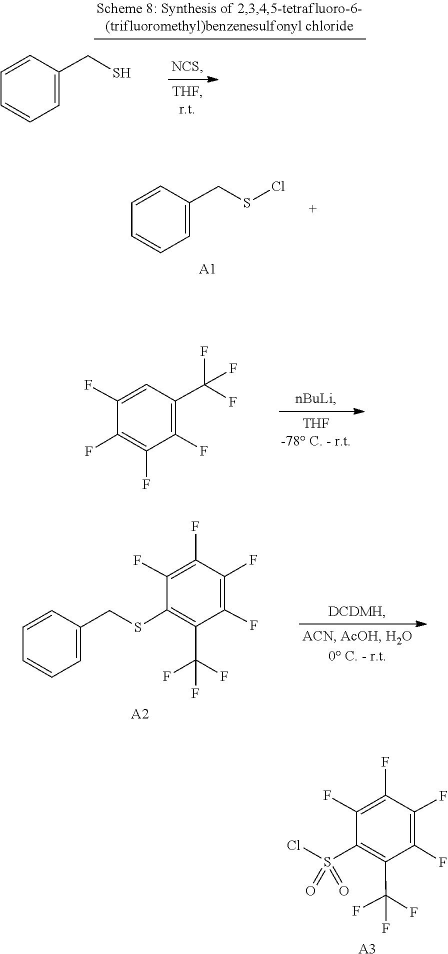

### Synthesis of A1

A dry 25 mL rbf was equipped with a stir bar, sealed with a rubber septum, and flushed with nitrogen for 5 min. After flushing, a solution of phenylmethanethiol (300 mg, 2.42 mmol, 283.55 μL) in THF (5.64 mL) was introduced into the flask. While stirring @ r.t., neat 1-chloropyrrolidine-2,5-dione (354.79 mg, 2.66 mmol, 215.02 μL) was added in one portion to prepare a pale-yellow mixture. After 1 hour, the reaction became a dark yellow solution. The solution was used in the next reaction without any further manipulation/purification.

### Synthesis of A2

An oven-dried 25 mL rbf was equipped with a stir bar, capped with a rubber septum, and flushed with dry argon for 10 min. at r.t. To the flask was added 1,2,3,4-tetrafluoro-5-(trifluoromethyl)benzene (479.76 mg, 2.2 mmol) and THF (10 mL) to prepare a colourless solution. The reaction was cooled to −78° C. before nBuLi (2.5 M in hexanes, 968.00 μL) was added to prepare the corresponding aryl-lithium species. After 20 min, the aryl lithium species (a faint purple colour) was added to a cold (0° C.) solution of benzylsulfinyl chloride (383.93 mg, 2.42 mmol) in THF (6 mL) via cannula. Extra caution was taken to ensure that the organolithium was added directly to the benzylsulfenyl chloride solution. After addition was complete, the rxn was warmed slowly to r.t. over 1 hours. After 2 hours, the reaction was quenched with a 1M HCl and the organic layer separated. The aqueous phase was extracted 2× with EtOAc and the combined organic extracts washed with brine, dried over anhydrous sodium sulfate, and concentrated under vacuum to afford the product (650 mg, 1.9 mmol, 87% yield) as a pale-yellow oil. The crude material was used in the next reaction without any further purification.

### Synthesis of A3: 2,3,4,5-tetrafluoro-6-(trifluoromethyl)benzenesulfonyl Chloride

Neat 1,3-dichloro-5,5-dimethyl-imidazolidine-2,4-dione (752.74 mg, 3.82 mmol, 501.82 μL) was added to an ice cold solution of 1-benzylsulfanyl-2,3,4,5-tetrafluoro-6-(trifluoromethyl)benzene (650 mg, 1.91 mmol) in CH₃CN/AcOH/H₂O (2 mL/0.075 mL/0.05 mL). The resulting pale yellow mixture was stirred at 0° C. for 4 hours, then warmed to r.t. overnight. After the overnight period, the rxn was partitioned between DCM and a saturated aqueous solution of NaHCO₃. The organic layer was removed and the remaining aqueous phase extracted 2× with DCM. The organic extracts were combined, washed with brine, dried over anhydrous sodium sulfate, and concentrated in vaccuo to afford 2,3,4,5-tetrafluoro-6-(trifluoromethyl)benzenesulfonyl chloride as a beige semi-solid. The crude material was used without any further purification ¹⁹F NMR (376 MHz, CDCl₃) δ −50.67 (d, J=37.7 Hz), −122.70 (ddd, J=23.5, 14.6, 8.8 Hz), −129.90 (ddt, J=37.7, 20.4, 9.6 Hz), −138.15 (td, J=20.6, 13.9 Hz), −142.22 (ddd, J=22.9, 19.8, 10.7 Hz).

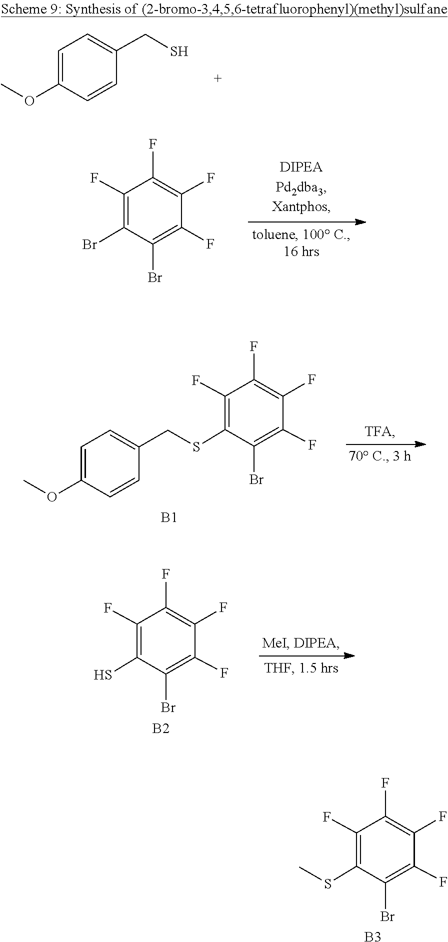

### Synthesis of B1

A solution of (4-methoxyphenyl)methanethiol (16 g, 51.94 mmol), 1,2-dibromo-3,4,5,6-tetrafluorobenzene (8 g, 51.94 mmol), and DIPEA (13.40 mL, 103.00 mmol), in toluene (150 mL) was purged with N₂ for 15 minutes. Once purged, Pd₂dba₃ (1.28 g, 1.40 mmol) and Xanthphos (1.23 g, 2.00 mol) were added at room temperature. The resulting mixture was heated to 100° C. overnight. After 16 hours, the mixture was diluted with water (100 m) and extracted with EtOAc (2×200 mE). The combined organic phases were dried over anhydrous Na₂SO₄, filtered, and concentrated under reduced pressure. The crude material was purified by flash column chromatography (0.25 EtOAc in hexane) to afford title compound as white solid (8.0 g, 20.99 mmol, 40% yield). ¹H NMR (400 MHz, DMSO-d6) δ 7.12 (d, J=8.8 Hz, 2H), 6.83 (d, J=8.8 Hz, 2H), 4.11 (s, 2H), 3.71 (s, 3H).

### Synthesis of B2

(2-bromo-3,4,5,6-tetrafluorophenyl)(4-methoxybenzyl)sulfane (8.7 g, 22.83 mmol) was added to ice-cold (61C) TFA (87 m). The reaction was gradually warmed to room temperature, then heated to 70° C. After 3 hours, reaction mixture was concentrated under reduced pressure and co-distilled with DCM (5×100 mL) to afford title compound as brown sticky oil (8 g, 30.79 mmol). The obtained material was used in next step without purification.

### Synthesis of B3: (2-bromo-3,4,5,6-tetrafluorophenyl)(methyl)sulfane

To a stirred solution of 2-bromo-3,4,5,6-tetrafluorobenzenethiol (8.0 g, 30.78 mmol) in THF (1.5 mE) at OTC was added DIPEA (16 mL, 92.37 mmol), followed by addition of Mel (2.8 mE, 46.17 mmol). The reaction was permitted to warm to room temperature. After 1.5 hours, mixture was concentrated via fractional distillation to remove THF. The crude material was purified by flash column chromatography (100% hexanes) to afford title compound as colourless liquid (4.5 g, 16.36 mmol, 53% yield). ¹H NMR (400 MHz, CDCl₃) δ 2.51 (s, 3H). ¹⁹F NMR (376 MHz, CDCl₃) δ−125.99-−126.08 (m, 1F), −127.66-−127.75 (m, 1F), −152.66-−152.78 (m, 1F), −154.53-−154.65 (m, 1F).

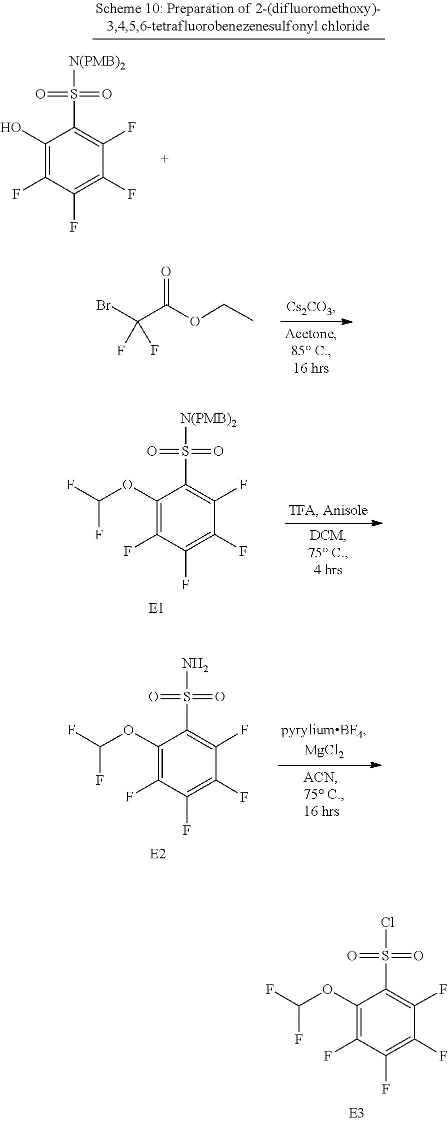

### Synthesis of E1: 2-(difluoromethoxy)-3,4,5,6-tetrafluoro-N,N-bis(4-methoxybenzyl) benzenesulfonamide

To a stirred solution of 2,3,4,5-tetrafluoro-6-hydroxy-N,N-bis(4-methoxybenzyl)benzenesulfonamide (7.0 g, 14.43 mmol) in Acetone (70 mL) was added Cs₂CO₃ (13.68 g, 43.29 mmol) and ethyl 2-bromo-2,2-difluoroacetate (8.78 g, 43.29 mmol). The resulting mixture was heated to 85° C. After 4 hrs, the reaction was cooled to room temperature and concentrated under reduced pressure. The crude material was purified by flash column chromatography (15% EtOAc in hexanes) to afford the title compound as a yellow solid (6.0 g, 11.20 mmol, 77% yield). ¹H NMR (400 MHz, CDCl₃) δ 7.07 (d, J=8.4 Hz, 4H), 6.81 (d, J=8.4 Hz, 4H), 6.69 (t, J=72 Hz, 1H), 4.44 (s, 4H), 3.8 (s, 6H).

### Synthesis of E2: 2-(difluoromethoxy)-3,4,5,6-tetrafluorobenzenesulfonamide

To a stirred solution of 2-(difluoromethoxy)-3,4,5,6-tetrafluoro-N,N-bis(4-methoxybenzyl) benzenesulfonamide (6.5 g, 12.13 mmol) in DCM (65 mL) was added anisole (5.25 g, 48.55 mmol) and the flask purged with N₂ for 10 min. Once flushed, TFA (60.5 mL) was introduced and the reaction heated to 75° C. After 16 hrs, the reaction was cooled to room temperature and concentrated under reduced pressure. The crude material was purified by flash column chromatography (20% EtOAc in hexanes) to afford the title compound as yellow solid (2.7 g, 9.14 mmol, 75% yield). ¹H NMR (400 MHz, DMSO-d₆) δ 8.30 (s, 2H), 7.07 (t, J=72 Hz, 1H), ¹⁹F NMR (400 MHz, DMSO-d₆) δ−81.82-−82.03 (m, 2F), −136.75-−136.85 (m, 1F), −149.04-−149.18 (m, 1F), −150.03-−150.17 (m, 1F), −152.17-−152.25 (m, 1F).

### Synthesis of E3: 2-(difluoromethoxy)-3,4,5,6-tetrafluorobenzenesulfonyl chloride

A suspension of 2-(difluoromethoxy)-3,4,5,6-tetrafluoro-benzenesulfonamide (700 mg, 2.37 mmol), pyrylium tetrafluoroborate (995.46 mg, 5.93 mmol) and magnesium chloride (677.41 mg, 7.11 mmol) in Acetonitrile (23.7 mL) was stirred for 10 minutes under a nitrogen atmosphere. To ensure that the reactants were solubilized, the mixture was sonicated for 5 minutes before being heated to 75° C. After 16 hours, the reaction mixture was cooled to room temperature and filtered through a small plug of silica using EtOAc as the eluent. The filtrate was concentrated down under vacuum and the crude material isolated by flash column chromatography (0-20% EtOAc in Hexanes). The desired compound was isolated as an oily solid (384 mg, 1.2 mmol, 51% yield) ¹H NMR (400 MHz, CDCl₃) δ 6.70 (t, J=72 Hz, 1H). ¹⁹F NMR (400 MHz, CDCl₃) δ −82.39-−82.62 (m, 2F), −131.17-−131.28 (m, 1F), −140.23-−140.36 (m, 1F), −145.88-−145.97 (m, 1F), −152.67-−152.80 (m, 1F).

### Synthesis of 2-(2,4-dichlorophenoxy)-1-(4-((perfluorophenyl)sulfonyl)piperazin-1-yl)ethan-1-one (Compound 49A)

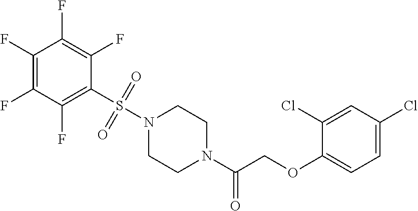 [SMILES: O=C(COc1ccc(Cl)cc1Cl)N1CCN(S(=O)(=O)c2c(F)c(F)c(F)c(F)c2F)CC1]

The title compound, 2-(2,4-dichlorophenoxy)-1-(4-((perfluorophenyl)sulfonyl)piperazin-1-yl)ethan-1-one, was prepared from 2-(2,4-dichlorophenoxy)-1-piperazin-1-yl-ethanone (0.089 g, 0.308 mmol) and 2,3,4,5,6-pentafluorobenzenesulfonyl chloride (0.09 g, 0.339 mmol) via Scheme 2 to yield the white solid (46 mg, 28%). ¹H NMR (400 MHz, CDCl₃) δ 7.39 (d, J=2.5 Hz, 1H), 7.20 (dd, J=8.8, 2.5 Hz, 1H), 6.94 (d, J=8.9 Hz, 1H), 4.77 (s, 2H), 3.85-3.76 (m, 4H), 3.29 (d, J=36.4 Hz, 4H). ¹⁹F NMR (376 MHz, CDCl₃) δ−134.13 (qd, J=14.0, 8.1 Hz, 2F), −143.92 (tt, J=21.2, 6.8 Hz, 1F), −157.44-−157.65 (m, 2F). LC-MS (ESI−) m/z calc'd for [C₁₈H₁₃Cl₂F₄N₂O₅S]⁻: 515.0, found: 515.0. LC-MS purity: 99.09%, 6.47 min.

### Synthesis of 2-(2,4-dichlorophenoxy)-N-(1-((perfluorophenyl)sulfonyl)azetidin-3-yl)acetamide (Compound 50A)

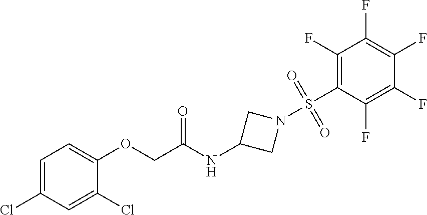 [SMILES: O=C(COc1ccc(Cl)cc1Cl)NC1CN(S(=O)(=O)c2c(F)c(F)c(F)c(F)c2F)C1]

The title compound, 2-(2,4-dichlorophenoxy)-N-(1-((perfluorophenyl)sulfonyl)azetidin-3-yl)acetamide, was prepared from N-(azetidin-3-yl)-2-(2,4-dichlorophenoxy)acetamide (124 mg, 0.453 mmol) and 2,3,4,5,6-pentafluorobenzenesulfonyl chloride (132 mg, 0.498 mmol) via Scheme 2 to yield the white solid (44 mg, 19%). ¹H NMR (400 MHz, CDCl₃) δ 7.47 (d, J=2.5 Hz, 1H), 7.29-7.25 (m, 1H), 7.15 (d, J=7.2 Hz, 1H), 6.86 (d, J=8.8 Hz, 1H), 4.70 (h, J=7.0 Hz, 1H), 4.50 (s, 2H), 4.40 (t, J=8.2 Hz, 2H), 4.24-4.15 (m, 2H). ¹⁹F NMR (376 MHz, CDCl₃) δ −134.46 (qd, J=13.6, 8.0 Hz, 2F), −145.02 (tt, J=21.3, 6.7 Hz, 1F), −158.14-−158.35 (m, 2F). LC-MS (ESI+) m/z calc'd for [C₁₇H₁₁Cl₂F₅N₂O₄S]⁺: 505.0, found: 505.2. LC-MS purity: 99.63%, 8.3 min.

### Synthesis of 6-fluoro-7-(2-fluoro-6-hydroxyphenyl)-1-(2-isopropyl-4-methylpyridin-3-yl)-4-((S)-2-methyl-4-((perfluorophenyl)sulfonyl)piperazin-1-yl)pyrido[2,3-d]pyrimidin-2(H)-one (Compound 1A)

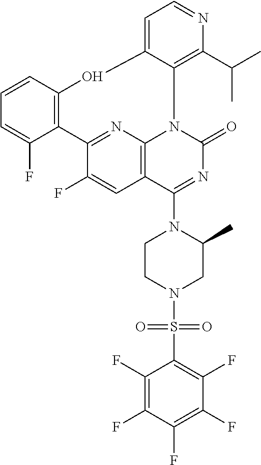 [SMILES: CC(C)c1nccc2c1-n1c(=O)nc(N3CCN(S(=O)(=O)c4c(F)c(F)c(F)c(F)c4F)C[C@@H]3C)c3cc(F)c(nc31)-c1c(F)cccc1O2]

The title compound, 6-fluoro-7-(2-fluoro-6-hydroxyphenyl)-1-(2-isopropyl-4-methylpyridin-3-yl)-4-((S)-2-methyl-4-((perfluorophenyl)sulfonyl)piperazin-1-yl)pyrido[2,3-d]pyrimidin-2(1H)-one, was prepared from 7-chloro-6-fluoro-1-(2-isopropyl-4-methyl-3-pyridyl)-4-[(2S,4S)-2-methyl-4-(2,3,4,5,6-pentafluorophenyl)sulfonyl-piperazin-1-yl]pyrido[2,3-d]pyrimidin-2-one (58 mg, 0.087 mmol) via Scheme 3 to yield the white solid (25 mg, 38%). ¹H NMR (400 MHz, CDCl₃) δ 9.34 (s, 1H), 8.67 (d, J=4.9 Hz, 1H), 7.37-7.30 (m, 1H), 7.24 (d, J=4.9 Hz, 1H), 6.73 (d, J=8.6 Hz, 2H), 4.98 (d, J=53.3 Hz, 1H), 4.55 (dd, J=51.4, 13.7 Hz, 1H), 4.06 (d, J=12.5 Hz, 1H), 3.89 (q, J=12.7 Hz, 2H), 3.24-3.01 (m, 2H), 2.76 (ddd, J=28.7, 13.5, 6.8 Hz, 1H), 2.06 (d, J=17.7 Hz, 3H), 1.76 (dd, J=25.0, 6.8 Hz, 3H), 1.25 (dd, J=6.7, 3.5 Hz, 3H), 1.08 (t, J=6.1 Hz, 3H). ¹⁹F NMR (376 MHz, CDCl₃) δ−107.28 (dq, J=81.2, 9.6, 9.1 Hz, 1F), −120.77 (dt, J=81.4, 8.3 Hz, 1F), −134.24 (dq, J=21.2, 7.2, 5.8 Hz, 2F), −143.72-−144.14 (m, 1F), −157.19-−157.57 (m, 2F). LC-MS (ESI+) m/z calc'd for [C₃₃H₂₇F₇N₆O₄S]⁺: 736.1, found: 737.3. LC-MS: 96.02%, 7.3 min.

### Synthesis of 7-chloro-6-fluoro-1-(2-isopropyl-4-methylpyridin-3-yl)-4-((S)-2-methyl-4-((perfluorophenyl)sulfonyl)piperazin-1-yl)pyrido[2,3-d]pyrimidin-2(H)-one (Compound 2A)

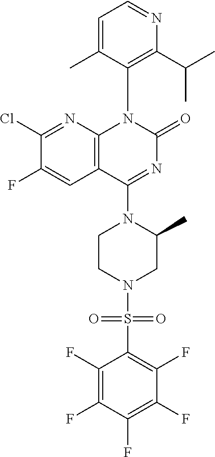 [SMILES: Cc1ccnc(C(C)C)c1-n1c(=O)nc(N2CCN(S(=O)(=O)c3c(F)c(F)c(F)c(F)c3F)C[C@@H]2C)c2cc(F)c(Cl)nc21]

The title compound, 7-chloro-6-fluoro-1-(2-isopropyl-4-methylpyridin-3-yl)-4-((S)-2-methyl-4-((perfluorophenyl)sulfonyl)piperazin-1-yl)pyrido[2,3-d]pyrimidin-2(1H)-one, was prepared from 7-chloro-6-fluoro-1-(2-isopropyl-4-methyl-3-pyridyl)-4-[(2S)-2-methylpiperazin-1-yl]pyrido[2,3-d]pyrimidin-2-one (51 mg, 0.118 mmol) and 2,3,4,5,6-pentafluorobenzenesulfonyl chloride (32 mg, 0.118 mmol) via Scheme 3. ¹H NMR (400 MHz, CDCl₃) δ 8.60 (d, J=4.9 Hz, 1H), 7.76 (d, J=7.4 Hz, 1H), 7.16 (d, J=4.9 Hz, 1H), 4.84 (s, 1H), 4.45-4.35 (m, 1H), 4.02 (d, J=12.6 Hz, 1H), 3.90-3.78 (m, 2H), 3.17 (d, J=10.9 Hz, 1H), 3.06 (dd, J=7.4, 3.6 Hz, OH), 2.58 (dp, J=20.5, 6.7 Hz, 1H), 2.03 (d, J=13.0 Hz, 3H), 1.70 (dd, J=6.9, 1.9 Hz, 3H), 1.32-1.25 (m, 2H), 1.22 (dd, J=6.7, 3.8 Hz, 3H), 1.12 (dd, J=8.8, 6.7 Hz, 3H). ¹⁹F NMR (376 MHz, CDCl₃) δ−125.32 (dd, J=7.5, 4.4 Hz, 1F), −134.33 (ddt, J=20.4, 8.0, 4.3 Hz, 2F), −143.85-−144.06 (m, 1F), −157.28-−157.50 (m, 2F). LC-MS (ESI−) m/z calc'd for [CC₂₇H₂₃ClF₆N₆O₃S]⁻: 661.0, found: 661.3. LC-MS purity: 100%, 4.7 min.

### Synthesis of 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-methoxyphenyl)sulfonyl)piperazin-1-yl)pyrido[2,3-d]pyrimidin-2(1H)-one (Compound 5A)

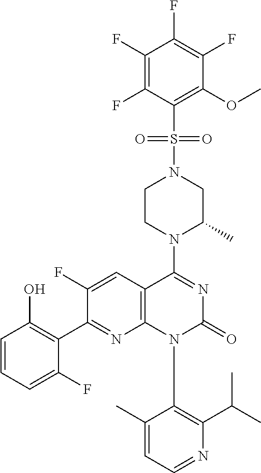 [SMILES: COc1c(F)c(F)c(F)c(F)c1S(=O)(=O)N1CCN(c2nc(=O)n(Cc3c(C)ccnc3C(C)C)c3nc(-c4c(O)cccc4F)c(F)cc23)[C@@H](C)C1]

The title compound, 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-methoxyphenyl)sulfonyl)piperazin-1-yl)pyrido[2,3-d]pyrimidin-2(1H)-one, was prepared from 7-chloro-6-fluoro-1-(2-isopropyl-4-methyl-3-pyridyl)-4-[(2S)-2-methyl-4-(2,3,4,5-tetrafluoro-6-methoxy-phenyl)sulfonyl-piperazin-1-yl]pyrido[2,3-d]pyrimidin-2-one (30 mg, 0.045 mmol) via Scheme 3. ¹H NMR (400 MHz, CDCl₃) δ 9.36 (s, 1H), 8.66 (d, J=4.9 Hz, 1H), 7.89 (dd, J=9.4, 4.4 Hz, 1H), 7.36-7.31 (m, 1H), 7.24 (d, J=5.0 Hz, 1H), 6.72 (d, J=8.5 Hz, 2H), 4.96 (d, J=53.1 Hz, 1H), 4.52 (dd, J=54.2, 13.7 Hz, 1H), 4.03 (d, J=12.9 Hz, 1H), 3.94-3.75 (m, 2H), 3.21-2.98 (m, 2H), 2.76 (dp, J=27.3, 6.8 Hz, 1H), 2.05 (d, J=12.0 Hz, 3H), 1.72 (dd, J=25.0, 6.8 Hz, 3H), 1.29-1.22 (m, 6H), 1.08 (t, J=6.1 Hz, 3H). ¹⁹F NMR (376 MHz, CDCl₃) δ−107.49-107.58 (m, 1F), −120.89-−121.02 (m, 1F), −134.54-−134.70 (m, 1F), −145.95-−146.18 (m, 1F), −151.92-−152.10 (m, 1F), −158.76-−158.98 (m, 1F). LC-MS (ESI+) m/z calc'd for [C₃₄H₃₀F₆N₆O₅S]⁺: 749.2, found: 749.3. LC-MS purity: 97.09%, 7.3 min.

### Synthesis of 2,3,4,5,6-pentafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)benzenesulfonamide (Compound 3A)

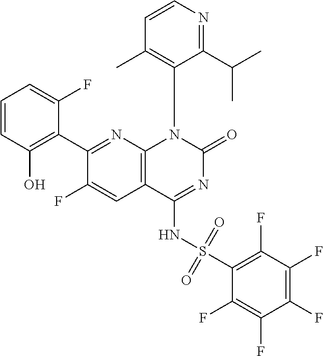 [SMILES: Cc1ccnc(C(C)C)c1Cn1c(=O)nc(NS(=O)(=O)c2c(F)c(F)c(F)c(F)c2F)c2cc(F)c(-c3c(O)cccc3F)nc21]

A mixture of N-[7-chloro-6-fluoro-1-(2-isopropyl-4-methyl-3-pyridyl)-2-oxo-pyrido[2,3-d]pyrimidin-4-yl]-2,3,4,5,6-pentafluoro-benzenesulfonamide (1.0 equiv), (2-fluoro-6-hydroxy-phenyl)boronic acid (4.0 equiv), SPhos Pd G3 (0.05 equiv) and Potassium Carbonate (4.0 equiv) in a microwave vial was evacuated and backfilled with argon. Then 1,2-Dimethoxyethane (0.04 M) and deionized water (0.43 M) were added, and the mixture was stirred at 85° C. for 16 hrs. After the vial was cooled down to rt, the reaction was quenched with saturated aqueous solution of sodium bicarbonate and then the aqueous layer was extracted three times with ethyl acetate. The combined organic layers were washed with saturated brine, dried over anhydrous sodium sulfate, filtered and all the volatiles was removed under reduced pressure. The obtained crude was purified by normal phase column chromatography, eluting 1:1 hexane:EtOAc to afford the pure product as a yellow solid. The product was further purified by prep HPLC eluting with 30-70% ACN in water (0.10% FA) over 30 min resulting in the targeted product as a yellow solid (26 mg, 15%). ¹H NMR (400 MHz, CD₃CN) δ 8.53 (d, J=4.9 Hz, 1H), 8.43 (d, J=8.4 Hz, 1H), 7.94 (s, 1H), 7.34 (td, J=8.4, 6.7 Hz, 1H), 7.24 (dd, J=4.9, 0.8 Hz, 1H), 6.78-6.68 (m, 2H), 2.96 (hept, J=6.6 Hz, 1H), 2.12-2.08 (m, 3H), 1.08 (dd, J=62.9, 6.7 Hz, 6H). ¹⁹F NMR (376 MHz, CD₃CN) δ −114.53 (dt, J=24.0, 8.2 Hz, 1F), −125.51 (dd, J=23.9, 8.3 Hz, 1F), −137.95 (ddt, J=18.8, 12.5, 7.2 Hz, 2F), −148.64 (1F), −161.58 (t, J=19.6 Hz, 2F). LC-MS (ESI+) m z calcd for [C₂₈H₁₉F₇N₅O₄S]⁺: 654.10, found: 654.05. HPLC t_{R}=7.778 min (96.6%).

### Synthesis of 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-(trifluoromethyl)phenyl)sulfonyl)piperazin-1-yl)pyrido[2,3-d]pyrimidin-2(1H)-one (Compound 6A)

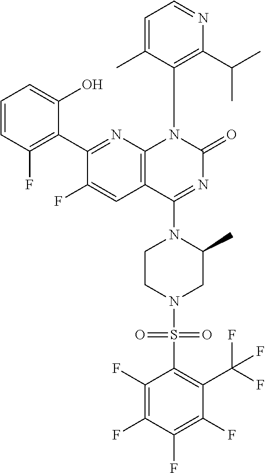 [SMILES: Cc1ccnc(C(C)C)c1Cn1c(=O)nc(N2CCN(S(=O)(=O)c3c(F)c(F)c(F)c(F)c3C(F)(F)F)C[C@@H]2C)c2cc(F)c(-c3c(O)cccc3F)nc21]

The title compound, 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-(trifluoromethyl)phenyl)sulfonyl)piperazin-1-yl)pyrido[2,3-d]pyrimidin-2(1H)-one, was prepared from 7-chloro-6-fluoro-1-(2-isopropyl-4-methyl-3-pyridyl)-4-[(2S)-2-methyl-4-[2,3,4,5-tetrafluoro-6-(trifluoromethyl)phenyl]sulfonyl-piperazin-1-yl]pyrido[2,3-d]pyrimidin-2-one (80 mg, 0.112 mmol) via Scheme 3 to yield the white solid (40 mg, 45%). ¹H NMR (400 MHz, CDCl₃) δ 9.37 (d, J=2.2 Hz, 1H), 8.67 (d, J=4.9 Hz, 1H), 7.90 (dd, J=9.3, 5.1 Hz, 1H), 7.38-7.30 (m, 1H), 7.24 (d, J=4.9 Hz, 1H), 6.73 (d, J=8.5 Hz, 2H), 4.97 (d, J=59.6 Hz, 1H), 4.65-4.42 (m, 1H), 4.07 (d, J=13.2 Hz, 1H), 3.91 (d, J=12.7 Hz, 1H), 3.81 (t, J=13.3 Hz, 1H), 3.53-3.28 (m, 2H), 2.78 (dt, J=30.4, 6.8 Hz, 1H), 2.14-2.01 (m, 3H), 1.73 (dd, J=25.5, 6.8 Hz, 3H), 1.27 (dd, J=6.6, 5.1 Hz, 4H), 1.09 (t, J=6.7 Hz, 3H). ¹⁹F NMR (376 MHz, CDCl₃) δ −51.74 (d, J=35.8 Hz, 3F), −107.29 (dq, J=81.4, 8.1 Hz, 1F), −120.41-−121.14 (m, 1F), −130.01 (ddd, J=45.5, 20.8, 10.1 Hz, 1F), −132.24 (td, J=22.8, 22.0, 10.5 Hz, 1F), −143.77 (tt, J=20.4, 10.0 Hz, 1F), −144.45 (t, J=29.8 Hz, 1F). LC-MS (ESI+) m/z calc'd for [C₃₄H₂₇F₉N₆O₄S]⁺: 787.0, found: 787.3. LC-MS purity: 97.7%, 4.5 min.

### Synthesis of 2-(2,4-dichlorophenoxy)-1-(4-((2,3,4,5-tetrafluoro-6-(trifluoromethyl)phenyl)sulfonyl)piperazin-1-yl)ethan-1-one (Compound 51A)

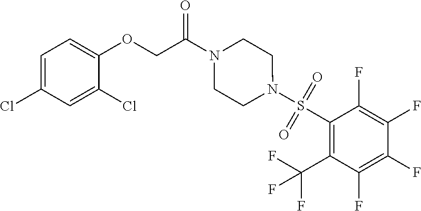 [SMILES: O=C(COc1ccc(Cl)cc1Cl)N1CCN(S(=O)(=O)c2c(F)c(F)c(F)c(F)c2C(F)(F)F)CC1]

The title compound, 2-(2,4-dichlorophenoxy)-1-(4-((2,3,4,5-tetrafluoro-6-(trifluoromethyl)phenyl)sulfonyl)piperazin-1-yl)ethan-1-one, was prepared from 2-(2,4-dichlorophenoxy)-1-piperazin-1-yl-ethanone (37 mg, 0.128 mmol) and 2,3,4,5-tetrafluoro-6-(trifluoromethyl)benzenesulfonyl chloride (45 mg, 0.140 mmol) via Scheme 2 to yield the product (23 mg, 30%). ¹H NMR (400 MHz, CDCl₃) δ 7.42 (d, J=2.6 Hz, 1H), 7.23 (dd, J=8.8, 2.5 Hz, 1H), 6.97 (d, J=8.9 Hz, 1H), 4.79 (s, 2H), 3.85-3.72 (m, 4H), 3.47 (d, J=30.8 Hz, 4H). ¹⁹F NMR (376 MHz, CDCl₃) δ −51.67 (d, J=36.4 Hz, 3F), −130.35 (qdt, J=35.8, 19.8, 9.6 Hz, 1F), −131.26 (dt, J=23.6, 10.0 Hz, 1F), −143.98 (td, J=20.5, 10.4 Hz, 1F), −144.57 (ddd, J=23.9, 20.3, 10.0 Hz, 1F). LC-MS (ESI+) m/z calc'd for [C₁₉H₁₃Cl₂F₇N₂O₄S]⁺: 569.0, found: 569.1. LC-MS purity: 98.8%, 6.8 min.

### Synthesis of 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-(fluoromethoxy)phenyl)sulfonyl)piperazin-1-yl)pyrido[2,3-d]pyrimidin-2(1H)-one (Compound 7A)

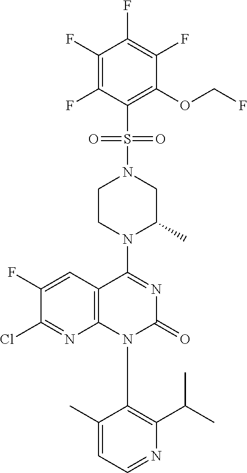 [SMILES: Cc1ccnc(C(C)C)c1Cn1c(=O)nc(N2CCN(S(=O)(=O)c3c(F)c(F)c(F)c(F)c3OCF)C[C@@H]2C)c2cc(F)c(Cl)nc21]

The intermediate, (S)-7-chloro-6-fluoro-1-(2-isopropyl-4-methylpyridin-3-yl)-4-(2-methyl-4-((2,3,4,5-tetrafluoro-6-(fluoromethoxy)phenyl)sulfonyl)piperazin-1-yl)pyrido[2,3-d]pyrimidin-2(1H)-one, was prepared via Scheme 3. ¹H NMR (400 MHz, CDCl₃) δ 8.58 (d, J=4.2 Hz, 1H), 7.76 (d, J=7.6 Hz, 1H), 7.15 (d, J=5.0 Hz, 1H), 5.69 (d, J=53.0, 2.1 Hz, 2H), 4.84 (d, J=8.0 Hz, 1H), 4.38 (t, J=11.6 Hz, 1H), 3.98 (d, J=12.4 Hz, 1H), 3.86-3.73 (m, 2H), 3.18-3.07 (m, 1H), 3.07-2.95 (m, 1H), 2.58 (dh, J=20.0, 6.5 Hz, 1H), 2.05-1.96 (m, 3H), 1.70-1.61 (m, 3H), 1.24-1.17 (m, 3H), 1.15-1.06 (m, 3H). ¹⁹F NMR (376 MHz, CDCl₃) δ−125.43-−125.55 (m, 1F), −133.02-−133.20 (m, 1F), −144.68 (td, J=21.3, 7.4 Hz, 1F), −147.94 (td, J=21.0, 9.2 Hz, 1F), −149.84 (td, J=52.9, 20.8 Hz, 1F), −155.24 (t, J=22.4 Hz, 1F).

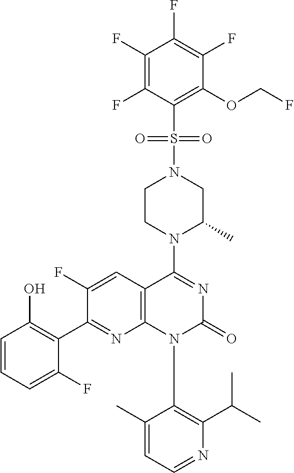 [SMILES: Cc1ccnc(C(C)C)c1Cn1c(=O)nc(N2CCN(S(=O)(=O)c3c(F)c(F)c(F)c(F)c3OCF)C[C@@H]2C)c2cc(F)c(-c3c(O)cccc3F)nc21]

The title compound, 6-fluoro-7-(2-fluoro-6-hydroxyphenyl)-1-(2-isopropyl-4-methylpyridin-3-yl)-4-((S)-2-methyl-4-((2,3,4,5-tetrafluoro-6-(fluoromethoxy)phenyl)sulfonyl)piperazin-1-yl)pyrido[2,3-d]pyrimidin-2(1H)-one, was prepared from 7-chloro-6-fluoro-1-(2-isopropyl-4-methyl-3-pyridyl)-4-[(2S)-2-methyl-4-[2,3,4,5-tetrafluoro-6-(fluoromethoxy)phenyl]sulfonyl-piperazin-1-yl]pyrido[2,3-d]pyrimidin-2-one (35 mg, 0.051 mmol) via Scheme 3 to yield the product (10 mg, 25%). ¹H NMR (400 MHz, CDCl₃) δ 9.34 (s, 1H), 8.67 (d, J=5.0 Hz, 1H), 7.88 (dd, J=9.4, 4.7 Hz, 1H), 7.33 (q, J=7.7 Hz, 1H), 7.24 (d, J=5.0 Hz, 1H), 6.74-6.68 (m, 2H), 5.70 (d, J=52.9 Hz, 2H), 5.03 (s, 1H), 4.90 (s, 1H), 4.59 (d, J=13.6 Hz, 1H), 4.46 (d, J=13.5 Hz, 1H), 4.02 (d, J=12.4 Hz, 1H), 3.95-3.75 (m, 3H), 3.10 (m, 3H), 2.76 (ddd, J=27.6, 13.7, 7.1 Hz, 2H), 2.06 (d, J=17.3 Hz, 3H), 1.72 (dd, J=24.7, 6.8 Hz, 4H), 1.25 (dd, J=6.8, 3.6 Hz, 3H), 1.08 (t, J=6.2 Hz, 3H). ¹⁹F NMR (376 MHz, CDCl₃) δ −107.34 (d, J=80.9 Hz, 1F), −120.73-−121.03 (m, 1F), −132.93-−133.09 (m, 1F), −144.52-−144.73 (m, 1F, −147.71-−147.95 (m, 1F), −149.85 (td, J=52.8, 20.8 Hz, 1F), −155.17 (t, J=22.5 Hz, 1F). LC-MS (ESI+) m/z calcd for [C₃₄H₂₉N₆F₇O₅S]⁺: 767.68, found: 767.20. Purity by LC-MS: 96% at 254 nm.

### Synthesis of N-[[7-(2,6-dimethylphenyl)-3-quinolyl]methyl]-2,3,4,5,6-pentafluoro-benzenesulfonamide (Compound 19A)

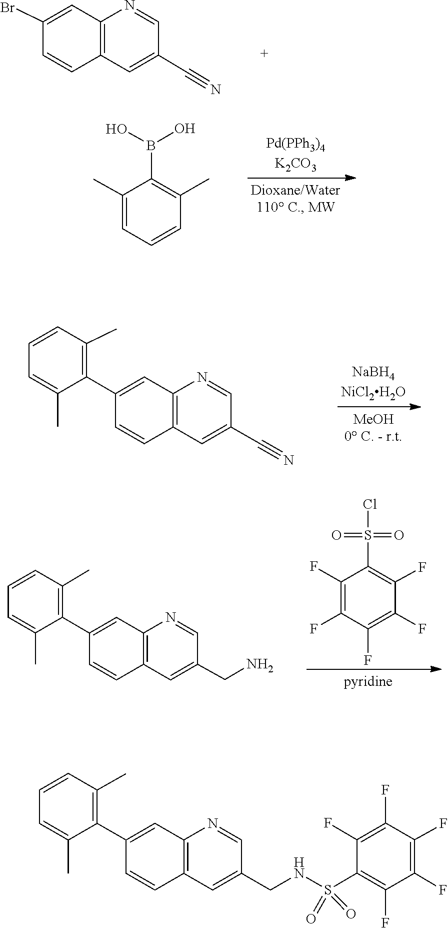

### Synthesis of 7-(2,6-dimethylphenyl)quinoline-3-carbonitrile

In a dry 30 mL microwave vial equipped with a stir bar were combined 7-bromoquinoline-3-carbonitrile (156.3 mg, 670.63 umol), (2,6-dimethylphenyl)boronic acid (150.87 mg, 1.01 mmol), Potassium Carbonate (185.38 mg, 1.34 mmol), Tetrakis(triphenylphosphine)palladium(0) (31.00 mg, 26.83 umol) and a mixture of Dioxane/water. The sealed vial was degassed, and then irradiated at 100° C. for 2.5 h. The reaction progress was monitored by TLC (Hex/EtOAc=2/1) and LC-MS. After 2.5 h, the reaction mixture was cooled down to r.t and quenched with saturated solution of NaHCO₃. The resulting aqueous layer was extracted with EtOAc three times and then the combined organic layers were washed with brine, dried over Na₂SO₄, filtered, and concentrated under reduced pressure to yield orange oil. The crude mixture was purified by C18 reverse phase column chromatography (MeCN/H₂O, 0.1% Formic Acid) to give 7-(2,6-dimethylphenyl)quinoline-3-carbonitrile (1, 140 mg, 541.97 mol, 80.81 yield). 1H NMR (400 MHz, CdC₃) δ 9.07 (d, J=2.1 Hz, 1H), 8.60 (d, J=2.1 Hz, 1H), 7.99-7.96 (m, 2H), 7.52 (dd, J=8.3 Hz, J=1.6 Hz, 1H), 7.27-7.23 (m, 1H), 7.18-7.16 (m, 2H), 2.05 (s, 6H).

### Synthesis of 7-(2,6-dimethylphenyl)-3-quinolylmethanamine

An oven-dried 5 mL microwave vial was charged with 7-(2,6-dimethylphenyl)quinoline-3-carbonitrile (67 mg, 259.37 μmol) and Methanol (1.5 mL). The solution was cooled down to 0° C., and then sodium borohydride (68.68 mg, 1.82 mmol, 63.95 μL) and nickel(II) chloride hexahydrate (246.60 mg, 1.04 mmol) were added in small portion. The reaction mixture was warmed up to ambient temperature and stirred for another 2 hr. The reaction mixture was quenched with NH₄OH (0.5 mL) and allowed to stir for another 10 minutes. The mixture was diluted with EtOAc and was washed with NaHCO₃ three times. The combined organic layers were washed with brine, dried over Na₂SO₄ and concentrated under reduced pressure to obtain yellow solid as the product [7-(2,6-dimethylphenyl)-3-quinolyl]methanamine (2, 60 mg, 228.70 μmol, 88.18% yield). LCMS measured m/z 263 [M+1]⁺.

### Synthesis of N-[[7-(2,6-dimethylphenyl)-3-quinolyl]methyl]-2,3,4,5,6-pentafluoro-benzenesulfonamide (Compound 19A)

To a solution of [7-(2,6-dimethylphenyl)-3-quinolyl]methanamine (30 mg, 114.35 umol) and pyridine (9.05 mg, 114.35 μmol, 9.25 μL) was added dropwise 2,3,4,5,6-pentafluorobenzenesulfonyl chloride (33.53 mg, 125.79 μmol, 18.63 μL) at 0° C. The mixture was warmed up to r.t. and stirred for another 2.5 hr. The reaction was quenched with 1M HCl and the mixture was extracted with EtOAc three times. The collected organic layers were washed with brine, dried over Na₂SO₄, filtered, and concentrated under reduced pressure. The crude mixture was purified by normal phase column chromatography eluting in gradient from 10%-40% EtOAc in Hexanes to afford N-[[7-(2,6-dimethylphenyl)-3-quinolyl]methyl]-2,3,4,5,6-pentafluoro-benzenesulfonamide (6 mg, 11.82 μmol, 10.33% yield, >97% purity). 1H NMR (400 MHz, CdCl₃) δ 8.78 (s, 1H), 8.15 (s, 1H), 7.87-7.85 (m, 2H), 7.42-7.40 (m, 1H), 7.24-7.21 (m, 1H), 7.17-7.15 (m, 2H), 5.60 (br, 1H), 4.63 (s, 2H), 2.05 (s, 6H). ¹⁹F NMR (376 MHz, CdCl₃) δ −136.67_-136.76 (2F), −145.47_-145.54 (1F), −158.26_-158.38 (2F). LC-MS (ESI+) m/z calcd for [C₂₄H₁₇F₅N₂O₂S]⁺: 493.1, found: 493.0.

### Synthesis of N-(7-chloro-6-fluoro-1-(2-isopropyl-4-methylpyridin-3-yl)-2-oxo-1,2-dihydropyrido[2,3-d]pyrimidin-4-yl)-2,3,4,5-tetrafluoro-6-methoxybenzenesulfonamide (Compound 8A)

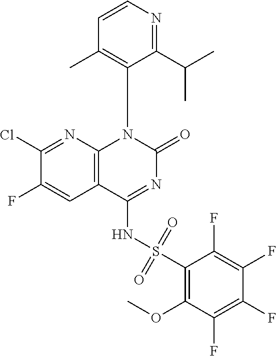 [SMILES: COc1c(F)c(F)c(F)c(F)c1S(=O)(=O)Nc1nc(=O)n(Cc2c(C)ccnc2C(C)C)c2nc(Cl)c(F)cc12]

The title compound, N-(7-chloro-6-fluoro-1-(2-isopropyl-4-methylpyridin-3-yl)-2-oxo-1,2-dihydropyrido[2,3-d]pyrimidin-4-yl)-2,3,4,5-tetrafluoro-6-methoxybenzenesulfonamide, was prepared from 4,7-dichloro-6-fluoro-1-(2-isopropyl-4-methyl-3-pyridyl)pyrido[2,3-d]pyrimidin-2-one (169 mg, 0.46 mmol) and 2,3,4,5-tetrafluoro-6-methoxy-benzenesulfonamide (119 mg, 0.46 mmol) via Scheme 4 to yield the product (19 mg, 7%). ¹H NMR (400 MHz, CDCl₃) δ 8.66 (d, J=4.9 Hz, 1H), 8.31 (d, J=6.9 Hz, 1H), 7.21 (dd, J=4.9, 0.8 Hz, 1H), 4.14 (d, J=1.7 Hz, 3H), 2.68 (hept, J=6.7 Hz, 1H), 2.13 (s, 3H), 1.21 (dd, J=36.4, 6.7 Hz, 6H). ¹⁹F NMR (376 MHz, CDCl₃) δ−122.21 (1F), −135.68-−135.89 (m, 1F), −146.04 (1F), −152.59 (dd, J=20.8, 9.3 Hz, 1F), −159.36 (dd, J=23.6, 21.0 Hz, 1F). LC-MS (ESI+) m/z calcd for [C₂₃H₁₈ClF₅N₅O₄S]⁺: 590.07, found: 590.05. HPLC t_{R}=6.356 min (96.6%).

### Synthesis of N-(7-chloro-6-fluoro-1-(2-isopropyl-4-methylpyridin-3-yl)-2-oxo-1,2-dihydropyrido[2,3-d]pyrimidin-4-yl)-2,3,4,5,6-pentafluorobenzenesulfonamide (Compound 4A)

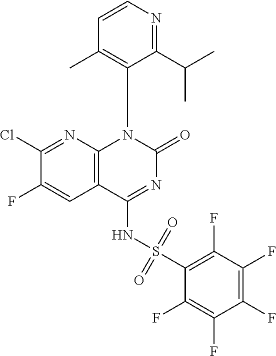 [SMILES: Cc1ccnc(C(C)C)c1Cn1c(=O)nc(NS(=O)(=O)c2c(F)c(F)c(F)c(F)c2F)c2cc(F)c(Cl)nc21]

The title compound, N-(7-chloro-6-fluoro-1-(2-isopropyl-4-methylpyridin-3-yl)-2-oxo-1,2-dihydropyrido[2,3-d]pyrimidin-4-yl)-2,3,4,5,6-pentafluorobenzenesulfonamide, was prepared from 4,7-dichloro-6-fluoro-1-(2-isopropyl-4-methyl-3-pyridyl)pyrido[2,3-d]pyrimidin-2-one (500 mg, 1.36 mmol) and 2,3,4,5,6-pentafluorobenzenesulfonamide (403 mg, 1.63 mmol) via Scheme 4 to yield the product (85 mg, 10%). ¹H NMR (400 MHz, CDCl₃) δ 8.58 (d, J=4.9 Hz, 1H), 8.07 (d, J=7.0 Hz, 1H), 7.15 (d, J=5.0 Hz, 1H), 2.64 (p, J=6.8 Hz, 1H), 2.00 (s, 3H), 1.09 (t, J=6.8 Hz, 6H). ¹⁹F NMR (376 MHz, CDCl₃) δ−124.25 (1F), −138.26 (2F), −147.04 (1F), −159.29 (2F). LC-MS (ESI−) m/z calcd for [C₂₂H₁₃ClF₆N₅O₃S]⁻: 576.03, found: 576.00. HPLC t_{R}=4.588 min (97.5%).

### Synthesis of 2,3,4,5-tetrafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-6-methoxybenzenesulfonamide (Compound 9A)

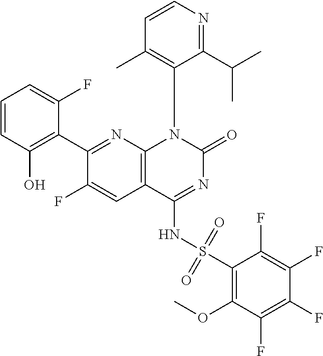 [SMILES: COc1c(F)c(F)c(F)c(F)c1S(=O)(=O)Nc1nc(=O)n(Cc2c(C)ccnc2C(C)C)c2nc(-c3c(O)cccc3F)c(F)cc12]

The title compound, 2,3,4,5-tetrafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-6-methoxybenzenesulfonamide, was prepared from N-[7-chloro-6-fluoro-1-(2-isopropyl-4-methyl-3-pyridyl)-2-oxo-pyrido[2,3-d]pyrimidin-4-yl]-2,3,4,5-tetrafluoro-6-methoxy-benzenesulfonamide (200 mg, 0.339 mmol) via Scheme 4 to yield (31 mg, 13%). ¹H NMR (400 MHz, CD₃CN) δ 11.02 (s, 1H), 8.54 (d, J=4.9 Hz, 1H), 8.40 (d, J=8.4 Hz, 1H), 7.93 (d, J=1.2 Hz, 1H), 7.34 (td, J=8.4, 6.7 Hz, 1H), 7.25 (dd, J=4.9, 0.8 Hz, 1H), 6.78-6.68 (m, 2H), 4.11 (d, J=2.0 Hz, 3H), 2.95 (hept, J=6.7 Hz, 1H), 2.12 (d, J=0.7 Hz, 3H), 1.10 (dd, J=60.7, 6.7 Hz, 6H). ¹⁹F NMR (376 MHz, CD₃CN) δ −114.37 (ddd, J=25.0, 9.9, 6.9 Hz, 1F), −125.28 (dd, J=25.1, 8.5 Hz, 1F), −138.87 (ddd, J=22.9, 9.0, 7.2 Hz, 1F), −149.87 (d, J=24.2 Hz, 1F), −154.82 (dd, J=19.6, 8.8 Hz, 1F), −163.19 (dd, J=22.9, 20.2 Hz, 1F). LC-MS (ESI+) m/z calcd for [C₂₉H₂₂F₆N₅O₅S]⁺: 666.12, found: 666.05. HPLC t_{R}=5.859 min (99.3%).

### Synthesis of 2,3,4,5-tetrafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-6-(fluoromethoxy)benzenesulfonamide (Compound 10A)

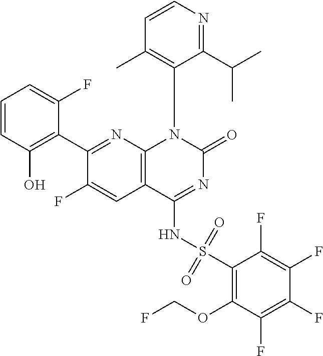 [SMILES: Cc1ccnc(C(C)C)c1Cn1c(=O)nc(NS(=O)(=O)c2c(F)c(F)c(F)c(F)c2OCF)c2cc(F)c(-c3c(O)cccc3F)nc21]

The title compound, 2,3,4,5-tetrafluoro-N-(6-fluoro-7-(2-fluoro-6-hydroxyphenyl)-1-(2-isopropyl-4-methylpyridin-3-yl)-2-oxo-1,2-dihydropyrido[2,3-d]pyrimidin-4-yl)-6-(fluoromethoxy)benzenesulfonamide, was prepared from N-[7-chloro-6-fluoro-1-(2-isopropyl-4-methyl-3-pyridyl)-2-oxo-pyrido[2,3-d]pyrimidin-4-yl]-2,3,4,5-tetrafluoro-6-(fluoromethoxy)benzenesulfonamide (75 mg, 0.123 mmol) via Scheme 4 to yield the product (7 mg, 8.3%). ¹H NMR (400 MHz, CD₃CN) δ 10.87 (s, 1H), 8.53 (d, J=4.8 Hz, 1H), 8.41 (d, J=8.4 Hz, 1H), 7.96 (s, 1H), 7.34 (td, J=8.4, 6.7 Hz, 1H), 7.24 (dd, J=4.9, 0.8 Hz, 1H), 6.78-6.68 (m, 2H), 5.92 (s, 1H), 5.79 (s, 1H), 3.00-2.89 (m, 1H), 2.10 (d, J=0.7 Hz, 3H), 1.08 (dd, J=61.0, 6.7 Hz, 6H). ¹⁹F NMR (376 MHz, CD₃CN) δ −114.38 (1F), −125.59 (1F), −136.46 (d, J=20.0 Hz, 1F), −149.24 (1F), −150.30 (td, J=52.8, 16.3 Hz, 1F), −152.85 (1F), −159.95 (1F). LC-MS (ESI+) m/z calcd for [C₂₉H₂₁F₇N₅O₅S]⁺: 684.12, found: 684.05. HPLC t_{R}=5.160 min (96.1%).

### Synthesis of N-[1-[2-[2,5-bis(chloranyl)phenoxy]acetyl]azetidin-3-yl]-2,3,4,5-tetrakis(fluoranyl)-6-methylsulfonyl-benzamide (Compound 52A)

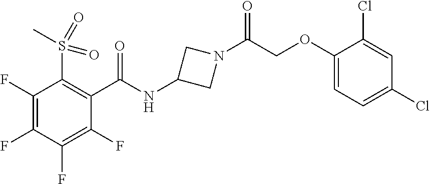 [SMILES: CS(=O)(=O)c1c(F)c(F)c(F)c(F)c1C(=O)NC1CN(C(=O)COc2ccc(Cl)cc2Cl)C1]

The title compound, N-[1-[2-[2,5-bis(chloranyl)phenoxy]acetyl]azetidin-3-yl]-2,3,4,5-tetrakis(fluoranyl)-6-methylsulfonyl-benzamide, was prepared via Scheme 5. 1H NMR (400 MHz, CdCl₃) δ 7.37 (d, J=2.6 Hz, 1H), 7.21-7.18 (dd, J=8.8 Hz, J=2.5 Hz, 1H), 6.80 (d, J=8.8 Hz, 1H), 6.77 (br, 1H), 4.84-4.76 (m, 2H), 4.62 (br, 2H), 4.50-4.45 (m, 2H), 4.12-4.09 (dd, J=11.4 Hz, J=4.8 Hz, 1H), 3.30 (s, 3H). ¹⁹F NMR (376 MHz, CdCl₃) δ −130.95-131.07 (1F), −137.13_-137.24 (1F), −142.02_-142.16 (1F), −148.49_-148.61 (1F). LCMS measured m/z 527 [M−1]⁻.

### Synthesis of 4-(4-((2-(difluoromethoxy)-3,4,5,6-tetrafluorophenyl)sulfonyl)piperazin-1-yl)-7-(naphthalen-1-yl)-5,6,7,8-tetrahydropyrido[3,4-d]pyrimidine (Compound 30A)

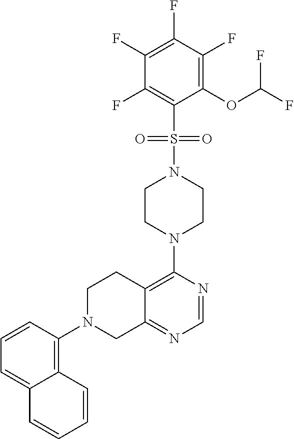 [SMILES: O=S(=O)(c1c(F)c(F)c(F)c(F)c1OC(F)F)N1CCN(c2ncnc3c2CCN(c2cccc4ccccc24)C3)CC1]

The title compound, 4-(4-((2-(difluoromethoxy)-3,4,5,6-tetrafluorophenyl)sulfonyl)piperazin-1-yl)-7-(naphthalen-1-yl)-5,6,7,8-tetrahydropyrido[3,4-d]pyrimidine, was prepared via General Procedure H. The corresponding amine, 7-(naphthalen-1-yl)-4-(piperazin-1-yl)-5,6,7,8-tetrahydropyrido[3,4-d]pyrimidine, was generated via Scheme 1 (9.3 mg, 12% yield). ¹H NMR (400 MHz, MeOD) δ 8.51 (s, 1H), 8.28-8.22 (m, 1H), 7.91-7.86 (m, 1H), 7.64 (d, J=8.2 Hz, 1H), 7.56-7.48 (m, 2H), 7.45 (t, J=7.8 Hz, 1H), 7.23 (d, J=7.4 Hz, 1H), 6.89 (t, J=73.6 Hz, 1H), 4.26 (s, 2H), 3.72 (t, J=5.0 Hz, 4H), 3.47 (t, J=5.0 Hz, 4H), 3.34 (s, 2H), 3.02 (s, 2H). ¹⁹F NMR (376 MHz, MeOD) δ −84.28 (dd, J=73.7, 13.2 Hz, 2F), −134.84 (dt, J=23.6, 8.1 Hz, 1F), −149.25 (td, J=20.4, 7.5 Hz, 1F), −150.12-−150.30 (m, 1F), −157.32-−157.58 (m, 1F). LC-MS (ESI+) m/z calcd for [C₂₈H₂₃N₅SO₃F₆]⁺: 624.57, found: 624.1. Purity by LC-MS: 99% at 254 nm.

### Synthesis of 2,3,4,5-tetrafluoro-6-((4-(7-(naphthalen-1-yl)-5,6,7,8-tetrahydropyrido[3,4-d]pyrimidin-4-yl)piperazin-1-yl)sulfonyl)phenol (Compound 31A)

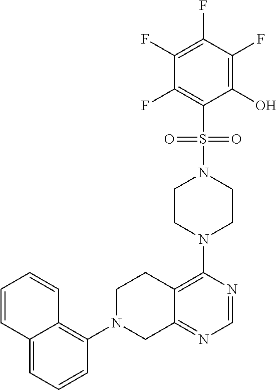 [SMILES: O=S(=O)(c1c(O)c(F)c(F)c(F)c1F)N1CCN(c2ncnc3c2CCN(c2cccc4ccccc24)C3)CC1]

The title compound, 2,3,4,5-tetrafluoro-6-((4-(7-(naphthalen-1-yl)-5,6,7,8-tetrahydropyrido[3,4-d]pyrimidin-4-yl)piperazin-1-yl)sulfonyl)phenol, was prepared via General Procedure H. The corresponding amine, 7-(naphthalen-1-yl)-4-(piperazin-1-yl)-5,6,7,8-tetrahydropyrido[3,4-d]pyrimidine, was generated via Scheme 1 (24.7 mg 4.9% yield). ¹H NMR (400 MHz, MeOD) δ 8.52 (s, 1H), 8.25 (d, J=7.9 Hz, 1H), 8.15 (s, 1H), 7.92-7.84 (m, 1H), 7.64 (d, J=8.2 Hz, 1H), 7.55-7.49 (m, 2H), 7.46 (t, J=7.8 Hz, 1H), 7.24 (d, J=7.5 Hz, 1H), 4.27 (s, 2H), 3.71 (d, J=5.2 Hz, 4H), 3.50 (t, J=5.0 Hz, 4H), 3.37 (s, 2H), 3.03 (s, 2H). ¹⁹F NMR (376 MHz, MeOD) δ −138.82 (m, 1F), −152.32 (m, 1F), −162.61 (m, 1F), −172.21 (m, 1F). MS (ESI+) m/z calcd for [C₂₇H₂₃F₄N₅O₃S]⁺: 574.15, found: 574.16. Purity by HPLC: 97.6% at 254 nm.

### Synthesis of 7-(8-chloronaphthalen-1-yl)-4-(4-((2,3,4,5-tetrafluoro-6-(trifluoromethyl)phenyl)sulfonyl)piperazin-1-yl)-5,6,7,8-tetrahydropyrido[3,4-d]pyrimidine (Compound 32A)

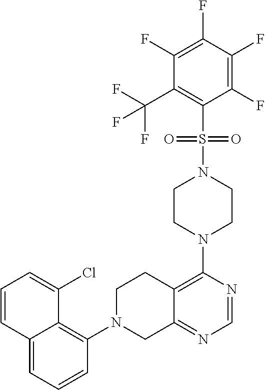 [SMILES: O=S(=O)(c1c(F)c(F)c(F)c(F)c1C(F)(F)F)N1CCN(c2ncnc3c2CCN(c2cccc4cccc(Cl)c24)C3)CC1]

The title compound, 7-(8-chloronaphthalen-1-yl)-4-(4-((2,3,4,5-tetrafluoro-6-(trifluoromethyl)phenyl)sulfonyl)piperazin-1-yl)-5,6,7,8-tetrahydropyrido[3,4-d]pyrimidine, was prepared via General Procedure H. The corresponding amine, 7-(naphthalen-1-yl)-4-(piperazin-1-yl)-5,6,7,8-tetrahydropyrido[3,4-d]pyrimidine, was generated via Scheme 1 (20 mgs, 16% yield). ¹H NMR (400 MHz, CDCl₃) δ 8.64 (s, 1H), 7.81-7.77 (m, 1H), 7.66 (d, J=8.1 Hz, 1H), 7.55 (d, J=7.4 Hz, 1H), 7.48 (t, J=7.8 Hz, 1H), 7.37 (t, J=7.8 Hz, 1H), 7.27 (d, J=8.6 Hz, 2H), 4.53 (d, J=17.8 Hz, 1H), 3.95 (d, J=17.7 Hz, 1H), 3.80-3.51 (m, 9H), 3.28-3.12 (m, 2H), 2.61 (d, J=14.3 Hz, 1H). ¹⁹F NMR (376 MHz, CDCl₃) δ −51.53 (d, J=36.9 Hz, 3F), −130.24 (dt, J=21.8, 9.7 Hz, 1F), −130.43-−130.86 (m, 1F), −144.42 (td, J=20.5, 10.2 Hz, 1F), −144.80 (ddd, J=23.7, 20.1, 9.7 Hz, 1F). MS (ESI+) m/z calcd for [C₂₈H₂₂N₅SO₂F₇Cl]⁺: 660.09, found: 660.09. Purity by HPLC: 95.0% at 254 nm.

### Synthesis of 7-(naphthalen-1-yl)-4-(4-((2,3,4,5-tetrafluoro-6-(trifluoromethyl)phenyl)sulfonyl)piperazin-1-yl)-5,6,7,8-tetrahydropyrido[3,4-d]pyrimidine (Compound 33A)

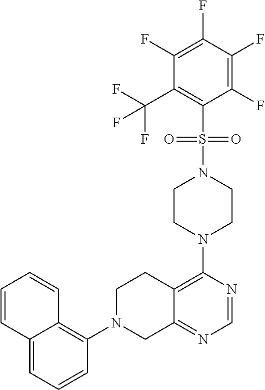 [SMILES: O=S(=O)(c1c(F)c(F)c(F)c(F)c1C(F)(F)F)N1CCN(c2ncnc3c2CCN(c2cccc4ccccc24)C3)CC1]

The title compound, 7-(naphthalen-1-yl)-4-(4-((2,3,4,5-tetrafluoro-6-(trifluoromethyl)phenyl)sulfonyl)piperazin-1-yl)-5,6,7,8-tetrahydropyrido[3,4-d]pyrimidine, was generated via Scheme 1 (20 mgs, 11.13% yield). ¹H NMR (400 MHz, CDCl₃) δ 8.66 (s, 1H), 8.26-8.20 (m, 1H), 7.92-7.87 (m, 1H), 7.65 (d, J=8.2 Hz, 1H), 7.53 (dd, J=6.4, 3.3 Hz, 2H), 7.47 (t, J=7.8 Hz, 1H), 7.19 (d, J=7.4 Hz, 1H), 4.38 (s, 2H), 3.66 (d, J=14.7 Hz, 8H), 3.42 (s, 2H), 2.95 (s, 2H). ¹⁹F NMR (376 MHz, CDCl₃) δ −51.52 (d, J=36.7 Hz, 3F), −130.23-−130.40 (m, 1F), −130.46-−130.69 (m, 1F), −144.23-−144.43 (m, 1F), −144.77 (td, J=22.3, 21.9, 9.7 Hz, 1F). MS (ESI+) m/z calcd for [C₂₈H₂₃N₅SO₂F₇]⁺: 626.56, found: 626.20. Purity by HPLC: 99.6% at 254 nm.

### Synthesis of 7-chloro-6-fluoro-1-(2-isopropyl-4-methylpyridin-3-yl)-4-((S)-2-methyl-4-((2,3,4,5-tetrafluoro-6-(trifluoromethyl)phenyl)sulfonyl)piperazin-1-yl)pyrido[2,3-d]pyrimidin-2(H)-one (Compound 11A)

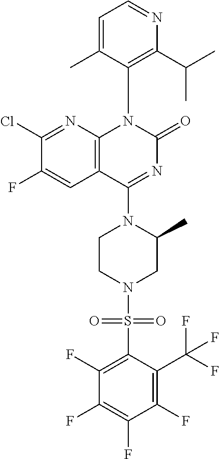 [SMILES: Cc1ccnc(C(C)C)c1-n1c(=O)nc(N2CCN(S(=O)(=O)c3c(F)c(F)c(F)c(F)c3C(F)(F)F)C[C@@H]2C)c2cc(F)c(Cl)nc21]

The title compound, 7-chloro-6-fluoro-1-(2-isopropyl-4-methylpyridin-3-yl)-4-((S)-2-methyl-4-((2,3,4,5-tetrafluoro-6-(trifluoromethyl)phenyl)sulfonyl)piperazin-1-yl)pyrido[2,3-d]pyrimidin-2(1H)-one, was prepared via Scheme 3 (20 mgs, 9.8% yield). ¹H NMR (400 MHz, CDCl₃) δ 8.60 (d, J=4.8 Hz, 1H), 7.78 (d, J=7.3 Hz, 1H), 7.17 (d, J=4.8 Hz, 1H), 4.84 (s, 1H), 4.45-4.34 (m, 1H), 4.03 (d, J=12.5 Hz, 1H), 3.91-3.77 (m, 2H), 3.43 (t, J=10.6 Hz, 1H), 3.33 (q, J=12.5, 10.9 Hz, 1H), 2.68-2.54 (m, 1H), 2.06 (s, 3H), 1.67 (dd, J=6.8, 2.1 Hz, 3H), 1.23 (t, J=6.1 Hz, 3H), 1.13 (dd, J=8.8, 6.7 Hz, 3H). ¹⁹F NMR (376 MHz, CDCl₃) δ −51.75 (d, J=35.8 Hz), −125.25-−125.54 (m), −129.75-−130.29 (m), −132.02-−132.38 (m), −143.56-−143.92 (m), −144.18-−144.61 (m). MS (ESI+) m/z calcd for [C₂₈H₂₄ClF₈N₆O₃S]⁺: 711.11, found: 711.06. Purity by HPLC: 97.4% at 254 nm.

### Synthesis of 2,3,4,5-tetrafluoro-N-(1-(7-(naphthalen-1-yl)-5,6,7,8-tetrahydropyrido[3,4-d]pyrimidin-4-yl)azetidin-3-yl)-6-(trifluoromethyl)benzenesulfonamide (Compound 35A)

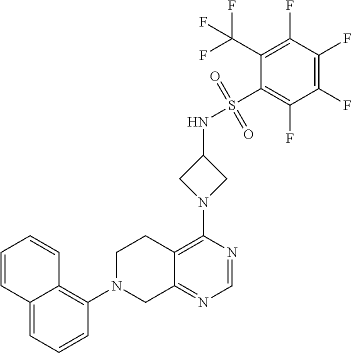 [SMILES: O=S(=O)(NC1CN(c2ncnc3c2CCN(c2cccc4ccccc24)C3)C1)c1c(F)c(F)c(F)c(F)c1C(F)(F)F]

The title compound, 2,3,4,5-tetrafluoro-N-(1-(7-(naphthalen-1-yl)-5,6,7,8-tetrahydropyrido[3,4-d]pyrimidin-4-yl)azetidin-3-yl)-6-(trifluoromethyl)benzenesulfonamide, was prepared via General Procedure H. The corresponding amine, 1-(7-(naphthalen-1-yl)-5,6,7,8-tetrahydropyrido[3,4-d]pyrimidin-4-yl)azetidin-3-amine, was generated via Scheme 1 (4 mg, 2.2% yield). ¹H NMR (400 MHz, CDCl₃) δ 8.46 (s, 1H), 8.20-8.10 (m, 1H), 7.88 (t, J=4.8 Hz, 1H), 7.63 (d, J=8.1 Hz, 1H), 7.56-7.48 (m, 2H), 7.44 (t, J=7.8 Hz, 1H), 7.13 (d, J=7.3 Hz, 1H), 4.73-4.53 (m, 4H), 4.36 (s, 2H), 4.27 (s, 2H), 3.40 (s, 2H), 2.93 (s, 2H). ¹⁹F NMR (376 MHz, CDCl₃) δ −51.54-−51.64 (3F), −130.01 (1F), −130.83 (1F), −143.80 (1F), −144.21 (1F). MS (ESI+) m/z calcd for [C₂₇H₂₁F₇N₅O₂S]⁺: 612.54, found: 612.16. Purity by HPLC: 96.9% at 254 nm.

### Synthesis of 2,3,4,5,6-pentafluoro-N-(1-(7-(naphthalen-1-yl)-5,6,7,8-tetrahydropyrido[3,4-d]pyrimidin-4-yl)azetidin-3-yl)benzenesulfonamide (Compound 20A)

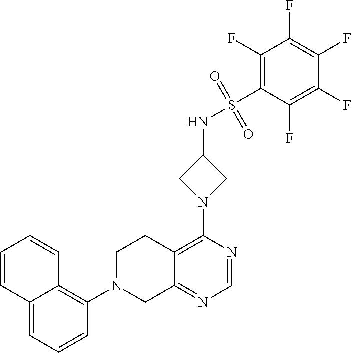 [SMILES: O=S(=O)(NC1CN(c2ncnc3c2CCN(c2cccc4ccccc24)C3)C1)c1c(F)c(F)c(F)c(F)c1F]

The title compound, 2,3,4,5,6-pentafluoro-N-(1-(7-(naphthalen-1-yl)-5,6,7,8-tetrahydropyrido[3,4-d]pyrimidin-4-yl)azetidin-3-yl)benzenesulfonamide, was prepared via General Procedure H (15 mg, 8.4% yield). ¹H NMR (400 MHz, CDCl₃) δ 8.45 (s, 1H), 8.17-8.10 (m, 1H), 7.90-7.85 (m, 1H), 7.63 (d, J=8.2 Hz, 1H), 7.56-7.48 (m, 2H), 7.43 (t, J=7.8 Hz, 1H), 7.12 (d, J=7.4 Hz, 1H), 4.64 (dt, J=18.4, 7.5 Hz, 3H), 4.38 (s, 2H), 4.26 (s, 2H), 3.38 (s, 2H), 2.93 (s, 2H). ¹⁹F NMR (376 MHz, CDCl₃) δ−136.51-−136.71 (m, 2F), −144.86-−145.06 (m, 1F), −157.73-−157.98 (m, 2F). LC-MS (ESI+) m/z calcd for [C₂₆H₁₉F₅N₅O₂S]⁻: 560.53, found: 560.20. Purity by HPLC: 90.6% at 254 nm.

### Synthesis of N-(1-(7-chloro-6-fluoro-1-(2-isopropyl-4-methylpyridin-3-yl)-2-oxo-1,2-dihydropyrido[2,3-d]pyrimidin-4-yl)azetidin-3-yl)-2,3,4,5-tetrafluoro-6-(trifluoromethyl)benzenesulfonamide (Compound 13A)

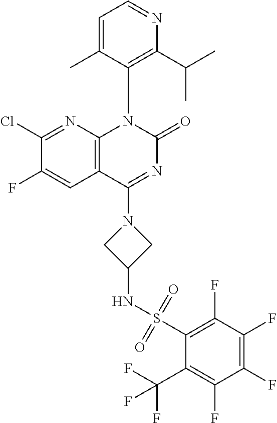 [SMILES: Cc1ccnc(C(C)C)c1-n1c(=O)nc(N2CC(NS(=O)(=O)c3c(F)c(F)c(F)c(F)c3C(F)(F)F)C2)c2cc(F)c(Cl)nc21]

The title compound, N-(1-(7-chloro-6-fluoro-1-(2-isopropyl-4-methylpyridin-3-yl)-2-oxo-1,2-dihydropyrido[2,3-d]pyrimidin-4-yl)azetidin-3-yl)-2,3,4,5-tetrafluoro-6-(trifluoromethyl)benzenesulfonamide, was prepared via Scheme 3. ¹H NMR (400 MHz, MeOD) δ 8.61 (d, J=8.2 Hz, 1H), 8.49 (d, J=5.0 Hz, 1H), 7.32 (d, J=5.0 Hz, 1H), 5.03 (t, J=6.8 Hz, 1H), 4.56 (dd, J=17.2, 9.1 Hz, 2H), 4.42-4.34 (m, 2H), 2.73 (t, J=6.9 Hz, 1H), 2.05 (s, 3H), 1.19 (d, J=6.8 Hz, 3H), 1.08 (d, J=6.8 Hz, 3H). ¹⁹F NMR (376 MHz, MeOD) δ −52.74 (d, J=37.2 Hz, 3F), −127.50 (d, J=8.1 Hz, 1F), −129.59 (dd, J=22.5, 10.5 Hz, 1F), −135.24 (dd, J=28.1, 9.3 Hz, 1F), −147.94-−148.16 (m, 1F), −148.34 (td, J=19.6, 11.0 Hz, 1F). MS (ESI+) m/z calcd for [C₂₆H₂₀ClF₈N₆O₃S]⁺: 683.08, found: 683.11. Purity by HPLC: 96.8% at 254 nm.

### Synthesis of 7-chloro-6-fluoro-1-(2-isopropyl-4-methylpyridin-3-yl)-4-((1-((2,3,4,5-tetrafluoro-6-(trifluoromethyl)phenyl)sulfonyl)azetidin-3-yl)amino)pyrido[2,3-d]pyrimidin-2(H)-one (Compound 14A)

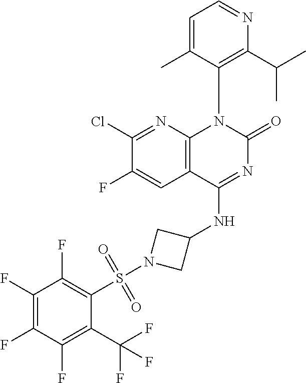 [SMILES: Cc1ccnc(C(C)C)c1-n1c(=O)nc(NC2CN(S(=O)(=O)c3c(F)c(F)c(F)c(F)c3C(F)(F)F)C2)c2cc(F)c(Cl)nc21]

The title compound, 7-chloro-6-fluoro-1-(2-isopropyl-4-methylpyridin-3-yl)-4-((1-((2,3,4,5-tetrafluoro-6-(trifluoromethyl)phenyl)sulfonyl)azetidin-3-yl)amino)pyrido[2,3-d]pyrimidin-2(1H)-one, was prepared via Scheme 3 (14.2 mg, 4.7% yield). ¹H NMR (400 MHz, CDCl₃) δ 8.63 (d, J=5.1 Hz, 1H), 8.14 (s, 1H), 7.78 (d, J=7.4 Hz, 1H), 7.22 (d, J=5.1 Hz, 1H), 4.80 (d, J=102.5 Hz, 5H), 2.66-2.52 (m, 1H), 2.01 (s, 3H), 1.09 (d, J=6.9 Hz, 6H). ¹⁹F NMR (376 MHz, CDCl₃) δ −51.53 (d, J=36.2 Hz, 3F), −123.87-−124.35 (m, 1F), −128.71-−129.34 (m, 1F), −130.99-−131.72 (m, 1F), −143.88-−144.27 (m, 1F), −144.36-−144.75 (m, 1F). MS (ESI+) m/z calcd for [C₂₆H₂₀ClF₈N₆O₃S]⁺: 683.08, found: 683.11. Purity by HPLC: 97.0% at 254 nm.

### Synthesis of 7-(3-methoxy-1-naphthyl)-4-[4-[2,3,4,5-tetrakis(fluoranyl)-6-[tris(fluoranyl)methyl]phenyl]sulfonylpiperazin-1-yl]-6,8-dihydro-5H-pyrido[3,4-d]pyrimidine (Compound 40A)

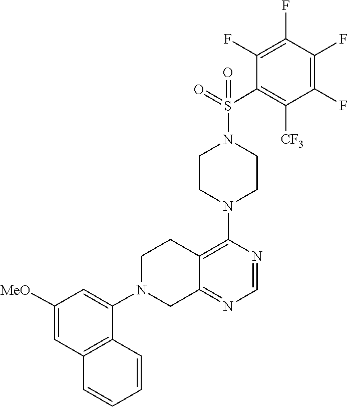 [SMILES: COc1cc(N2CCc3c(ncnc3N3CCN(S(=O)(=O)c4c(F)c(F)c(F)c(F)c4C(F)(F)F)CC3)C2)c2ccccc2c1]

The title compound, 7-(3-methoxy-1-naphthyl)-4-[4-[2,3,4,5-tetrakis(fluoranyl)-6-[tris(fluoranyl)methyl]phenyl]sulfonylpiperazin-1-yl]-6,8-dihydro-5H-pyrido[3,4-d]pyrimidine, was prepared via General Procedure H. The corresponding amine, 7-(3-methoxynaphthalen-1-yl)-4-(piperazin-1-yl)-5,6,7,8-tetrahydropyrido[3,4-d]pyrimidine, was generated via Scheme 1. ¹H NMR (400 MHz, CDCl3) δ 8.65 (s, 1H), 8.10 (d, J=8.4 Hz, 1H), 7.78 (d, J=8.1 Hz, 1H), 7.48 (t, J=7.5 Hz, 1H), 7.37 (t, 1H, J=8.3 Hz), 6.94 (d, J=2.3 Hz, 1H), 6.85 (d, J=2.3 Hz, 1H), 4.34 (s, 2H), 3.95 (s, 3H), 3.69-3.65 (m, 4H), 3.65-3.61 (m, 4H), 3.40 (s, 2H), 2.94 (s, 2H). ¹⁹F NMR (376 MHz, CDCl3) δ 51.52 (d, 3F), 130.28 (dt, 1H), 130.62 (m, 1H), 144.37 (m, 1H), 144.78 (m, 1H). ESI-MS [M+H]⁺: 656.400

### Synthesis of 6-fluoro-7-(2-fluoro-6-hydroxyphenyl)-1-(2-isopropyl-4-methylpyridin-3-yl)-4-((1-((2,3,4,5-tetrafluoro-6-(trifluoromethyl)phenyl)sulfonyl)azetidin-3-yl)amino)pyrido[2,3-d]pyrimidin-2(1H)-one (Compound 15A)

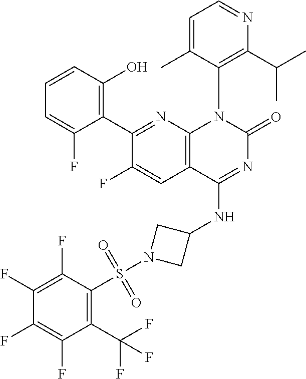 [SMILES: Cc1ccnc(C(C)C)c1-n1c(=O)nc(NC2CN(S(=O)(=O)c3c(F)c(F)c(F)c(F)c3C(F)(F)F)C2)c2cc(F)c(-c3c(O)cccc3F)nc21]

The title compound, 6-fluoro-7-(2-fluoro-6-hydroxyphenyl)-1-(2-isopropyl-4-methylpyridin-3-yl)-4-((1-((2,3,4,5-tetrafluoro-6-(trifluoromethyl)phenyl)sulfonyl)azetidin-3-yl)amino)pyrido[2,3-d]pyrimidin-2(1H)-one, was prepared via Scheme 3. ¹H NMR (400 MHz, CD₃CN) δ 8.47 (d, J=4.9 Hz, 1H), 8.02 (d, J=9.3 Hz, 1H), 7.38-7.27 (m, 1H), 7.19 (d, J=4.9 Hz, 1H), 6.72 (dt, J=9.1, 4.9 Hz, 2H), 4.75 (d, J=97.9 Hz, 9H), 2.83-2.70 (m, 1H), 1.12 (dd, J=6.7, 1.4 Hz, 3H), 0.98 (dd, J=6.7, 1.4 Hz, 3H). ¹⁹F NMR (376 MHz, CD₃CN) δ −52.18 (d, J=36.8 Hz, 3F), −113.18-−113.57 (m, 1F), −127.51 (dd, J=37.6, 9.3 Hz, 1F), −130.69-−131.08 (m, 1F), −134.00-−134.70 (m, 1F), −146.75 (ddd, J=23.0, 19.3, 10.3 Hz, 1F), −147.71 (td, J=19.7, 11.0 Hz, 1F). LC-MS (ESI+) m/z calcd for [C₃₂H₂₃F₉N₆O₄S]⁻: 757.13, found: 757.1. Purity by HPLC: 97.0% at 254 nm.

### Synthesis of 4-(4-(4-((2,3,4,5-tetrafluoro-6-(trifluoromethyl)phenyl)sulfonyl)piperazin-1-yl)-5,8-dihydropyrido[3,4-d]pyrimidin-7(6H)-yl)naphthalen-2-ol (Compound 41A)

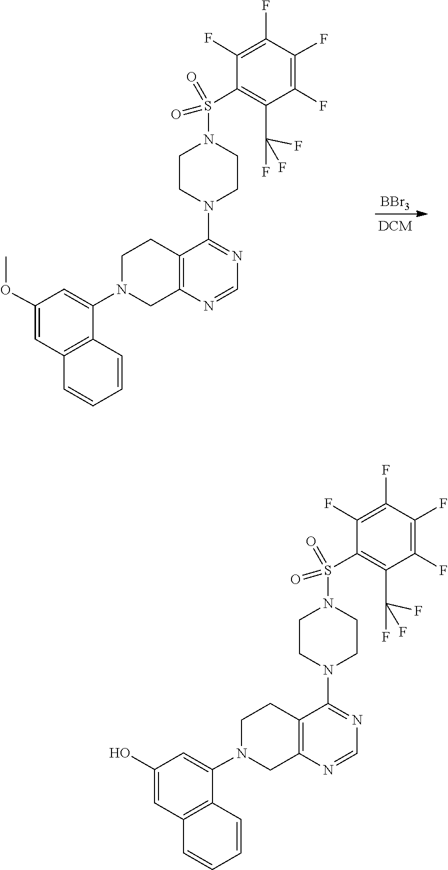 [SMILES: COc1cc(N2CCc3c(ncnc3N3CCN(S(=O)(=O)c4c(F)c(F)c(F)c(F)c4C(F)(F)F)CC3)C2)c2ccccc2c1 ; "BBr3" ; O=S(=O)(c1c(F)c(F)c(F)c(F)c1C(F)(F)F)N1CCN(c2ncnc3c2CCN(c2cc(O)cc4ccccc24)C3)CC1]

In a microwave vial equipped with a stir bar and a nitrogen-filled balloon, 7-(3-methoxy-1-naphthyl)-4-[4-[2,3,4,5-tetrakis(fluoranyl)-6-[tris(fluoranyl)methyl]phenyl]sulfonylpiperazin-1-yl]-6,8-dihydro-5H-pyrido[3,4-d]pyrimidine (10 mg, 15.25 μmol, 1 eq.) was dissolved in DCM (3 mL, 0.22 M) at r.t. under N₂. The resulting solution was then cooled to −78° C. using Acetone-dry ice bath and then added with boron tribromide (1 M, 22.88 μL, 1.5 eq.) in a dropwise manner. The reaction mixture was stirred at r.t. for overnight. The reaction mixture was quenched with 1M HCl and extracted three times with DCM. The collected organic layers were washed once with saturated sodium chloride, dried with sodium sulfate, filtered, and evaporated under reduced pressure. The mixture was separated on a pad of silica using Biotage Isolera 50 g cartridge eluting in gradient from 10% to 15% EtOAc in Hexanes to afford the product 4-[4-[4-[2,3,4,5-tetrakis(fluoranyl)-6-[tris(fluoranyl)methyl]phenyl]sulfonylpiperazin-1-yl]-6,8-dihydro-5H-pyrido[3,4-d]pyrimidin-7-yl]naphthalen-2-ol (8 mg, 12.47 μmol, 81.75% yield). ¹H NMR (400 MHz, CDCl₃) δ 8.66 (s, 1H), 8.09 (d, J=8.5 Hz, 1H), 7.71 (d, J=8.1 Hz, 1H), 7.47 (m, 1H), 7.37 (m, 1H), 6.95 (s, 1H), 6.83 (s, 1H), 4.36 (s, 2H), 3.68 (m, 4H), 3.64 (m, 6H), 2.94 (s, 2H). ¹⁹F NMR (376 MHz, CD₃CN) δ −52.18 (3F), −131.2 (1F), −133.12 (1F), −147.21 (1F), −148.2 (1F). LC-MS: 98.8%, 4.68 min, found 642.2 (+)

### Synthesis of N-(1-(7-(8-chloronaphthalen-1-yl)-5,6,7,8-tetrahydropyrido[3,4-d]pyrimidin-4-yl)azetidin-3-yl)-2,3,4,5,6-pentafluorobenzenesulfonamide (Compound 21A)

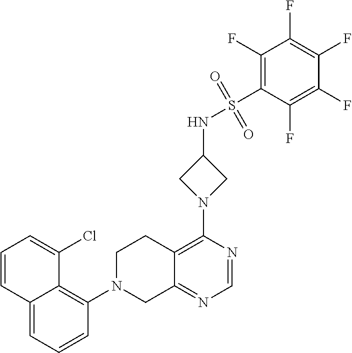 [SMILES: O=S(=O)(NC1CN(c2ncnc3c2CCN(c2cccc4cccc(Cl)c24)C3)C1)c1c(F)c(F)c(F)c(F)c1F]

The title compound, N-(1-(7-(8-chloronaphthalen-1-yl)-5,6,7,8-tetrahydropyrido[3,4-d]pyrimidin-4-yl)azetidin-3-yl)-2,3,4,5,6-pentafluorobenzenesulfonamide, was prepared via General Procedure H. The corresponding amine, 1-(7-(8-chloronaphthalen-1-yl)-5,6,7,8-tetrahydropyrido[3,4-d]pyrimidin-4-yl)azetidin-3-amine, was generated via Scheme 1. ¹H NMR (400 MHz, CDCl₃) δ 8.50 (s, 1H), 7.77 (dd, J=8.4, 1.3 Hz, 1H), 7.64 (dd, J=8.2, 1.2 Hz, 1H), 7.54 (dd, J=7.5, 1.3 Hz, 1H), 7.47 (t, J=7.8 Hz, 1H), 7.35 (dd, J=8.1, 7.4 Hz, 1H), 7.25 (dd, J=7.5, 1.2 Hz, 1H), 5.69 (p, J=7.7 Hz, 1H), 4.96-4.90 (m, 1H), 4.86-4.79 (m, 1H), 4.75 (t, J=8.7 Hz, 1H), 4.67 (t, J=8.7 Hz, 1H), 4.37 (d, J=17.2 Hz, 1H), 3.93 (d, J=17.2 Hz, 1H), 3.62 (d, J=7.4 Hz, 1H), 3.23-3.12 (m, 2H), 2.70-2.64 (m, 1H). ¹⁹F NMR (376 MHz, CDCl₃) δ −134.80 (dt, J=17.5, 10.4 Hz), −140.98 (tt, J=21.5, 8.2 Hz), −156.53-−157.10 (m). LC-MS (ESI+) m z calcd for [C₂₆H₁₈ClF₅N₅O₂S]⁻: 594.08, found: 594.20. Purity by HPLC: 95.1% at 254 nm.

### Synthesis of (S)-6-chloro-7-(2,6-dimethylphenyl)-4-(2-methyl-4-(2,3,4,5-tetrafluoro-6-(methylsulfonyl)benzyl)piperazin-1-yl)quinoline (Compound 45A)

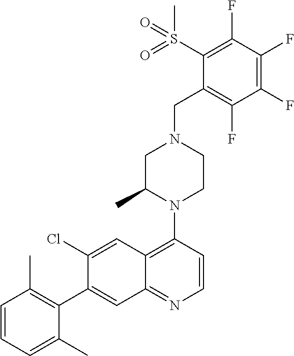 [SMILES: Cc1cccc(C)c1-c1cc2nccc(N3CCN(Cc4c(F)c(F)c(F)c(F)c4S(C)(=O)=O)C[C@@H]3C)c2cc1Cl]

The title compound, (S)-6-chloro-7-(2,6-dimethylphenyl)-4-(2-methyl-4-(2,3,4,5-tetrafluoro-6-(methylsulfonyl)benzyl)piperazin-1-yl)quinoline, was prepared via Scheme 6. ¹H NMR (400 MHz, DMSO-d₆) δ 8.78 (d, J=4.8 Hz, 1H), 8.22 (s, 1H), 7.77 (s, 1H), 7.23-7.18 (m, 4H), 4.03-4.02 (m, 2H), 3.59 (s, 3H), 2.73-2.89 (m, 4H), 2.89-2.67 (m, 6H), 1.95 (d, J=8.8 Hz, 6H), 0.95 (d, J=6.0 Hz, 3H). ¹⁹F NMR (376 MHz, DMSO-d₆) δ −131.36_131.45 (1F), −138.94_-139.02 (1F), −147.23_147.28 (1F), −153.84_-153.96 (1F). LCMS: METHOD II, RT—2.428 ESI-MS: measured m/z 606.4 [M+1]⁺. HPLC: METHOD I. RT 6.670, 95.05%.

### Synthesis of 2-(6-chloro-8-fluoro-4-((S)-2-methyl-4-(2,3,5,6-tetrafluoro-4-(methylsulfonyl)phenyl)piperazin-1-yl)quinolin-7-yl)-3-fluorophenol (Compound 24A, Compound 25A, Compound 26A) and 2-(6-chloro-8-fluoro-4-((S)-2-methyl-4-(2,3,5,6-tetrafluoro-4-(methylthio)phenyl)piperazin-1-yl)quinolin-7-yl)-3-fluorophenol (Compound 29A)

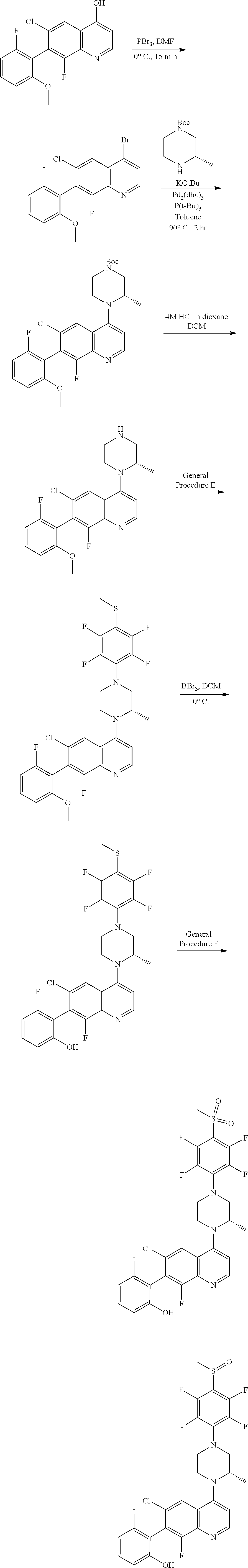

The title compound, 2-(6-chloro-8-fluoro-4-((S)-2-methyl-4-(2,3,5,6-tetrafluoro-4-(methylsulfonyl)phenyl)piperazin-1-yl)quinolin-7-yl)-3-fluorophenol, was prepared via Scheme 7.

Synthesis of 4-bromo-6-chloro-8-fluoro-7-(2-fluoro-6-methoxyphenyl)quinoline. To a stirred solution of 6-chloro-8-fluoro-7-(2-fluoro-6-methoxyphenyl)quinolin-4-ol (5.0 g, 15.57 mmol) in DMF (60 mL) was added Phosphorus tribromide (10.51 g, 38.94 mmol) at 0° C. The resulting reaction mixture was stirred at room temperature for 15 min. After completion of reaction, the reaction mixture was poured in to an ice cold water. The obtained precipitate was filtered off. Isolated solid was dissolved in EtOAc (250 mL×2). The combined organic phases were dried over anhydrous Na₂SO₄, filtered and concentrated under reduced pressure. The resulting crude was purified by silica gel column chromatography, eluted with 28% EtOAc in hexane to afford title compound as a yellow solid (2.12 g, 5.53 mmol, 36% yield). ¹H NMR (400 MHz, DMSO-d6) δ 8.83 (d, J=4.4 Hz, 1H), 8.18-8.16 (m, 2H), 7.62-7.56 (m, 1H), 7.12-7.02 (m, 2H), 3.78 (s, 3H). LCMS: METHOD II, RT—2.791 ESI-MS: measured m/z 384.2, 386.2 [M+1], [M+3]⁺.

Synthesis of tert-butyl (3S)-4-(6-chloro-8-fluoro-7-(2-fluoro-6-methoxyphenyl)quinolin-4-yl)-3-methylpiperazine-1-carboxylate. To a stirred solution of tert-butyl (S)-3-methylpiperazine-1-carboxylate (0.79 g, 3.95 mmol) in toluene (5 mL) were added Potassium tert-butoxide (0.55 g, 4.94 mmol) and 4-bromo-6-chloro-8-fluoro-7-(2-fluoro-6-methoxyphenyl)quinoline (1.26 g, 6.59 mmol) at room temperature. The resulting reaction mixture was purged with N₂ for 15 minutes followed by addition of Pd₂(dba)₃ (0.30 g, 0.32 mmol) and Tri-tert-butylphosphine (0.066 g, 0.32 mmol) at room temperature. The resulting reaction mixture was stirred at 90° C. for 2 h. After completion of reaction, the reaction mixture was cooled to ambient temperature and diluted with water (100 mL). The resulting suspension was extracted with EtOAc (3×100 mL). The combined organic phases were dried over anhydrous Na₂SO₄, filtered and concentrated under reduced pressure. The resulting crude was purified by silica gel column chromatography, eluted with 28% EtOAc in hexane to afford title compound as a yellow solid (1.32 g, 2.62 mmol, 40% yield). ¹H NMR (400 MHz, DMSO-d₆) δ 8.82 (d, J=5.2 Hz, 1H), 8.04 (s, 1H), 7.59-7.53 (q, J₁=8.4, J₂=15.6, 1H), 7.29 (d, J=5.2, 1H), 7.10-7.00 (m, 2H), 3.81-3.77 (m, 5H), 3.76-3.36 (m, 4H), 2.90-2.89 (m, 1H), 1.44 (s, 9H), 0.95-0.91 (m, 3H). LCMS: METHOD II, RT—2.573 ESI-MS: measured m/z 504.5 [M+1]⁺, 506.5 [M+3]⁺.

Synthesis of 6-chloro-8-fluoro-7-(2-fluoro-6-methoxyphenyl)-4-((S)-2-methylpiperazin-1-yl)quinolone. To a stirred solution of tert-butyl (3S)-4-(6-chloro-8-fluoro-7-(2-fluoro-6-methoxy phenyl)quinolin-4-yl)-3-methylpiperazine-1-carboxylate (1.32 g, 2.62 mmol) in DCM (7 mL) was added 4M HCl in 1,4-dioxane (5.6 mL) at room temperature. The resulting reaction mixture was stirred at room temperature for 2 h. After the completion of reaction, the reaction mixture was concentrated under reduce vacuum. The obtained residue was diluted with aq NaHCO₃ (50 mL) and extracted with EtOAc (3×50 mL). The combined organic phases were dried over anhydrous Na₂SO₄, filtered and concentrated under reduced pressure, to afford title compound as a brown solid (0.99 g, 2.47 mmol, Quantitative). ¹H NMR (400 MHz, DMSO-d₆) δ 8.80 (d, J=5.2 Hz, 1H), 8.0 (s, 1H), 7.59-7.53 (q, J₁=8.4 Hz, J₂=15.2 Hz, 1H), 7.25-7.23 (m, 1H), 7.09-7.00 (m, 3H), 3.80-3.77 (m, 4H), 3.63 (brs, 1H), 2.77-3.15 (m, 5H), 0.97 (d, J=6.0 Hz, 3H). ¹⁹F NMR (376 MHz, DMSO-d₆) δ −113.49_113.57 (1F), −118.27_-118.37 (1F). LCMS: METHOD II, RT—1.682 ESI-MS: measured m/z 404.4 [M+1]⁺, 406.4 [M+3]⁺.

Synthesis of 6-chloro-8-fluoro-7-(2-fluoro-6-methoxyphenyl)-4-((S)-2-methyl-4-(2,3,5,6-tetrafluoro-4-(methylthio)phenyl)piperazin-1-yl)quinoline. The product was prepared using General Procedure E. ¹H NMR (400 MHz, DMSO-d₆) δ 8.84 (d, J=4.8 Hz, 1H), 8.08 (d, J=1.2 Hz, 1H), 7.37-7.35 (m, 1H), 7.10-7.00 (m, 3H), 3.90 (brs, 1H), 3.79 (d, J=8.4 Hz, 3H), 3.41-3.70 (m, 4H), 3.03-3.08 (m, 1H), 2.49 (s, 3H), 1.07 (d, J=6.4 Hz, 3H). ¹⁹F NMR (376 MHz, DMSO-d₆) δ −113.60_113.49 (1F), −118.29_-118.18 (1F), −136.66_136.16 (2F), −150.33_-150.20 (2F). LCMS: UC02_FAR1, RT 3.092 ESI-MS: m/z 598.3 [M+1]⁺, 600.2 [M+3]⁺.

Synthesis of 2-(6-chloro-8-fluoro-4-((S)-2-methyl-4-(2,3,5,6-tetrafluoro-4-(methylthio)phenyl) piperazin-1-yl)quinolin-7-yl)-3-fluorophenol (Compound 29A). To the stirred solution of 6-chloro-8-fluoro-7-(2-fluoro-6-methoxyphenyl)-4-((S)-2-methyl-4-(2,3,5,6-tetrafluoro-4-(methylthio)phenyl)piperazin-1-yl)quinoline (0.30 g, 0.50 mmol) in DCM (3 mL) was added BBr₃ (0.18 g, 0.75 mmol) at 0° C. The resulting reaction mixture was stirred at room temperature for 3 h. After the completion of reaction, the reaction mixture was diluted with saturated solution of NaHCO₃ (50 mL) and extracted with DCM (3×50 mL). The combined organic phases were dried over anhydrous Na₂SO₄, filtered and concentrated under reduced pressure, to afford title compound as an off white solid (0.25 g, 0.43 mmol, 86% yield). ¹H NMR (400 MHz, DMSO-d₆) δ 10.23 (brs, 1H), 8.84 (d, J=4.4 Hz, 1H), 8.07 (s, 1H), 7.36 (m, 2H), 6.87-6.80 (m, 2H), 3.90 (brs, 1H), 3.71 (brs, 1H), 3.57-3.50 (m, 3H), 3.06 (brs, 1H), 2.45 (s, 3H), 1.06 (d, J=5.6 Hz, 3H). ¹⁹F NMR (376 MHz, DMSO-d₆) δ −113.63_113.67 (1F), −117.99_-118.17 (1F), −136.57_136.66 (2F), −150.25_-150.33 (2F). LCMS: METHOD II, RT₁—2.777 RT₂—2.810 ESI-MS: measured m/z 584.3 [M+1]⁺, 586.3 [M+3]⁺, HPLC: METHOD I. RT₁ 6.952, 8.75%, RT₂ 7.142, 88.61%.

Synthesis of 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 24A). The product was prepared using General Procedure F. 1H NMR (400 MHz, DMSO-d₆) δ 10.27 (s, 1H), 8.84 (d, J=4.8 Hz, 1H), 8.09 (s, 1H), 7.36-7.34 (m, 2H), 6.87-6.79 (m, 2H), 3.91-3.83 (m, 2H), 3.70-3.58 (m, 4H), 3.50 (s, 3H), 3.45 (s, 3H), 3.05 (d, J=11.6 Hz, 1H), 1.04 (d, J=6.0 Hz, 3H). ¹⁹F NMR (376 MHz, DMSO-d₆) δ −113.64_-113.68 (1F), −117.99_-118.17 (1F), −140.60_-140.64 (2F), −150.24_-150.29 (2F). LCMS: METHOD II, RT₁—2.237, RT2—2.300 ESI-MS: measured m/z 616.3 [M+1]⁺, 618.3 [M+3]⁺. HPLC: METHOD I. RT 6.280, 92.53%, Chiral HPLC RT 4.98, 34.40% (Compound 25A), RT 6.50, 43.12% (Compound 26A). Atropisomers were separated by Waters SFC eluting with 50% of liquid CO₂ in MeOH over 20 minutes on CHIRALPAK IG 250×50 mm 5 um. Characterizations of 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 25A) & (Compound 26A)

Compound 25A: White solid (0.0068 g, 0.011 mmol, 16.21% yield). ¹H NMR (400 MHz, DMSO-d₆) δ 10.99 (s, 1H), 8.83 (d, J=4.8 Hz, 1H), 8.07 (s, 1H), 7.34 (d, J=4.8 Hz, 1H), 7.28 (brs, 1H), 6.81 (brs, 1H), 6.70 (brs, 1H), 3.58-3.91 (m, 6H), 3.45 (s, 3H), 3.06-3.04 (brs, 1H), 1.03 (d, J=6.4 Hz, 3H). ¹⁹F NMR (376 MHz, DMSO-d₆) δ −113.89 (1F), −118.19 (1F), −140.60_140.65 (2F), −150.25_-150.29 (2F). LCMS: METHOD II, RT₁—2.247, RT₂—2.306 ESI-MS: measured m/z 616.3 [M+1]⁺, 618.4 [M+3]⁺ HPLC: METHOD I. RT 6.232, 100%, Chiral HPLC RT 4.85, 95.38% (FR-1). Compound 26A: Off White solid (0.0071 g, 0.011 mmol, 17% yield). ¹H NMR (400 MHz, DMSO-d₆) δ 10.99 (s, 1H), 8.83 (d, J=4.8 Hz, 1H), 8.07 (s, 1H), 7.34 (d, J=4.8 Hz, 1H), 7.28 (brs, 1H), 6.81 (brs, 1H), 6.70 (brs, 1H), 3.58-3.91 (m, 6H), 3.45 (s, 3H), 3.06-3.04 (brs, 1H), 1.03 (d, J=6.4 Hz, 3H). ¹⁹F NMR (376 MHz, DMSO-d₆) δ −113.89 (1F), −118.19 (1F), −140.60_140.65 (2F), −150.25_-150.29 (2F). LCMS: METHOD II, RT—2.304, ESI-MS: measured m/z 616.3, 618.4 [M+1]⁺, [M+3]⁺ HPLC: METHOD I. RT 6.257, 98.64%, Chiral HPLC RT 6.36, 97.64% (FR-2).

### Synthesis of 1-(6-chloro-7-(2,6-dimethylphenyl)quinolin-4-yl)-N-(2,3,5,6-tetrafluoro-4-(methylsulfonyl)phenyl)azetidin-3-amine (Compound 22A) & 1-(6-chloro-7-(2,6-dimethylphenyl)quinolin-4-yl)-N-(2,3,5,6-tetrafluoro-4-(methylsulfinyl)phenyl)azetidin-3-aminephenyl) pyrimidine-2,4-diamine (Compound 27A)

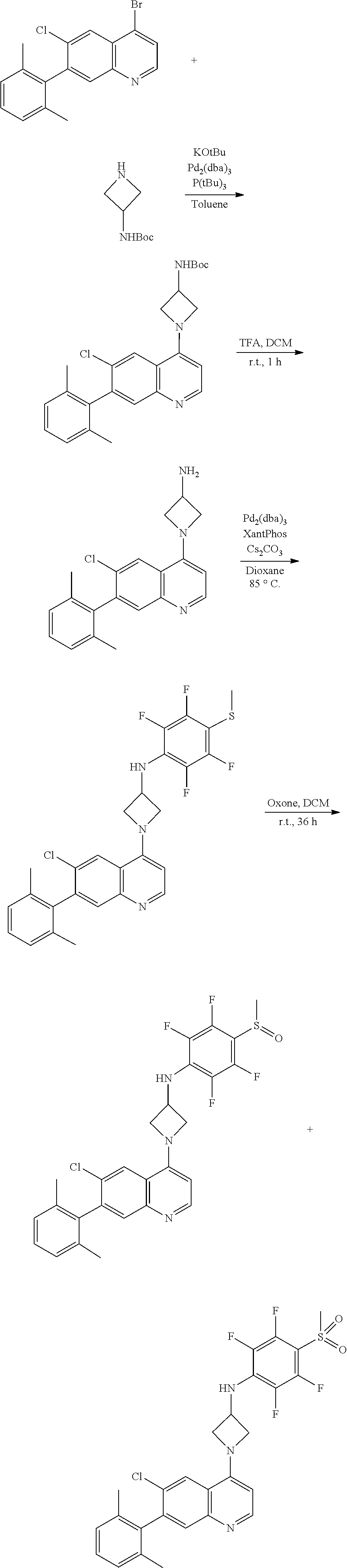

The title compounds, -(6-chloro-7-(2,6-dimethylphenyl)quinolin-4-yl)-N-(2,3,5,6-tetrafluoro-4-(methylsulfonyl)phenyl)azetidin-3-amine and 1-(6-chloro-7-(2,6-dimethylphenyl)quinolin-4-yl)-N-(2,3,5,6-tetrafluoro-4-(methylsulfinyl)phenyl)azetidin-3-aminephenyl) pyrimidine-2,4-diamine, were prepared via Scheme 7.

Synthesis of tert-butyl (1-(6-chloro-7-(2,6-dimethylphenyl)quinolin-4-yl)azetidin-3-yl)carbamate (step 1). To a stirred solution of tert-butyl azetidin-3-ylcarbamate (1 eq.) in toluene (0.8 M) were added Potassium tert-butoxide (1.3 eq.) and 4-bromo-6-chloro-7-(2,6-dimethylphenyl)quinoline (1.7 eq.) at room temperature. The resulting reaction mixture was purged with N₂ for 15 minutes followed by addition of Pd₂(dba)₃ (0.08 eq.) and Tri-tert-butylphosphine (0.08 eq.) at room temperature. The resulting reaction mixture was stirred at 90° C. for 2 h. After completion of reaction, the reaction mixture was cooled to ambient temperature and diluted with water. The resulting suspension was extracted with EtOAc. The combined organic phases were dried over anhydrous Na₂SO₄, filtered and concentrated under reduced pressure. The resulting crude was purified by silica gel column chromatography, eluted with 28% EtOAc in hexane to afford title compound. ¹H NMR (400 MHz, DMSO-d6) δ 8.45 (d, J=5.2 Hz, 1H), 8.07 (s, 1H), 7.66 (d, J=7.2 Hz, 1H), 7.61 (s, 1H), 7.26-7.22 (m, 1H), 7.17 (d, J=7.6 Hz, 2H), 6.36 (d, J=5.6 Hz, 1H), 4.67 (t, J=8.0 Hz, 2H), 4.52-4.50 (brs, 1H), 4.19-4.15 (m, 2H), 1.95 (s, 6H), 1.44 (s, 9H). LCMS: Method-METHOD II, RT—2.104 ESI-MS: measured m/z 438.4, 440.5 [M+1]⁺, [M+3]⁺.

Synthesis of 1-(6-chloro-7-(2,6-dimethylphenyl)quinolin-4-yl)azetidin-3-amine (step 2). The intermediate was prepared via General Procedure D to yield the product. ¹H NMR (400 MHz, DMSO-d6) δ 8.43 (d, J=5.2 Hz, 1H), 8.11 (s, 1H), 7.60 (s, 1H), 7.26-7.22 (m, 1H), 7.18-7.16 (m, 2H), 6.34 (d, J=5.6 Hz, 1H), 4.62 (t, J=8.0 Hz, 2H), 4.02-3.95 (m, 3H), 3.52 (brs, 2H), 1.95 (s, 6H). LCMS: Method-METHOD II, RT—2.104 ESI-MS: measured m/z 438.4, 440.5 [M+1, M+3]⁺.

Synthesis of 1-(6-chloro-7-(2,6-dimethylphenyl)quinolin-4-yl)-N-(2,3,5,6-tetrafluoro-4-(methylthio)phenyl)azetidin-3-amine (step 3). The intermediate was prepared via General Procedure E to yield the product. ¹H NMR (400 MHz, DMSO-d6) δ 8.47 (d, J=5.2 Hz, 1H), 8.12 (s, 1H), 7.62 (s, 1H), 7.26-7.22 (m, 1H), 7.19-7.17 (m, 2H), 6.86 (brs, 1H), 6.41 (d, J=5.2 Hz, 1H), 4.73 (d, J=4.4 Hz, 3H), 4.45 (d, J=4.8 Hz, 2H), 2.37 (s, 3H), 1.95 (s, 6H). ¹⁹F NMR (376 MHz, DMSO-d6) δ −137.39_-137.49 (2F), −159.14_-159.2s4 (2F). LCMS: Method-METHOD II, RT—2.452 ESI-MS: measured m/z 532.4, 534.4 [M+1]⁺, [M+3]⁺.

Synthesis of 1-(6-chloro-7-(2,6-dimethylphenyl)quinolin-4-yl)-N-(2,3,5,6-tetrafluoro-4-(methylsulfonyl)phenyl)azetidin-3-amine (step 4). The intermediate was prepared via General Procedure F to yield the product. ¹H NMR (400 MHz, DMSO-d6) δ 8.48 (d, J=5.2 Hz, 1H), 8.11 (s, 1H), 7.63 (s, 1H), 7.57-7.55 (m, 1H), 7.26-7.22 (m, 1H), 7.18 (d, J=7.6 Hz, 2H), 6.42 (d, J=5.6 Hz, 1H), 4.80 (brs, 1H), 4.73 (t, J=8.4 Hz, 2H), 4.52-4.49 (m, 2H), 3.39 (s, 3H) 1.95 (s, 6H). ¹⁹F NMR (376 MHz, DMSO-d6) δ −141.66_-136.71 (2F), −160.02_-160.07 (2F). LCMS: Method-METHOD II, RT—2.107 ESI-MS: measured m/z 564.3, 566.3 [M+1]⁺, [M+3]⁺. HPLC: Method-I RT 6.389, 99.15%.

Synthesis of 1-(6-chloro-7-(2,6-dimethylphenyl)quinolin-4-yl)-N-(2,3,5,6-tetrafluoro-4-(methylsulfinyl)phenyl)azetidin-3-aminephenyl) pyrimidine-2,4-diamine (step 4). The intermediate was prepared via General Procedure F to yield the product. ¹H NMR (400 MHz, DMSO-d6) δ 8.47 (d, J=5.2 Hz, 1H), 8.11 (s, 1H), 7.62 (s, 1H), 7.28-7.22 (m, 2H), 7.18 (d, J=7.2 Hz, 2H), 6.41 (d, J=5.2 Hz, 1H), 4.74-4.71 (m, 3H), 4.49-4.48 (m, 2H), 3.12 (s, 3H) 1.95 (s, 6H). ¹⁹F NMR (376 MHz, DMSO-d6) δ −143.46_-143.50 (2F), −159.58_-159.63 (2F). LCMS: Method-II, RT—1.992 ESI-MS: measured m/z 548.4, 550.4 [M+1]⁺, [M+3]⁺. HPLC: Method-1 RT 6.231, 97.06%.

### Synthesis of 4-((S)-4-((2-(difluoromethoxy)-3,4,5,6-tetrafluorophenyl) sulfonyl)-2-methylpiperazin-1-yl)-6-fluoro-7-(2-fluoro-6-hydroxyphenyl)-1-(2-isopropyl-4-methylpyridin-3-yl) pyrido[2,3-d]pyrimidin-2(1H)-one (Compound 16A, Compound 17A, Compound 18A)

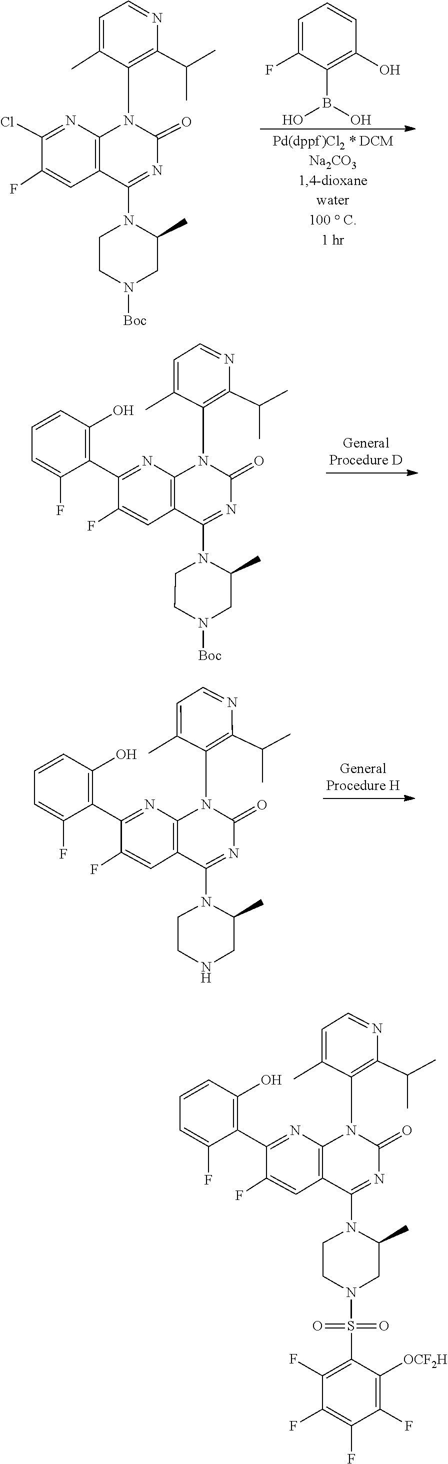

Synthesis of tert-butyl (3S)-4-(6-fluoro-7-(2-fluoro-6-hydroxyphenyl)-1-(2-isopropyl-4-methylpyridin-3-yl)-2-oxo-1,2-dihydropyrido[2,3-d] pyrimidin-4-yl)-3-methylpiperazine-1-carboxylate. To a stirred solution of tert-butyl (S)-4-(7-chloro-6-fluoro-1-(2-isopropyl-4-methylpyridin-3-yl)-2-oxo-1,2-dihydropyrido[2,3-d]pyrimidin-4-yl)-3-methylpiperazine-1-carboxylate (0.8 g, 1.5 mmol) in 1,4-dioxane:water (5:1, 3 mL) were added (2-fluoro-6-hydroxyphenyl) boronic acid (0.47 g, 3.0 mmol) and Cs₂CO₃ (0.48 g, 4.5 mmol) at room temperature. The resulting reaction mixture was purged with N₂ for 10 minutes followed by addition of Pd(dppf)Cl₂·DCM complex (0.13 g, 0.15 mmol) at room temperature. The reaction mixture was heated to 80° C. for 16 h. After completion of the reaction, the mixture was cooled to room temperature. Reaction mixture was diluted with water (100 mL) and extracted with EtOAc (2×50 mL). The combined organic phases were dried over anhydrous Na₂SO₄, filtered and concentrated under reduced pressure. The resulting crude was purified by revers phase chromatography, product eluted with 47% EtOAc in hexane to afford title compound as a brown solid (1.2 g, 1.97 mmol, 70% yield). LCMS: METHOD III, RT—2.66 & 2.90 ESI-MS: measured m/z 607.4 [M+1]⁺. (Mixture of atropisomers).

Synthesis of 6-fluoro-7-(2-fluoro-6-hydroxyphenyl)-1-(2-isopropyl-4-methylpyridin-3-yl)-4-((S)-2-methylpiperazin-1-yl) pyrido[2,3-d] pyrimidin-2(1H)-one. The product was prepared using General Procedure D. LCMS: METHOD III, RT—2.07 & 2.27 ESI-MS: measured m/z 507.37 [M+1]⁺. (Mixture of atropisomers).

Synthesis of 4-((S)-4-((2-(difluoromethoxy)-3,4,5,6-tetrafluorophenyl) sulfonyl)-2-methylpiperazin-1-yl)-6-fluoro-7-(2-fluoro-6-hydroxyphenyl)-1-(2-isopropyl-4-methyl pyridin-3-yl) pyrido[2,3-d] pyrimidin-2(1H)-one (Compound 16A). The product was prepared using General Procedure H. ¹H NMR (400 MHz, MeOD) δ 8.32-8.28 (m, 1H), 8.23-8.15 (m, 1H), 7.21 (q, J₁=8 Hz, J₂=12.4 Hz, 1H), 7.14-7.08 (m, 1H), 6.92-6.88 (m, 1H), 6.75-6.68 (m, 2H), 5.097 (s, 1H), 4.55-4.40 (m, 1H), 4.40-4.36 (m, 3H), 3.33-3.30 (m, 2H), 2.27-2.26 (m, 1H), 1.89 (d, J=6.4 Hz, 3H), 1.63-1.57 (m, 3H), 1.14-1.10 (m, 3H), 0.83 (d, J=6.4 Hz, 3H). ¹⁹F NMR (400 MHz, MeOD) δ −84.213-−84.285 (2F), −112.729-−112.874 (1F), −134.962-−135.064 (1F), −145.659-−145.693 (1F), −149.131-−149.238 (1F), −150.284 (1F), 157.457-−157.571 (1F). LCMS: UC05_FRA1, RT—2.54, ESI-MS: measured m/z 785.3 [M+1]⁺. HPLC: UC07_TFRA1, RT 6.62, 92.22% Atropisomers were separated by Waters SFC eluting with 50% of liquid CO₂ in MeOH over 20 minutes on CHIRALPAK IG 250×50 mm 5 um. 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 17A). ¹H NMR (400 MHz, MeOD) δ 8.29 (d, J=4.8 Hz, 1H), 8.21 (d, J=10 Hz, 1H), 7.23-7.18 (m, 1H), 7.12 (d, J=4.8 Hz, 1H), 6.92-6.87 (m, 1H), 6.76-6.73 (m, 1H), 6.71-6.68 (m, 1H), 5.11 (br, 1H), 4.43-4.41 (m, 1H), 3.96-3.79 (m, 2H), 3.76 (d, J=12.8 Hz, 2H), 3.22-3.15 (m, 2H), 3.08-3.01 (m, 1H), 2.67-2.57 (m, 1H), 1.90 (s, 3H), 1.57 (d, J=6.8 Hz, 3H), 1.10 (d, J=6.8 Hz, 3H), 0.83 (d, J=6.8 Hz, 3H). ¹⁹F NMR (400 MHz, MeOD) δ −84.250_-84.286 (2F), −112.745 (1F), −134.935_-135.018 (1F), −145.690 (1F), 149.102_-149.232 (1F), −150.224_-150.282 (1F), 157.449_-157.565 (1F). LCMS: UC05_FRA1 RT—2.54, ESI-MS: measured m/z 785.4 [M+1]⁺. HPLC: Method I, RT 6.65, 98.10%. 2-(6-chloro-8-fluoro-4-((S)-2-methyl-4-(2,3,5,6-tetrafluoro-4-(methylsulfonyl) phenyl)piperazin-1-yl)quinolin-7-yl)-3-fluorophenol (Compound 18A). ¹H NMR (400 MHz, MeOD) δ 8.29 (d, J=4.8 Hz, 1H), 8.16 (d, J=10 Hz, 1H), 7.23-7.18 (m, 1H), 7.12 (d, J=4.8 Hz, 1H), 6.92-6.87 (m, 1H), 6.76-6.68 (m, 2H), 4.91 (s, 1H), 4.50 (d, J=13.6 Hz, 1H), 3.96 (d, J=12 Hz, 2H), 3.77 (d, J=12.8 Hz, 2H), 3.19-3.09 (m, 2H), 2.67-2.60 (m, 1H), 1.89 (s, 3H), 1.62 (d, J=6.8 Hz, 3H), 1.11 (d, J=6.8 Hz, 3H), 0.83 (d, J=6.8 Hz, 3H). ¹⁹F NMR (400 MHz, MeOD) δ −84.185_-84.277 (2F), −112.733_-112.757 (1F), −134.959_-135.063 (1F), −145.654-145.725 (1F), −149.136_-149.262 (1F), −150.243_-150.353 (1F), 157.463-157.579 (1F). LCMS: UC05_FRA1, RT—2.53, ESI-MS: measured m/z 785.4 [M+1]⁺. HPLC: HP07_TFRA1, RT 6.64, 92.42%.

### Synthesis of (S)-6-chloro-7-(2-fluorophenyl)-4-(2-methyl-4-(2,3,4,5-tetrafluoro-6-(methylsulfonyl)phenyl)piperazin-1-yl)quinazoline (Compound 34A), 6-chloro-7-(2-fluorophenyl)-4-((2S)-2-methyl-4-(2,3,4,5-tetrafluoro-6-(methylsulfinyl)phenyl)piperazin-1-yl)quinazoline (Compound 38A) & (Compound 39A)

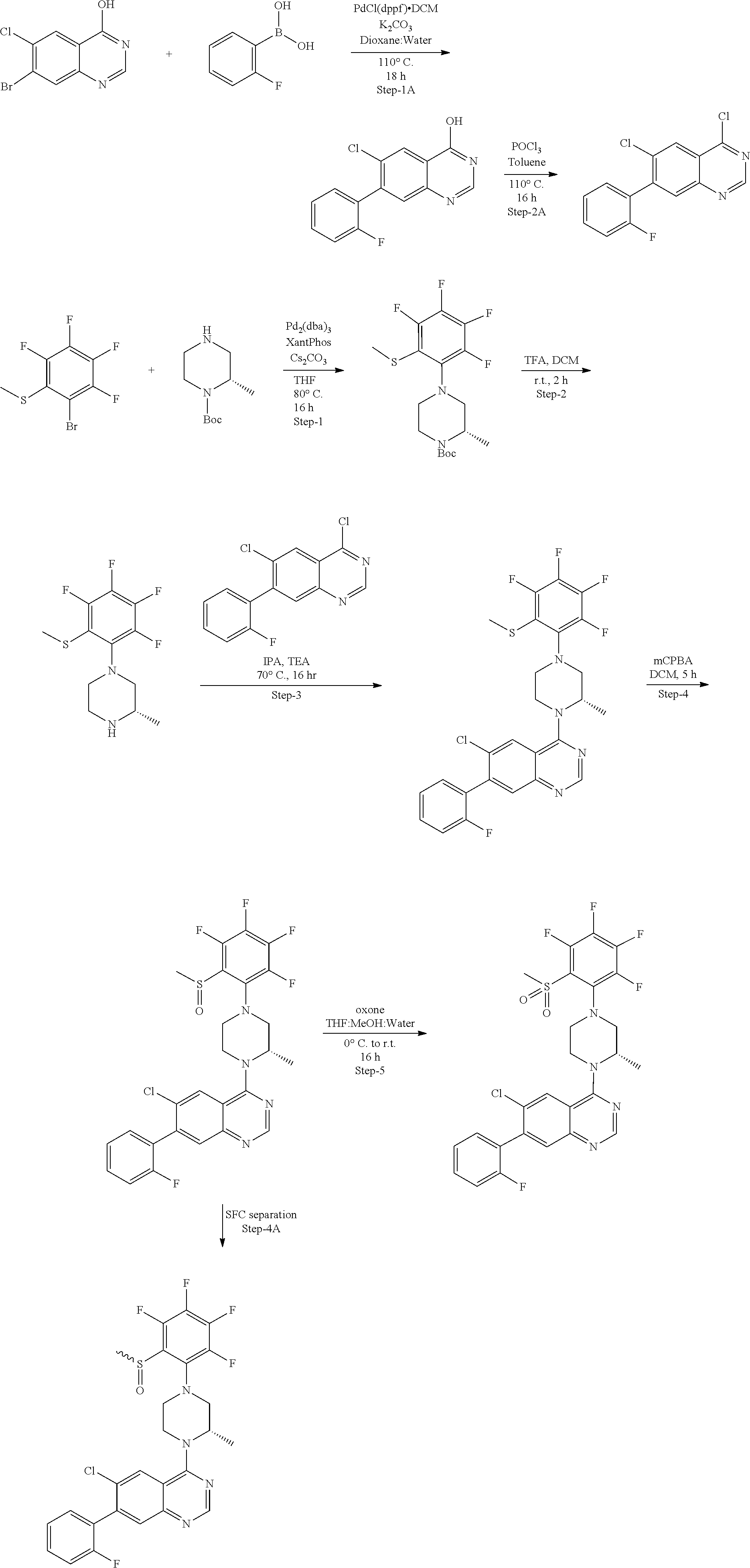

Step-1A: 6-chloro-7-(2-fluorophenyl)quinazolin-4-ol. To a stirred suspension of 7-bromo-6-chloroquinazolin-4-ol (20.0 g, 77.07 mmol) in Dioxane:Water (200 ml:40 mL) were added (2-fluorophenyl)boronic acid (26.96 g, 192.68 mmol) and K₂CO₃ (31.90 g, 231.22 mmol). The resulting reaction mixture was purged with N₂ for 30 minutes followed by addition of PdCl₂(dppf)-DCM (6.30 g, 7.70 mmol). The reaction mixture was stirred at 110° C. for 16 hours. After completion of the reaction, reaction mixture was allowed to cool to ambient temperature and diluted with EtOAc (100 mL). The resulting reaction mixture was filtered over celite bed and washed with 1,4-Dioxane (300 mL). The combined organic phases were concentrated under reduced pressure and azeotropically distilled with toluene (2×50 mL). The resulting crude was purified by flash column chromatography, eluted at 50% EtOAc in Hexane to afford title compound as off-white solid (14.0 g, 51.09 mmol, 66% yield). ¹H NMR (400 MHz, DMSO-d₆) δ 12.50 (s, 1H), 8.20, (s, 1H), 8.18, (s, 1H), 7.71 (s, 1H), 7.58-7.53 (m, 1H), 7.49-7.45 (m, 1H), 7.40-7.34 (m, 2H). LCMS: Method-UC04_FAR1, RT—1.705 ESI-MS: measured m/z 275.2 [M+1]⁺.

Step-2A: 4,6-dichloro-7-(2-fluorophenyl)quinazoline. To a stirred suspension of 6-chloro-7-(2-fluorophenyl)quinazolin-4-ol (1.0 g, 3.64 mmol) in toluene (10 mL) was added POCl₃ (2.23 g, 14.59 mmol) dropwise at room temperature. The resulting reaction mixture was stirred at 110° C. for 16 h. After the completion of reaction, the reaction mixture was evaporated and azeotropically distilled with toluene (2×10 mL). Obtained crude was diluted with ice-cold water (100 mL) and extracted with EtOAc (3×70 mL). The combined organic phases were washed with saturated NaHCO₃ (4×70 mL) solution and dried over anhydrous Na₂SO₄, filtered and concentrated under reduced pressure. the obtained crude was purified by flash column chromatography, eluted at 15% EtOAc in hexane to afford title compound as a white solid (0.4 g, 1.36 mmol, 37% yield). ¹H NMR (400 MHz, DMSO-d₆) δ 8.35 (s, 1H), 8.22 (s, 1H), 7.74 (s, 1H), 7.58-7.54 (m, 1H), 7.49-7.45 (m, 1H), 7.40-7.34 (m, 2H). LCMS: Method-UC04_FAR1, RT-2.383 ESI-MS: measured m/z 293.25 [M+1]⁺.

Step-1: tert-butyl (S)-2-methyl-4-(2,3,4,5-tetrafluoro-6-(methylthio)phenyl)piperazine-1-carboxylate. To a stirred solution of (2-bromo-3,4,5,6-tetrafluorophenyl)(methyl)sulfane (1.0 g, 3.63 mmol) in THF (10 mL) were added tert-butyl (S)-2-methylpiperazine-1-carboxylate (0.72 g, 3.63 mmol) and Cs₂CO₃ (3.53 g, 1.08 mmol). The reaction mixture was purged with N₂ for 15 minutes followed by addition of Pd₂dba₃ (0.33 g, 0.36 mmol) and Xanthphos (0.20 g, 0.36 mmol) at rt. The resulting reaction mixture was stirred for 16 h at 80° C. After completion of reaction, reaction mixture was allowed to cool to ambient temperature and diluted with water (70 mL). The resulting suspension was extracted with EtOAc (3×50 mL). The combined organic phases were dried over anhydrous Na₂SO₄, filtered, and concentrated under reduced pressure. The resulting crude was purified by flash column chromatography, eluted at 17% EtOAc in hexane to afford title compound as a brown sticky solid (0.5 g, 1.26 mmol, 35% yield). LCMS: Method-UC04_FAR1, RT—2.882 ESI-MS: measured m/z 339.2 [M−56]⁺.

Step-2: (S)-3-methyl-1-(2,3,4,5-tetrafluoro-6-(methylthio)phenyl)piperazine. To a stirred solution of tert-butyl (S)-2-methyl-4-(2,3,4,5-tetrafluoro-6-(methylthio)phenyl)piperazine-1-carboxylate (0.30 g, 0.76 mmol) in DCM (3 mL) was added TFA (1.5 mL) dropwise at room temperature. The resulting reaction mixture was stirred at room temperature for 2 h. After the completion of reaction, the reaction mixture was evaporated and azeotropically distilled with DCM (3×10 mL) to afford title compound as a brown sticky solid (0.42 g, 1.42 mmol, quantitative yield). LCMS: Method-METHOD III, RT—2.65 ESI-MS: measured m/z 295.13 [M+1]⁺.

Step-3: (S)-6-chloro-7-(2-fluorophenyl)-4-(2-methyl-4-(2,3,4,5-tetrafluoro-6-(methylthio)phenyl)piperazin-1-yl)quinazoline(DO-000-185-D2). To a stirred solution of (S)-3-methyl-1-(2,3,4,5-tetrafluoro-6-(methylthio)phenyl)piperazine (0.42 g, 1.02 mmol) in IPA (4 mL) were added TEA (0.51 g, 5.14 mmol) and 4,6-dichloro-7-(2-fluorophenyl)quinazoline. The reaction mixture was stirred at 70° C. for 16 h. After completion of reaction, reaction mixture was allowed to cool to ambient temperature and diluted with water (50 mL). The resulting suspension was extracted with EtOAc (3×50 mL). The combined organic phases were dried over anhydrous Na₂SO₄, filtered and concentrated under reduced pressure. The resulting crude was purified by flash column chromatography, eluted at 19% EtOAc in hexane to afford title compound as a yellow solid (0.27 g, 0.49 mmol, 13% yield). ¹H NMR (400 MHz, DMSO-d₆) δ 8.70, (s, 1H), 8.11 (s, 1H), 7.85 (s, 1H), 7.60-7.54 (m, 1H), 7.52-7.48 (m, 1H), 7.41-7.36 (m, 2H), 4.81-4.79 (m, 1H), 4.21 (d, J=13.6 Hz, 1H), 3.87 (t, J=11.6 Hz, 1H), 3.57 (d, J=11.6 Hz, 1H), 3.21 (d, J=11.2 Hz, 1H), 3.05 (d, J=11.6 Hz, 1H), 2.55 (s, 3H), 1.59 (d, J=6.8 Hz, 3H). ¹⁹F NMR (376 MHz, DMSO-d₆) δ −114.189 (1F), −134.168 (1F), −148.043 (1F), −157.590 (1F), −160.524 (1F). LCMS: Method-UC05 FAR1, RT 2.925, ESI-MS: measured m/z 551.3 [M+1]⁺, HPLC: Method-METHOD I RT 7.305 (97.22%).

Step-4: 6-chloro-7-(2-fluorophenyl)-4-((2S)-2-methyl-4-(2,3,4,5-tetrafluoro-6-(methylsulfinyl)phenyl)piperazin-1-yl)quinazoline. To a stirred solution of (S)-6-chloro-7-(2-fluorophenyl)-4-(2-methyl-4-(2,3,4,5-tetrafluoro-6-(methylthio)phenyl)piperazin-1-yl)quinazoline (0.15 g, 0.27 mmol) in DCM (2 mL) was added m-CPBA (0.093 g, 0.54 mmol) at 0° C. The resulting reaction mixture was stirred at room temperature for 5 h. After the completion of reaction, the reaction mixture was diluted with saturated solution of NaHCO₃ (40 mL) and extracted with EtOAc (3×40 mL). The combined organic phases were dried over anhydrous Na₂SO₄, filtered, and concentrated under reduced pressure. The obtained crude was purified by Prep. TLC eluting with 50% EtOAc in Hexane to afford title compound as a yellow solid (0.090 g, 0.15 mmol, 58% yield). Diastereomers were separated by Chiral SFC eluting with 50% liquid CO₂ in 50% of mixture of 7:3=IPA:MeCN on CHIRALPAK IG 250×50 mm 5 um.

Step-4A: Characterizations of 6-chloro-7-(2-fluorophenyl)-4-((2S)-2-methyl-4-(2,3,4,5-tetrafluoro-6-(methylsulfinyl)phenyl) piperazin-1-yl)quinazoline (Compound 38A). Off-white solid (0.01 g, 1.31 mmol, 94% yield). ¹H NMR (400 MHz, DMSO-d₆) δ 8.71 (s, 1H), 8.11 (s, 1H), 7.86 (s, 1H), 7.60-7.55 (m, 1H), 7.52-7.48 (m, 1H), 7.41-7.36 (m, 2H), 4.81 (s, 1H), 4.20 (d, J=13.2 Hz, 1H), 3.84-3.37 (m, 1H), 3.52-3.49 (m, 2H), 3.13 (s, 3H), 2.98 (d, J=11.6 Hz, 2H), 1.53 (d, J=6.8 Hz, 3H). ¹⁹F NMR (376 MHz, DMSO-d₆) δ −114.186 (1F), −143.785 (1F), −145.401_-145.482 (1F), −151.426_-151.539 (1F), −157.231 (1F). LCMS: Method-UC05 FAR1, RT 2.157, ESI-MS: measured m/z 567.3 [M+1]⁺, HPLC: Method-METHOD I RT 6.166 (99.30%).

Characterizations of 6-chloro-7-(2-fluorophenyl)-4-((2S)-2-methyl-4-(2,3,4,5-tetrafluoro-6-(methylsulfinyl)phenyl) piperazin-1-yl)quinazoline (Compound 39A). Off-white solid (0.008 g, 1.31 mmol, 94% yield). ¹H NMR (400 MHz, DMSO-d₆) δ 8.71 (s, 1H), 8.12 (s, 1H), 7.86 (s, 1H), 7.60-7.55 (m, 1H), 7.52-7.48 (m, 1H), 7.41-7.36 (m, 2H), 4.82 (s, 1H), 4.14 (d, J=12.8 Hz, 1H), 3.83 (t, J=10.8 Hz, 2H), 3.55-3.47 (m, 1H), 3.18 (s, 3H), 3.04-3.03 (m, 2H), 1.54 (d, J=6.4 Hz, 3H). ¹⁹F NMR (376 MHz, DMSO-d₆) δ −114.194 (1F), −143.308 (1F), −146.029_-146.108 (1F), −151.337_-151.437 (1F), −158.068 (1F). LCMS: Method-UC05 FAR1, RT 2.156, ESI-MS: measured m/z 567.3 [M+1]⁺, HPLC: Method-METHOD I RT 6.166 (99.23%).

Step-5: (S)-6-chloro-7-(2-fluorophenyl)-4-(2-methyl-4-(2,3,4,5-tetrafluoro-6-(methylsulfonyl)phenyl)piperazin-1-yl)quinazoline (Compound 34A). To a stirred solution of 6-chloro-7-(2-fluorophenyl)-4-((2S)-2-methyl-4-(2,3,4,5-tetrafluoro-6-(methylsulfinyl)phenyl)piperazin-1-yl)quinazoline (0.025 g, 0.04 mmol) in THF:MeOH:Water (0.8 ml:0.2 ml:0.2 ml) was added oxone (0.13 g, 0.44 mmol) at 0° C. The resulting reaction mixture was stirred at room temperature for 16 h. After the completion of reaction, the reaction mixture was diluted with saturated solution of NaHCO₃ (20 mL) and extracted with EtOAc (2×20 mL). The combined organic phases were dried over anhydrous Na₂SO₄, filtered, and concentrated under reduced pressure. The obtained crude was purified by trituration using n-pentane at −78° C. to afford title compound as a yellow solid (0.04 g, 0.06 mmol, 78% yield). ¹H NMR (400 MHz, DMSO-d₆) δ 8.70 (s, 1H), 8.11 (s, 1H), 7.86 (s, 1H), 7.58-7.55 (m, 1H), 7.52-7.48 (m, 1H), 7.39-7.36 (m, 1H), 4.87 (br s, 1H), 4.28-4.22 (m, 1H), 3.87 (t, J=12.8 Hz, 1H), 3.59 (s, 3H), 3.58-3.50 (m, 2H), 1.57 (d, J=6.4 Hz, 3H). ¹⁹F NMR (376 MHz, DMSO-d₆) δ −114.174 (1F), −136.241_-136.305 (1F), −141.691_-141.765 (1F), −147.948_-148.003 (1F), −155.955_-156.078 (1F). LCMS: Method-METHOD II, RT 2.390, ESI-MS: measured m/z 583.3 [M+1]⁺, HPLC: M ethod-METHOD I RT 6.535 (97.74%).

### Synthesis of (S)-7-chloro-4-(4-((2-(difluoromethoxy)-3,4,5,6-tetrafluorophenyl) sulfonyl)-2-methylpiperazin-1-yl)-6-fluoro-1-(2-isopropyl-4-methylpyridin-3-yl)pyrido[2,3-d]pyrimidin-2(1H)-one (Compound 12A)

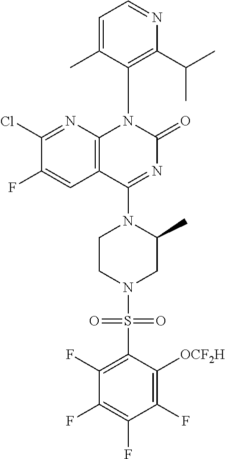

The title compound, 7-chloro-4-((S)-4-((2-(difluoromethoxy)-3,4,5,6-tetrafluorophenyl)sulfonyl)-2-methylpiperazin-1-yl)-6-fluoro-1-(2-isopropyl-4-methylpyridin-3-yl)pyrido[2,3-d]pyrimidin-2(1H)-one, was prepared via Scheme 3. ¹H NMR (400 MHz, DMSO-d₆) δ 8.48 (d, J=4.8 Hz, 1H), 8.36-3.33 (m, 1H), 7.15 (t, J=72.4 Hz, 1H), 7.26 (d, J=4.8 Hz, 1H), 4.96 (br s, 1H), 4.32-4.24 (m, 1H), 3.78-3.76 (m, 2H), 3.58 (d, J=11.6 Hz, 1H), 3.18-3.15 (m, 1H), 3.04-2.99 (m, 1H), 2.60 (br s, 1H), 1.92 (s, 3H), 1.41 (t, J=5.6 Hz, 3H), 1.04 (d, J=5.6 Hz, 3H), 1.00-0.97 (m, 3H). ¹⁹F NMR (376 MHz, DMSO-d₆) δ −81.678_-81.764 (m, 2F), −128.184-−128.215 (m, 1F), −133.542-−133.611 (m, 1F), −147.041 (m, 1F), 149.189-−149.217 (m, 1F), 155.037-−155.162 (m, 1F), LCMS: UC05_FRA1, RT—2.45, ESI-MS: measured m/z 709.2 [M+1]⁺. HPLC: HP07_TFRA1, RT 6.51, 93.78%

### Synthesis of 1-(6-chloro-7-(2-fluorophenyl)quinazolin-4-yl)-N-(2,3,4,5-tetrafluoro-6-(methylsulfonyl)phenyl)azetidin-3-amine (Compound 36A) & 1-(6-chloro-7-(2-fluorophenyl)quinazolin-4-yl)-N-(2,3,4,5-tetrafluoro-6-(methylsulfinyl)phenyl)azetidin-3-amine (Compound 37A)

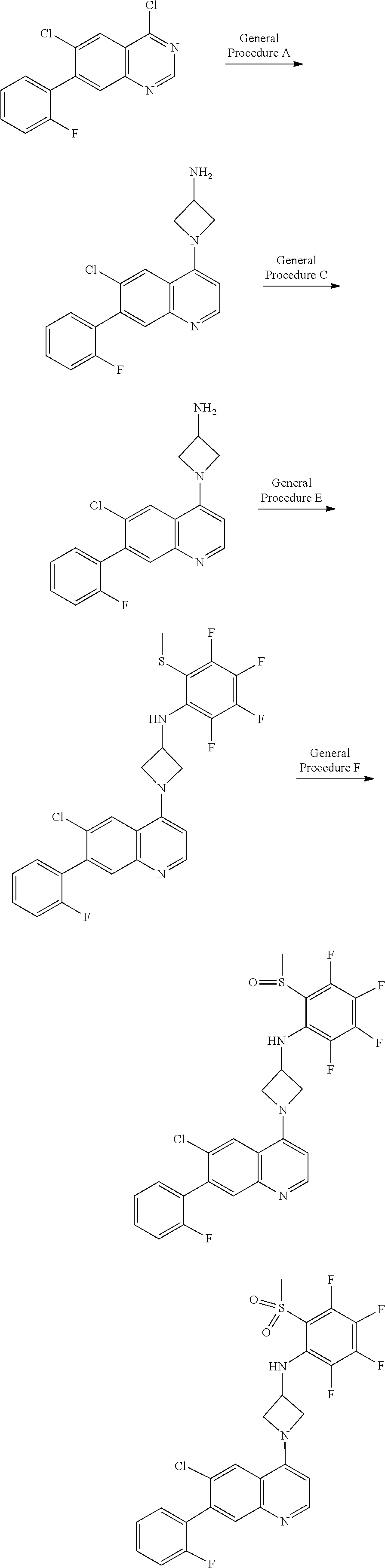 [SMILES: Fc1ccccc1-c1cc2ncnc(Cl)c2cc1Cl ; NC1CN(c2ccnc3cc(-c4ccccc4F)c(Cl)cc23)C1 ; NC1CN(c2ccnc3cc(-c4ccccc4F)c(Cl)cc23)C1 ; CSc1c(F)c(F)c(F)c(F)c1NC1CN(c2ccnc3cc(-c4ccccc4F)c(Cl)cc23)C1 ; CS(=O)c1c(F)c(F)c(F)c(F)c1NC1CN(c2ccnc3cc(-c4ccccc4F)c(Cl)cc23)C1 ; CS(=O)(=O)c1c(F)c(F)c(F)c(F)c1NC1CN(c2ccnc3cc(-c4ccccc4F)c(Cl)cc23)C1]

Synthesis of tert-butyl (1-(6-chloro-7-(2-fluorophenyl)quinazolin-4-yl)azetidin-3-yl)carbamate. The product was prepared by using General Procedure A. ¹H NMR (400 MHz, DMSO-d₆) δ 8.51 (s, 1H), 8.01 (s, 1H), 7.73 (s, 1H), 7.70 (d, J=7.2 Hz, 1H), 7.57-7.54 (m, 1H), 7.50-7.45 (m, 1H), 7.40-7.34 (m, 1H), 4.79 (br s, 2H), 4.52-4.50 (m, 1H), 4.37 (br s, 2H), 1.41 (s, 9H). LCMS: Method-METHOD II, RT—1.971 ESI-MS: measured m/z 429.3 [M+1]⁺.

Synthesis of 1-(6-chloro-7-(2-fluorophenyl)quinazolin-4-yl)azetidin-3-amine The product was prepared by using General Procedure C. ¹H NMR (400 MHz, DMSO-d₆) δ 8.54 (s, 1H), 8.02 (s, 1H), 7.75, (s, 1H), 7.59-7.54 (m, 1H), 7.50-7.46 (m, 1H), 7.40-7.34 (m, 1H), 6.45-6.42 (br s, 2H), 4.80 (br s, 2H), 4.35 (s, 2H), 4.12-4.05 (m, 1H). LCMS: Method-METHOD II, RT—1.201 ESI-MS: measured m/z 329.3 [M+1]⁺.

Synthesis of 1-(6-chloro-7-(2-fluorophenyl)quinazolin-4-yl)-N-(2,3,4,5-tetrafluoro-6-(methylthio)phenyl)azetidin-3-amine. The product was prepared by using General Procedure E. ¹H NMR (400 MHz, DMSO-d₆) δ 8.53 (s, 1H), 8.07 (s, 1H), 7.74 (s, 1H), 7.57-7.54 (m, 1H), 7.50-7.46 (m, 1H), 7.40-7.34 (m, 2H), 6.13 (d, J=6.4 Hz, 1H), 4.87 (br s, 2H), 4.72 (br s, 1H), 4.60 (br s, 2H), 2.32 (s, 3H). LCMS: Method-METHOD II, RT—2.461 ESI-MS: measured m/z 523.3 [M+1]⁺.

Synthesis of 1-(6-chloro-7-(2-fluorophenyl)quinazolin-4-yl)-N-(2,3,4,5-tetrafluoro-6-(methylsulfonyl)phenyl)azetidin-3-amine (Compound 36A). The product was prepared by using General Procedure F. ¹H NMR (400 MHz, DMSO-d₆) δ 8.54 (s, 1H), 8.07 (s, 1H), 7.74 (s, 1H), 7.59-7.53 (m, 1H), 7.50-7.40 (m, 1H), 7.38-7.34 (m, 2H), 7.24 (d, J=5.6 Hz, 1H), 4.90 (br s, 2H), 4.70-4.66 (m, 2H), 4.50, (br s, 2H), 3.48 (s, 3H). ¹⁹F NMR (376 MHz, DMSO-d₆) δ −114.286 (1F), −135.33_-135.44 (1F), −148.23_-148.37 (1F), −157.23_-157.32 (1F), −171.14_-171.28 (1F). LCMS: Method-METHOD II, RT 2.199, ESI-MS: measured m/z 555.3 [M+1]⁺, HPLC: Method-METHOD I RT 6.40 (100%).

Synthesis of 1-(6-chloro-7-(2-fluorophenyl)quinazolin-4-yl)-N-(2,3,4,5-tetrafluoro-6-(methylsulfinyl)phenyl)azetidin-3-amine (Compound 37A). The product was prepared by using General Procedure F. ¹H NMR (400 MHz, DMSO-d₆) δ 8.54 (s, 1H), 8.07 (s, 1H), 7.74 (s, 1H), 7.59-7.53 (m, 1H), 7.50-7.40 (m, 1H), 7.38-7.34 (m, 3H), 4.90 (br s, 2H), 4.69-4.64 (m, 1H), 4.46 (br s, 1H), 3.16 (s, 1H). ¹⁹F NMR (376 MHz, DMSO-d₆) δ −114.278 (1F), −141.17_-141.26 (1F), −152.05_-152.18 (1F), −158.95_-159.04 (1F), −171.69_-171.83 (1F). LCMS: Method-METHOD II, RT 2.161, ESI-MS: measured m/z 539.2 [M+1]⁺.

### Synthesis of (7R)-7-(8-chloro-1-naphthyl)-2-[[(1S,2S)-1-methylpyrrolidin-2-yl]methoxy]-4-[4-[2,3,4,5-tetrafluoro-6-(trifluoromethyl)phenyl]sulfonylpiperazin-1-yl]-6,8-dihydro-5H-pyrido[3,4-d]pyrimidine (Compound 42A)

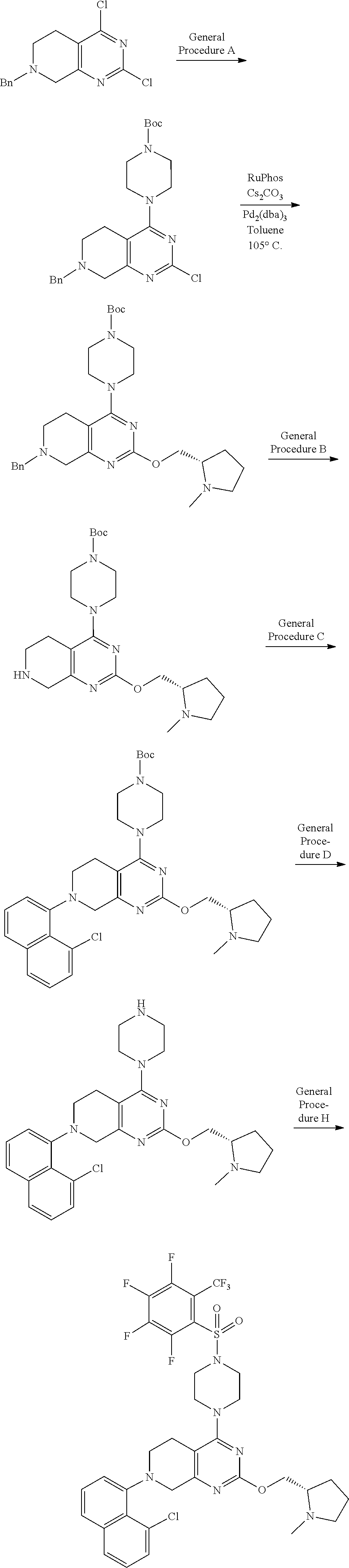

Synthesis of 1,1-di(methyl)ethyl 4-[2-chloranyl-7-(phenylmethyl)-6,8-dihydro-5H-pyrido[3,4-d]pyrimidin-4-yl]piperazine-1-carboxylate. The product was prepared using General Procedure A. 1H NMR (400 MHz, CdCl₃) δ 7.36-7.25 (m, 5H), 3.67 (s, 2H), 3.60 (s, 2H), 3.52-3.46 (m, 8H), 2.65 (s, 4H), 1.47 (s, 9H).

Synthesis of 1,1-di(methyl)ethyl 4-[2-[[(1S,2S)-1-methylpyrrolidin-2-yl]methoxy]-7-(phenylmethyl)-6,8-dihydro-5H-pyrido[3,4-d]pyrimidin-4-yl]piperazine-1-carboxylate. To a round-bottom flask charged with 1,1-di(methyl)ethyl 4-[2-chloranyl-7-(phenylmethyl)-6,8-dihydro-5H-pyrido[3,4-d]pyrimidin-4-yl]piperazine-1-carboxylate (1 g, 2.25 mmol), [(1S,2R)-1-methylpyrrolidin-2-yl]methanol (715.99 mg, 6.22 mmol), 2-Dicyclohexylphosphino-2′,6′-diisopropoxybiphenyl (182.88 mg, 391.92 μmol), cesium carbonate (2.20 g, 6.76 mmol) and Pd₂(dba)₃ (358.89 mg, 391.92 μmol) was added anhydrous toluene (60 mL). The solution was degassed and heated at 105° C. for 12 hr under reflux. Then, the flask was cooled down to r.t., and water was added to quench the reaction. The resulting suspension was extracted with EtOAc thrice, and the combined organic layers was washed with water, brine, dried over Na₂SO₄, filtered and all the volatiles was removed under reduced pressure. The crude was purified by normal phase column chromatography eluting in gradient from 1%-15% MeOH in DCM to give 1,1-di(methyl)ethyl 4-[2-[[(1S,2S)-1-methylpyrrolidin-2-yl]methoxy]-7-(phenylmethyl)-6,8-dihydro-5H-pyrido[3,4-d]pyrimidin-4-yl]piperazine-1-carboxylate (2, 870 mg, 1.66 mmol, 73.90% yield). 1H NMR (400 MHz, CDCl₃) δ 7.37-7.27 (m, 5H), 4.37-4.33 (m, 1H), 4.12-4.08 (m, 1H), 3.66 (s, 2H), 3.56 (s, 2H), 3.50-3.47 (m, 4H), 3.43-3.40 (m, 4H), 3.09 (t, J=8 Hz, 1H), 2.64-2.62 (m, 4H), 2.47 (s, 3H), 2.29-2.27 (m, 1H), 2.08-1.99 (m, 1H), 1.85-1.73 (m, 4H), 1.47 (s, 9H). LCMS measured m/z 523 [M+1]⁺.

Synthesis of 1,1-di(methyl)ethyl 4-[2-[[(1R,2R)-1-methylpyrrolidin-2-yl]methoxy]-5,6,7,8-tetrahydropyrido[3,4-d]pyrimidin-4-yl]piperazine-1-carboxylate. The product was prepared using General Procedure B (743 mg, 1.72 mmol, 91.52% yield). 1H NMR (400 MHz, CdCl₃) δ 4.36-4.32 (m, 1H), 4.09-4.06 (m, 1H), 3.91 (s, 2H), 3.51-3.48 (m, 4H), 3.41-3.38 (m, 4H), 3.09-3.04 (m, 1H), 3.01-2.98 (m, 2H), 2.65-2.60 (m, 1H), 2.55-2.52 (m, 2H), 2.45 (s, 3H), 2.29-2.22 (m, 1H), 1.87-1.69 (m, 5H), 1.47 (s, 9H).

Synthesis of 1,1-di(methyl)ethyl 4-[(7R)-7-(8-chloro-1-naphthyl)-2-[[(1S,2S)-1-methylpyrrolidin-2-yl]methoxy]-6,8-dihydro-5H-pyrido[3,4-d]pyrimidin-4-yl]piperazine-1-carboxylate. The product was prepared using General Procedure C (239 mg, 402.93 μmol, 24.22% yield). LCMS measured m/z 593 [M+1]⁺.

Synthesis of (7R)-7-(8-chloro-1-naphthyl)-2-[[(1S,2S)-1-methylpyrrolidin-2-yl]methoxy]-4-piperazin-1-yl-6,8-dihydro-5H-pyrido[3,4-d]pyrimidine. The product was prepared using General Procedure D (185 mg, 375.22 μmol, 96.95% yield). The crude was directly used for next step without any further purification. LCMS measured m/z 493 [M+1]⁺

Synthesis of (7R)-7-(8-chloro-1-naphthyl)-2-[[(1S,2S)-1-methylpyrrolidin-2-yl]methoxy]-4-[4-[2,3,4,5-tetrafluoro-6-(trifluoromethyl)phenyl]sulfonylpiperazin-1-yl]-6,8-dihydro-5H-pyrido[3,4-d]pyrimidine (Compound 42A). The product was prepared using General Procedure H (8.4 mg, 10.43 μmol, 3.96% yield, 96% purity). 1H NMR (400 MHz, CdCl₃) δ 7.76 (d, J=8.2 Hz, 1H), 7.62 (d, J=8.2 Hz, 1H), 7.51 (t, J=8.0 Hz, 1H), 7.47-7.42 (m, 1H), 7.36-7.31 (m, 1H), 7.25-7.21 (m, 1H), 4.84 (br, 2H), 4.56-4.52 (dd, J=12.6 Hz, J=3.5 Hz, 1H), 4.41-4.36 (m, 1H), 3.98-3.54 (m, 10H), 3.13 (d, J=8.9 Hz, 2H), 3.08 (s, 3H), 2.52-2.49 (m, 1H), 2.33 (br, 2H), 2.13 (br, 2H), 0.89-0.81 (m, 2H). ¹⁹F NMR (376 MHz, CdCl3) δ −51.49_-51.62, −51.99_-52.09, −129.98_-131.00, −135.04_-135.38, −144.62_-145.00, −147.52_-147.66, −151.06_-151.19. LCMS measured m/z 773 [M+1]⁺

### Synthesis of 7-(8-chloronaphthalen-1-yl)-4-(4-(2,3,4,5-tetrafluoro-6-(methylsulfinyl)phenyl)piperazin-1-yl)-5,6,7,8-tetrahydropyrido[3,4-d]pyrimidine Compound 43A

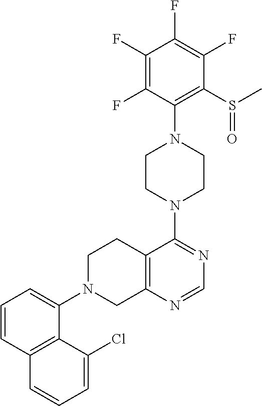 [SMILES: CS(=O)c1c(F)c(F)c(F)c(F)c1N1CCN(c2ncnc3c2CCN(c2cccc4cccc(Cl)c24)C3)CC1]

The title compound, 7-(8-chloronaphthalen-1-yl)-4-(4-(2,3,4,5-tetrafluoro-6-(methylsulfinyl)phenyl)piperazin-1-yl)-5,6,7,8-tetrahydropyrido[3,4-d]pyrimidine (Compound 43A), was prepared via Scheme 1. The detailed methods and characterizations are described below.

Synthesis of tert-butyl 4-(7-benzyl-5,6,7,8-tetrahydropyrido[3,4-d]pyrimidin-4-yl)piperazine-1-carboxylate. The intermediate, tert-butyl 4-(7-benzyl-5,6,7,8-tetrahydropyrido[3,4-d]pyrimidin-4-yl)piperazine-1-carboxylate, was prepared via General Procedure A to yield the product. ¹H NMR (400 MHz, CDCl₃) δ 8.44 (s, 1H), 7.30-7.12 (m, 5H), 3.57 (d, J=11.7 Hz, 4H), 3.43 (dd, J=6.8, 3.5 Hz, 4H), 3.34-3.26 (m, 4H), 2.58 (dt, J=8.1, 4.2 Hz, 4H), 1.39 (d, J=2.1 Hz, 9H).

Synthesis of 1,1-di(methyl)ethyl 4-(5,6,7,8-tetrahydropyrido[3,4-d]pyrimidin-4-yl)piperazine-1-carboxylate. The intermediate, 1,1-di(methyl)ethyl 4-(5,6,7,8-tetrahydropyrido[3,4-d]pyrimidin-4-yl)piperazine-1-carboxylate, was prepared via General Procedure B. ¹H NMR (400 MHz, CDCl₃) δ 8.57 (s, 1H), 4.05 (s, 2H), 3.59-3.52 (m, 5H), 3.50 (s, 2H), 3.44-3.37 (m, 5H), 3.07 (t, J=5.4 Hz, 2H), 2.64 (t, J=5.5 Hz, 2H), 1.50 (s, 9H).

Synthesis of 1,1-di(methyl)ethyl 4-[7-(8-chloranyl-1-naphthyl)-6,8-dihydro-5H-pyrido[3,4-d]pyrimidin-4-yl]piperazine-1-carboxylate. The intermediate, 1,1-di(methyl)ethyl 4-[7-(8-chloranyl-1-naphthyl)-6,8-dihydro-5H-pyrido[3,4-d]pyrimidin-4-yl]piperazine-1-carboxylate, was prepared via General Procedure C. ¹H NMR (400 MHz, CDCl₃) δ 8.61 (d, J=2.5 Hz, 1H), 7.75 (d, J=8.1 Hz, 1H), 7.61 (d, J=8.1 Hz, 1H), 7.52 (dq, J=7.4, 1.4 Hz, 1H), 7.45 (td, J=7.8, 2.2 Hz, 1H), 7.33 (td, J=7.8, 2.2 Hz, 1H), 7.24 (d, J=7.6 Hz, 1H), 4.50 (d, J=17.5 Hz, 1H), 3.92 (d, J=17.7 Hz, 1H), 3.68-3.47 (m, 8H), 3.38 (dd, J=10.1, 6.9 Hz, 2H), 3.17 (dddd, J=23.5, 12.5, 9.9, 4.2 Hz, 2H), 2.60 (dd, J=14.9, 3.3 Hz, 1H), 1.51 (s, 9H).

Synthesis of 7-(8-chloranyl-1-naphthyl)-4-piperazin-1-yl-6,8-dihydro-5H-pyrido[3,4-d]pyrimidine. The intermediate, 7-(8-chloranyl-1-naphthyl)-4-piperazin-1-yl-6,8-dihydro-5H-pyrido[3,4-d]pyrimidine, was prepared via General Procedure D to yield the product. ¹H NMR (400 MHz, MeOD) δ 8.45 (s, 1H), 7.81 (dd, J=8.1, 1.4 Hz, 1H), 7.67 (d, J=8.1 Hz, 1H), 7.54-7.45 (m, 3H), 7.36 (t, J=7.8 Hz, 1H), 7.32-7.27 (m, 1H), 4.39 (d, J=17.4 Hz, 1H), 3.74 (d, J=17.5 Hz, 1H), 3.61 (ddd, J=13.1, 7.0, 3.0 Hz, 2H), 3.56-3.48 (m, 1H), 3.41 (ddd, J=13.2, 6.8, 3.1 Hz, 2H), 3.29-3.18 (m, 1H), 3.15-3.05 (m, 1H), 3.00 (ddd, J=12.6, 6.8, 3.1 Hz, 2H), 2.90 (ddd, J=12.5, 7.0, 3.1 Hz, 2H), 2.65-2.56 (m, 1H).

Synthesis of 7-(8-chloranyl-1-naphthyl)-4-[4-[2,3,4,5-tetrakis(fluoranyl)-6-methylsulfanyl-phenyl]piperazin-1-yl]-6,8-dihydro-5H-pyrido[3,4-d]pyrimidine. The intermediate, 7-(8-chloranyl-1-naphthyl)-4-[4-[2,3,4,5-tetrakis(fluoranyl)-6-methylsulfanyl-phenyl]piperazin-1-yl]-6,8-dihydro-5H-pyrido[3,4-d]pyrimidine, was prepared via General Procedure E to yield the product. ¹H NMR (400 MHz, CDCl₃) δ 8.66 (s, 1H), 7.79 (dd, J=8.2, 1.3 Hz, 2H), 7.70-7.62 (m, 2H), 7.56 (dd, J=7.4, 1.3 Hz, 1H), 7.48 (t, J=7.8 Hz, 2H), 7.38 (d, J=7.8 Hz, 1H), 7.31-7.27 (m, 2H), 4.55 (d, J=17.8 Hz, 1H), 4.43 (dd, J=11.7, 5.7 Hz, 1H), 3.81 (s, 2H), 3.70-3.58 (m, 3H), 3.40-3.24 (m, 6H), 3.24-3.16 (m, 1H), 2.67 (dd, J=15.1, 3.4 Hz, 1H), 2.55 (s, 4H), 1.19 (d, J=6.1 Hz, 1H), 1.03 (d, J=6.0 Hz, 1H); ¹⁹F NMR (376 MHz, CDCl₃) δ−133.65-−133.85 (m), −148.22 (dd, J=20.3, 8.7 Hz), −156.86 (td, J=20.3, 2.2 Hz), −159.64 (dd, J=24.0, 20.5 Hz).

Synthesis of 7-(8-chloranyl-1-naphthyl)-4-[4-[2,3,4,5-tetrakis(fluoranyl)-6-methylsulfinyl-phenyl]piperazin-1-yl]-6,8-dihydro-5H-pyrido[3,4-d]pyrimidine. The title product was prepared via General Procedure F to yield the product. ¹H NMR (400 MHz, CDCl₃) δ 8.66 (s, 1H), 7.80 (dd, J=8.2, 1.3 Hz, 1H), 7.66 (d, J=8.1 Hz, 1H), 7.56 (dt, J=7.5, 1.6 Hz, 1H), 7.49 (t, J=7.8 Hz, 1H), 7.38 (d, J=7.8 Hz, 1H), 7.28 (d, J=3.6 Hz, 1H), 4.54 (d, J=17.7 Hz, 1H), 3.97 (dd, J=17.7, 8.1 Hz, 1H), 3.62 (d, J=9.1 Hz, 4H), 3.39 (d, J=29.5 Hz, 3H), 3.23 (d, J=12.3 Hz, 3H), 3.17 (s, 3H), 2.65 (s, 1H), 1.46 (s, 1H); ¹⁹F NMR (376 MHz, CDCl₃) δ −140.77 (dd, J=55.0, 21.3 Hz, 1F), −144.75 (ddd, J=53.5, 19.3, 10.8 Hz, 1F), −148.84 (tdd, J=19.0, 11.5, 5.9 Hz, 1F), −155.28 (q, J=21.7 Hz, 1F); ESI-MS [M+H]⁺: 590.200

### Synthesis of 6-chloro-7-(2-fluorophenyl)-N-(1-(2,3,4,5-tetrafluoro-6-(methylsulfinyl)phenyl)azetidin-3-yl)quinazolin-4-amine (Compound 48A) and 6-chloro-7-(2-fluorophenyl)-N-(1-(2,3,4,5-tetrafluoro-6-(methylthio)phenyl)azetidin-3-yl)quinazolin-4-amine (Compound 47A)

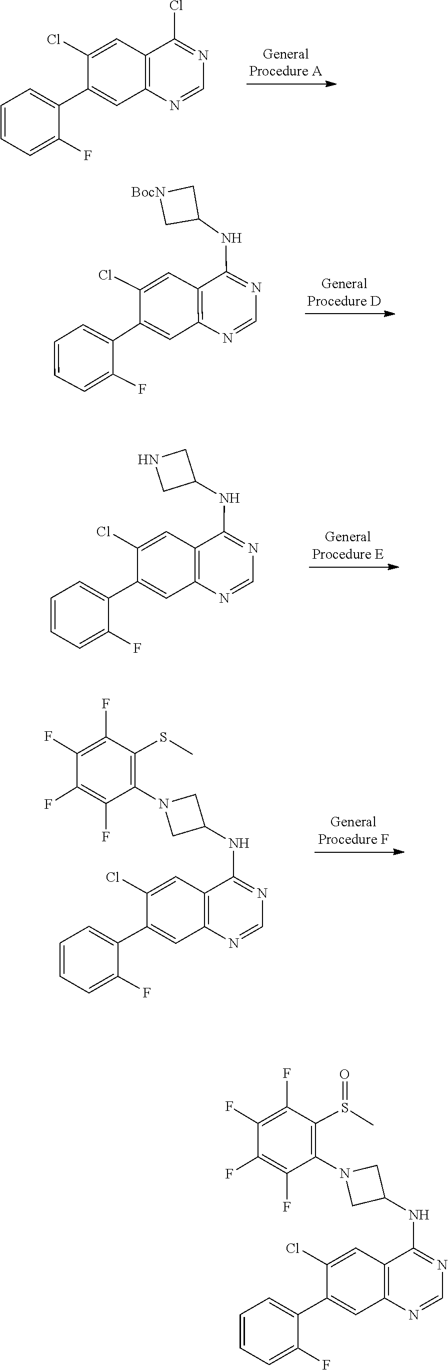 [SMILES: Fc1ccccc1-c1cc2ncnc(Cl)c2cc1Cl ; CC(C)(C)OC(=O)N1CC(Nc2ncnc3cc(-c4ccccc4F)c(Cl)cc23)C1 ; Fc1ccccc1-c1cc2ncnc(NC3CNC3)c2cc1Cl ; CSc1c(F)c(F)c(F)c(F)c1N1CC(Nc2ncnc3cc(-c4ccccc4F)c(Cl)cc23)C1 ; CS(=O)c1c(F)c(F)c(F)c(F)c1N1CC(Nc2ncnc3cc(-c4ccccc4F)c(Cl)cc23)C1]

Synthesis of tert-butyl 3-((6-chloro-7-(2-fluorophenyl)quinazolin-4-yl)amino)azetidine-1-carboxylate. The product was prepared using General Procedure A (2.0 g, 4.67 mmol, 85% yield). ¹H NMR (400 MHz, DMSO-d₆) δ 8.83 (d, J=6 Hz, 1H), 8.64 (s, 1H), 8.54 (s, 1H), 7.72 (s, 1H), 7.59-7.53 (m, 1H), 7.50-7.46 (m, 1H), 7.40-7.34 (m, 2H), 4.88 (dd, J=11.2 Hz, 1H), 4.24 (s, 2H), 3.97-3.93 (m, 2H), 1.41 (s, 9H). LCMS: Method-METHOD II, RT-2.101 ESI-MS: measured m/z 429.4[M+1]⁺.

Synthesis of N-(azetidin-3-yl)-6-chloro-7-(2-fluorophenyl)quinazolin-4-amine. The product was prepared using General Procedure D (1.1 g, 3.35 mmol, 72% yield). ¹H NMR (400 MHz, DMSO-d₆) δ 8.83 (d, J=5.6 Hz, 1H), 8.66 (s, 1H), 8.53 (s, 1H), 7.72 (s, 1H), 7.57-7.53 (m, 1H), 7.50-7.44 (1H), 7.40-7.35 (m, 2H), 4.99-4.97 (m, 1H), 4.04 (s, 1H), 3.92 (t, J=9.2 Hz, 2H), 3.84-3.77 (m, 2H). LCMS: Method-METHOD II, RT—1.351 ESI-MS: measured m/z 329.3[M+1]⁺.

Synthesis of 6-chloro-7-(2-fluorophenyl)-N-(1-(2,3,4,5-tetrafluoro-6-(methylthio)phenyl)azetidin-3-yl)quinazolin-4-amine. The product was prepared using General Procedure E (0.12 g, 0.22 mmol, 22% yield). ¹H NMR (400 MHz, DMSO-d₆) δ 8.90 (s, J=5.2 Hz, 1H), 8.67 (s, 1H), 8.56 (s, 1H), 7.73 (s, 1H), 7.59-7.53 (m, 1H), 7.50-7.46 (m, 1H), 7.40-7.34 (m, 2H), 4.89-4.88 (m, 1H), 4.84-4.79 (m, 2H), 4.44-4.39 (m, 2H), 2.29 (s, 3H). ¹⁹F NMR (376 MHz, DMSO-d₆) δ −114.49 (1F), −132.11_-132.20 (1F), −156.55_156.66 (1F), −160.27_-160.34 (1F), −171.17_-171.24 (1F). LCMS: Method-METHOD II, RT—2.679 ESI-MS: measured m/z 523.3[M+1]⁺, 525.3[M+3]⁺ HPLC: METHOD I. RT 7.008, 96.56%.

Synthesis of 6-chloro-7-(2-fluorophenyl)-N-(1-(2,3,4,5-tetrafluoro-6-(methylsulfinyl)phenyl)azetidin-3-yl)quinazolin-4-amine. The product was prepared using General Procedure F (0.016 g, 0.029 mmol, 16% yield). ¹H NMR (400 MHz, DMSO-d₆) δ 8.92 (d, J=5.2 Hz, 1H), 8.66 (s, 1H), 8.56 (s, 1H), 7.74 (s, 1H), 7.57-7.53 (m, 1H), 7.50-7.47 (m, 1H), 7.40-7.34 (m, 2H), 4.94-4.91 (m, 1H), 4.76-4.68 (m, 2H), 4.45-4.43 (m, 1H), 4.35-4.33 (m, 1H), 3.14 (s, 3H). ¹⁹F NMR (376 MHz, DMSO-d₆) δ −114.33_114.36 (1F), −140.84_-140.92 (1F), −152.72_152.84 (1F), −159.69_-159.78 (1F), −170.77_-170.91 (1F). LCMS: METHOD II, RT-2.053 ESI-MS: measured m/z 539.3 [M+1]⁺, 541.3 [M+1]. HPLC: METHOD I. RT 6.014, 93.62%.

### Synthesis of (S)-6-chloro-7-(2,6-dimethylphenyl)-4-(2-methyl-4-(2,3,4,5-tetrafluoro-6-(methylsulfonyl) phenyl)piperazin-1-yl)quinoline (Compound 44A)

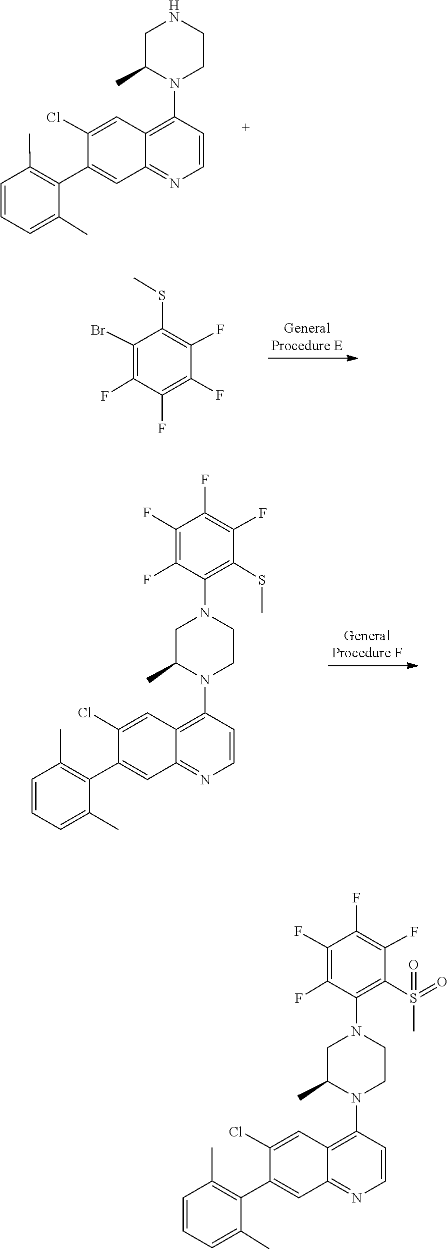 [SMILES: Cc1cccc(C)c1-c1cc2nccc(N3CCNC[C@@H]3C)c2cc1Cl ; CSc1c(F)c(F)c(F)c(F)c1Br ; CSc1c(F)c(F)c(F)c(F)c1N1CCN(c2ccnc3cc(-c4c(C)cccc4C)c(Cl)cc23)[C@@H](C)C1 ; Cc1cccc(C)c1-c1cc2nccc(N3CCN(c4c(F)c(F)c(F)c(F)c4S(C)(=O)=O)C[C@@H]3C)c2cc1Cl]

Synthesis of (S)-6-chloro-7-(2,6-dimethylphenyl)-4-(2-methyl-4-(2,3,4,5-tetrafluoro-6-(methyl thio)phenyl)piperazin-1-yl)quinoline. The product was prepared by using the General Procedure E (0.12 g, 0.21 mmol, 10% yield). LCMS: LC05_MSR2, RT 13.017 ESI-MS: m/z 560.5 [M+1]⁺.

Synthesis of (S)-6-chloro-7-(2,6-dimethylphenyl)-4-(2-methyl-4-(2,3,4,5-tetrafluoro-6-(methylsulfonyl)phenyl)piperazin-1-yl)quinoline. The product was prepared by using the General Procedure F (0.018 g, 0.03 mmol, 16% yield). ¹H NMR (400 MHz, DMSO-d₆) δ 8.90 (d, J=4.8 Hz, 1H), 8.39 (s, 1H), 8.18 (s, 1H), 7.81 (brs, 1H), 7.77 (brs, 1H), 7.46 (d, J=4.4 Hz, 1H), 7.28-712 (m, 3H), 4.10 (s, 1H), 3.80 (s, 1H), 3.62-3.58 (m, 3H), 3.21 (brs, 1H), 3.11 (brs, 1H), 2.93 (m, 1H), 1.96 (s, 6H), 1.18 (d, J=6.4 Hz, 1H), 0.96 (d, J=6.0 Hz, 2H). ¹⁹F NMR (376 MHz, DMSO-d₆) δ −136.20-−137.36 (1F), −141.55-−143.08 (1F), −148.00-−148.09 (1F), −156.31-−156.52 (1F), LCMS: METHOD II, RT—2.414 ESI-MS: measured m/z 592.4 [M+1]⁺, 594.4 [M+3], HPLC: METHOD I, RT 6.804, 95.54%, Chiral HPLC: RT 3.49, 78.91%.

### Synthesis of 1-(6-chloro-7-(2-fluorophenyl)quinazolin-4-yl)-N-(2,3,5,6-tetrafluoro-4-(methylsulfinyl)phenyl)azetidin-3-amine (Compound 28A) & 1-(6-chloro-7-(2-fluorophenyl)quinazolin-4-yl)-N-(2,3,5,6-tetrafluoro-4-(methylsulfonyl)phenyl)azetidin-3-amine (Compound 23A)

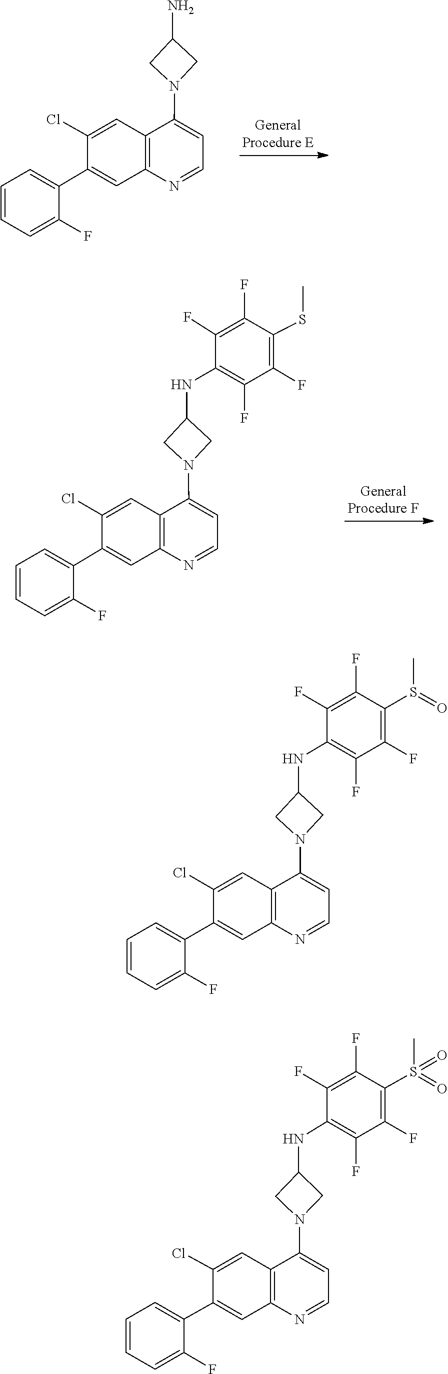 [SMILES: NC1CN(c2ccnc3cc(-c4ccccc4F)c(Cl)cc23)C1 ; CSc1c(F)c(F)c(NC2CN(c3ccnc4cc(-c5ccccc5F)c(Cl)cc34)C2)c(F)c1F ; CS(=O)c1c(F)c(F)c(NC2CN(c3ccnc4cc(-c5ccccc5F)c(Cl)cc34)C2)c(F)c1F ; CS(=O)(=O)c1c(F)c(F)c(NC2CN(c3ccnc4cc(-c5ccccc5F)c(Cl)cc34)C2)c(F)c1F]

Synthesis of 1-(6-chloro-7-(2-fluorophenyl)quinazolin-4-yl)-N-(2,3,5,6-tetrafluoro-4-(methylthio)phenyl)azetidin-3-amine. The product was prepared by using the General Procedure E (0.23 g, 0.44 mmol, 386 yield). ¹H NMR (400 MHz, DMSO-d₆) δ 8.53 (s, 1H), 8.05 (s, 1H), 7.74 (s, 1H), 7.59-7.53 (s, 1H), 7.50-7.46 (i, 1H), 7.40-7.34 (m, 2H), 6.90 (d, J=67 Hz, 1H), 4.85 (br s, 2H), 4.73, (br s, 1H), 4.61 (s, 2H), 2.37 (s, 3H). ¹⁹H NMR (400 MHz, DMSO-d₆) δ −114.29 (1F), −137.39_-137.44 (2F), −159.15_-159.20 (2F). LCMS: Method-METHOD II, RT-2.375 ESI-MS: measured m/z 523.3[M+1]⁺.

Synthesis of 1-(6-chloro-7-(2-fluorophenyl)quinazolin-4-yl)-N-(2,3,5,6-tetrafluoro-4-(methylsulfinyl)phenyl)azefidin-3-amine (Compound 28A) & 1-(6-chloro-7-(2-fluorophenyl)quinazolin-4-yl)-N-(2,3,5,6-tetrafluoro-4-(methylsulfonyl)phenyl)azetidin-3-amine (Compound 23A). The products were prepared by using the General Procedure F. 1-(6-chloro-7-(2-fluorophenyl)quinazolin-4-yl)-N-(2,3,5,6-tetrafluoro-4-(methylsulfinyl)phenyl)azetidin-3-amine (0.013 g, 0.3 mmol, 600 yield). ¹H NMR (400 MHz, DMSO-d₆) δ 8.54 (s, 1H), 8.05 (s, 1H), 7.75 (s, 1H), 7.75-7.46 (m, 2H), 7.40-7.35 (m, 2H), 7.34 (br s, 1H), 4.86 (br s, 3H), 4.66 (br s, 2H), 3.13 (s, 3H). ¹⁹F NMR (376 MHz, DMSO-d₆) δ −114.30 (1F), −143.43-−143.47 (2F), −159.54-−159.59 (2F). LCMS: Method-UC05 FAR1, RT 1.857, ESI-MS: measured m/z 539.3 [M+1]⁺, HPLC: Method-METHOD I RT 5.782 (99.54). 1-(6-chloro-7-(2-fluorophenyl)quinazolin-4-yl)-N-(2,3,5,6-tetrafluoro-4-(methylsulfonyl) phenyl)azetidin-3-amine (0.02 g, 0.03 mmol, 10% yield). ¹H NMR (400 MHz, DMSO-d₆) δ 8.54 (s, 1H), 8.04 (s, 1H), 7.75 (s, 1H), 7.59-7.55 (m, 2H), 7.48-7.46 (m, 1H), 7.40-7.34 (m, 2H), 4.83 (br s, 3H), 4.66 (s, 2H) 3.39 (s, 3H). ¹⁹F NMR (376 MHz, DMSO-d₆) δ −114.30 (1F), −141.62-−141.67 (2F), −159.98-−160.03 (2F). LCMS: Method-METHOD II, RT 2.012, ESI-MS: measured m/z 555.3 [M+1]⁺. HPLC: Method-METHOD I RT 5.98 (96.83%).

### Synthesis of 6-chloro-7-(2-fluorophenyl)-4-((2S)-2-methyl-4-(2, 3, 4,5-tetrafluoro-6-(methylsulfinyl)benzyl)piperazin-1-yl)quinazoline (Compound 46A)

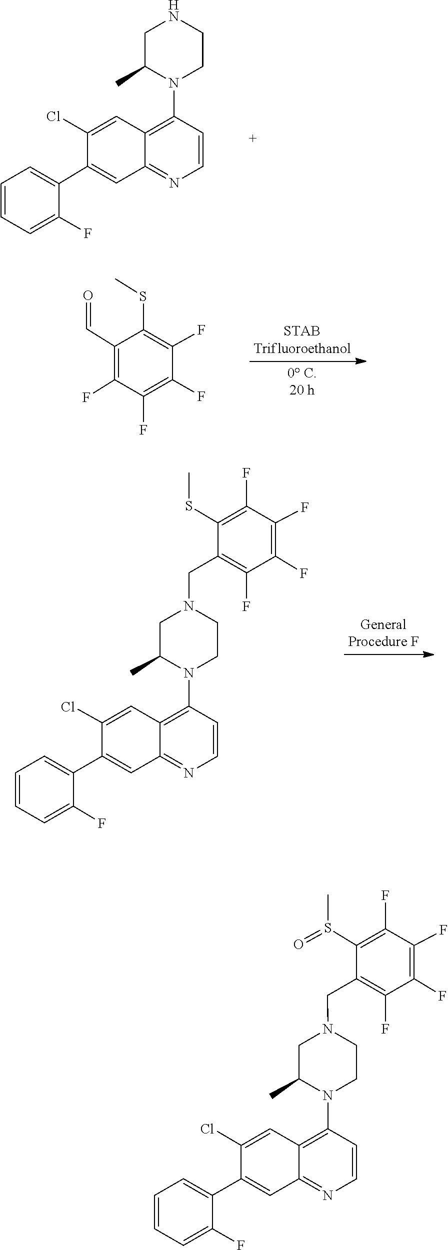 [SMILES: C[C@H]1CNCCN1c1ccnc2cc(-c3ccccc3F)c(Cl)cc12 ; CSc1c(F)c(F)c(F)c(F)c1C=O ; CSc1c(F)c(F)c(F)c(F)c1CN1CCN(c2ccnc3cc(-c4ccccc4F)c(Cl)cc23)[C@@H](C)C1 ; C[C@H]1CN(Cc2c(F)c(F)c(F)c(F)c2S(C)=O)CCN1c1ccnc2cc(-c3ccccc3F)c(Cl)cc12]

The title compound, 7-(8-chloronaphthalen-1-yl)-4-(4-(2,3,4,5-tetrafluoro-6-(methylsulfinyl)phenyl)piperazin-1-yl)-5,6,7,8-tetrahydropyrido[3,4-d]pyrimidine (Compound 46A), was prepared via Scheme 6.

Synthesis of (S)-6-chloro-7-(2-fluorophenyl)-4-(2-methyl-4-(2,3,4,5-tetrafluoro-6-(methylthio)benzyl)piperazin-1-yl)quinazoline. To a stirred solution of (S)-6-chloro-7-(2,6-dimethylphenyl)-4-(2-methylpiperazin-1-yl)quinoline (0.45 g, 1.23 mmol) in 2,2,2 Trifluroethanol (4.0 mL) was added 2,3,4,5-tetrafluoro-6-(methylthio)benzaldehyde (0.96 g, 4.31 mmol) at room temperature. The resulting reaction mixture was stirred at room temperature for 16 h. After consumption of both starting material (imine formation), sodium tri-acetoxyborohydride (0.78 g, 3.69 mmol) was added at 0° C. The resulting reaction mixture was allowed to stir for another 16 h. After completion of reaction, the mixture was concentrated under reduced pressure. The resulting crude was purified by flash column chromatography, eluted with 15-20% EtOAc in hexane to afford title compound as a brown sticky liquid (0.4 g, 0.69 mmol, 57% Yield) (0.20 g, 0.35 mmol, 23% yield). ¹H NMR (400 MHz, DMSO-d₆) δ 8.65 (s, 1H), 8.03 (s, 1H), 7.82 (s, 1H), 7.57-7.55 (m, 1H), 7.50-7.47 (m, 1H), 7.40-7.34 (m, 2H), 4.70 (br s, 1H), 4.08 (d, J=14 Hz, 1H), 3.68 (s, 2H), 3.46-39 (m, 2H), 2.83-2.67 (m, 4H), 2.47 (s, 3H), 1.34 (d, J=6.4 Hz, 3H). LCMS: Method-METHOD II, RT—2.824 ESI-MS: measured m/z 565.4 [M+1]⁺.

Synthesis of 6-chloro-7-(2-fluorophenyl)-4-((2S)-2-methyl-4-(2,3,4,5-tetrafluoro-6-(methylsulfinyl)benzyl)piperazin-1-yl)quinazoline (Compound 46A). The product was prepared using General Procedure F (0.025 g, 0.04 mmol, 24% yield). ¹H NMR (400 MHz, DMSO-d₆) δ 8.67-8.66 (m, 1H), 8.05 (s, 1H), 7.83 (s, 1H), 7.58-7.54 (m, 1H), 7.51-7.47 (m, 1H), 7.41-7.35 (m, 2H), 4.74 (br s, 1H), 4.03 (t, J=13.2 Hz, 2H), 3.38-3.37 (m, 1H), 3.36-3.35 (m, 3H), 3.18 (s, 3H), 2.93-2.82 (m, 2H), 1.40 (d, J=8 Hz, 1H), 1.31 (d, J=6.8 Hz, 2H). ¹⁹F NMR (376 MHz, DMSO-d₆) δ −114.19 (1F), −140.53_-141.07 (1F), −142.13_-142.28 (1F), −151.52_-151.77 (1F), −154.46_-154.75 (1F). LCMS: Method-METHOD II, RT 2.181, ESI-MS: measured m/z.

Synthesis of 1,1-di(methyl)ethyl N-[1-[7-(phenylmethyl)-6,8-dihydro-5H-pyrido[3,4-d]pyrimidin-4-yl]azetidin-3-yl]carbamate. The product was prepared using General Procedure A. ¹H NMR (400 MHz, MeOD) δ 8.21 (s, 1H), 7.42-7.27 (m, 6H), 4.87 (s, 2H) 4.54 (t, J=8.2 Hz, 3H), 3.71 (s, 2H), 3.47 (s, 2H), 2.74 (q, J=5.3, 4.6 Hz, 4H), 1.46 (s, 9H).

Synthesis of 1,1-di(methyl)ethyl N-[1-(5,6,7,8-tetrahydropyrido[3,4-d]pyrimidin-4-yl)azetidin-3-yl]carbamate. The product was prepared using General Procedure B (77.79% yield). ¹H NMR (400 MHz, CDCl₃) δ 8.29 (s, 1H), 6.01-5.88 (m, 1H), 4.57-4.37 (m, 3H), 4.02-3.91 (m, 2H), 3.82 (s, 2H), 3.00 (t, J=5.7 Hz, 2H), 2.75 (s, 2H), 2.52 (d, J=11.6 Hz, 2H), 1.39 (s, 9H).

Synthesis of 1,1-di(methyl)ethyl N-[1-[7-(8-chloranyl-1-naphthyl)-6,8-dihydro-5H-pyrido[3,4-d]pyrimidin-4-yl]azetidin-3-yl]carbamate. The product was prepared using General Procedure C (296 mg, 635.23 umol, 77.59%) as a brown oil: ¹H NMR (400 MHz, CDCl₃) δ 8.49 (s, 1H), 7.77 (dd, J=8.2, 1.3 Hz, 1H), 7.63 (d, J=8.1 Hz, 1H), 7.54 (dd, J=7.4, 1.3 Hz, 1H), 7.46 (t, J=7.8 Hz, 1H), 7.35 (t, J=7.8 Hz, 2H), 7.27-7.23 (m, 1H), 4.60 (dd, J=30.7, 8.1 Hz, 3H), 4.18-4.11 (m, 1H), 3.91 (d, J=17.1 Hz, 1H), 3.59 (d, J=12.3 Hz, 2H), 3.16 (td, J=12.3, 11.1, 8.5 Hz, 3H), 2.63 (t, J=11.9 Hz, 2H), 1.48 (s, 9H).

Synthesis of 1-[7-(8-chloranyl-1-naphthyl)-6,8-dihydro-5H-pyrido[3,4-d]pyrimidin-4-yl]azetidin-3-amine. The product was prepared using General Procedure D (83.30% yield).

¹H NMR (400 MHz, CDCl₃) δ 8.44 (s, 1H), 7.71 (dd, J=8.2, 1.3 Hz, 1H), 7.56 (dd, J=8.2, 1.2 Hz, 1H), 7.48 (dd, J=7.5, 1.3 Hz, 1H), 7.44-7.37 (m, 2H), 7.28 (t, J=7.8 Hz, 1H), 7.19 (dd, J=7.5, 1.2 Hz, 1H), 4.53-4.39 (m, 2H), 4.30 (d, J=17.0 Hz, 1H), 3.93-3.81 (m, 5H), 3.54-3.48 (m, 1H), 3.17-3.03 (m, 2H), 2.63-2.55 (m, 1H).

Synthesis of 1-[7-(8-chloranyl-1-naphthyl)-6,8-dihydro-5H-pyrido[3,4-d]pyrimidin-4-yl]-N-[2,3,4,5-tetrakis(fluoranyl)-6-methylsulfanyl-phenyl]azetidin-3-amine. The product was prepared using General Procedure E (84.53% yield). ¹H NMR (400 MHz, CDCl₃) δ 8.52 (s, 1H), 8.22 (s, 1H), 7.77 (dd, J=8.1, 1.3 Hz, 1H), 7.64 (dd, J=8.2, 1.2 Hz, 1H), 7.53 (dd, J=7.4, 1.3 Hz, 1H), 7.46 (t, J=7.8 Hz, 1H), 7.35 (t, J=7.8 Hz, 1H), 7.29-7.24 (m, 2H), 4.81-4.60 (m, 3H), 4.38 (d, J=17.3 Hz, 1H), 4.25 (ddd, J=26.0, 9.4, 4.5 Hz, 2H), 3.97 (dt, J=17.5, 1.7 Hz, 1H), 3.62-3.55 (m, 1H), 3.31-3.10 (m, 2H), 2.70 (d, J=14.7 Hz, 1H), 2.34 (s, 3H); ¹⁹F NMR (376 MHz, CDCl₃) δ−130.82 (ddd, J=25.1, 8.8, 4.7 Hz, 1F), −153.97 (td, J=20.7, 4.6 Hz, 1F), −160.83 (ddt, J=19.4, 9.4, 4.9 Hz, 1F), −169.07 (ddd, J=25.9, 21.3, 5.3 Hz, 1F).

### II. Biological Evaluation

### Examples B1: In Vitro Cell Viability Studies

Anti-cancer efficacy of exemplary compounds of this application is assessed in vitro in different cancer cell lines. Cell viability is examined following treatment at various concentration of inhibitor (0.097656-50 μM) using a cell Titer-Blue cell viability assay. 1×104 cells (NHF cells)/well are plated in 96-well assay plates in culture medium. All cells are grown in DMEM, IMDM and RPMI-1640 supplemented with 10% FBS. After 24 hrs, test compounds and vehicle controls are added to appropriate wells so the final volume is 100 μl in each well. The cells are cultured for the desired test exposure period (72 hrs) at 37° C. and 5% CO₂. The assay plates are removed from 37° C. incubator and 20 μl/well of CellTiter-Blue® Reagent is added. The plates are incubated using standard cell culture conditions for 1-4 hours and the plates are shaken for 10 seconds and record fluorescence at 560/590 nm.

### Examples B2 Reactivity Profiling with Glutathione

The experiment is started by placing 1 μL of 1 mM stocking solution of the test compound in DMSO in 199 μL of PBS buffer at pH 7.4 with 5 mM GSH to reach a final concentration of 5 μM. The final DMSO concentration is 0.5%. The solution is then incubated at 25° C. at 600 rpm, and is quenched with 600 μL solution of acetonitrile at 0, 30, 60 and 120 minutes. The quenched solution is vortexed for 10 minutes and centrifuged for 40 minutes at 3,220 g. An aliquot of 100 μL of the supernatant is diluted by 100 μL ultra-pure water, and the mixture is used for LC/MS/MS analysis. The data is processed and analyzed using Microsoft Excel.

### Examples B3 Parallel Artificial Membrane Permeability Assay (PAMPA)

The stock solutions of positive controls are prepared in DMSO at the concentration of 10 μM. Testosterone and methotrexate are used as control compounds in this assay. Prepare a stock solution of compounds in DMSO at the concentration of 10 mM, and further dilute with PBS (pH 7.4). The final concentration of the test compound is 10 μM.

Assay Procedures. 1) Prepare a 1.8% solution (w/v) of lecithin in dodecane, and sonicate the mixture to ensure a complete dissolution. 2) Carefully pipette 5 μL of the lecithin/dodecane mixture into each acceptor plate well (top compartment), avoiding pipette tip contact with the membrane. 3) Immediately after the application of the artificial membrane (within 10 minutes), add 300 μL of PBS (pH 7.4) solution to each well of the acceptor plate. Add 300 μL of drug-containing solutions to each well of the donor plate (bottom compartment) in triplicate. 4) Slowly and carefully place the acceptor plate into the donor plate, making sure the underside of the membrane is in contact with the drug-containing solutions in all wells. 5) Replace the plate lid and incubate at 25° C., 60 rpm for 16 hours. 6) After incubation, aliquots of 50 μL from each well of acceptor and donor plate are transferred into a 96-well plate. Add 200 μL of methanol (containing IS: 100 nM Alprazolam, 200 nM Labetalol and 2 μM Ketoprofen) into each well. 7) Cover with plate lid. Vortex at 750 rpm for 100 seconds. Samples are centrifuged at 3,220 g for 20 minutes. Determine the compound concentrations by LC/MS/MS.

### Examples B4: Coupled Nucleotide Exchange Assay

Method I: The inhibition of the SOS1-catalyzed nucleotide exchange activity of KRAS (e.g., KRAS G12C/C118A) was measured using Alpha (Amplified Luminescent Proximity Homogeneous Assay) technology. 20 nM GDP-bound human KRAS (G12C/C118A) protein was incubated with two-fold serially-diluted test compound or DMSO for 5 minutes at ambient temperature in reaction buffer (25 mM HEPES, pH 7.4; 10 mM MgCl2; 0.01% Triton X-100). For all subsequent steps, DTT was added to the reaction buffer at a final concentration of 1 mM. Next, GTP and SOS-1 were added in DTT-reaction buffer at final concentrations of 1.25 mM or 500 nM, respectively, and incubated at RT for 30 minutes. Finally, c-RAF RBD (50 nM final), Alpha glutathione donor beads (PerkinElmer; 20 μg/mL final), and AlphaLISAnickel chelate acceptor beads (PerkinElmer; 20 μg/mL final), all diluted in DTT-reaction buffer, were added. The reaction mixture was incubated at RT for 5 min, and then the plates were read on an EnVision® Multilabel Reader using the AlphaScreen protocol. Luminescence signal was measured at 570 nm following a 180 ms excitation at 680 nm. Signal intensity corresponded with the association of c-RAF RBD with GTP-bound KRAS G12C/C118A and was normalized to DMSO control.

### Intact Mass Analysis

The covalent modification of the proteins with the compounds were evaluated using intact mass analysis by liquid chromatography-mass spectrometry instrument (LC-MS/MS).

The reaction solution (20 μL) was prepared in 96-well plate and contained the protein (2 μM), the compound (100 μM), HEPES buffer (20 mM, pH 8), 2% DMSO, 2% glycerol, and 150 mM NaCl. The reaction was allowed to proceed for 24 h at 25° C. The reaction solution (1 μL) was injected into the LC/MS/MS without any further sample preparation.

The LC-MS/MS instrument comprises of a Waters G2-XS quadrupole-time of flight (QTof) mass spectrometer and a Waters Acuity I-class Ultra-High Performance Liquid Chromatography (UPLC) system. The I-class UPLC system includes a binary solvent manager (BSM), and an Acquity sample manager (SM). The mobile phase consisted of: A) 0.1% (v/v) formic acid in MilliQ water; B) 0.1% (v/v) formic acid acetonitrile. Gradients were run over 5 min and proceeded as follows: A:B, 85:15, 0.0-0.7 min, 85:15→15:85, 0.7-1.5 min, 10:90, 1.5-4 min, 10:90→85:15, 4-4.5 min, 85:15, 4.5-5 min. The analytical column was a Waters BEH C4 column 1.7 μm (50×1 mm) column with pore sizes of 300 Å. The TOF MS data was collected in positive ion mode (m/z of 400-2000 Da) using MassLynx software (Waters).

The spectral deconvolution was performed using UNIFI software (Waters). The added mass of the protein upon covalent modification to cysteine residues were specified. Multiple modification of up to 8 cysteine was allowed. All the adducts with signal intensities of <2% of the base peak were ignored. The % modification was calculated as the adduct signal intensity over the total intensities of the protein peaks and the adducts.

### Peptide Mapping

The site(s) of compounds covalent modification on proteins were identified using a peptide mapping analysis by liquid chromatography-mass spectrometry instrument (LC-MS/MS).

The reaction solution (100 μL) was prepared in a 1.5-mL Eppendorf tube and contained protein (2-10 μM), the compound (10-100 μM), HEPES buffer (20 mM, pH 8), 2% DMSO, 2% glycerol, and 150 mM NaCl. The reaction was allowed to proceed for 5-24 h at 25° C. or 37° C. Thereafter, the reaction was quenched by the addition of 500 μL of cold acetone and incubated at −20° C. for 2 h. Then, the tube was centrifuged for 10 min at 10,000×g, and the supernatant was discarded. The pellet was washed by adding 200 μL of cold acetone and centrifugation at 10,000×g for 10 min. The pellet was re-dissolved in 50 μL of ammonium bicarbonate solution (ABC, 100 mM, pH 7.9) containing 8 M urea. The tube was centrifuged for 10 min at 10,000×g, and the supernatant was transferred to a new tube. The protein was first reduced by adding 1.25 μL of 200 mM DTT and incubation at 37° C. for 30 min, then alkylated by adding 1.5 μL of 400 mM iodoacetamide incubation at room temperature for another 20 min. Then the solution was diluted 8 times in ammonium bicarbonate. Sequencing-grade trypsin (Promega) was added at an enzyme-to-protein ratio of 1:50, and the tube was incubated overnight at 37° C. After digestion, the solution was acidified by trifluoracetic acid at 0.1%, and tubes were centrifuged at 10,000×g for 10 min. The supernatant was transferred to an autosampler vial, and 2 μL was injected into the LC-MS/MS for peptide mapping analysis.

The LC-MS/MS instrument comprises of a Waters G2-XS quadrupole-time of flight (QTof) mass spectrometer and a Waters Acuity M-class Ultra-High Performance Liquid Chromatography (UPLC) system. The M-class UPLC system includes a micro binary solvent manager (μBSM), a micro sample manager (μSM), and an IonKey (iKey) separation system. The mobile phase consisted of: A) 0.1% (v/v) formic acid in MilliQ water; B) 0.1% (v/v) formic acid acetonitrile. Gradients were run over 20 min and proceeded as follows: A:B, 97:3, 0.0-1 min, 97:3→60:40, 1-12 min, 60:40→15:85, 12-12.5 min, 15:85, 12.5-17 min, 15:85→97:3, 17.5-20 min. The analytical column was a Waters BEH C18 iKey 1.7 μm (50×0.15 mm) column with pore sizes of 150 Å. The TOF MSE data was collected in positive ion mode (m/z of 350-2000 Da) using MassLynx software (Waters).

The peptide mapping analysis was performed using UNIFI software (Waters). Carbamidomethyl (+57 Da) and the compound mass addition upon covalent modification were specified as variable modification to cysteine residues.

### Sample Preparation for Differential Scanning Fluorimetry

KRas proteins at 0.05 mg/ml in buffer Hepes, 147 mM NaCl, 2% glycerol, 5 mM MgCl₂, 1 mM EDTA, 10 μM GDP, pH 8.0 were incubated in the presence desired concentration of compounds (5% DMSO final concentration) at 37° C. for 5 hours. After incubation, Sypro Oange to a final concentration of 5× was added to each sample. 20 μl aliquots of samples were transferred PCR tubes, sealed with caps, and run in a BioRad CFX96 RTPCR thermocycler. In the instrument, the samples were heated at a rate of 1° C./min from 25 to 90 degrees. Fluorescence readings were taken every 1 degree. The data generated was exported and analyzed using GraphPad-Prism software. The denaturation curves were fit using a Boltzmann sigmoidal equation to calculate melting temperatures (Tm).

### Sample Preparation for Isothermal Denaturation

Protein samples were prepared by diluting stock protein to 0.2 mg/ml in buffer (20 mM Hepes, 147 mM HaCl, 2% glycerol, 5 mM MgCl₂, 1 mM EDTA, 1 mM TCEP, pH 8.0). Compounds and controls were added to the desired concentrations keeping DMSO constant at 5%. Sypro Orange to a final concentration of 5× was added to all samples and 50 ul transferred to a 384 well plate (black, flat bottom). A citation instrument was used to monitor the kinetic of protein unfolding by setting the temperature at 44° C. and collecting datapoints every 2 minutes for 18 hours using excitation wavelength of 470 nm and emission 580 nm. The data generated was then plotted and analyzed using GraphPad Prism and fit to a single exponential function to calculate rates of unfolding and half lives.

### Cell Viability Assay MIA PaCa-2

MIA PaCa-2 cells were cultured in Dulbecco's Modified Eagle's Medium (Wisent) supplemented with 10% FBS, 2.5% horse serum, and 1% penicillin/streptomycin. Cells were seeded in 96-well plates at a density of 2,500 cells/well and incubated at 37° C., 5% CO₂ for 16 hours. Serially-diluted compounds or DMSO alone were added to cells and incubated at 37° C., 5% CO2 for 72 hours. Cell viability was measured using the CellTiter-Glo® Luminescent Cell Viability Assay (Promega) according to the manufacturer's protocol. The luminescence signal of each treated well was normalized to the DMSO control well and the media-only background was subtracted. Cell viability curves and IC50 values were visualized using Prism (GraphPad).

### ERK1/2 Phosphorylation and Total KRAS Assays

### MIA PaCa-2 Cell Lysis and Immunoassay to Detect KRas and Phospho-ERK1/2

MIA PaCa-2 cells were cultured in Dulbecco's Modified Eagle's Medium (Wisent) supplemented with 10% FBS, 2.5% horse serum, and 1% penicillin/streptomycin. Cells were seeded in 6-well plates at 500,000 cells/well and incubated at 37° C., 5% CO₂ for 16 hours. Cells were treated with the indicated concentrations of compound or DMSO alone and incubated at 37° C., 5% CO2 for 6 or 24 hours. Conditioned media was discarded, and adherent cells were washed with PBS before they were scraped. Cell suspensions were centrifugated at 2,000 RPM for 5 minutes at 4° C. and supernatants were discarded. Cell pellets were resuspended with 1×RIPA buffer (Millipore) and incubated on ice for 15 minutes. Protein lysates were extracted by centrifugating at 20,000 RPM for 20 minutes at 4° C. Proteins were separated and total KRas protein levels were quantified by Simple Western Immunoassay (ProteinSimple) using the 2-40 kDa Separation Module for Jess according to the manufacturer's protocol, using a protein concentration of 0.5 μg/μl and antibodies targeting KRas (clone #4F3, Sigma) and GAPDH (clone #14C10, Cell Signaling) diluted to 1:10 and 1:1600, respectively, where the latter was used as a loading control. A 3:1 ratio of rabbit to mouse HRP-conjugated secondary antibodies was used for detection. KRas protein levels were quantified relative to GAPDH loading levels and subsequently normalized to the DMSO control. Phosphorylated ERK1/2 levels were quantified by Simple Western Immunoassay (ProteinSimple) using the Protein Normalization Assay Module for Jess according to the manufacturer's protocol using a protein concentration of 0.5 μg/μl and an antibody targeting phosphor-ERK1/2 (clone #D13.14.4E, Cell Signalling) diluted to 1:10. Phosphorylated ERK1/2 levels were normalized to the Jess Protein Normalization Reagent loading control and subsequently to the DMSO control.

MIA PaCa-2 cells were cultured in Dulbecco's Modified Eagle's Medium (Wisent) supplemented with 10% FBS, 2.5% horse serum, and 1% penicillin/streptomycin. Cells were seeded in 96-well plates at a density of 25,000 cells/well and incubated at 37° C., 5% CO₂ for 24 hours. The next day, cells were starved in cell culture media containing 1% FBS only at 37° C., 5% CO₂ for 16 hours. Following starvation, serially-diluted compounds or DMSO alone were added to cells and incubated at 37° C., 5% CO₂ for 1 or 3 hours. Prior to cell lysis, 25 ng/ml human epidermal growth factor (hEGF) (Sigma) was added to the cells and incubated at 37° C., 5% CO₂ for 10 minutes. Conditioned media was discarded, and adherent cells lysed and basal ERK1/2 phosphorylation levels were measured using the Phospho-ERK (Thr202/Tyr204) Cellular HTRF kit (Perkin Elmer) according to the manufacturer's protocol. The fluorescent signal of the acceptor antibody at a wavelength of 665 nm was normalized to that of the donor antibody at 620 nm. HTRF ratios were plotted and relative IC₅₀ values were obtained using Prism (GraphPad).

### KRas^{G12C} Coupled Nucleotide Exchange Assay

Method II: Inhibition of the SOS1-catalyzed nucleotide exchange activity of KRAS^{G12C} was measured using Alpha technology (PerkinElmer). 50 nM GDP-bound human KRAS^{G12C} protein was incubated with serially-diluted compound or DMSO for 2 hrs at RT in RBD-RAS binding buffer (BPSBioscience). 1 mM GTP and 500 nM SOS-1 were added to the reaction mixture and incubated at RT for 30 min. GST-tagged RBD-cRAF was then added to the reaction mixture at a final concentration of 2.5 nM and incubated at RT for 30 min. Finally, Alpha Glutathione acceptor beads and Nickel chelate donor beads (PerkinElmer) were diluted to 1:500 and 1:250, respectively, and added to the reaction mixture. The final reaction was incubated at RT for 30 min. Plates were read on a TECAN SPARK® Multiplate Reader. The luminescence signal was measured at 615 nm following excitation at 680 nm. Signal intensity was normalized to DMSO control.

In some instances, Table 9 represents the activity of a compound provided herein.

In some instances, Table 9 represents the extent to which a compound binds to KRAS G12C (SEQ ID NO:1 or SEQ ID NO:2) or KRAS G12C Lite (SEQ ID NO:3). In some instances, in vitro binding to KRAS G12C (SEQ ID NO:1 or SEQ ID NO:2) and KRAS G12C LITE (SEQ ID NO:3) shows the extent to which a compound binds to KRAS G12C (SEQ ID NO:1 or SEQ ID NO:2) or KRAS G12C Lite (SEQ ID NO:3). In some embodiments, Table 9 shows the in vitro KRAS intact mass spectrometry studies.

In some instances, Table 9 represents the in vitro target engagement with KRAS G12C showing functional inhibition of KRAS GDP-GTP cycling in the presence of SOS1. In some instances, Table 9 shows the extent to which a compound binds and modulates KRAS G12C. In some instances, Table 9 shows inhibition of SOS1 mediated KRAS nucleotide exchange assay.

In some instances, Table 9 represents the in cellulo target engagement with KRAS G12C showing functional inhibition of KRAS G12C function in MIA PACA-2 cells which express KRAS G12C. In some instances, Table 9 shows the extent to which a compound binds and modulates KRAS G12C in human cells. In some instances, Table 9 shows in cellulo inhibition of KRAS G12C functional activity.

TABLE 9

| | | KRAS | KRAS-SOS1 | pERK - MIA |
|---|---|---|---|---|
| Compound | KRAS Lite | G12C | Nucleotide | PaCa-2 - (6 |
| Number | Modification | Modification | Exchange | hours, 5 uM) |
| 11A | a | b | n.d. | n.d. |
| 52A | a | b | n.d. | n.d. |
| 14A | a | b | A | X |
| 13A | a | b | A | n.d. |
| 2A | a | b | A | n.d. |
| 15A | a | b | A | X |
| 12A | a | b | A | n.d. |
| 6A | a | b | A | X |
| 42A | a | b | A | n.d. |
| 1A | a | b | A | X |
| 3A | a | b | A | X |
| 19A | a | b | n.d. | X |
| 7A | a | b | A | X |
| 8A | a | n.d. | n.d. | n.d. |
| 43A | a | n.d. | n.d. | n.d. |
| 16A | a | b | A | n.d. |
| 49A | a | b | n.d. | n.d. |
| 4A | a | n.d. | A | X |
| 17A | a | b | A | X |
| 18A | a | b | A | X |
| 9A | a | n.d. | n.d. | X |
| 31A | a | n.d. | A | n.d. |
| 37A | n.d. | n.d. | n.d. | n.d. |
| 39A | n.d. | n.d. | n.d. | n.d. |
| 50A | n.d. | b | n.d. | n.d. |
| 5A | n.d. | b | A | X |
| 51A | n.d. | b | n.d. | n.d. |
| 30A | n.d. | n.d. | A | n.d. |
| 32A | n.d. | n.d. | n.d. | X |
| 33A | n.d. | n.d. | n.d. | X |
| 34A | n.d. | n.d. | n.d. | X |
| 35A | n.d. | n.d. | A | X |
| 20A | n.d. | n.d. | A | X |
| 36A | n.d. | n.d. | n.d. | X |
| 38A | n.d. | n.d. | A | n.d. |
| 40A | n.d. | n.d. | n.d. | X |
| 41A | n.d. | n.d. | A | X |
| 21A | n.d. | n.d. | A | n.d. |
| 45A | n.d. | n.d. | A | n.d. |
| 24A | n.d. | n.d. | A | n.d. |
| 22A | n.d. | n.d. | A | n.d. |
| 48A | n.d. | n.d. | A | X |
| 10A | n.d. | n.d. | A | X |
| 44A | n.d. | n.d. | A | X |
| 23A | n.d. | n.d. | A | n.d. |
| 27A | n.d. | n.d. | A | n.d. |
| 46A | n.d. | n.d. | A | X |
| 28A | n.d. | n.d. | A | n.d. |
| 47A | n.d. | n.d. | A | n.d. |
| 29A | n.d. | n.d. | A | n.d. |
| 25A | n.d. | n.d. | A | n.d. |
| 26A | n.d. | n.d. | A | n.d. |

Shows % modification of KRAS G12C Lite, KRAS G12C FL at the denoted concentration, temperature, and incubation time.
n.d.: not determined
a: KRAS Lite modified
b: KRAS G12C modified
A: KRAS G12C bound and modulated
X: KRAS G12C bound and modulated in cells

In some instances, Table 10 represents the in vitro target engagement with KRAS G12C and G12C Lite. In some instances, such as in the presence of a fluorescent dye and upon heating, KRAS G12C protein is destabilised and denatured and a melting temperature of the protein determined, such as shown in Table 10. In some instances, a compound's effects on the destabilisation—either acceleration or deceleration—correspond to the effect of interaction with the protein.

TABLE 10

| Compound | KRAS G12C Lite DSF | |
|---|---|---|
| Number | ΔTm | KRAS G12C DSF ΔTm |
| 14A | Y | Y |
| 13A | Y | Y |
| 11A | Y | Y |
| 52A | Y | Y |
| 15A | Y | Y |
| 42A | Y | n.d. |
| 12A | Y | Y |
| Control 1 | N | N |
| Control 2 | N | n.d. |

Control 1: 1-[4-[6-chloro-8-fluoro-7-(2-fluoro-6-hydroxyphenyl)quinazolin-4-yl]piperazin-1-yl]prop-2-en-1-one
Control 2: 6-fluoro-7-(2-fluoro-6-hydroxyphenyl)-1-(4-methyl-2-propan-2-ylpyridin-3-yl)-4-[(2S)-2-methyl-4-prop-2-enoylpiperazin-1-yl]pyrido[2,3-d]pyrimidin-2-one
n.d.: not determined
Y: protein destabilized
N: protein not destabilized

### III. Preparation of Pharmaceutical Dosage Forms

### Example P1: Solution for Injection

The active ingredient is a compound of Table 1, Table 8, or a pharmaceutically acceptable salt or tautomer or regioisomer thereof. A solution for intraperitoneal administration is prepared by mixing 1-1000 mg of active ingredient with 10-50 mL of a solvent mix made up by 25% dimethylacetamide, 50% propylene glycol and 25% Tween 80. Filter through millipore sterilizing filter and then distribute in 1 mL amber glass ampoules, performing all the operations under sterile conditions and under nitrogen atmosphere. 1 mL of such solution is mixed with 100 or 200 mL of sterile 5% glucose solution before using intraperitoneally.

The examples and embodiments described herein are for illustrative purposes only and various modifications or changes suggested to persons skilled in the art are to be included within the spirit and purview of this application and scope of the appended claims.

1-50. (canceled)

51. A compound having a structure represented by Formula (I-A):
D1-L-D2
Formula (I-A) wherein:
D1 is a radical of a KRAS-binding ligand;
D2 is a warhead radical; and
L is a linker, or a pharmaceutically acceptable salt or solvate thereof.

52. The compound of claim 51, wherein D2 is a selective warhead radical.

53. The compound according of claim 51, wherein D2 is selective for KRAS.

54. The compound of claim 51, wherein D2 covalently modifies KRAS and/or mutant KRAS G12C Lit.

55. The compound of claim 51, wherein D2 does not covalently modify KRAS WT protein.

56. The compound of claim 51, wherein D2 binds to, disrupts, and/or modifies KRAS G12C (SEQ ID NO:1 or SEQ ID NO:2) and/or mutant KRAS G12C Lite (SEQ ID NO:3).

57. The compound of claim 51, wherein D2 comprises one or more warhead group, each warhead group being independently selected from the group consisting of substituted or unsubstituted sulfonamide, sulfone, sulfoxide, substituted or unsubstituted amino, or substituted aryl.

58. The compound of claim 51, wherein D2 comprises an aryl substituted with one or more substituent, each substituent being independently selected from sulfone, sulfoxide, halogen, hydroxy, substituted or unsubstituted alkoxy, and substituted or unsubstituted alkyl.

59. The compound of claim 51, wherein D2 comprises a sulfone, a sulfoxide, or a sulfonamide.

60. The compound of claim 51, wherein D2 comprises a sulfone, a sulfoxide, or a sulfonamide; and an aryl substituted with one or more substituent, each substituent being independently selected from halogen, hydroxy, substituted or unsubstituted alkoxy, and substituted or unsubstituted alkyl.

61. (canceled)

62. (canceled)

63. The compound of claim 51, wherein D2 is or comprises an aryl substituted with halogen.

64. The compound of claim 51, wherein D2 is or comprises an aryl substituted with halogen and alkyl substituted with halogen; hydroxy, unsubstituted alkoxy, or alkoxy substituted with halogen.

65. (canceled)

66. (canceled)

67. (canceled)

68. The compound of claim 51, wherein D2 is or comprises an aryl substituted with halogen; and sulfone or sulfoxide.

69. (canceled)

70. (canceled)

71. (canceled)

72. (canceled)

73. The compound of claim 51, or a pharmaceutically acceptable salt or solvate thereof, wherein the linker is a non-releasable linker.

74. The compound of claim 51, or a pharmaceutically acceptable salt or solvate thereof, wherein the linker comprises one or more linker group, each linker group being independently selected from the group consisting of —O—, amino, substituted or unsubstituted alkylene, substituted or unsubstituted heteroalkylene, and substituted or unsubstituted alkoxy.

75. (canceled)

76. (canceled)

77. (canceled)

78. (canceled)

79. (canceled)

80. (canceled)

81. The compound of claim 51, or a pharmaceutically acceptable salt or solvate thereof, wherein D1 has a structure represented by:

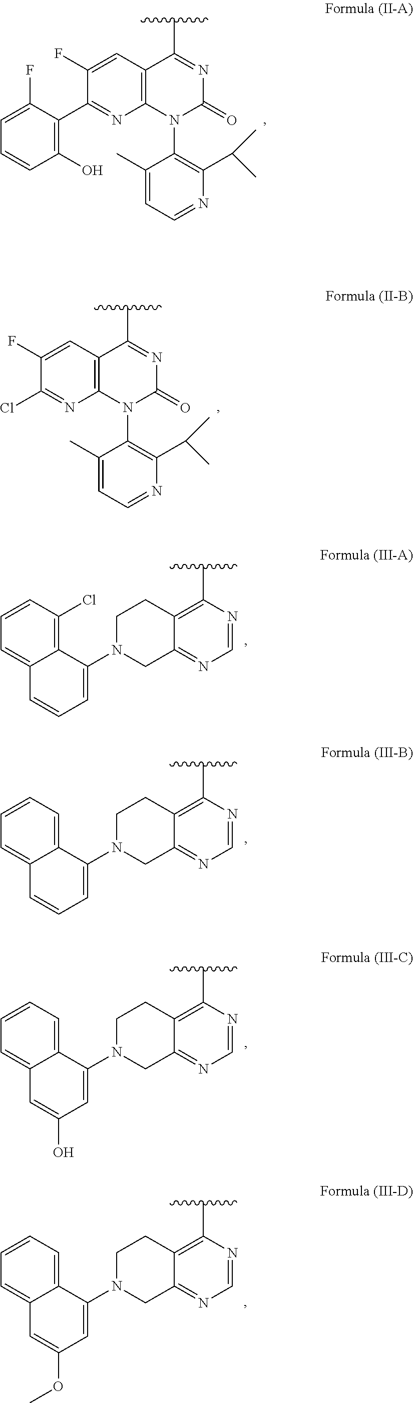 [SMILES: *c1nc(=O)n(-c2c(C)ccnc2C(C)C)c2nc(-c3c(O)cccc3F)c(F)cc12 ; *c1nc(=O)n(-c2c(C)ccnc2C(C)C)c2nc(Cl)c(F)cc12 ; *c1ncnc2c1CCN(c1cccc3cccc(Cl)c13)C2 ; *c1ncnc2c1CCN(c1cccc3ccccc13)C2 ; *c1ncnc2c1CCN(c1cc(O)cc3ccccc13)C2 ; *c1ncnc2c1CCN(c1cc(OC)cc3ccccc13)C2]

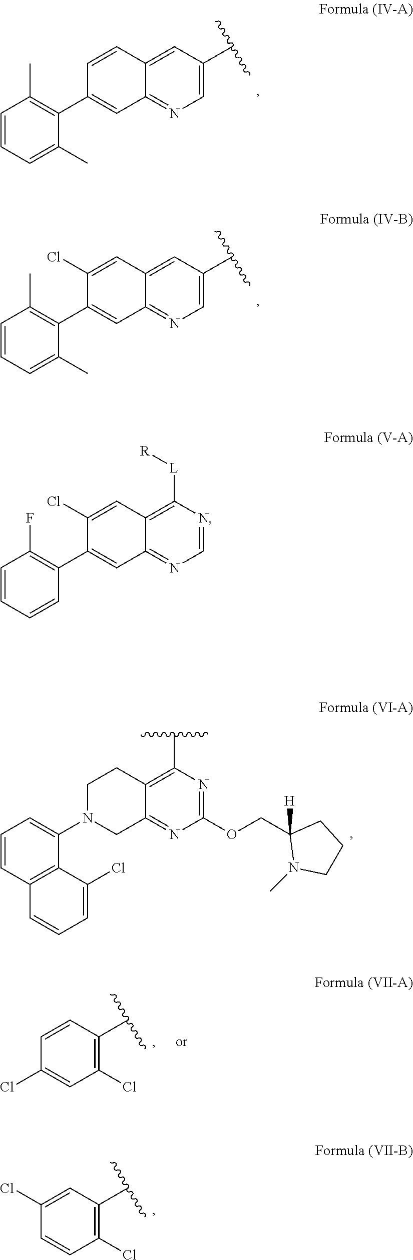

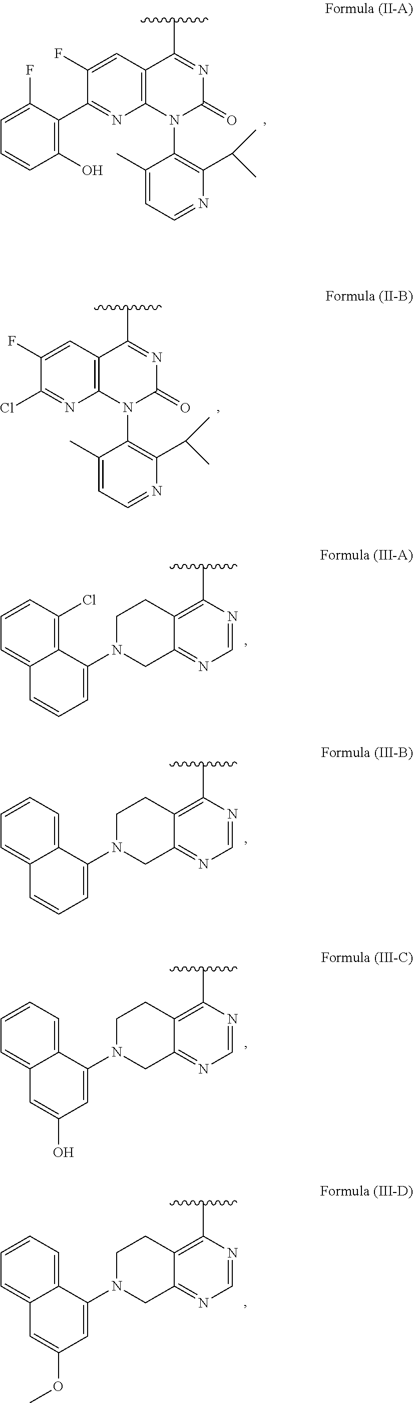 [SMILES: *c1nc(=O)n(-c2c(C)ccnc2C(C)C)c2nc(-c3c(O)cccc3F)c(F)cc12 ; *c1nc(=O)n(-c2c(C)ccnc2C(C)C)c2nc(Cl)c(F)cc12 ; *c1ncnc2c1CCN(c1cccc3cccc(Cl)c13)C2 ; *c1ncnc2c1CCN(c1cccc3ccccc13)C2 ; *c1ncnc2c1CCN(c1cc(O)cc3ccccc13)C2 ; *c1ncnc2c1CCN(c1cc(OC)cc3ccccc13)C2]

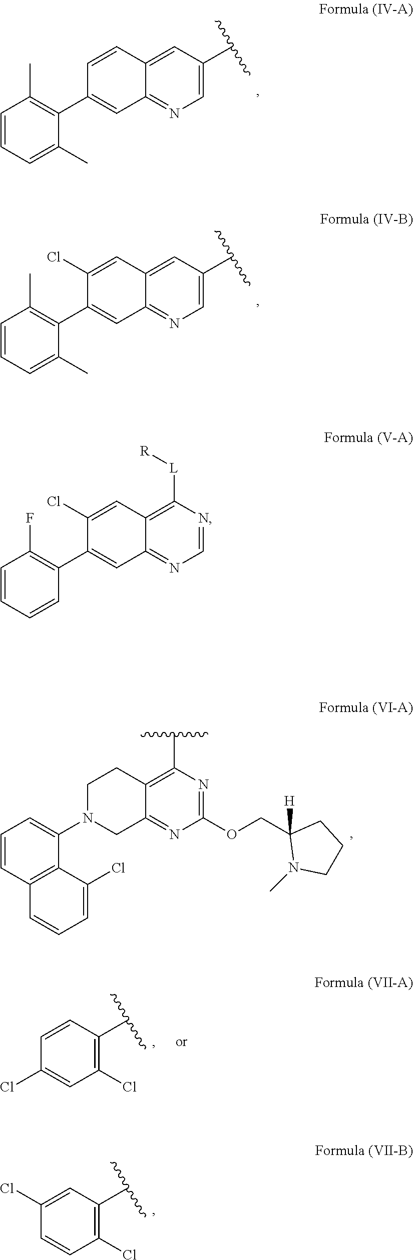

82. A compound selected from Table 8.

83. A pharmaceutically acceptable composition comprising a compound of claim 51, or a salt or solvate thereof, and one or more of pharmaceutically acceptable excipients.

84. A KRAS protein or an active fragment thereof modified with a compound of claim 51, or a salt or solvate thereof, wherein the compound forms a covalent bond with a sulfur atom of a cysteine residue of the KRAS protein or an active fragment thereof.

85. A method of modifying, binding, or disrupting KRAS protein or an active fragment thereof with a compound, comprising contacting the polypeptide with a compound of claim 51, or a salt or solvate thereof, to form a covalent bond with a sulfur atom of a cysteine residue of the KRAS protein or an active fragment thereof.

86. (canceled)

87. (canceled)


SEQUENCE LISTING (3 sequences)

SEQ ID NO: 1 - protein, 187 aa, Unknown
  Description of Unknown KRAS G12C In-House sequence
MHHHHHHSSGRENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 2 - protein, 186 aa, Unknown
  Description of Unknown KRAS G12C Commercial sequence
HHHHHHSSGRENLYFQGMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 3 - protein, 187 aa, Unknown
  Description of Unknown KRAS Lite sequence
MHHHHHHSSGRENLYFQGMTEYKLVVVGACGVGKSALTIQLIQNHFVDEYDPTIEDCYRKQVVIDGETSLLDILDTAGQEEYSAMRDQYMRTGEGFLSVFAINNTKSFEDIHHYREQIKRVKDSEDVPMVLVGNKSDLPSRTVDTKQAQDLARPYGIPFIETSAKTRQGVDDAFYTLVREIRKHKEK